SEQUENCE LISTING (167 sequences)

SEQ ID NO: 1 - protein, 914 aa, Homo sapiens
  REGION 1..914: note = misc_feature - Human cMET-ECD-his
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTHHHHHH

SEQ ID NO: 2 - protein, 1137 aa, Homo sapiens
  REGION 1..1137: note = misc_feature - Human cMET-ECD-mFc
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGGGGSCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 914 aa, Macaca fascicularis
  REGION 1..914: note = misc_feature - cyno cMET-ECD-his
ECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTHHHHHH

SEQ ID NO: 4 - protein, 121 aa, synthetic construct
  REGION 1..121: note = Synthetic: VH (AA)
QVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRNMGYSGWLDYWGQGTLVTVSS

SEQ ID NO: 5 - DNA, 363 bp, synthetic construct
  misc_feature 1..363: note = Synthetic: VH (DNA)
  source 1..363: mol_type = other DNA
caggtgcagctggtggagtctgggggaggcgtggtccagcctgggaggtccctgagactctcctgtgcagcctctggattcaccttcagtagccatggcatgcactgggtccgccaggctccaggcaaggggctggagtgggtggcagttatatcatatgatgaaagtgataaatattatgcagactccgtgaagggccgattcaccatctccagagaccattccaagaacacgctgtatctgcaaatgaacagcctgagagctgaggacacggctgtatattactgtgtgaaagatcggaatatgggatatagtggctggcttgactactggggccagggaaccctggtcaccgtctcctca

SEQ ID NO: 6 - protein, 5 aa, synthetic construct
  REGION 1..5: note = Synthetic: HCDR1 (Kabat)
SHGMH

SEQ ID NO: 7 - protein, 17 aa, synthetic construct
  REGION 1..17: note = Synthetic: HCDR2 (Kabat)
VISYDESDKYYADSVKG

SEQ ID NO: 8 - protein, 12 aa, synthetic construct
  REGION 1..12: note = Synthetic: HCDR3 (Kabat)
DRNMGYSGWLDY

SEQ ID NO: 9 - protein, 121 aa, synthetic construct
  REGION 1..121: note = Synthetic: VH (AA)
QVQLVESGGGVVQPGKSLRLSCAASGFTFSSSGLHWVRQAPGKGLEWVAVISYDTSNKYYADSVKGRFTISRDNSKNTLYLQMNSLRAEDTAVYYCAKDLNRGYDWGFDYWGQGTLVTVSS

SEQ ID NO: 10 - DNA, 363 bp, synthetic construct
  misc_feature 1..363: note = Synthetic: VH (DNA)
  source 1..363: mol_type = other DNA
caggtgcagctggtggagtctgggggaggcgtggtccagcctgggaagtccctgagactctcctgtgcagcctctggattcaccttcagtagttctggcttgcactgggtccgccaggctccaggcaagggactggagtgggtggcagttatatcatatgatacaagtaataaatactatgcagactccgtgaagggccgattcaccatctccagagacaattccaagaacacgctgtatctgcaaatgaacagcctgagagctgaggacacggctgtatattactgtgcgaaagatctaaatcgtggctatgattgggggtttgactactggggccagggaaccctggtcaccgtctcctca

SEQ ID NO: 11 - protein, 5 aa, synthetic construct
  REGION 1..5: note = Synthetic: HCDR1 (Kabat)
SSGLH

SEQ ID NO: 12 - protein, 17 aa, synthetic construct
  REGION 1..17: note = Synthetic: HCDR2 (Kabat)
VISYDTSNKYYADSVKG

SEQ ID NO: 13 - protein, 12 aa, synthetic construct
  REGION 1..12: note = Synthetic: HCDR3 (Kabat)
DLNRGYDWGFDY

SEQ ID NO: 14 - protein, 120 aa, synthetic construct
  REGION 1..120: note = Synthetic: VH (AA)
QVQLVESGGGVVQPGRSLRLSCAASGFTFSNYGMHWVRQAPGKGLEWVAVISYDGVDKYYADSVKGRFTISRDNSKNTLYLQMNSLRAEDTAMYYCAKDRISYNWNLDYWGQGTLVTVSS

SEQ ID NO: 15 - DNA, 360 bp, synthetic construct
  misc_feature 1..360: note = Synthetic: VH (DNA)
  source 1..360: mol_type = other DNA
caggtgcagctggtggagtctgggggaggcgtggtccagcctgggaggtccctgagactctcctgtgcagcctctggattcaccttcagtaactatggcatgcactgggtccgccaggctccaggcaaggggctggagtgggtggcagttatatcatatgatggcgttgataagtactatgcagactccgtgaagggccgattcaccatctccagagacaattccaagaacacgctgtatctgcaaatgaacagcctgagagctgaggacacggctatgtattactgtgcgaaagatcggatttcctataactggaaccttgactactggggccagggaaccctggtcaccgtctcctca

SEQ ID NO: 16 - protein, 5 aa, synthetic construct
  REGION 1..5: note = Synthetic: HCDR1 (Kabat)
NYGMH

SEQ ID NO: 17 - protein, 17 aa, synthetic construct
  REGION 1..17: note = Synthetic: HCDR2 (Kabat)
VISYDGVDKYYADSVKG

SEQ ID NO: 18 - protein, 11 aa, synthetic construct
  REGION 1..11: note = Synthetic: HCDR3 (Kabat)
DRISYNWNLDY

SEQ ID NO: 19 - protein, 121 aa, synthetic construct
  REGION 1..121: note = Synthetic: VH (AA)
QVQLVESGGGVVQPGRSLRLSCVASGFTFSNYGLHWVRQAPGKGLEWVAAISYDGSDKYYADPVKGRFTISRDTTKNTLSLQMNSLRAEDTAVYYCAKERYRGYDWDFDYWGQGTLVTVSS

SEQ ID NO: 20 - DNA, 363 bp, synthetic construct
  misc_feature 1..363: note = Synthetic: VH (DNA)
  source 1..363: mol_type = other DNA
caggtgcagttggtggagtctgggggaggcgtggtccagcctgggaggtccctgagactctcctgtgtagcctctggattcaccttcagtaactatggcctacactgggtccgccaggctccaggcaaggggctggagtgggtggcagctatatcatatgatggaagtgataaatactatgcagaccccgtgaagggccgattcaccatctccagagacacgaccaagaacacgctgtctctgcaaatgaacagtctgagagctgaggacacggctgtgtattactgtgcgaaagaaagatatcgtggctacgattgggactttgactactggggccagggaaccctggtcaccgtctcctca

SEQ ID NO: 21 - protein, 5 aa, synthetic construct
  REGION 1..5: note = Synthetic: HCDR1 (Kabat)
NYGLH

SEQ ID NO: 22 - protein, 17 aa, synthetic construct
  REGION 1..17: note = Synthetic: HCDR2 (Kabat)
AISYDGSDKYYADPVKG

SEQ ID NO: 23 - protein, 12 aa, synthetic construct
  REGION 1..12: note = Synthetic: HCDR3 (Kabat)
ERYRGYDWDFDY

SEQ ID NO: 24 - protein, 121 aa, synthetic construct
  REGION 1..121: note = Synthetic: VH (AA)
QVQLVESGGGVVQPGRSLRLSCAASGFTFSSYGLHWVRQAPGKGLEWVAVISFDGSNKYYADSVKGRFTISRDTSKNTLSLQMNSLRAEDTAVYYCAKERSRGYDWDFDYWGQGILVTVSS

SEQ ID NO: 25 - DNA, 363 bp, synthetic construct
  misc_feature 1..363: note = Synthetic: VH (DNA)
  source 1..363: mol_type = other DNA
caggtgcagctggtggagtctgggggaggcgtggtccagcctgggaggtccctgagactctcctgtgcagcctctggattcaccttcagtagctatggcctacactgggtccgccaggctccaggcaaggggctggagtgggtggcagttatatcatttgatggaagtaataaatactatgcagactccgtgaagggccgattcaccatctccagagacacttccaagaacacgctgtctctgcaaatgaacagcctgagagctgaggacacggctgtgtattactgtgcgaaagaaagatctcgtggctacgattgggactttgactactggggccagggaatcctggtcaccgtctcctca

SEQ ID NO: 26 - protein, 5 aa, synthetic construct
  REGION 1..5: note = Synthetic: HCDR1 (Kabat)
SYGLH

SEQ ID NO: 27 - protein, 17 aa, synthetic construct
  REGION 1..17: note = Synthetic: HCDR2 (Kabat)
VISFDGSNKYYADSVKG

SEQ ID NO: 28 - protein, 12 aa, synthetic construct
  REGION 1..12: note = Synthetic: HCDR3 (Kabat)
ERSRGYDWDFDY

SEQ ID NO: 29 - protein, 121 aa, synthetic construct
  REGION 1..121: note = Synthetic: VH (AA)
QVRLVESGGGVVQPGRSLRLSCAASGFTFSNYGMHWVRQAPGKGLEWVAVISYDGSDKYYPDSVKGRFTISRDNSRNTLYLQMNSLSTEDTAVYYCAKDQYRGYDGTFDYWGQGTLVTVSS

SEQ ID NO: 30 - DNA, 363 bp, synthetic construct
  misc_feature 1..363: note = Synthetic: VH (DNA)
  source 1..363: mol_type = other DNA
caggtgcggctggtggagtcggggggaggcgtggtccagcctgggaggtccctgagactctcctgtgcagcctctggattcaccttcagtaactatggcatgcactgggtccgccaggctccaggcaaggggctggagtgggtggcagttatatcatatgatggaagtgataagtactatccagactccgtgaagggccgcttcaccatctccagagacaattccagaaacacgctgtatctgcaaatgaacagcctgagtactgaggacacggctgtttattactgtgcgaaagatcaatatcgtggctacgatgggacttttgactactggggccagggaaccctggtcaccgtctcctca

SEQ ID NO: 31 - protein, 5 aa, synthetic construct
  REGION 1..5: note = Synthetic: HCDR1 (Kabat)
NYGMH

SEQ ID NO: 32 - protein, 17 aa, synthetic construct
  REGION 1..17: note = Synthetic: HCDR2 (Kabat)
VISYDGSDKYYPDSVKG

SEQ ID NO: 33 - protein, 12 aa, synthetic construct
  REGION 1..12: note = Synthetic: HCDR3 (Kabat)
DQYRGYDGTFDY

SEQ ID NO: 34 - protein, 120 aa, synthetic construct
  REGION 1..120: note = Synthetic: VH (AA)
QVQLVESGGGVVQPGRSLRLSCAASGFIFSSYNMHWVRQAPGKGLEWVAVISYDRVNKYYADSVKGRFTISRDNSKNTLYLQMNSLRVEDTAVYYCAKERERYFDWLDYWGQGTLVTVSS

SEQ ID NO: 35 - DNA, 360 bp, synthetic construct
  misc_feature 1..360: note = Synthetic: VH (DNA)
  source 1..360: mol_type = other DNA
caggtgcagctggtggagtctgggggaggcgtggtccagcctgggaggtccctgagactctcctgtgcagcctctggattcatcttcagtagctataacatgcactgggtccgccaggctccaggcaaggggctggagtgggtggcagttatatcatatgatagagttaataaatactatgcagactccgtgaagggccgattcaccatctccagagacaattccaagaacacgctgtatctgcaaatgaacagcctgagagttgaggacacggctgtgtattactgtgcgaaagaaagggaacgatattttgactggttggactactggggccagggaaccctggtcaccgtctcctca

SEQ ID NO: 36 - protein, 5 aa, synthetic construct
  REGION 1..5: note = Synthetic: HCDR1 (Kabat)
SYNMH

SEQ ID NO: 37 - protein, 17 aa, synthetic construct
  REGION 1..17: note = Synthetic: HCDR2 (Kabat)
VISYDRVNKYYADSVKG

SEQ ID NO: 38 - protein, 11 aa, synthetic construct
  REGION 1..11: note = Synthetic: HCDR3 (Kabat)
ERERYFDWLDY

SEQ ID NO: 39 - protein, 121 aa, synthetic construct
  REGION 1..121: note = Synthetic: VH (AA)
QVQLVESGGGVVQPGRSLRLSCAASGFTFSSYGMHWVRQAPGKGLEWVAVISNDGSYKYYVDSVTGRFTISRDNSKNTLYLQMNSLRAEDTAVYYCAKDRNMGYSGWFDYWGQGSLVTVSS

SEQ ID NO: 40 - DNA, 363 bp, synthetic construct
  misc_feature 1..363: note = Synthetic: VH (DNA)
  source 1..363: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggaagttataaatactatgtagactccgtgacgggccgattcaccatctccagagacaattccaagaacacgctgtatctgcaaatgaacagcctgagagctgaggacacggctgtgtattactgtgcgaaagatcggaatatgggatatagtggctggtttgactactggggccagggatccctggtcaccgtctcctca

SEQ ID NO: 41 - protein, 5 aa, synthetic construct
  REGION 1..5: note = Synthetic: HCDR1 (Kabat)
SYGMH

SEQ ID NO: 42 - protein, 17 aa, synthetic construct
  REGION 1..17: note = Synthetic: HCDR2 (Kabat)
VISNDGSYKYYVDSVTG

SEQ ID NO: 43 - protein, 12 aa, synthetic construct
  REGION 1..12: note = Synthetic: HCDR3 (Kabat)
DRNMGYSGWFDY

SEQ ID NO: 44 - protein, 121 aa, synthetic construct
  REGION 1..121: note = Synthetic: VH (AA)
QVQLVESGGGVVQPGRSLRLSCAASGFTFSSHGMHWVRQAPGKGLEWVAVISNDGSYKYYADSVTGRFTISRDNSKNTLYLQMNSLRAEDTAVYYCAKDRNMGYSGWFDYWGQGSLVTVSS

SEQ ID NO: 45 - DNA, 363 bp, synthetic construct
  misc_feature 1..363: note = Synthetic: VH (DNA)
  source 1..363: mol_type = other DNA
caggtgcagctggtggagtctgggggaggcgtggtccagcctgggaggtccctgagactctcctgtgcagcctctggattcaccttcagtagccatggcatgcactgggtccgccaggctccaggcaaggggctggagtgggtggcagttatatcaaatgatggaagttataaatactatgcagactccgtgacgggccgattcaccatctccagagacaattccaagaacacgctgtatctgcaaatgaacagcctgagagctgaggacacggctgtgtattactgtgcgaaagatcggaatatgggatacagtggctggtttgactactggggccagggatccctggtcaccgtctcctca

SEQ ID NO: 46 - protein, 5 aa, synthetic construct
  REGION 1..5: note = Synthetic: HCDR1 (Kabat)
SHGMH

SEQ ID NO: 47 - protein, 17 aa, synthetic construct
  REGION 1..17: note = Synthetic: HCDR2 (Kabat)
VISNDGSYKYYADSVTG

SEQ ID NO: 48 - protein, 12 aa, synthetic construct
  REGION 1..12: note = Synthetic: HCDR3 (Kabat)
DRNMGYSGWFDY

SEQ ID NO: 49 - protein, 122 aa, synthetic construct
  REGION 1..122: note = Synthetic: VH (AA)
QVQLVESGGGVVQPGRSLRLSCAASGFTFSNYGMHWVRQAPGKGLEWVAVISNDGSEKYYADSVKGRFTISRDSSKNTLYLQMNSLRAEDTAVYYCAKAPWGSGTTGRLDYWGQGTLVTVSS

SEQ ID NO: 50 - DNA, 366 bp, synthetic construct
  misc_feature 1..366: note = Synthetic: VH (DNA)
  source 1..366: mol_type = other DNA
caggtgcagctggtggagtctgggggaggcgtggtccagcctgggaggtccctgagactctcctgtgcagcctctggattcaccttcagtaactatggcatgcactgggtccgccaggctccaggcaaggggctggagtgggtggctgttatatcaaatgatggaagtgaaaaatactatgcagactccgtgaagggccgattcaccatctccagagacagttccaagaacacgctctatctacagatgaacagcctgagagctgaggacacggctgtgtattactgtgcgaaagccccttggggaagtggaacgacggggaggcttgactactggggccagggaaccctggtcaccgtctcctca

SEQ ID NO: 51 - protein, 5 aa, synthetic construct
  REGION 1..5: note = Synthetic: HCDR1 (Kabat)
NYGMH

SEQ ID NO: 52 - protein, 17 aa, synthetic construct
  REGION 1..17: note = Synthetic: HCDR2 (Kabat)
VISNDGSEKYYADSVKG

SEQ ID NO: 53 - protein, 13 aa, synthetic construct
  REGION 1..13: note = Synthetic: HCDR3 (Kabat)
APWGSGTTGRLDY

SEQ ID NO: 54 - protein, 121 aa, synthetic construct
  REGION 1..121: note = Synthetic: VH (AA)
QFQLQESGPGLVKPSETLSLTCTVSGGSISTSSYYWGWIRQPPGKGLEWIGTIYYSGTTYYNPSLKSRVTISVDTSKNQFSLKLKSVTAADTSIYYCARHWPKYNSWFDPWGQGILVTVSS

SEQ ID NO: 55 - DNA, 363 bp, synthetic construct
  misc_feature 1..363: note = Synthetic: VH (DNA)
  source 1..363: mol_type = other DNA
caatttcagctacaggagtcgggcccaggactggtgaagccttcggagaccctgtccctcacctgcactgtctctggtggctccatcagcactagtagttactactggggctggatccgccagcccccagggaaggggctggagtggattgggactatctattatagtgggaccacctactacaacccgtccctcaagagtcgagtcaccatatccgtagacacgtccaagaaccagttctccctgaagctgaagtctgtgaccgccgcagacacgtctatctattactgtgcgagacattggcccaagtacaactcctggttcgacccctggggccagggaattctggtcaccgtctcctca

SEQ ID NO: 56 - protein, 7 aa, synthetic construct
  REGION 1..7: note = Synthetic: HCDR1 (Kabat)
TSSYYWG

SEQ ID NO: 57 - protein, 16 aa, synthetic construct
  REGION 1..16: note = Synthetic: HCDR2 (Kabat)
TIYYSGTTYYNPSLKS

SEQ ID NO: 58 - protein, 11 aa, synthetic construct
  REGION 1..11: note = Synthetic: HCDR3 (Kabat)
HWPKYNSWFDP

SEQ ID NO: 59 - protein, 121 aa, synthetic construct
  REGION 1..121: note = Synthetic: VH (AA)
QFQLQESGPGLVKPSETLSLTCTVSGGSISTSSYYWGWIRQPPGKGLEWIGTIYFSGTTYYNPSLTSRVSMSVDTSKNQFSLKLNSVTAADTSIYYCARHWPKYNSWFDPWGQGFLVTVSS

SEQ ID NO: 60 - DNA, 363 bp, synthetic construct
  misc_feature 1..363: note = Synthetic: VH (DNA)
  source 1..363: mol_type = other DNA
caatttcaactacaggagtcgggcccaggactggtgaagccttcggagaccctgtccctcacctgcactgtctctggtggctccatcagcactagtagttactactggggctggatccgccagcccccagggaaggggctggagtggattgggactatctattttagtgggaccacctactacaacccgtccctcacgagtcgagtcagcatgtccgtagacacgtccaagaaccagttctccctgaagctgaactctgtgaccgccgcagacacgtctatctattactgtgcgagacattggcccaagtacaactcctggttcgacccctggggccagggatttctggtcaccgtctcctca

SEQ ID NO: 61 - protein, 7 aa, synthetic construct
  REGION 1..7: note = Synthetic: HCDR1 (Kabat)
TSSYYWG

SEQ ID NO: 62 - protein, 16 aa, synthetic construct
  REGION 1..16: note = Synthetic: HCDR2 (Kabat)
TIYFSGTTYYNPSLTS

SEQ ID NO: 63 - protein, 11 aa, synthetic construct
  REGION 1..11: note = Synthetic: HCDR3 (Kabat)
HWPKYNSWFDP

SEQ ID NO: 64 - protein, 107 aa, synthetic construct [sequence omitted]

SEQ ID NO: 65 - DNA, 321 bp, synthetic construct
  misc_feature 1..321: note = Synthetic: VL (DNA)
  source 1..321: mol_type = other DNA
gaaattgtgttgacacagtctccagccaccctgtctttgtctccaggggaaagagccaccctctcctgcagggccagtcagagtgttagtagttacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaa

SEQ ID NO: 66 - protein, 11 aa, synthetic construct
  REGION 1..11: note = Synthetic: LCDR1 (Kabat)
RASQSVSSYLA

SEQ ID NO: 67 - protein, 7 aa, synthetic construct
  REGION 1..7: note = Synthetic: LCDR2 (Kabat)
DASNRAT

SEQ ID NO: 68 - protein, 9 aa, synthetic construct
  REGION 1..9: note = Synthetic: LCDR3 (Kabat)
QQRSNWPPT

SEQ ID NO: 69 - protein, 1390 aa, Homo sapiens
  REGION 1..1390: note = misc_feature - Human cMET full length
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 70 - protein, 330 aa, Homo sapiens
  REGION 1..330: note = misc_feature - human IgG1 constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 107 aa, Homo sapiens [sequence omitted]

SEQ ID NO: 72 - protein, 121 aa, synthetic construct
  REGION 1..121: note = Synthetic: VH (AA)
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRNMGYSGWLDYWGQGTLVTVSS

SEQ ID NO: 73 - protein, 121 aa, synthetic construct
  REGION 1..121: note = Synthetic: VH (AA)
EVQLVESGGGVVQPGKSLRLSCAASGFTFSSSGLHWVRQAPGKGLEWVAVISYDTSNKYYADSVKGRFTISRDNSKNTLYLQMNSLRAEDTAVYYCAKDLNRGYDWGFDYWGQGTLVTVSS

SEQ ID NO: 74 - protein, 120 aa, synthetic construct
  REGION 1..120: note = Synthetic: VH (AA)
EVQLVESGGGVVQPGRSLRLSCAASGFTFSNYGMHWVRQAPGKGLEWVAVISYDGVDKYYADSVKGRFTISRDNSKNTLYLQMNSLRAEDTAMYYCAKDRISYNWNLDYWGQGTLVTVSS

SEQ ID NO: 75 - protein, 121 aa, synthetic construct
  REGION 1..121: note = Synthetic: VH (AA)
EVQLVESGGGVVQPGRSLRLSCVASGFTFSNYGLHWVRQAPGKGLEWVAAISYDGSDKYYADPVKGRFTISRDTTKNTLSLQMNSLRAEDTAVYYCAKERYRGYDWDFDYWGQGTLVTVSS

SEQ ID NO: 76 - protein, 121 aa, synthetic construct
  REGION 1..121: note = Synthetic: VH (AA)
EVQLVESGGGVVQPGRSLRLSCAASGFTFSSYGLHWVRQAPGKGLEWVAVISFDGSNKYYADSVKGRFTISRDTSKNTLSLQMNSLRAEDTAVYYCAKERSRGYDWDFDYWGQGILVTVSS

SEQ ID NO: 77 - protein, 121 aa, synthetic construct
  REGION 1..121: note = Synthetic: VH (AA)
EVRLVESGGGVVQPGRSLRLSCAASGFTFSNYGMHWVRQAPGKGLEWVAVISYDGSDKYYPDSVKGRFTISRDNSRNTLYLQMNSLSTEDTAVYYCAKDQYRGYDGTFDYWGQGTLVTVSS

SEQ ID NO: 78 - protein, 120 aa, synthetic construct
  REGION 1..120: note = Synthetic: VH (AA)
EVQLVESGGGVVQPGRSLRLSCAASGFIFSSYNMHWVRQAPGKGLEWVAVISYDRVNKYYADSVKGRFTISRDNSKNTLYLQMNSLRVEDTAVYYCAKERERYFDWLDYWGQGTLVTVSS

SEQ ID NO: 79 - protein, 121 aa, synthetic construct
  REGION 1..121: note = Synthetic: VH (AA)
EVQLVESGGGVVQPGRSLRLSCAASGFTFSSYGMHWVRQAPGKGLEWVAVISNDGSYKYYVDSVTGRFTISRDNSKNTLYLQMNSLRAEDTAVYYCAKDRNMGYSGWFDYWGQGSLVTVSS

SEQ ID NO: 80 - protein, 121 aa, synthetic construct
  REGION 1..121: note = Synthetic: VH (AA)
EVQLVESGGGVVQPGRSLRLSCAASGFTFSSHGMHWVRQAPGKGLEWVAVISNDGSYKYYADSVTGRFTISRDNSKNTLYLQMNSLRAEDTAVYYCAKDRNMGYSGWFDYWGQGSLVTVSS

SEQ ID NO: 81 - protein, 122 aa, synthetic construct
  REGION 1..122: note = Synthetic: VH (AA)
EVQLVESGGGVVQPGRSLRLSCAASGFTFSNYGMHWVRQAPGKGLEWVAVISNDGSEKYYADSVKGRFTISRDSSKNTLYLQMNSLRAEDTAVYYCAKAPWGSGTTGRLDYWGQGTLVTVSS

SEQ ID NO: 82 - protein, 121 aa, synthetic construct
  REGION 1..121: note = Synthetic: VH (AA)
EFQLQESGPGLVKPSETLSLTCTVSGGSISTSSYYWGWIRQPPGKGLEWIGTIYYSGTTYYNPSLKSRVTISVDTSKNQFSLKLKSVTAADTSIYYCARHWPKYNSWFDPWGQGILVTVSS

SEQ ID NO: 83 - protein, 121 aa, synthetic construct
  REGION 1..121: note = Synthetic: VH (AA)
EFQLQESGPGLVKPSETLSLTCTVSGGSISTSSYYWGWIRQPPGKGLEWIGTIYFSGTTYYNPSLTSRVSMSVDTSKNQFSLKLNSVTAADTSIYYCARHWPKYNSWFDPWGQGFLVTVSS

SEQ ID NO: 84 - protein, 12 aa, synthetic construct
  REGION 1..12: note = Synthetic: HCDR3 (Kabat)
DRWEGYSGWLDY

SEQ ID NO: 85 - protein, 121 aa, synthetic construct
  REGION 1..121: note = Synthetic: VH (AA)
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRWEGYSGWLDYWGQGTLVTVSS

SEQ ID NO: 86 - protein, 12 aa, synthetic construct
  REGION 1..12: note = Synthetic: HCDR3 (Kabat)
DRYEGYSGWLDY

SEQ ID NO: 87 - protein, 121 aa, synthetic construct
  REGION 1..121: note = Synthetic: VH (AA)
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRYEGYSGWLDYWGQGTLVTVSS

SEQ ID NO: 88 - protein, 12 aa, synthetic construct
  REGION 1..12: note = Synthetic: HCDR3 (Kabat)
DRHEGYSGWLDY

SEQ ID NO: 89 - protein, 121 aa, synthetic construct
  REGION 1..121: note = Synthetic: VH (AA)
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRHEGYSGWLDYWGQGTLVTVSS

SEQ ID NO: 90 - protein, 12 aa, synthetic construct
  REGION 1..12: note = Synthetic: HCDR3 (Kabat)
DRFEGYSGWLDY

SEQ ID NO: 91 - protein, 121 aa, synthetic construct
  REGION 1..121: note = Synthetic: VH (AA)
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRFEGYSGWLDYWGQGTLVTVSS

SEQ ID NO: 92 - protein, 12 aa, synthetic construct
  REGION 1..12: note = Synthetic: HCDR3 (Kabat)
DRNEGYSGWLDY

SEQ ID NO: 93 - protein, 121 aa, synthetic construct
  REGION 1..121: note = Synthetic: VH (AA)
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRNEGYSGWLDYWGQGTLVTVSS

SEQ ID NO: 94 - protein, 12 aa, synthetic construct
  REGION 1..12: note = Synthetic: HCDR3 (Kabat)
  VARIANT 3: note = X = W, Y, H, F or N
  VARIANT 4: note = X = E
DRXXGYSGWLDY

SEQ ID NO: 95 - protein, 330 aa, synthetic construct
  REGION 1..330: note = Synthetic: Variant of human IgG1 constant region with Knob mutation T366W
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 330 aa, synthetic construct
  REGION 1..330: note = Synthetic: Variant of human IgG1 constant region with hole mutations T366S, L368A, and Y407V
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97-101 - skipped (5 entries)

SEQ ID NO: 102 - protein, 4 aa, synthetic construct
  REGION 1..4: note = Synthetic: linker
GGGS

SEQ ID NO: 103 - protein, 4 aa, synthetic construct
  REGION 1..4: note = Synthetic: linker
SGGG

SEQ ID NO: 104 - protein, 5 aa, synthetic construct
  REGION 1..5: note = Synthetic: linker
GGGGS

SEQ ID NO: 105 - protein, 7 aa, synthetic construct
  REGION 1..7: note = Synthetic: linker
GGGGSGS

SEQ ID NO: 106 - protein, 7 aa, synthetic construct
  REGION 1..7: note = Synthetic: linker
GGGGSGS

SEQ ID NO: 107 - protein, 8 aa, synthetic construct
  REGION 1..8: note = Synthetic: linker
GGGGSGGS

SEQ ID NO: 108 - protein, 10 aa, synthetic construct
  REGION 1..10: note = Synthetic: linker
GGGGSGGGGS

SEQ ID NO: 109 - protein, 15 aa, synthetic construct
  REGION 1..15: note = Synthetic: linker
GGGGSGGGGSGGGGS

SEQ ID NO: 110 - protein, 16 aa, synthetic construct
  REGION 1..16: note = Synthetic: linker
AKTTPKLEEGEFSEAR

SEQ ID NO: 111 - protein, 17 aa, synthetic construct
  REGION 1..17: note = Synthetic: linker
AKTTPKLEEGEFSEARV

SEQ ID NO: 112 - protein, 9 aa, synthetic construct
  REGION 1..9: note = Synthetic: linker
AKTTPKLGG

SEQ ID NO: 113 - protein, 10 aa, synthetic construct
  REGION 1..10: note = Synthetic: linker
SAKTTPKLGG

SEQ ID NO: 114 - protein, 17 aa, synthetic construct
  REGION 1..17: note = Synthetic: linker
AKTTPKLEEGEFSEARV

SEQ ID NO: 115 - protein, 6 aa, synthetic construct
  REGION 1..6: note = Synthetic: linker
SAKTTP

SEQ ID NO: 116 - protein, 10 aa, synthetic construct
  REGION 1..10: note = Synthetic: linker
SAKTTPKLGG

SEQ ID NO: 117 - protein, 6 aa, synthetic construct
  REGION 1..6: note = Synthetic: linker
RADAAP

SEQ ID NO: 118 - protein, 9 aa, synthetic construct
  REGION 1..9: note = Synthetic: linker
RADAAPTVS

SEQ ID NO: 119 - protein, 12 aa, synthetic construct
  REGION 1..12: note = Synthetic: linker
RADAAAAGGPGS

SEQ ID NO: 120 - protein, 27 aa, synthetic construct
  REGION 1..27: note = Synthetic: linker
RADAAAAGGGGSGGGGSGGGGSGGGGS

SEQ ID NO: 121 - protein, 6 aa, synthetic construct
  REGION 1..6: note = Synthetic: linker
SAKTTP

SEQ ID NO: 122 - protein, 10 aa, synthetic construct
  REGION 1..10: note = Synthetic: linker
SAKTTPKLGG

SEQ ID NO: 123 - protein, 18 aa, synthetic construct
  REGION 1..18: note = Synthetic: linker
SAKTTPKLEEGEFSEARV

SEQ ID NO: 124 - protein, 5 aa, synthetic construct
  REGION 1..5: note = Synthetic: linker
ADAAP

SEQ ID NO: 125 - protein, 12 aa, synthetic construct
  REGION 1..12: note = Synthetic: linker
ADAAPTVSIFPP

SEQ ID NO: 126 - protein, 5 aa, synthetic construct
  REGION 1..5: note = Synthetic: linker
TVAAP

SEQ ID NO: 127 - protein, 12 aa, synthetic construct
  REGION 1..12: note = Synthetic: linker
TVAAPSVFIFPP

SEQ ID NO: 128 - protein, 6 aa, synthetic construct
  REGION 1..6: note = Synthetic: linker
QPKAAP

SEQ ID NO: 129 - protein, 13 aa, synthetic construct
  REGION 1..13: note = Synthetic: linker
QPKAAPSVTLFPP

SEQ ID NO: 130 - protein, 6 aa, synthetic construct
  REGION 1..6: note = Synthetic: linker
AKTTPP

SEQ ID NO: 131 - protein, 13 aa, synthetic construct
  REGION 1..13: note = Synthetic: linker
AKTTPPSVTPLAP

SEQ ID NO: 132 - protein, 6 aa, synthetic construct
  REGION 1..6: note = Synthetic: linker
AKTTAP

SEQ ID NO: 133 - protein, 13 aa, synthetic construct
  REGION 1..13: note = Synthetic: linker
AKTTAPSVYPLAP

SEQ ID NO: 134 - protein, 6 aa, synthetic construct
  REGION 1..6: note = Synthetic: linker
ASTKGP

SEQ ID NO: 135 - protein, 13 aa, synthetic construct
  REGION 1..13: note = Synthetic: linker
ASTKGPSVFPLAP

SEQ ID NO: 136 - protein, 15 aa, synthetic construct
  REGION 1..15: note = Synthetic: linker
GENKVEYAPALMALS

SEQ ID NO: 137 - protein, 15 aa, synthetic construct
  REGION 1..15: note = Synthetic: linker
GPAKELTPLKEAKVS

SEQ ID NO: 138 - protein, 15 aa, synthetic construct
  REGION 1..15: note = Synthetic: linker
GHEAAAVMQVQYPAS

SEQ ID NO: 139 - protein, 20 aa, synthetic construct
  REGION 1..20: note = Synthetic: linker
GGGGSGGGGSGGGGSGGGGS

SEQ ID NO: 140 - protein, 118 aa, synthetic construct
  REGION 1..118: note = Synthetic: VH of Tislelizumab
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 141 - protein, 107 aa, synthetic construct
  REGION 1..107: note = Synthetic: VL of Tislelizumab
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 142 - protein, 125 aa, synthetic construct [sequence omitted]

SEQ ID NO: 143 - protein, 107 aa, synthetic construct [sequence omitted]

SEQ ID NO: 144 - protein, 121 aa, synthetic construct
  REGION 1..121: note = Synthetic: VH (AA) (063Ab10910-P19 + charge engineering in FR)
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSS

SEQ ID NO: 145 - protein, 121 aa, synthetic construct
  REGION 1..121: note = Synthetic: VH (AA) (063Ab03210-V1 + charge engineering)
EFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSS

SEQ ID NO: 146 - protein, 723 aa, synthetic construct
  REGION 1..723: note = Synthetic: Polypeptide 1 (Knob chain)
EFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 147 - protein, 723 aa, synthetic construct
  REGION 1..723: note = Synthetic: Polypeptide 2 (Hole chain) with BGA-109 and EGFR arm
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 148 - protein, 214 aa, synthetic construct
  REGION 1..214: note = Synthetic: Polypeptide 3 (Common LC)
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 451 aa, synthetic construct
  REGION 1..451: note = Synthetic: Polypeptide 1 (Knob chain) with BGA-032
EFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 451 aa, synthetic construct
  REGION 1..451: note = Synthetic: Polypeptide 2 (Hole chain)
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 151 - protein, 486 aa, synthetic construct
  REGION 1..486: note = Synthetic: Polypeptide 3 with common LC and EGFR arm
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 152 - protein, 486 aa, synthetic construct
  REGION 1..486: note = Synthetic: Polypeptide 3 EGFR arm and common LC
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 723 aa, synthetic construct
  REGION 1..723: note = Synthetic: Polypeptide 1 (Knob chain) with EGFR arm and BGA-032
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGGGSEFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 723 aa, synthetic construct
  REGION 1..723: note = Synthetic: Polypeptide 2 (Hole chain) with EGFR arm and BGA-109
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGGGSEVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 155 - protein, 5 aa, synthetic construct
  REGION 1..5: note = Synthetic: HCDR1 (Kabat)
TYGMH

SEQ ID NO: 156 - protein, 17 aa, synthetic construct
  REGION 1..17: note = Synthetic: HCDR2 (Kabat)
VIWDDGSYKYYGDSVKG

SEQ ID NO: 157 - protein, 16 aa, synthetic construct
  REGION 1..16: note = Synthetic: HCDR3 (Kabat)
DGITMVRGVMKDYFDY

SEQ ID NO: 158 - protein, 11 aa, synthetic construct
  REGION 1..11: note = Synthetic: LCDR1 (Kabat)
RASQDISSALV

SEQ ID NO: 159 - protein, 7 aa, synthetic construct
  REGION 1..7: note = Synthetic: LCDR2 (Kabat)
DASSLES

SEQ ID NO: 160 - protein, 9 aa, synthetic construct
  REGION 1..9: note = Synthetic: LCDR3 (Kabat)
QQFNSYPLT

SEQ ID NO: 161 - protein, 252 aa, synthetic construct
  REGION 1..252: note = Synthetic: ScFv (VL-VH)
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 162 - DNA, 363 bp, synthetic construct
  misc_feature 1..363: note = Synthetic: VH (DNA)
  source 1..363: mol_type = other DNA
gaagtgcagctggtggagtccgggggcgaggtggtccaacccggggagagcctgagactgagctgcgccgctagcggcttcaccttcagcagccacggcatgcactgggtgagacaagcccccggcaagggcctggagtgggtggccgtgatcagctacgacgagagcgacaagtactacgccgacagcgtgaagggcagattcaccatcagcagagaccacagcaagaacaccctgtacctggagatgaacagcctgagagccgaggacaccgccgtgtactactgcgtgaaggacagaaacgagggctacagcggctggctggactactggggccaaggcaccctggtgaccgtgagcagc

SEQ ID NO: 163 - DNA, 363 bp, synthetic construct
  misc_feature 1..363: note = Synthetic: VH (DNA)
  source 1..363: mol_type = other DNA
gagtttcagctgcaagagagcggccccggcctggtgaagcctagcgagaccctgagactgacctgcaccgtgagcggcggcagcatcagcacaagcagctactactggggctggatcagacagccccccggcaagggcctggagtggatcggcaccatctactacagcggcaccacctactacaaccctagcctgaagagcagagtgaccatcagcgtggacacaagcaagaatcagttcagcctgaagctgaagagcgtgagagccgccgacacaagcatctactactgcgctagacactggcccaagtacaacagctggttcgacccctggggccaaggcatcctggtgaccgtgagcagc

SEQ ID NO: 164 - DNA, 2169 bp, synthetic construct
  misc_feature 1..2169: note = Synthetic: Polypeptide 1 (Knob chain) with EGFR arm and BGA-032 DNA
  source 1..2169: mol_type = other DNA
gccattcagctgacacagagccctagcagcctgagcgctagcgtgggcgacagagtgacaatcacctgcagagctagccaagacatcagcagcgccctggtgtggtatcagcagaagcccggcaaggcccccaagctgctgatctacgacgctagcagcctcgaaagcggcgtgcctagcagatttagcggcagcgagagcggcaccgacttcaccctgaccatcagcagcctgcagcccgaggacttcgccacctactactgtcagcagttcaacagctaccccctgaccttcggcgggggcacaaaagtggaaattaagggtggaggcggttcaggcggaggtggctctggcggtggcggatcgggtggcggaggctcacaagtgcaactggtcgaaagcggcgggggggtggtgcagcctgggagaagcctgagactgagctgcgccgctagcggcttcaccttcagcacctacggcatgcactgggtgagacaagcccccggcaaaggcctggaatgggtggccgtgatctgggacgacggcagctacaagtactacggcgacagcgtgaagggcagattcaccatcagcagagacaacagcaagaacaccctgtacctgcagatgaacagcctgagagccgaggacaccgccgtgtactactgcgctagagacggcatcaccatggtgagaggcgtgatgaaggactactttgactattgggggcaaggcaccctggtgaccgtgagcagcggtggaggcggttcaggcggaggtggctctggcggtggcggatcgggtggcggaggctcagagtttcagctgcaagagtccggccccggcctggtgaagcctagcgagaccctgagactgacctgcaccgtgagcggcggcagcatcagcacaagcagctactactggggctggatcagacagcctcccggcaagggcctggagtggatcggcaccatctactacagcggcaccacctactacaaccctagcctgaagagcagagtgaccatcagcgtggacacaagcaagaatcagttcagcctgaagctgaagagcgtgagagccgccgacacaagcatctattactgcgcccggcactggcccaagtacaacagctggttcgacccctggggccaaggcatcctggtgaccgtcagcagcgcctccacaaaggggcctagcgtgttccctctggcccctagcagcaaaagcacaagcggcggcaccgctgccctgggctgcctggtcaaggattacttccccgagcccgtgaccgtgagctggaatagcggcgctctcacaagcggcgtgcacaccttccccgccgtgctgcagagcagcggcctgtacagcctgagcagcgtggtgaccgtgcctagcagcagcctgggcacacagacctacatctgcaacgtgaaccacaagcctagcaacaccaaggtggacaaaaaggtggagcccaagtcctgtgacaagacccacacctgccccccctgtcccgcccccgaactcctgggcggccctagcgtgttcctgttcccccccaagcccaaggacaccctgatgatcagcagaacccccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagaggagatgaccaagaaccaagtgagcctgtggtgcctggtgaaggggttttaccctagcgacatcgccgtggagtgggagagcaacgggcagcccgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgagcctgagccccggcaag

SEQ ID NO: 165 - DNA, 1353 bp, synthetic construct
  misc_feature 1..1353: note = Synthetic: Polypeptide 2 (Hole chain) with BGA-109 DNA
  source 1..1353: mol_type = other DNA
gaggtgcagctggtcgagagcgggggcgaagtggtccaacccggcgagagcctgagactgtcctgcgccgctagcggcttcaccttcagcagccacggcatgcactgggtgagacaagcccccggcaagggcctggagtgggtggccgtgatcagctacgacgagagcgacaagtactacgccgacagcgtgaagggcagattcaccatcagcagagaccacagcaagaacaccctgtacctggagatgaacagcctgagagccgaggacaccgccgtgtactactgcgtgaaggacagaaacgagggctacagcggctggctggactactggggccaaggcaccctggtgaccgtgtcctccgctagcaccaagggccctagcgtgtttcccctggcccctagcagcaagtccacctccggcgggacagccgctctgggctgcctggtgaaggactacttccccgagcccgtgacagtgagctggaactccggcgctctgacaagcggcgtgcataccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcccaaatcctgcgataaaacccacacatgccccccctgccccgcccccgagctgctgggcggcccctccgtcttcctgttcccccccaagcccaaggacaccctgatgatcagcagaacccccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagaggagatgaccaagaaccaagtgagcctgagctgcgccgtgaagggcttctaccctagcgacatcgccgtggagtgggagagcaacgggcagcccgagaacaactacaagaccaccccccccgtgctggacagcgacggcagcttcttcctggtgagcaagctgaccgtggacaagagcagatggcagcaaggcaacgtgttcagctgcagcgtgatgcacgaggccctgcacaaccactacacacagaagagcctgagcctgagccccggcaag

SEQ ID NO: 166 - DNA, 642 bp, synthetic construct
  misc_feature 1..642: note = Synthetic: Polypeptide 3 (Common LC) DNA
  source 1..642: mol_type = other DNA
gagattgtgctgacacagagccccgccaccctgtccctgtcccccggggagagagccaccctgagctgcagagctagccaaagcgtgagcagctacctggcctggtatcagcagaagcccggccaagcccctagactgctgatctacgacgctagcaacagagccaccggcatccccgctagattcagcggcagcggcagcggcaccgacttcaccctgaccatcagcagcctggagcccgaggacttcgccgtgtactactgtcagcagagaagcaactggccccccaccttcggccaaggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 167 - protein, 1210 aa, Homo sapiens [sequence omitted]

(19) United States
(12) Patent Application Publication
(10) Pub. No.: US 2025/0154265 A1
(43) Pub. Date: May 15, 2025

(54) Anti-cMET Antibodies and Methods of Use

(71) Applicant: BeiGene Ltd., Grand Cayman (KY)

(72) Inventors: Renke Li, Shanghai (CN); Xiaoyan Tang, Beijing (CN); Mengran Qian, Shanghai (CN); Jie Pan, Shanghai (CN); Zhao Zhao, Shanghai (CN); Xi Wu, Beijing (CN); Ming Jiang, Beijing (CN); Hui Li, Beijing (CN); Xiaona Sun, Beijing (CN); Yanzhou Zhang, Shanghai (CN); Mengmeng Lu, Shanghai (CN); Yun Chen, Shanghai (CN)

(73) Assignee: BeiGene Ltd., Grand Cayman (KY)

(21) Appl. No.: 18/957,490

(22) Filed: Nov. 22, 2024

Related U.S. Application Data
(63) Continuation of application No. PCT/CN2024/072952, filed on Jan. 18, 2024.

(30) Foreign Application Priority Data
Jan. 19, 2023 (WO) PCT/CN2023/073074
Jun. 28, 2023 (WO) PCT/CN2023/103335
Jun. 28, 2023 (WO) PCT/CN2023/103351

(51) Int. Cl. C07K 16/28; A61K 39/00; A61P 35/00

(52) U.S. Cl. CPC C07K 16/2863; A61P 35/00; A61K 2039/505; C07K 2317/31; C07K 2317/41; C07K 2317/52; C07K 2317/565; C07K 2317/622

(57) ABSTRACT

The present disclosure provides for antibody or antigen-binding fragment thereof that specifically binds to human cMET and multispecific antibody or antigen-binding fragment thereof, comprising a first antigen binding domain that specifically binds a first epitope of human cMET; a second antigen binding domain that specifically binds to a second epitope of human cMET; and a third antigen binding domain that specifically binds to human EGFR; wherein the first epitope is distinct from the second epitope, or wherein the first antigen binding domain does not compete with the second antigen binding domain. The present disclosure also provides for the use of the antibodies or multispecific antibodies for treating a disease, such as cancer.

### CROSS-REFERENCE TO RELATED APPLICATIONS

This application is a continuation of International Application No. PCT/CN2024/072952, filed on Jan. 18, 2024, which claims priority to International Application No. PCT/CN2023/073074, filed on Jan. 19, 2023, International Application No. PCT/CN2023/103351, filed on Jun. 28, 2023, and International Application No. PCT/CN2023/103335, filed on Jun. 28, 2023, the disclosures of each of which are hereby incorporated by reference in their entireties.

### FIELD OF THE DISCLOSURE

Disclosed herein are antibodies that specifically bind to human cMET, multispecific antibodies that specifically bind to two distinct epitopes of human cMET, multispecific antibodies or antigen-binding fragments thereof that specifically bind to human EGFR and human cMET, as well as isolated nucleic acids, vectors and host cells. Lastly, the antibodies or antigen-binding fragments thereof disclosed herein can be used in the treatment of various cancers.

### SEQUENCE LISTING

The instant application contains a Sequence Listing, which has been submitted electronically in XML format and is hereby incorporated by reference in its entirety. Said XML copy, created on Feb. 1, 2024, is named “01368-0057-00PCT_SL_xml” and is 230,959 bytes in size.

### BACKGROUND

The receptor tyrosine kinase mesenchymal-epithelial transition factor (c-MET, also known as cMET or MET, hepatocyte growth factor receptor, or HGFR) and its ligand hepatocyte growth factor (HGF), play crucial roles in multiple cellular processes that stimulate cellular proliferation, survival, invasion, angiogenesis.

cMET is composed of an extracellular α-subunit and a transmembrane β-subunit that are linked by disulfide bonds. The extracellular region consists of a Semaphorin (SEMA) domain, a plexin-semaphorin-integrin (PSI) domain, and four consecutive immunoglobulin-plexin-transcription factors (IPT1-4) domains. The binding of HGF to cMET induces the dimerization of cMET that enables its intracellular kinase domains (KDs) to undergo autophosphorylation¹, which in turn creates active docking sites for proteins which mediate activation of downstream signaling pathways such as mitogen-activated protein kinase (MAPK), phosphatidylinositol 3-kinase (PI3K)-AKT pathways.

Dysregulated HGF-cMET signaling has been observed in multiple solid cancer types including gastric cancer, colorectal cancer, lung cancer, liver cancer, head and neck cancer, breast cancer, brain cancer, etc. Aberrant cMET activation in an HGF-independent manner can be induced by multiple mechanisms, including cMET overexpression, genomic amplification, mutation and alternative splicing². Given its important role in cellular process, oncogenesis, and cancer progression, c-MET is considered as a promising target for cancer treatment.

Both small molecules and antibodies targeting cMET have been developed and under evaluation in clinical trials. However, in most trials, cMET inhibitors didn't achieve promising results³.

Activating mutations in the kinase domain of epidermal growth factor receptor (EGFR) occur in a substantial percentage of non-small cell lung cancer (NSCLC) patients.⁴⁻⁵ Their presence is associated with sensitivity to EGFR tyrosine kinase inhibitors (TKIs). Despite the initial efficacy of TKI in treating EGFR mutation harboring NSCLC, most patients inevitably experience acquired resistance in about a year after initial treatment.^{6.7}

The acquired resistance mechanisms generally fall into several categories: EGFR mutations, gene amplifications, MAPK-PI3K mutations, oncogene fusions, cell cycle gene alterations, and others. The evolution of the EGFR kinase inhibitors overcomes the emerging EGFR mutation mechanisms. However, overcoming therapeutic resistance remains a major challenge given a large portion of resistances are caused by non-EGFR mutation alteration. Among these, hepatocyte growth factor receptor (cMET) amplification is one of the major alterations that lead to resistance after EGFR TKI treatment. For example, it was found in 5-50% of patients who develop resistance to second-line osimertinib treatment and 7-15% of first-line treatment.⁸

Mechanistically, both EGFR and cMET signal are through ERK and AKT pathway, therefore the cMET signaling activation compensates for the loss of EGFR-driven signaling when EGFR is inhibited. This makes the strategy of combining EGFR and cMET targeting especially attractive to overcome the cMET amplification-driven compensatory resistance. This strategy has been applied in JNJ-61186372 (Amivantamab, or abbreviated as JNJ-372), an EGFR×c-MET bispecific antibody.⁹

Given the above reason, there remains an unmet medical need for therapeutics targeting EGFR and/or cMET.

### SUMMARY OF THE DISCLOSURE

The disclosure contains antibodies or antibody fragment thereof that specifically bind to human cMET and multispecific antibodies or antibody fragment thereof that specifically bind to two distinct epitopes of human cMET (e.g., non-overlapping). In addition, the antibodies and antibody fragments thereof disclosed herein can be used to construct multispecific antibodies with other modalities such as a second tumor associated antigen (TAA), immune checkpoints or immune stimulators, or to construct antibody drug conjugates (ADC) or to form fusion proteins. cMET antibodies alone or in combination with other therapeutics could potentially be used for the treatment or prevention of cancer.

The disclosure contains multispecific antibodies or antibody fragment thereof that specifically bind to human EGFR and two distinct epitopes of human cMET (e.g., non-overlapping). The multispecific antibodies or antibody fragment thereof alone or in combination with other therapeutics could potentially be used for the treatment or prevention of cancer.

The present disclosure encompasses the following embodiments.

Embodiment 1: An antibody or antigen-binding fragment thereof which specifically binds human cMET, comprising:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(13) a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68;
(14) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(15) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(16) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(17) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(18) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68.

Embodiment 2: The antibody or antigen-binding fragment thereof of embodiment 1, that comprises:
(1) a heavy chain variable region (VH) comprising an amino acid sequence at least 90, 91, 92, 93, 94, 95, 96, 97, 98 or 99% identical to SEQ ID NO: 85, SEQ ID NO: 87, SEQ ID NO: 89, SEQ ID NO: 91, or SEQ ID NO: 93, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 4 or SEQ ID NO: 72, and a light chain variable region (VL) comprising an amino acid sequence at least 90, 91, 92, 93, 94, 95, 96, 97, 98 or 99% identical to SEQ ID NO: 64;
(3) a heavy chain variable region (VH) comprising an amino acid sequence at least 90, 91, 92, 93, 94, 95, 96, 97, 98 or 99% identical to SEQ ID NO: 9 or SEQ ID NO: 73, and a light chain variable region (VL) comprising an amino acid sequence at least 90, 91, 92, 93, 94, 95, 96, 97, 98 or 99% identical to SEQ ID NO: 64;
(4) a heavy chain variable region (VH) comprising an amino acid sequence at least 90, 91, 92, 93, 94, 95, 96, 97, 98 or 99% identical to SEQ ID NO: 54, or SEQ ID NO: 82, and a light chain variable region (VL) comprising an amino acid sequence at least 90, 91, 92, 93, 94, 95, 96, 97, 98 or 99% identical to SEQ ID NO: 64;
(5) a heavy chain variable region (VH) comprising an amino acid sequence at least 90, 91, 92, 93, 94, 95, 96, 97, 98 or 99% identical to SEQ ID NO: 14, or SEQ ID NO: 74, and a light chain variable region (VL) comprising an amino acid sequence at least 90, 91, 92, 93, 94, 95, 96, 97, 98 or 99% identical to SEQ ID NO: 64;
(6) a heavy chain variable region (VH) comprising an amino acid sequence at least 90, 91, 92, 93, 94, 95, 96, 97, 98 or 99% identical to SEQ ID NO: 19, or SEQ ID NO: 75, and a light chain variable region (VL) comprising an amino acid sequence at least 90, 91, 92, 93, 94, 95, 96, 97, 98 or 99% identical to SEQ ID NO: 64;
(7) a heavy chain variable region (VH) comprising an amino acid sequence at least 90, 91, 92, 93, 94, 95, 96, 97, 98 or 99% identical to SEQ ID NO: 24, or SEQ ID NO: 76, and a light chain variable region (VL) comprising an amino acid sequence at least 90, 91, 92, 93, 94, 95, 96, 97, 98 or 99% identical to SEQ ID NO: 64;
(8) a heavy chain variable region (VH) comprising an amino acid sequence at least 90, 91, 92, 93, 94, 95, 96, 97, 98 or 99% identical to SEQ ID NO: 29, or SEQ ID NO: 77, and a light chain variable region (VL) comprising an amino acid sequence at least 90, 91, 92, 93, 94, 95, 96, 97, 98 or 99% identical to SEQ ID NO: 64;
(9) a heavy chain variable region (VH) comprising an amino acid sequence at least 90, 91, 92, 93, 94, 95, 96, 97, 98 or 99% identical to SEQ ID NO: 34, or SEQ ID NO: 78, and a light chain variable region (VL) comprising an amino acid sequence at least 90, 91, 92, 93, 94, 95, 96, 97, 98 or 99% identical to SEQ ID NO: 64;
(10) a heavy chain variable region (VH) comprising an amino acid sequence at least 90, 91, 92, 93, 94, 95, 96, 97, 98 or 99% identical to SEQ ID NO: 39, or SEQ ID NO: 79, and a light chain variable region (VL) comprising an amino acid sequence at least 90, 91, 92, 93, 94, 95, 96, 97, 98 or 99% identical to SEQ ID NO: 64;
(11) a heavy chain variable region (VH) comprising an amino acid sequence at least 90, 91, 92, 93, 94, 95, 96, 97, 98 or 99% identical to SEQ ID NO: 44, or SEQ ID NO: 80, and a light chain variable region (VL) comprising an amino acid sequence at least 90, 91, 92, 93, 94, 95, 96, 97, 98 or 99% identical to SEQ ID NO: 64;
(12) a heavy chain variable region (VH) comprising an amino acid sequence at least 90, 91, 92, 93, 94, 95, 96, 97, 98 or 99% identical to SEQ ID NO: 49, or SEQ ID NO: 81, and a light chain variable region (VL) comprising an amino acid sequence at least 90, 91, 92, 93, 94, 95, 96, 97, 98 or 99% identical to SEQ ID NO: 64;
(13) a heavy chain variable region (VH) comprising an amino acid sequence at least 90, 91, 92, 93, 94, 95, 96, 97, 98 or 99% identical to SEQ ID NO: 59, or SEQ ID NO: 83, and a light chain variable region (VL) comprising an amino acid sequence at least 90, 91, 92, 93, 94, 95, 96, 97, 98 or 99% identical to SEQ ID NO: 64;
(14) a heavy chain variable region (VH) comprising an amino acid sequence at least 90, 91, 92, 93, 94, 95, 96, 97, 98 or 99% identical to SEQ ID NO: 144, and a light chain variable region (VL) comprising an amino acid sequence at least 90, 91, 92, 93, 94, 95, 96, 97, 98 or 99% identical to SEQ ID NO: 64; or
(15) a heavy chain variable region (VH) comprising an amino acid sequence at least 90, 91, 92, 93, 94, 95, 96, 97, 98 or 99% identical to SEQ ID NO: 145, and a light chain variable region (VL) comprising an amino acid sequence at least 90, 91, 92, 93, 94, 95, 96, 97, 98 or 99% identical to SEQ ID NO: 64.

Embodiment 3: The antibody or antigen-binding fragment thereof of any one of the preceding embodiments, wherein one, two, three, four, five, six, seven, eight, nine, or ten amino acids within SEQ ID NO: 85, SEQ ID NO: 87, SEQ ID NO: 89, SEQ ID NO: 91, SEQ ID NO: 93, SEQ ID NO: 4, SEQ ID NO: 72, SEQ ID NO: 9, SEQ ID NO: 73, SEQ ID NO: 54, SEQ ID NO: 82, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81, SEQ ID NO: 59, SEQ ID NO: 83, SEQ ID NO: 144, SEQ ID NO: 145, or SEQ ID NO: 64 have been inserted, deleted or substituted.

Embodiment 4: The antibody or antigen-binding fragment thereof of any one of the preceding embodiments, that comprises:
(1) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64;
(2) a heavy chain variable region (VH) comprising an amino acid sequence of SEQ ID NO: 4 or SEQ ID NO: 72, and a light chain variable region (VL) comprising an amino acid sequence of SEQ ID NO: 64;
(3) a heavy chain variable region (VH) comprising an amino acid sequence of SEQ ID NO: 9 or SEQ ID NO: 73, and a light chain variable region (VL) comprising an amino acid sequence of SEQ ID NO: 64;
(4) a heavy chain variable region (VH) comprising an amino acid sequence of SEQ ID NO: 54, or SEQ ID NO: 82, and a light chain variable region (VL) comprising an amino acid sequence of SEQ ID NO: 64;
(5) a heavy chain variable region (VH) comprising an amino acid sequence of SEQ ID NO: 14, or SEQ ID NO: 74, and a light chain variable region (VL) comprising an amino acid sequence of SEQ ID NO: 64;
(6) a heavy chain variable region (VH) comprising an amino acid sequence of SEQ ID NO: 19, or SEQ ID NO: 75, and a light chain variable region (VL) comprising an amino acid sequence of SEQ ID NO: 64;
(7) a heavy chain variable region (VH) comprising an amino acid sequence of SEQ ID NO: 24, or SEQ ID NO: 76, and a light chain variable region (VL) of SEQ ID NO: 64;
(8) a heavy chain variable region (VH) comprising an amino acid sequence of SEQ ID NO: 29, or SEQ ID NO: 77, and a light chain variable region (VL) comprising an amino acid sequence of SEQ ID NO: 64;
(9) a heavy chain variable region (VH) comprising an amino acid sequence of SEQ ID NO: 34, or SEQ ID NO: 78, and a light chain variable region (VL) comprising an amino acid sequence of SEQ ID NO: 64;
(10) a heavy chain variable region (VH) comprising an amino acid sequence of SEQ ID NO: 39, or SEQ ID NO: 79, and a light chain variable region (VL) comprising an amino acid sequence of SEQ ID NO: 64;
(11) a heavy chain variable region (VH) comprising an amino acid sequence of SEQ ID NO: 44, or SEQ ID NO: 80, and a light chain variable region (VL) comprising an amino acid sequence of SEQ ID NO: 64;
(12) a heavy chain variable region (VH) comprising an amino acid sequence of SEQ ID NO: 49, or SEQ ID NO: 81, and a light chain variable region (VL) comprising an amino acid sequence of SEQ ID NO: 64;
(13) a heavy chain variable region (VH) comprising an amino acid sequence of SEQ ID NO: 59, or SEQ ID NO: 83, and a light chain variable region (VL) comprising an amino acid sequence of SEQ ID NO: 64;
(14) a heavy chain variable region (VH) comprising an amino acid sequence of SEQ ID NO: 144, and a light chain variable region (VL) comprising an amino acid sequence of SEQ ID NO: 64; or
(15) a heavy chain variable region (VH) comprising an amino acid sequence of SEQ ID NO: 145, and a light chain variable region (VL) comprising an amino acid sequence of SEQ ID NO: 64.

Embodiment 5: The antibody or antigen-binding fragment thereof of any one of the preceding embodiments, which is a monoclonal antibody, a human engineered antibody, a single chain antibody (scFv), a Fab fragment, a Fab′ fragment, or a F(ab′)2 fragment.

Embodiment 6: A multispecific antibody or antigen-binding fragment thereof, comprising
a first antigen binding domain that specifically binds a first epitope of human cMET, and
a second antigen binding domain that specifically binds to a second epitope of human cMET,
wherein the first epitope is distinct from the second epitope, or wherein the first antigen binding domain is distinct from the second antigen binding domain, or wherein the first antigen binding domain does not compete with the second antigen binding domain.

Embodiment 7: The multispecific antibody or antigen-binding fragment thereof of embodiment 6, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(13) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(14) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(15) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(16) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68.

Embodiment 8: The multispecific antibody or antigen-binding fragment thereof of any one of embodiments 6-7, wherein the first antigen binding domain that specifically binds to the first epitope of human cMET comprises:
(1) a heavy chain variable region (VH) comprising an amino acid sequence at least 90, 91, 92, 93, 94, 95, 96, 97, 98 or 99% identical to SEQ ID NO: 85, SEQ ID NO: 87, SEQ ID NO: 89, SEQ ID NO: 91, or SEQ ID NO: 93, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 4 or SEQ ID NO: 72, and a light chain variable region (VL) comprising an amino acid sequence at least 90, 91, 92, 93, 94, 95, 96, 97, 98 or 99% identical to SEQ ID NO: 64;
(3) a heavy chain variable region (VH) comprising an amino acid sequence at least 90, 91, 92, 93, 94, 95, 96, 97, 98 or 99% identical to SEQ ID NO: 9 or SEQ ID NO: 73, and a light chain variable region (VL) comprising an amino acid sequence at least 90, 91, 92, 93, 94, 95, 96, 97, 98 or 99% identical to SEQ ID NO: 64;
(4) a heavy chain variable region (VH) comprising an amino acid sequence at least 90, 91, 92, 93, 94, 95, 96, 97, 98 or 99% identical to SEQ ID NO: 14, or SEQ ID NO: 74, and a light chain variable region (VL) comprising an amino acid sequence at least 90, 91, 92, 93, 94, 95, 96, 97, 98 or 99% identical to SEQ ID NO: 64;
(5) a heavy chain variable region (VH) comprising an amino acid sequence at least 90, 91, 92, 93, 94, 95, 96, 97, 98 or 99% identical to SEQ ID NO: 19, or SEQ ID NO: 75, and a light chain variable region (VL) comprising an amino acid sequence at least 90, 91, 92, 93, 94, 95, 96, 97, 98 or 99% identical to SEQ ID NO: 64;
(6) a heavy chain variable region (VH) comprising an amino acid sequence at least 90, 91, 92, 93, 94, 95, 96, 97, 98 or 99% identical to SEQ ID NO: 24, or SEQ ID NO: 76, and a light chain variable region (VL) comprising an amino acid sequence at least 90, 91, 92, 93, 94, 95, 96, 97, 98 or 99% identical to SEQ ID NO: 64;
(7) a heavy chain variable region (VH) comprising an amino acid sequence at least 90, 91, 92, 93, 94, 95, 96, 97, 98 or 99% identical to SEQ ID NO: 29, or SEQ ID NO: 77, and a light chain variable region (VL) comprising an amino acid sequence at least 90, 91, 92, 93, 94, 95, 96, 97, 98 or 99% identical to SEQ ID NO: 64;
(8) a heavy chain variable region (VH) comprising an amino acid sequence at least 90, 91, 92, 93, 94, 95, 96, 97, 98 or 99% identical to SEQ ID NO: 34, or SEQ ID NO: 78, and a light chain variable region (VL) comprising an amino acid sequence at least 90, 91, 92, 93, 94, 95, 96, 97, 98 or 99% identical to SEQ ID NO: 64;
(9) a heavy chain variable region (VH) comprising an amino acid sequence at least 90, 91, 92, 93, 94, 95, 96, 97, 98 or 99% identical to SEQ ID NO: 39, or SEQ ID NO: 79, and a light chain variable region (VL) comprising an amino acid sequence at least 90, 91, 92, 93, 94, 95, 96, 97, 98 or 99% identical to SEQ ID NO: 64;
(10) a heavy chain variable region (VH) comprising an amino acid sequence at least 90, 91, 92, 93, 94, 95, 96, 97, 98 or 99% identical to SEQ ID NO: 44, or SEQ ID NO: 80, and a light chain variable region (VL) comprising an amino acid sequence at least 90, 91, 92, 93, 94, 95, 96, 97, 98 or 99% identical to SEQ ID NO: 64;
(11) a heavy chain variable region (VH) comprising an amino acid sequence at least 90, 91, 92, 93, 94, 95, 96, 97, 98 or 99% identical to SEQ ID NO: 49, or SEQ ID NO: 81, and a light chain variable region (VL) comprising an amino acid sequence at least 90, 91, 92, 93, 94, 95, 96, 97, 98 or 99% identical to SEQ ID NO: 64; or
(12) a heavy chain variable region (VH) comprising an amino acid sequence at least 90, 91, 92, 93, 94, 95, 96, 97, 98 or 99% identical to SEQ ID NO: 144, and a light chain variable region (VL) comprising an amino acid sequence at least 90, 91, 92, 93, 94, 95, 96, 97, 98 or 99% identical to SEQ ID NO: 64.

Embodiment 9: The multispecific antibody or antigen-binding fragment thereof of embodiment 8, wherein in the first antigen binding domain that specifically binds to the first epitope of human cMET, one, two, three, four, five, six, seven, eight, nine, or ten amino acids within SEQ ID NO: 85, SEQ ID NO: 87, SEQ ID NO: 89, SEQ ID NO: 91, SEQ ID NO: 93, SEQ ID NO: 4, SEQ ID NO: 72, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81, SEQ ID NO: 144 or SEQ ID NO: 64 have been inserted, deleted or substituted.

Embodiment 10: The multispecific antibody or antigen-binding fragment thereof of any one of embodiments 6-9, wherein the first antigen binding domain that specifically binds to the first epitope of human cMET comprises:
(1) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64;
(2) a heavy chain variable region (VH) comprising an amino acid sequence of SEQ ID NO: 4 or SEQ ID NO: 72, and a light chain variable region (VL) comprising an amino acid sequence of SEQ ID NO: 64;
(3) a heavy chain variable region (VH) comprising an amino acid sequence of SEQ ID NO: 9 or SEQ ID NO: 73, and a light chain variable region (VL) comprising an amino acid sequence of SEQ ID NO: 64;
(4) a heavy chain variable region (VH) comprising an amino acid sequence of SEQ ID NO: 14, or SEQ ID NO: 74, and a light chain variable region (VL) comprising an amino acid sequence of SEQ ID NO: 64;
(5) a heavy chain variable region (VH) comprising an amino acid sequence of SEQ ID NO: 19, or SEQ ID NO: 75, and a light chain variable region (VL) comprising an amino acid sequence of SEQ ID NO: 64;
(6) a heavy chain variable region (VH) comprising an amino acid sequence of SEQ ID NO: 24, or SEQ ID NO: 76, and a light chain variable region (VL) of SEQ ID NO: 64;
(7) a heavy chain variable region (VH) comprising an amino acid sequence of SEQ ID NO: 29, or SEQ ID NO: 77, and a light chain variable region (VL) comprising an amino acid sequence of SEQ ID NO: 64;
(8) a heavy chain variable region (VH) comprising an amino acid sequence of SEQ ID NO: 34, or SEQ ID NO: 78, and a light chain variable region (VL) comprising an amino acid sequence of SEQ ID NO: 64;
(9) a heavy chain variable region (VH) comprising an amino acid sequence of SEQ ID NO: 39, or SEQ ID NO: 79, and a light chain variable region (VL) comprising an amino acid sequence of SEQ ID NO: 64;
(10) a heavy chain variable region (VH) comprising an amino acid sequence of SEQ ID NO: 44 or SEQ ID NO: 80, and a light chain variable region (VL) comprising an amino acid sequence of SEQ ID NO: 64;
(11) a heavy chain variable region (VH) comprising an amino acid sequence of SEQ ID NO: 49 or SEQ ID NO: 81, and a light chain variable region (VL) comprising an amino acid sequence of SEQ ID NO: 64; or
(12) a heavy chain variable region (VH) comprising an amino acid sequence of SEQ ID NO: 144, and a light chain variable region (VL) comprising an amino acid sequence of SEQ ID NO: 64.

Embodiment 11: The multispecific antibody or antigen-binding fragment thereof of any one of embodiments 6-10, wherein the second antigen binding domain that specifically binds to the second epitope of human cMET comprises:
(1) a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; or
(2) a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68.

Embodiment 12: The multispecific antibody or antigen-binding fragment thereof of any one of embodiments 6-11, wherein the second antigen binding domain that specifically binds to the second epitope of human cMET comprises:
(1) a heavy chain variable region (VH) comprising an amino acid sequence at least 90, 91, 92, 93, 94, 95, 96, 97, 98 or 99% identical to SEQ ID NO: 54, or SEQ ID NO: 82, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 59, or SEQ ID NO: 83, and a light chain variable region (VL) comprising an amino acid sequence at least 90, 91, 92, 93, 94, 95, 96, 97, 98 or 99% identical to SEQ ID NO: 64; or
(3) a heavy chain variable region (VH) comprising an amino acid sequence at least 90, 91, 92, 93, 94, 95, 96, 97, 98 or 99% identical to SEQ ID NO: 145, and a light chain variable region (VL) comprising an amino acid sequence at least 90, 91, 92, 93, 94, 95, 96, 97, 98 or 99% identical to SEQ ID NO: 64.

Embodiment 13: The multispecific antibody or antigen-binding fragment thereof of embodiment 12, wherein in the second antigen binding domain that specifically binds to the second epitope of human cMET, one, two, three, four, five, six, seven, eight, nine, or ten amino acids within SEQ ID NO: 54, SEQ ID NO: 82, SEQ ID NO: 59, SEQ ID NO: 83, SEQ ID NO: 145 or SEQ ID NO: 64 have been inserted, deleted or substituted.

Embodiment 14: The multispecific antibody or antigen-binding fragment thereof of any one of embodiments 6-13, wherein the second antigen binding domain that specifically binds to the second epitope of human cMET comprises:
(1) a heavy chain variable region (VH) comprising an amino acid sequence of SEQ ID NO: 54, or SEQ ID NO: 82, and a light chain variable region (VL) comprising an amino acid sequence of SEQ ID NO: 64;
(2) a heavy chain variable region (VH) comprising an amino acid sequence of SEQ ID NO: 59, or SEQ ID NO: 83, and a light chain variable region (VL) comprising an amino acid sequence of SEQ ID NO: 64; or
(3) a heavy chain variable region (VH) comprising an amino acid sequence of SEQ ID NO: 145, and a light chain variable region (VL) comprising an amino acid sequence of SEQ ID NO: 64.

Embodiment 15: The multispecific antibody or antigen-binding fragment thereof of any one of embodiments 6-14, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(13) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(14) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(15) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(16) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; and
wherein the second antigen binding domain that specifically binds to the second epitope of human cMET comprises:
a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68.

Embodiment 16: The multispecific antibody or antigen-binding fragment thereof of any one of embodiments 6-15, wherein the first antigen binding domain that specifically binds to the first epitope of human cMET comprises:
(1) a heavy chain variable region (VH) comprising an amino acid sequence of SEQ ID NO: 4 or SEQ ID NO: 72, and a light chain variable region (VL) comprising an amino acid sequence of SEQ ID NO: 64;
(2) a heavy chain variable region (VH) comprising an amino acid sequence of SEQ ID NO: 9 or SEQ ID NO: 73, and a light chain variable region (VL) comprising an amino acid sequence of SEQ ID NO: 64;
(3) a heavy chain variable region (VH) comprising an amino acid sequence of SEQ ID NO: 14, or SEQ ID NO: 74, and a light chain variable region (VL) comprising an amino acid sequence of SEQ ID NO: 64;
(4) a heavy chain variable region (VH) comprising an amino acid sequence of SEQ ID NO: 19, or SEQ ID NO: 75, and a light chain variable region (VL) comprising an amino acid sequence of SEQ ID NO: 64;
(5) a heavy chain variable region (VH) comprising an amino acid sequence of SEQ ID NO: 24, or SEQ ID NO: 76, and a light chain variable region (VL) of SEQ ID NO: 64;
(6) a heavy chain variable region (VH) comprising an amino acid sequence of SEQ ID NO: 29, or SEQ ID NO: 77, and a light chain variable region (VL) comprising an amino acid sequence of SEQ ID NO: 64;
(7) a heavy chain variable region (VH) comprising an amino acid sequence of SEQ ID NO: 34, or SEQ ID NO: 78, and a light chain variable region (VL) comprising an amino acid sequence of SEQ ID NO: 64;
(8) a heavy chain variable region (VH) comprising an amino acid sequence of SEQ ID NO: 39, or SEQ ID NO: 79, and a light chain variable region (VL) comprising an amino acid sequence of SEQ ID NO: 64;
(9) a heavy chain variable region (VH) comprising an amino acid sequence of SEQ ID NO: 44, or SEQ ID NO: 80, and a light chain variable region (VL) comprising an amino acid sequence of SEQ ID NO: 64;
(10) a heavy chain variable region (VH) comprising an amino acid sequence of SEQ ID NO: 49, or SEQ ID NO: 81, and a light chain variable region (VL) comprising an amino acid sequence of SEQ ID NO: 64;
(11) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64; or
(12) a heavy chain variable region (VH) comprising an amino acid sequence of SEQ ID NO: 144, and a light chain variable region (VL) comprising an amino acid sequence of SEQ ID NO: 64; and
wherein the second antigen binding domain that specifically binds to the second epitope of human cMET comprises:
a heavy chain variable region (VH) comprising an amino acid sequence of SEQ ID NO: 54, SEQ ID NO: 82, or SEQ ID NO: 145, and a light chain variable region (VL) comprising an amino acid sequence of SEQ ID NO: 64.

Embodiment 17: The multispecific antibody or antigen-binding fragment thereof of any one of embodiments 6-14, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(13) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(14) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(15) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(16) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; and
wherein the second antigen binding domain that specifically binds to the second epitope of human cMET comprises:
a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68.

Embodiment 18: The multispecific antibody or antigen-binding fragment thereof of any one of embodiments 6-14 and 17, wherein the first antigen binding domain that specifically binds to the first epitope of human cMET comprises:
(1) a heavy chain variable region (VH) comprising an amino acid sequence of SEQ ID NO: 4 or SEQ ID NO: 72, and a light chain variable region (VL) comprising an amino acid sequence of SEQ ID NO: 64;
(2) a heavy chain variable region (VH) comprising an amino acid sequence of SEQ ID NO: 9 or SEQ ID NO: 73, and a light chain variable region (VL) comprising an amino acid sequence of SEQ ID NO: 64;
(3) a heavy chain variable region (VH) comprising an amino acid sequence of SEQ ID NO: 14, or SEQ ID NO: 74, and a light chain variable region (VL) comprising an amino acid sequence of SEQ ID NO: 64;
(4) a heavy chain variable region (VH) comprising an amino acid sequence of SEQ ID NO: 19, or SEQ ID NO: 75, and a light chain variable region (VL) comprising an amino acid sequence of SEQ ID NO: 64;
(5) a heavy chain variable region (VH) comprising an amino acid sequence of SEQ ID NO: 24, or SEQ ID NO: 76, and a light chain variable region (VL) of SEQ ID NO: 64;
(6) a heavy chain variable region (VH) comprising an amino acid sequence of SEQ ID NO: 29, or SEQ ID NO: 77, and a light chain variable region (VL) comprising an amino acid sequence of SEQ ID NO: 64;
(7) a heavy chain variable region (VH) comprising an amino acid sequence of SEQ ID NO: 34, or SEQ ID NO: 78, and a light chain variable region (VL) comprising an amino acid sequence of SEQ ID NO: 64;
(8) a heavy chain variable region (VH) comprising an amino acid sequence of SEQ ID NO: 39, or SEQ ID NO: 79, and a light chain variable region (VL) comprising an amino acid sequence of SEQ ID NO: 64;
(9) a heavy chain variable region (VH) comprising an amino acid sequence of SEQ ID NO: 44, or SEQ ID NO: 80, and a light chain variable region (VL) comprising an amino acid sequence of SEQ ID NO: 64;
(10) a heavy chain variable region (VH) comprising an amino acid sequence of SEQ ID NO: 49, or SEQ ID NO: 81, and a light chain variable region (VL) comprising an amino acid sequence of SEQ ID NO: 64;
(11) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64; or
(12) a heavy chain variable region (VH) comprising an amino acid sequence of SEQ ID NO: 144, and a light chain variable region (VL) comprising an amino acid sequence of SEQ ID NO: 64; and
wherein the second antigen binding domain that specifically binds to the second epitope of human cMET comprises:
a heavy chain variable region (VH) comprising an amino acid sequence of SEQ ID NO: 59, or SEQ ID NO: 83, and a light chain variable region (VL) comprising an amino acid sequence of SEQ ID NO: 64.

Embodiment 19: The multispecific antibody or antigen-binding fragment thereof of any one of embodiments 6-18, wherein the multispecific antibody or antigen-binding fragment thereof is a monoclonal antibody, a human engineered antibody, a single chain antibody (scFv), a Fab fragment, a Fab′ fragment, or a F(ab′)2 fragment.

Embodiment 20: The multispecific antibody or antigen-binding fragment thereof of any one of embodiments 6-19, wherein the first antigen binding domain that specifically binds to the first epitope of human cMET is a monoclonal antibody, a human engineered antibody, a single chain antibody (scFv), a Fab fragment, a Fab′ fragment, or a F(ab′)2 fragment; and
the second antigen binding domain that specifically binds to the second epitope of human cMET is a monoclonal antibody, a human engineered antibody, a single chain antibody (scFv), a Fab fragment, a Fab′ fragment, or a F(ab′)2 fragment.

Embodiment 21: The multispecific antibody or antigen-binding fragment thereof of any one of embodiments 6-20, wherein the multispecific antibody is a bispecific antibody, or trispecific antibody.

Embodiment 22: The multispecific antibody or antigen-binding fragment thereof of any one of embodiments 6-21, wherein the first antigen binding domain that specifically binds to the first epitope of human cMET comprises a first heavy chain constant region comprising SEQ ID NO: 95 and the second antigen binding domain that specifically binds to the second epitope of human cMET comprises a second heavy chain constant region comprising SEQ ID NO: 96; or
wherein the first antigen binding domain that specifically binds to the first epitope of human cMET comprises a first heavy chain constant region comprising SEQ ID NO: 96 and the second antigen binding domain that specifically binds to the second epitope of human cMET comprises a second heavy chain constant region comprising SEQ ID NO: 95.

Embodiment 23: The multispecific antibody or antigen-binding fragment thereof of any one of embodiments 6-22, further comprising a third antigen binding domain that specifically binds to a human tumor-associated antigen (TAA).

Embodiment 24: The multispecific antibody or antigen-binding fragment thereof of any one of embodiments 23, wherein the TAA is EGFR.

Embodiment 25: The multispecific antibody or antigen-binding fragment thereof of any one of embodiments 6-24, further comprising an amino acid linker, wherein the amino acid linker is any sequence of SEQ ID NO: 97 to SEQ ID NO: 139.

Embodiment 26: The antibody, multispecific antibody or antigen-binding fragment thereof of any one of the preceding embodiments, wherein the antibody or antigen-binding fragment thereof comprises a heavy chain constant region of the subclass of IgG1, IgG2, IgG3, or IgG4, and/or a light chain constant region of the type of kappa or lambda.

Embodiment 27: The antibody, multispecific antibody or antigen-binding fragment thereof of any one of the preceding embodiments, wherein the antibody or antigen-binding fragment thereof comprises a heavy chain constant region of the subclass of IgG1, and a light chain constant region of the type of kappa.

Embodiment 28: The antibody, multispecific antibody or antigen-binding fragment thereof of any one of the preceding embodiments, wherein the antibody or antigen-binding fragment thereof has antibody dependent cellular cytotoxicity (ADCC), antibody-dependent cellular phagocytosis (ADCP) or complement dependent cytotoxicity (CDC).

Embodiment 29: The antibody, multispecific antibody or antigen-binding fragment thereof of any one of the preceding embodiments, wherein the antibody or antigen-binding fragment thereof has reduced glycosylation or no glycosylation or is hypofucosylated.

Embodiment 30: The antibody, multispecific antibody or antigen-binding fragment thereof of any one of the preceding embodiments, wherein the antibody or antigen-binding fragment thereof is afucosylated.

Embodiment 31: The antibody, multispecific antibody or antigen-binding fragment thereof of any one of the preceding embodiments, wherein the antibody or antigen-binding fragment thereof comprises increased bisecting GlcNac structures.

Embodiment 32: The antibody, multispecific antibody or antigen-binding fragment thereof of any one of the preceding embodiments, wherein the antibody or antigen-binding fragment thereof comprises a Fc domain, and wherein the Fc domain is an IgG1 Fc with extended half-life.

Embodiment 33: The antibody, multispecific antibody or antigen-binding fragment thereof of any one of the preceding embodiments, wherein the antibody or antigen-binding fragment thereof is conjugated to a cytotoxin.

Embodiment 34: The antibody, multispecific antibody or antigen-binding fragment thereof of any one of the preceding embodiments, wherein the antibody or antigen-binding fragment thereof is conjugated to a cytotoxin via a cytotoxin linker.

Embodiment 35: A pharmaceutical composition comprising the antibody, multispecific antibody or antigen-binding fragment thereof of any one of the preceding embodiments and a pharmaceutically acceptable carrier.

Embodiment 36: A method of treating a cancer comprising administering to a patient in need a therapeutically effective amount of the antibody, multispecific antibody or antigen-binding fragment thereof of any one of embodiments 1-34, or the pharmaceutical composition of embodiment 35.

Embodiment 37: The method of embodiment 36, wherein the cancer harbors a cMET genetic alteration and/or the growth of the cancer cell is driven by cMET signaling.

Embodiment 38: The method of embodiment 37, wherein the cMET signaling is ligand independent.

Embodiment 39: The method of embodiment 37, wherein the cMET signaling is ligand dependent.

Embodiment 40: The method of embodiment 37, wherein the cMET genetic alteration is cMET overexpression, genomic amplification, and/or mutation, which results in constitutively active cMET signaling.

Embodiment 41: The method of any one of embodiments 36-40, wherein the cancer is gastric cancer, colorectal cancer, lung cancer, liver cancer, head and neck cancer, kidney cancer, breast cancer, or brain cancer.

Embodiment 42: The method of embodiment 41, wherein the lung cancer is non-small cell lung cancer (NSCLC) or small cell lung cancer (SCLC).

Embodiment 43: The method of embodiment 42, wherein the non-small cell lung cancer is squamous non-small cell lung cancer.

Embodiment 44: The method of embodiment 41, wherein the liver cancer is hepatocellular carcinoma.

Embodiment 45: The method of embodiment 41, wherein the head and neck cancer is head and neck squamous cell carcinoma.

Embodiment 46: The method of any one of embodiments 36-45, wherein the antibody or antigen-binding fragment thereof is administered in combination with another therapeutic agent.

Embodiment 47: The method of embodiment 46, wherein the therapeutic agent is an immune checkpoint inhibitor.

Embodiment 48: The method of embodiment 46, wherein the therapeutic agent is an anti-PD-1 antibody.

Embodiment 49: The method of embodiment 48, wherein the anti-PD1 antibody is Tislelizumab.

Embodiment 50: A multispecific antibody or antigen-binding fragment thereof, comprising
a first antigen binding domain that specifically binds a first epitope of human cMET;
a second antigen binding domain that specifically binds to a second epitope of human cMET; and
a third antigen binding domain that specifically binds to human EGFR;
wherein the first epitope is distinct from the second epitope, or wherein the first antigen binding domain does not compete with the second antigen binding domain.

Embodiment 51: The multispecific antibody or antigen-binding fragment thereof of embodiment 50, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region that comprises (a) a HCDR1 of SEQ ID NO: 6, (b) a HCDR2 of SEQ ID NO: 7, (c) a HCDR3 of SEQ ID NO: 84 and a light chain variable region that comprises: (d) a LCDR1 of SEQ ID NO: 66, (e) a LCDR2 of SEQ ID NO: 67, and (f) a LCDR3 of SEQ ID NO: 68;
(13) a heavy chain variable region that comprises (a) a HCDR1 of SEQ ID NO: 6, (b) a HCDR2 of SEQ ID NO: 7, (c) a HCDR3 of SEQ ID NO: 86 and a light chain variable region that comprises: (d) a LCDR1 of SEQ ID NO: 66, (e) a LCDR2 of SEQ ID NO: 67, and (f) a LCDR3 of SEQ ID NO: 68;
(14) a heavy chain variable region that comprises (a) a HCDR1 of SEQ ID NO: 6, (b) a HCDR2 of SEQ ID NO: 7, (c) a HCDR3 of SEQ ID NO: 88 and a light chain variable region that comprises: (d) a LCDR1 of SEQ ID NO: 66, (e) a LCDR2 of SEQ ID NO: 67, and (f) a LCDR3 of SEQ ID NO: 68;
(15) a heavy chain variable region that comprises (a) a HCDR1 of SEQ ID NO: 6, (b) a HCDR2 of SEQ ID NO: 7, (c) a HCDR3 of SEQ ID NO: 90 and a light chain variable region that comprises: (d) a LCDR1 of SEQ ID NO: 66, (e) a LCDR2 of SEQ ID NO: 67, and (f) a LCDR3 of SEQ ID NO: 68; or
(16) a heavy chain variable region that comprises (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68.

Embodiment 52: The multispecific antibody or antigen-binding fragment thereof of any one of embodiments 50-51, wherein the first antigen binding domain that specifically binds to the first epitope of human cMET comprises:
(1) a heavy chain variable region (VH) comprising an amino acid sequence at least 90, 91, 92, 93, 94, 95, 96, 97, 98 or 99% identical to SEQ ID NO: 85, SEQ ID NO: 87, SEQ ID NO: 89, SEQ ID NO: 91, or SEQ ID NO: 93, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 4 or SEQ ID NO: 72, and a light chain variable region (VL) comprising an amino acid sequence at least 90, 91, 92, 93, 94, 95, 96, 97, 98 or 99% identical to SEQ ID NO: 64;
(3) a heavy chain variable region (VH) comprising an amino acid sequence at least 90, 91, 92, 93, 94, 95, 96, 97, 98 or 99% identical to SEQ ID NO: 9 or SEQ ID NO: 73, and a light chain variable region (VL) comprising an amino acid sequence at least 90, 91, 92, 93, 94, 95, 96, 97, 98 or 99% identical to SEQ ID NO: 64;
(4) a heavy chain variable region (VH) comprising an amino acid sequence at least 90, 91, 92, 93, 94, 95, 96, 97, 98 or 99% identical to SEQ ID NO: 14 or SEQ ID NO: 74, and a light chain variable region (VL) comprising an amino acid sequence at least 90, 91, 92, 93, 94, 95, 96, 97, 98 or 99% identical to SEQ ID NO: 64;
(5) a heavy chain variable region (VH) comprising an amino acid sequence at least 90, 91, 92, 93, 94, 95, 96, 97, 98 or 99% identical to SEQ ID NO: 19 or SEQ ID NO: 75, and a light chain variable region (VL) comprising an amino acid sequence at least 90, 91, 92, 93, 94, 95, 96, 97, 98 or 99% identical to SEQ ID NO: 64;
(6) a heavy chain variable region (VH) comprising an amino acid sequence at least 90, 91, 92, 93, 94, 95, 96, 97, 98 or 99% identical to SEQ ID NO: 24 or SEQ ID NO: 76, and a light chain variable region (VL) comprising an amino acid sequence at least 90, 91, 92, 93, 94, 95, 96, 97, 98 or 99% identical to SEQ ID NO: 64;
(7) a heavy chain variable region (VH) comprising an amino acid sequence at least 90, 91, 92, 93, 94, 95, 96, 97, 98 or 99% identical to SEQ ID NO: 29 or SEQ ID NO: 77, and a light chain variable region (VL) comprising an amino acid sequence at least 90, 91, 92, 93, 94, 95, 96, 97, 98 or 99% identical to SEQ ID NO: 64;
(8) a heavy chain variable region (VH) comprising an amino acid sequence at least 90, 91, 92, 93, 94, 95, 96, 97, 98 or 99% identical to SEQ ID NO: 34 or SEQ ID NO: 78, and a light chain variable region (VL) comprising an amino acid sequence at least 90, 91, 92, 93, 94, 95, 96, 97, 98 or 99% identical to SEQ ID NO: 64;
(9) a heavy chain variable region (VH) comprising an amino acid sequence at least 90, 91, 92, 93, 94, 95, 96, 97, 98 or 99% identical to SEQ ID NO: 39 or SEQ ID NO: 79, and a light chain variable region (VL) comprising an amino acid sequence at least 90, 91, 92, 93, 94, 95, 96, 97, 98 or 99% identical to SEQ ID NO: 64;
(10) a heavy chain variable region (VH) comprising an amino acid sequence at least 90, 91, 92, 93, 94, 95, 96, 97, 98 or 99% identical to SEQ ID NO: 44 or SEQ ID NO: 80, and a light chain variable region (VL) comprising an amino acid sequence at least 90, 91, 92, 93, 94, 95, 96, 97, 98 or 99% identical to SEQ ID NO: 64;
(11) a heavy chain variable region (VH) comprising an amino acid sequence at least 90, 91, 92, 93, 94, 95, 96, 97, 98 or 99% identical to SEQ ID NO: 49 or SEQ ID NO: 81, and a light chain variable region (VL) comprising an amino acid sequence at least 90, 91, 92, 93, 94, 95, 96, 97, 98 or 99% identical to SEQ ID NO: 64; or
(12) a heavy chain variable region (VH) comprising an amino acid sequence at least 90, 91, 92, 93, 94, 95, 96, 97, 98 or 99% identical to SEQ ID NO: 144, and a light chain variable region (VL) comprising an amino acid sequence at least 90, 91, 92, 93, 94, 95, 96, 97, 98 or 99% identical to SEQ ID NO: 64.

Embodiment 53: The multispecific antibody or antigen-binding fragment thereof of embodiment 52, wherein in the first antigen binding domain that specifically binds to the first epitope of human cMET, one, two, three, four, five, six, seven, eight, nine, or ten amino acids within SEQ ID NO: 85, SEQ ID NO: 87, SEQ ID NO: 89, SEQ ID NO: 91, SEQ ID NO: 93, SEQ ID NO: 4, SEQ ID NO: 72, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81, SEQ ID NO: 144, or SEQ ID NO: 64 have been inserted, deleted or substituted.

Embodiment 54: The multispecific antibody or antigen-binding fragment thereof of any one of embodiments 50-53, wherein the first antigen binding domain that specifically binds to the first epitope of human cMET comprises:
(1) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64;
(2) a heavy chain variable region (VH) comprising an amino acid sequence of SEQ ID NO: 4 or SEQ ID NO: 72, and a light chain variable region (VL) comprising an amino acid sequence of SEQ ID NO: 64;
(3) a heavy chain variable region (VH) comprising an amino acid sequence of SEQ ID NO: 9 or SEQ ID NO: 73, and a light chain variable region (VL) comprising an amino acid sequence of SEQ ID NO: 64;
(4) a heavy chain variable region (VH) comprising an amino acid sequence of SEQ ID NO: 14, or SEQ ID NO: 74, and a light chain variable region (VL) comprising an amino acid sequence of SEQ ID NO: 64;
(5) a heavy chain variable region (VH) comprising an amino acid sequence of SEQ ID NO: 19, or SEQ ID NO: 75, and a light chain variable region (VL) comprising an amino acid sequence of SEQ ID NO: 64;
(6) a heavy chain variable region (VH) comprising an amino acid sequence of SEQ ID NO: 24, or SEQ ID NO: 76, and a light chain variable region (VL) of SEQ ID NO: 64;
(7) a heavy chain variable region (VH) comprising an amino acid sequence of SEQ ID NO: 29, or SEQ ID NO: 77, and a light chain variable region (VL) comprising an amino acid sequence of SEQ ID NO: 64;
(8) a heavy chain variable region (VH) comprising an amino acid sequence of SEQ ID NO: 34, or SEQ ID NO: 78, and a light chain variable region (VL) comprising an amino acid sequence of SEQ ID NO: 64;
(9) a heavy chain variable region (VH) comprising an amino acid sequence of SEQ ID NO: 39, or SEQ ID NO: 79, and a light chain variable region (VL) comprising an amino acid sequence of SEQ ID NO: 64;
(10) a heavy chain variable region (VH) comprising an amino acid sequence of SEQ ID NO: 44 or SEQ ID NO: 80, and a light chain variable region (VL) comprising an amino acid sequence of SEQ ID NO: 64;
(11) a heavy chain variable region (VH) comprising an amino acid sequence of SEQ ID NO: 49 or SEQ ID NO: 81, and a light chain variable region (VL) comprising an amino acid sequence of SEQ ID NO: 64; or
(12) a heavy chain variable region (VH) comprising an amino acid sequence of SEQ ID NO: 144, and a light chain variable region (VL) comprising an amino acid sequence of SEQ ID NO: 64.

Embodiment 55: The multispecific antibody or antigen-binding fragment thereof of any one of embodiments 50-54, wherein the second antigen binding domain that specifically binds to the second epitope of human cMET comprises:
(1) a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; or
(2) a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68.

Embodiment 56: The multispecific antibody or antigen-binding fragment thereof of any one of embodiments 50-55, wherein the second antigen binding domain that specifically binds to the second epitope of human cMET comprises:
(1) a heavy chain variable region (VH) comprising an amino acid sequence at least 90, 91, 92, 93, 94, 95, 96, 97, 98 or 99% identical to SEQ ID NO: 54, or SEQ ID NO: 82, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 59, or SEQ ID NO: 83, and a light chain variable region (VL) comprising an amino acid sequence at least 90, 91, 92, 93, 94, 95, 96, 97, 98 or 99% identical to SEQ ID NO: 64; or
(3) a heavy chain variable region (VH) comprising an amino acid sequence at least 90, 91, 92, 93, 94, 95, 96, 97, 98 or 99% identical to SEQ ID NO: 145, and a light chain variable region (VL) comprising an amino acid sequence at least 90, 91, 92, 93, 94, 95, 96, 97, 98 or 99% identical to SEQ ID NO: 64.

Embodiment 57: The multispecific antibody or antigen-binding fragment thereof of embodiment 56, wherein in the second antigen binding domain that specifically binds to the second epitope of human cMET, one, two, three, four, five, six, seven, eight, nine, or ten amino acids within SEQ ID NO: 54, SEQ ID NO: 82, SEQ ID NO: 59, SEQ ID NO: 83, SEQ ID NO: 145, or SEQ ID NO: 64 have been inserted, deleted or substituted.

Embodiment 58: The multispecific antibody or antigen-binding fragment thereof of any one of embodiments 50-57, wherein the second antigen binding domain that specifically binds to the second epitope of human cMET comprises:
(1) a heavy chain variable region (VH) comprising an amino acid sequence of SEQ ID NO: 54, or SEQ ID NO: 82, and a light chain variable region (VL) comprising an amino acid sequence of SEQ ID NO: 64;
(2) a heavy chain variable region (VH) comprising an amino acid sequence of SEQ ID NO: 59, or SEQ ID NO: 83, and a light chain variable region (VL) comprising an amino acid sequence of SEQ ID NO: 64; or
(3) a heavy chain variable region (VH) comprising an amino acid sequence of SEQ ID NO: 145, and a light chain variable region (VL) comprising an amino acid sequence of SEQ ID NO: 64.

Embodiment 59: The multispecific antibody or antigen-binding fragment thereof of any one of embodiments 24 and 50-58, wherein the third antigen binding domain that specifically binds to human EGFR comprises:
a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

Embodiment 60: The multispecific antibody or antigen-binding fragment thereof of any one of embodiments 24 and 50-59, wherein the third antigen binding domain that specifically binds to human EGFR comprises:
a heavy chain variable region (VH) comprising an amino acid sequence of SEQ ID NO: 142, and
a light chain variable region (VL) comprising an amino acid sequence of SEQ ID NO: 143.

Embodiment 61: The multispecific antibody or antigen-binding fragment thereof of any one of embodiments 24 and 50-60, wherein
the first antigen binding domain that specifically binds to the first epitope of human cMET comprises:
(1) a heavy chain variable region (VH) that comprises (a) a HCDR1 of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region that comprises (a) a HCDR1 of SEQ ID NO: 6, (b) a HCDR2 of SEQ ID NO: 7, (c) a HCDR3 of SEQ ID NO: 84 and a light chain variable region that comprises: (d) a LCDR1 of SEQ ID NO: 66, (e) a LCDR2 of SEQ ID NO: 67, and (f) a LCDR3 of SEQ ID NO: 68;
(13) a heavy chain variable region that comprises (a) a HCDR1 of SEQ ID NO: 6, (b) a HCDR2 of SEQ ID NO: 7, (c) a HCDR3 of SEQ ID NO: 86 and a light chain variable region that comprises: (d) a LCDR1 of SEQ ID NO: 66, (e) a LCDR2 of SEQ ID NO: 67, and (f) a LCDR3 of SEQ ID NO: 68;
(14) a heavy chain variable region that comprises (a) a HCDR1 of SEQ ID NO: 6, (b) a HCDR2 of SEQ ID NO: 7, (c) a HCDR3 of SEQ ID NO: 88 and a light chain variable region that comprises: (d) a LCDR1 of SEQ ID NO: 66, (e) a LCDR2 of SEQ ID NO: 67, and (f) a LCDR3 of SEQ ID NO: 68;
(15) a heavy chain variable region that comprises (a) a HCDR1 of SEQ ID NO: 6, (b) a HCDR2 of SEQ ID NO: 7, (c) a HCDR3 of SEQ ID NO: 90 and a light chain variable region that comprises: (d) a LCDR1 of SEQ ID NO: 66, (e) a LCDR2 of SEQ ID NO: 67, and (f) a LCDR3 of SEQ ID NO: 68; or
(16) a heavy chain variable region that comprises (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises:
a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises:
a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

Embodiment 62: The multispecific antibody or antigen-binding fragment thereof of any one of embodiments 24 and 50-61, wherein
the first antigen binding domain that specifically binds to the first epitope of human cMET comprises:
(1) a heavy chain variable region (VH) comprising an amino acid sequence of SEQ ID NO: 4 or SEQ ID NO: 72, and a light chain variable region (VL) comprising an amino acid sequence of SEQ ID NO: 64;
(2) a heavy chain variable region (VH) comprising an amino acid sequence of SEQ ID NO: 9 or SEQ ID NO: 73, and a light chain variable region (VL) comprising an amino acid sequence of SEQ ID NO: 64;
(3) a heavy chain variable region (VH) comprising an amino acid sequence of SEQ ID NO: 14, or SEQ ID NO: 74, and a light chain variable region (VL) comprising an amino acid sequence of SEQ ID NO: 64;
(4) a heavy chain variable region (VH) comprising an amino acid sequence of SEQ ID NO: 19, or SEQ ID NO: 75, and a light chain variable region (VL) comprising an amino acid sequence of SEQ ID NO: 64;
(5) a heavy chain variable region (VH) comprising an amino acid sequence of SEQ ID NO: 24, or SEQ ID NO: 76, and a light chain variable region (VL) of SEQ ID NO: 64;
(6) a heavy chain variable region (VH) comprising an amino acid sequence of SEQ ID NO: 29, or SEQ ID NO: 77, and a light chain variable region (VL) comprising an amino acid sequence of SEQ ID NO: 64;
(7) a heavy chain variable region (VH) comprising an amino acid sequence of SEQ ID NO: 34, or SEQ ID NO: 78, and a light chain variable region (VL) comprising an amino acid sequence of SEQ ID NO: 64;
(8) a heavy chain variable region (VH) comprising an amino acid sequence of SEQ ID NO: 39, or SEQ ID NO: 79, and a light chain variable region (VL) comprising an amino acid sequence of SEQ ID NO: 64;
(9) a heavy chain variable region (VH) comprising an amino acid sequence of SEQ ID NO: 44, or SEQ ID NO: 80, and a light chain variable region (VL) comprising an amino acid sequence of SEQ ID NO: 64;
(10) a heavy chain variable region (VH) comprising an amino acid sequence of SEQ ID NO: 49, or SEQ ID NO: 81, and a light chain variable region (VL) comprising an amino acid sequence of SEQ ID NO: 64;
(11) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64; or
(12) a heavy chain variable region (VH) comprising an amino acid sequence of SEQ ID NO: 144, and a light chain variable region (VL) comprising an amino acid sequence of SEQ ID NO: 64;
the second antigen binding domain that specifically binds to the second epitope of human cMET comprises:
a heavy chain variable region (VH) comprising an amino acid sequence of SEQ ID NO: 54, SEQ ID NO: 82, or SEQ ID NO: 145, and a light chain variable region (VL) comprising an amino acid sequence of SEQ ID NO: 64; and
the third antigen binding domain that specifically binds to human EGFR comprises:
a heavy chain variable region (VH) comprising an amino acid sequence of SEQ ID NO: 142, and
a light chain variable region (VL) comprising an amino acid sequence of SEQ ID NO: 143.

Embodiment 63: The multispecific antibody or antigen-binding fragment thereof of any one of embodiments 24 and 50-60, wherein
the first antigen binding domain that specifically binds to the first epitope of human cMET comprises:
(1) a heavy chain variable region (VH) that comprises (a) a HCDR1 of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region that comprises (a) a HCDR1 of SEQ ID NO: 6, (b) a HCDR2 of SEQ ID NO: 7, (c) a HCDR3 of SEQ ID NO: 84 and a light chain variable region that comprises: (d) a LCDR1 of SEQ ID NO: 66, (e) a LCDR2 of SEQ ID NO: 67, and (f) a LCDR3 of SEQ ID NO: 68;
(13) a heavy chain variable region that comprises (a) a HCDR1 of SEQ ID NO: 6, (b) a HCDR2 of SEQ ID NO: 7, (c) a HCDR3 of SEQ ID NO: 86 and a light chain variable region that comprises: (d) a LCDR1 of SEQ ID NO: 66, (e) a LCDR2 of SEQ ID NO: 67, and (f) a LCDR3 of SEQ ID NO: 68;
(14) a heavy chain variable region that comprises (a) a HCDR1 of SEQ ID NO: 6, (b) a HCDR2 of SEQ ID NO: 7, (c) a HCDR3 of SEQ ID NO: 88 and a light chain variable region that comprises: (d) a LCDR1 of SEQ ID NO: 66, (e) a LCDR2 of SEQ ID NO: 67, and (f) a LCDR3 of SEQ ID NO: 68;
(15) a heavy chain variable region that comprises (a) a HCDR1 of SEQ ID NO: 6, (b) a HCDR2 of SEQ ID NO: 7, (c) a HCDR3 of SEQ ID NO: 90 and a light chain variable region that comprises: (d) a LCDR1 of SEQ ID NO: 66, (e) a LCDR2 of SEQ ID NO: 67, and (f) a LCDR3 of SEQ ID NO: 68; or
(16) a heavy chain variable region that comprises (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises:
a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises:
a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

Embodiment 64: The multispecific antibody or antigen-binding fragment thereof of any one of embodiments 24, 50-60 and 63, wherein
the first antigen binding domain that specifically binds to the first epitope of human cMET comprises:
(1) a heavy chain variable region (VH) comprising an amino acid sequence of SEQ ID NO: 4 or SEQ ID NO: 72, and a light chain variable region (VL) comprising an amino acid sequence of SEQ ID NO: 64;
(2) a heavy chain variable region (VH) comprising an amino acid sequence of SEQ ID NO: 9 or SEQ ID NO: 73, and a light chain variable region (VL) comprising an amino acid sequence of SEQ ID NO: 64;
(3) a heavy chain variable region (VH) comprising an amino acid sequence of SEQ ID NO: 14, or SEQ ID NO: 74, and a light chain variable region (VL) comprising an amino acid sequence of SEQ ID NO: 64;
(4) a heavy chain variable region (VH) comprising an amino acid sequence of SEQ ID NO: 19, or SEQ ID NO: 75, and a light chain variable region (VL) comprising an amino acid sequence of SEQ ID NO: 64;
(5) a heavy chain variable region (VH) comprising an amino acid sequence of SEQ ID NO: 24, or SEQ ID NO: 76, and a light chain variable region (VL) of SEQ ID NO: 64;
(6) a heavy chain variable region (VH) comprising an amino acid sequence of SEQ ID NO: 29, or SEQ ID NO: 77, and a light chain variable region (VL) comprising an amino acid sequence of SEQ ID NO: 64;
(7) a heavy chain variable region (VH) comprising an amino acid sequence of SEQ ID NO: 34, or SEQ ID NO: 78, and a light chain variable region (VL) comprising an amino acid sequence of SEQ ID NO: 64;
(8) a heavy chain variable region (VH) comprising an amino acid sequence of SEQ ID NO: 39, or SEQ ID NO: 79, and a light chain variable region (VL) comprising an amino acid sequence of SEQ ID NO: 64;
(9) a heavy chain variable region (VH) comprising an amino acid sequence of SEQ ID NO: 44, or SEQ ID NO: 80, and a light chain variable region (VL) comprising an amino acid sequence of SEQ ID NO: 64;
(10) a heavy chain variable region (VH) comprising an amino acid sequence of SEQ ID NO: 49, or SEQ ID NO: 81, and a light chain variable region (VL) comprising an amino acid sequence of SEQ ID NO: 64;
(11) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64; or
(12) a heavy chain variable region (VH) comprising an amino acid sequence of SEQ ID NO: 144, and a light chain variable region (VL) comprising an amino acid sequence of SEQ ID NO: 64;
the second antigen binding domain that specifically binds to the second epitope of human cMET comprises:
a heavy chain variable region (VH) comprising an amino acid sequence of SEQ ID NO: 59, or SEQ ID NO: 83, and a light chain variable region (VL) comprising an amino acid sequence of SEQ ID NO: 64; and
the third antigen binding domain that specifically binds to human EGFR comprises:
a heavy chain variable region (VH) comprising an amino acid sequence of SEQ ID NO: 142, and
a light chain variable region (VL) comprising an amino acid sequence of SEQ ID NO: 143.

Embodiment 65: The multispecific antibody or antigen-binding fragment thereof of any one of embodiments 24 and 50-64, wherein
the first antigen binding domain that specifically binds to the first epitope of human cMET comprises: a heavy chain variable region that comprises (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises: a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises: a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

Embodiment 66: The multispecific antibody or antigen-binding fragment thereof of any one of embodiments 24 and 50-65, wherein
the first antigen binding domain that specifically binds to the first epitope of human cMET comprises: a heavy chain variable region (VH) comprising an amino acid sequence of SEQ ID NO: 144, and a light chain variable region (VL) comprising an amino acid sequence of SEQ ID NO: 64;
the second antigen binding domain that specifically binds to the second epitope of human cMET comprises: a heavy chain variable region (VH) comprising an amino acid sequence of SEQ ID NO: 145, and a light chain variable region (VL) comprising an amino acid sequence of SEQ ID NO: 64; and
the third antigen binding domain that specifically binds to human EGFR comprises: a heavy chain variable region (VH) comprising an amino acid sequence of SEQ ID NO: 142, and a light chain variable region (VL) comprising an amino acid sequence of SEQ ID NO: 143.

Embodiment 67: The multispecific antibody or antigen-binding fragment thereof of any one of embodiments 50-66, wherein the multispecific antibody or antigen-binding fragment thereof is a monoclonal antibody, a human engineered antibody, a single chain antibody (scFv), a Fab fragment, a Fab′ fragment, or a F(ab′)2 fragment.

Embodiment 68: The multispecific antibody or antigen-binding fragment thereof of any one of embodiments 24 and 50-66, wherein the first antigen binding domain that specifically binds to the first epitope of human cMET is a monoclonal antibody, a human engineered antibody, a single chain antibody (scFv), a Fab fragment, a Fab′ fragment, or a F(ab′)2 fragment;
the second antigen binding domain that specifically binds to the second epitope of human cMET is a monoclonal antibody, a human engineered antibody, a single chain antibody (scFv), a Fab fragment, a Fab′ fragment, or a F(ab′)2 fragment; and
the third antigen binding domain that specifically binds to human EGFR is a monoclonal antibody, a human engineered antibody, a single chain antibody (scFv), a Fab fragment, a Fab′ fragment, or a F(ab′)2 fragment.

Embodiment 69: The multispecific antibody or antigen-binding fragment thereof of any one of embodiments 24 and 50-68, wherein the third antigen binding domain that specifically binds to human EGFR comprises a scFv comprising a VH having the amino acid of SEQ ID NO: 142 and a VL having an amino acid of SEQ ID NO: 143;
optionally the VH and VL are connected via a first amino acid linker;
optionally the first amino acid linker is any sequence of SEQ ID NO: 97 to SEQ ID NO: 139;
preferably, the first amino acid linker is SEQ ID NO: 139.

Embodiment 70: The multispecific antibody or antigen-binding fragment thereof of any one of embodiments 24 and 50-69, wherein the third antigen binding domain that specifically binds to human EGFR comprises a scFv having the amino acid sequence of SEQ ID NO: 161.

Embodiment 71: The multispecific antibody or antigen-binding fragment thereof of any one of embodiments 24 and 50-70, wherein the multispecific antibody is a trispecific antibody.

Embodiment 72: The multispecific antibody or antigen-binding fragment thereof of any one of embodiments 50-71, wherein the multispecific antibody or antigen-binding fragment thereof comprises a heavy chain constant region of the subclass of IgG1, IgG2, IgG3, or IgG4, and/or a light chain constant region of the type of kappa or lambda.

Embodiment 73: The multispecific antibody or antigen-binding fragment thereof of any one of embodiments 50-72, wherein the multispecific antibody or antigen-binding fragment thereof comprises a heavy chain constant region of the subclass of IgG1, and a light chain constant region of the type of kappa.

Embodiment 74: The multispecific antibody or antigen-binding fragment thereof of any one of embodiments 50-73, wherein the multispecific antibody or antigen-binding fragment thereof has antibody dependent cellular cytotoxicity (ADCC), antibody-dependent cellular phagocytosis (ADCP) or complement dependent cytotoxicity (CDC).

Embodiment 75: The multispecific antibody or antigen-binding fragment thereof of any one of embodiments 50-74, wherein the multispecific antibody or antigen-binding fragment thereof has reduced glycosylation or no glycosylation or is hypofucosylated.

Embodiment 76: The multispecific antibody or antigen-binding fragment thereof of any one of embodiments 50-75, wherein the multispecific antibody or antigen-binding fragment thereof is afucosylated.

Embodiment 77: The multispecific antibody or antigen-binding fragment thereof of any one of embodiments 50-76, wherein the multispecific antibody or antigen-binding fragment thereof comprises increased bisecting GlcNac structures.

Embodiment 78: The multispecific antibody or antigen-binding fragment thereof of any one of embodiments 50-77, wherein the multispecific antibody or antigen-binding fragment thereof comprises a Fc domain, and wherein the Fc domain is an IgG1 Fc with extended half-life.

Embodiment 79: The multispecific antibody or antigen-binding fragment thereof of any one of embodiments 50-78, wherein the first antigen binding domain that specifically binds to the first epitope of human cMET comprises a first heavy chain constant region comprising SEQ ID NO: 95, and the second antigen binding domain that specifically binds to the second epitope of human cMET comprises a second heavy chain constant region comprising SEQ ID NO: 96; or
wherein the first antigen binding domain that specifically binds to the first epitope of human cMET comprises a first heavy chain constant region comprising SEQ ID NO: 96, and the second antigen binding domain that specifically binds to the second epitope of human cMET comprises a second heavy chain constant region comprising SEQ ID NO: 95.

Embodiment 80: The multispecific antibody or antigen-binding fragment thereof of any one of embodiments 50-79, wherein the first antigen binding domain that specifically binds to the first epitope of human cMET comprises a first light chain constant region, and the second antigen binding domain that specifically binds to the second epitope of human cMET comprises a second light chain constant region;
optionally, the first light chain constant region and the second light chain constant region are different; or
optionally, the first light chain constant region and the second light chain constant region are the same.

Embodiment 81: The multispecific antibody or antigen-binding fragment thereof of any one of embodiments 50-80, further comprising a second amino acid linker, wherein the second amino acid linker is any sequence of SEQ ID NO: 97 to SEQ ID NO: 139, preferably, the second amino acid linker is SEQ ID NO: 139.

Embodiment 82: The multispecific antibody or antigen-binding fragment thereof of any one of embodiments 50-81, comprising a first polypeptide, a second polypeptide, a third polypeptide, and a fourth polypeptide, wherein
(1) a VL of the third antigen binding domain that specifically binds to human EGFR, optionally the first amino acid linker, a VH of the third antigen binding domain that specifically binds to human EGFR, optionally the second amino acid linker, a VH of the second antigen binding domain that specifically binds to the second epitope of human cMET, and the second heavy chain constant region are arranged in the first polypeptide in the direction of N terminal to C terminal;
(2) a VH of the first antigen binding domain that specifically binds to the first epitope of human cMET, and the first heavy chain constant region are arranged in the second polypeptide in the direction of N terminal to C terminal, or
a VL of the third antigen binding domain that specifically binds to human EGFR, optionally the first amino acid linker, a VH of the third antigen binding domain that specifically binds to human EGFR, optionally the second amino acid linker, a VH of the first antigen binding domain that specifically binds to the first epitope of human cMET, and the first heavy chain constant region are arranged in the second polypeptide in the direction of N terminal to C terminal;
(3) a VL of the second antigen binding domain that specifically binds to the second epitope of human cMET, and a second light chain constant region are arranged in the third polypeptide in the direction of N terminal to C terminal; and
(4) a VL of the first antigen binding domain that specifically binds to the first epitope of human cMET, and a first light chain constant region are arranged in the fourth polypeptide in the direction of N terminal to C terminal;
optionally, the second heavy chain constant region is SEQ ID NO: 95, and the first heavy chain constant region is SEQ ID NO: 96; or the second heavy chain constant region is SEQ ID NO: 96, and the first heavy chain constant region is SEQ ID NO: 95;
optionally, the first amino acid linker is SEQ ID NO: 139;
optionally, the second amino acid linker is SEQ ID NO: 139;
optionally, a VL of the first antigen binding domain and a VL of the second antigen binding domain are the same;
optionally, the first light chain constant region and the second light chain constant region are the same;
optionally, the third polypeptide and the fourth polypeptide are the same.

Embodiment 83: The multispecific antibody or antigen-binding fragment thereof of any one of embodiments 50-82, comprising a first polypeptide, a second polypeptide, a third polypeptide, and a fourth polypeptide, wherein
(1) a VL of the third antigen binding domain that specifically binds to human EGFR, optionally the first amino acid linker, a VH of the third antigen binding domain that specifically binds to human EGFR, optionally the second amino acid linker, a VH of the first antigen binding domain that specifically binds to the first epitope of human cMET, and the first heavy chain constant region are arranged in the first polypeptide in the direction of N terminal to C terminal;
(2) a VH of the second antigen binding domain that specifically binds to the second epitope of human cMET, and the second heavy chain constant region are arranged in the second polypeptide in the direction of N terminal to C terminal, or
a VL of the third antigen binding domain that specifically binds to human EGFR, optionally the first amino acid linker, a VH of the third antigen binding domain that specifically binds to human EGFR, optionally the second amino acid linker, a VH of the second antigen binding domain that specifically binds to the second epitope of human cMET, and the second heavy chain constant region are arranged in the second polypeptide in the direction of N terminal to C terminal;
(3) a VL of the first antigen binding domain that specifically binds to the first epitope of human cMET, and a first light chain constant region are arranged in the third polypeptide in the direction of N terminal to C terminal; and
(4) a VL of the second antigen binding domain that specifically binds to the second epitope of human cMET, and a second light chain constant region are arranged in the fourth polypeptide in the direction of N terminal to C terminal;
optionally, the second heavy chain constant region is SEQ ID NO: 95, and the first heavy chain constant region is SEQ ID NO: 96; or the second heavy chain constant region is SEQ ID NO: 96, and the first heavy chain constant region is SEQ ID NO: 95;
optionally, the first amino acid linker is SEQ ID NO: 139;
optionally, the second amino acid linker is SEQ ID NO: 139;
optionally, a VL of the first antigen binding domain and a VL of the second antigen binding domain are the same;
optionally, the first light chain constant region and the second light chain constant region are the same;
optionally, the third polypeptide and the fourth polypeptide are the same.

Embodiment 84: The multispecific antibody or antigen-binding fragment thereof of any one of embodiments 50-83, comprising a first polypeptide, a second polypeptide, a third polypeptide, and a fourth polypeptide, wherein
(1) a VL of the third antigen binding domain that specifically binds to human EGFR, optionally the first amino acid linker, a VH of the third antigen binding domain that specifically binds to human EGFR, optionally the second amino acid linker, a VH of the second antigen binding domain that specifically binds to the second epitope of human cMET, and the second heavy chain constant region are arranged in the first polypeptide in the direction of N terminal to C terminal;
(2) a VH of the first antigen binding domain that specifically binds to the first epitope of human cMET and the first heavy chain constant region are arranged in the second polypeptide in the direction of N terminal to C terminal;
(3) a VL of the second antigen binding domain that specifically binds to the second epitope of human cMET and a second light chain constant region are arranged in the third polypeptide in the direction of N terminal to C terminal; and
(4) a VL of the first antigen binding domain that specifically binds to the first epitope of human cMET and a first light chain constant region are arranged in the fourth polypeptide in the direction of N terminal to C terminal;
optionally, the second heavy chain constant region is SEQ ID NO: 95, and the first heavy chain constant region is SEQ ID NO: 96; or the second heavy chain constant region is SEQ ID NO: 96, and the first heavy chain constant region is SEQ ID NO: 95;
optionally, the first amino acid linker is SEQ ID NO: 139;
optionally, the second amino acid linker is SEQ ID NO: 139;
optionally, a VL of the first antigen binding domain and a VL of the second antigen binding domain are the same;
optionally, the first light chain constant region and the second light chain constant region are the same;
optionally, the third polypeptide and the fourth polypeptide are the same.

Embodiment 85: The multispecific antibody or antigen-binding fragment thereof of any one of embodiments 50-84, comprising a first polypeptide, a second polypeptide, and a third polypeptide, wherein
(1) the first polypeptide has an amino acid sequence of SEQ ID NO: 153, the second polypeptide has an amino acid sequence of SEQ ID NO: 150, and the third polypeptide has an amino acid sequence of SEQ ID NO: 148;
(2) the first polypeptide has an amino acid sequence of SEQ ID NO: 146, the second polypeptide has an amino acid sequence of SEQ ID NO: 147, and the third polypeptide has an amino acid sequence of SEQ ID NO: 148;
(3) the first polypeptide has an amino acid sequence of SEQ ID NO: 149, the second polypeptide has an amino acid sequence of SEQ ID NO: 150, and the third polypeptide has an amino acid sequence of SEQ ID NO: 151;
(4) the first polypeptide has an amino acid sequence of SEQ ID NO: 149, the second polypeptide has an amino acid sequence of SEQ ID NO: 150, and the third polypeptide has an amino acid sequence of SEQ ID NO: 152;
(5) the first polypeptide has an amino acid sequence of SEQ ID NO: 153, the second polypeptide has an amino acid sequence of SEQ ID NO: 154, and the third polypeptide has an amino acid sequence of SEQ ID NO: 148; or
(6) the first polypeptide has an amino acid sequence of SEQ ID NO: 146, the second polypeptide has an amino acid sequence of SEQ ID NO: 150, and the third polypeptide has an amino acid sequence of SEQ ID NO: 148.

Embodiment 86: The multispecific antibody or antigen-binding fragment thereof of any one of embodiments 50-85, wherein the multispecific antibody or antigen-binding fragment thereof comprises
(1) a first polypeptide of SEQ ID NO: 153, the second polypeptide of SEQ ID NO: 150, and the third polypeptide of SEQ ID NO: 148;
(2) the first polypeptide of SEQ ID NO: 146, the second polypeptide of SEQ ID NO: 147, and the third polypeptide of SEQ ID NO: 148;
(3) the first polypeptide of SEQ ID NO: 149, the second polypeptide of SEQ ID NO: 150, and the third polypeptide of SEQ ID NO: 151;
(4) the first polypeptide of SEQ ID NO: 149, the second polypeptide of SEQ ID NO: 150, and the third polypeptide of SEQ ID NO: 152;
(5) the first polypeptide of SEQ ID NO: 153, the second polypeptide of SEQ ID NO: 154, and the third polypeptide of SEQ ID NO: 148; or
(6) the first polypeptide of SEQ ID NO: 146, the second polypeptide of SEQ ID NO: 150, and the third polypeptide of SEQ ID NO: 148.

Embodiment 87: A pharmaceutical composition comprising the multispecific antibody or antigen-binding fragment thereof of any one of embodiments 50-86 and a pharmaceutically acceptable carrier.

Embodiment 88: A method of treating a cancer comprising administering to a patient in need a therapeutically effective amount of the multispecific antibody or antigen-binding fragment thereof of any one of embodiments 50-86, or the pharmaceutical composition of embodiment 87.

Embodiment 89: The method of embodiment 88, wherein the cancer harbors a cMET genetic alteration and/or the growth of the cancer cell is driven by cMET signaling.

Embodiment 90: The method of embodiment 89, wherein the cMET signaling is ligand independent.

Embodiment 91: The method of embodiment 89, wherein the cMET signaling is ligand dependent.

Embodiment 92: The method of embodiment 89, wherein the cMET genetic alteration is cMET overexpression, genomic amplification, and/or mutation, which results in constitutively active cMET signaling.

Embodiment 93: The method of embodiment 88, wherein the cancer harbors an EGFR activating mutation and/or the growth of the cancer cell is driven by EGFR signaling; optionally, the EGFR activating mutation is deletion or point mutation.

Embodiment 94: The method of embodiment 93, wherein the EGFR signaling is ligand independent.

Embodiment 95: The method of embodiment 93, wherein the EGFR signaling is ligand dependent.

Embodiment 96: The method of any one of embodiments 88-95, wherein the cancer is gastric cancer, colorectal cancer, lung cancer, liver cancer, head and neck cancer, kidney cancer, breast cancer, or brain cancer.

Embodiment 97: The method of embodiment 96, wherein the lung cancer is non-small cell lung cancer (NSCLC) or small cell lung cancer (SCLC).

Embodiment 98: The method of embodiment 97, wherein the non-small cell lung cancer is squamous non-small cell lung cancer.

Embodiment 99: The method of embodiment 96, wherein the liver cancer is hepatocellular carcinoma.

Embodiment 100: The method of embodiment 96, wherein the head and neck cancer is head and neck squamous cell carcinoma.

Embodiment 101: The method of any one of embodiments 88-100, wherein the multispecific antibody or antigen-binding fragment thereof is administered in combination with another therapeutic agent.

Embodiment 102: The method of embodiment 101, wherein the therapeutic agent is an immune checkpoint inhibitor.

Embodiment 103: The method of embodiment 101, wherein the therapeutic agent is an anti-PD-1 antibody.

Embodiment 104: The method of embodiment 103, wherein the anti-PD1 antibody is Tislelizumab.

Embodiment 105: An isolated nucleic acid that encodes the antibody, multispecific antibody or antigen-binding fragment thereof of any one of embodiments 1 to 34 and 50-86.

Embodiment 106: A vector comprising the nucleic acid of embodiment 105.

Embodiment 107: A host cell comprising the nucleic acid of embodiment 105 or the vector of embodiment 106.

Embodiment 108: A process for producing an antibody, multispecific antibody or antigen-binding fragment thereof comprising cultivating the host cell of embodiment 107 and recovering the antibody, multispecific antibody or antibody fragment from the culture.

In some embodiments, the present disclosure provides anti-human cMET antibodies or antigen-binding fragments thereof having high binding affinity to human cMET and/or able to block ligand dependent signaling.

In some embodiments, the present disclosure provides multispecific antibody or antigen-binding fragment thereof which binds to two distinct epitopes of human cMET (e.g., non-overlapping) or wherein the first antigen binding domain thereof targeting human cMET does not compete with the second antigen binding domain thereof targeting human cMET. Such multispecific antibodies or antigen-binding fragments thereof having any one or more of the following features:
(1) able to block ligand independent signaling;
(2) able to inhibit cancer cell proliferation; and
(3) able to block ligand induced signaling.

In some embodiments, the present disclosure provides multispecific antibodies or antigen-binding fragments thereof that specifically bind to human EGFR and two distinct epitopes of human cMET (e.g., non-overlapping) or multispecific antibodies or antigen-binding fragments thereof that specifically bind to human EGFR and human cMET, wherein the first antigen binding domain thereof targeting human cMET does not compete with the second antigen binding domain thereof targeting human cMET (e.g, EGFRxcMET biparatopic antibodies or antigen-binding fragments thereof) having high binding affinity to human cMET and human EGFR.

In some embodiments, the present disclosure provides EGFRxcMET biparatopic trispecific antibodies or antigen-binding fragments thereof having great or superior ADCC activity to cancer cells, including EGFR mutation/signaling driven cancer cells and/or cMET amplification/signaling driven cancer cells.

In some embodiments, the present disclosure provides EGFRxcMET biparatopic trispecific antibodies or antigen-binding fragments thereof having great ADCP activity to cancer cells, including EGFR mutation/signaling driven cancer cells and/or cMET amplification/signaling driven cancer cells.

In some embodiment, the present disclosure provides EGFRxcMET biparatopic trispecific antibodies or antigen-binding fragments thereof significantly blocking EGFR signaling and/or cMET signaling by down-regulating the receptor on the cell surface (e.g., by internalization). In addition, EGFRxcMET biparatopic trispecific antibodies or antigen-binding fragments thereof of the present disclosure have great anti-proliferation activity to EGFR mutation/signaling driven cancer cells, and/or have superior anti-proliferation activity to cMET amplification/signaling driven cancer cells.

In some embodiment, the present disclosure provides EGFRxcMET biparatopic trispecific antibodies or antigen-binding fragments thereof exhibiting great or superior killing effect on cancer cells, including EGFR mutation/signaling driven cancer cells and especially cMET amplification/signaling driven cancer cells.

In some embodiments, the present disclosure provides EGFRxcMET biparatopic trispecific antibodies or antigen-binding fragments thereof exhibiting great tumor growth inhibition in EGFR mutation/signaling driven tumor, and/or exhibit superior tumor growth inhibition in cMET amplification/signaling driven tumor.

In some embodiments, the present disclosure provides EGFRxcMET biparatopic trispecific antibodies or antigen-binding fragments thereof exhibiting less toxicity and/or better safety.

In some embodiments, the present disclosure provides multispecific antibodies or antigen-binding fragments thereof that specifically bind to human EGFR and two distinct epitopes of human cMET (e.g., non-overlapping), or multispecific antibodies or antigen-binding fragments thereof that specifically bind to human EGFR and human cMET, wherein the first antigen binding domain thereof targeting human cMET does not compete with the second antigen binding domain thereof targeting human cMET (e.g, EGFRxcMET biparatopic antibodies or antigen-binding fragments thereof) having any one or more of the following features:
(1) great or superior ADCC activity to cancer cells, including EGFR mutation/signaling driven cancer cells and/or cMET amplification/signaling driven cancer cells;
(2) great ADCP activity to cancer cells, including EGFR mutation/signaling driven cancer cells and/or cMET amplification/signaling driven cancer cells;
(3) blocking EGFR signaling and/or cMET signaling by down-regulating the receptor on the cell surface (e.g., by internalization);
(4) great anti-proliferation activity to EGFR mutation/signaling driven cancer cells, and/or have superior anti-proliferation activity to cMET amplification/signaling driven cancer cells;
(5) great or superior killing effect on cancer cells, including EGFR mutation/signaling driven cancer cells and especially cMET amplification/signaling driven cancer cells;
(6) great tumor growth inhibition in EGFR mutation/signaling driven tumor, and/or superior tumor growth inhibition in cMET amplification/signaling driven tumor; and
(7) less toxicity and/or better safety.

In some embodiment, the present disclosure provides EGFRxcMET biparatopic trispecific antibodies or antigen-binding fragments thereof exhibiting excellent killing effect/tumor growth inhibition to both EGFR mutation/signaling driven tumor and cMET amplification/signaling driven tumor, and thus able to overcome cMET amplification-driven compensatory resistance or EGFR mutation-driven compensatory resistance.

### BRIEF DESCRIPTION OF THE DRAWINGS

FIG. 1A-1L show the SPR binding kinetics curve of representative monoclonal anti-cMET antibodies. FIG. 1A: 063Ab10910; FIG. 1B: 061Ab15310; FIG. 1C: 063Ab16010; FIG. 1D: 063Ab02110; FIG. 1E: 063Ab15210; FIG. 1F: 062Ab16310; FIG. 1G: 063Ab05510; FIG. 1H: 063Ab07710; FIG. 1I: 063Ab14710; FIG. 1J: 061Ab05110; FIG. 1K: 063Ab03210; FIG. 1L: 063Ab16720.

FIG. 2 shows the comparison of ligand-induced signaling blocking activity of representative monoclonal anti-cMET antibodies.

FIG. 3 shows the epitope binning results of representative monoclonal anti-cMET antibodies.

FIG. 4 shows the inhibition of ligand-independent signaling by bispecific anti-cMET antibodies in Hs746T cells.

FIG. 5 shows the anti-proliferation activity of bispecific anti-cMET antibodies against Hs746T cells.

FIG. 6 shows the ligand-induced signaling blocking of bispecific anti-cMET antibodies in H596 cells.

FIG. 7. Different EGFR×cMET×cMET trispecific antibody formats, with different EGFR scFv valency and orientation to cMET arms.

FIG. 8. ADCC activity of EGFR×cMET×cMET trispecific antibodies on H1975 cells without (FIG. 8A) and with serum (FIG. 8B) and Hs746T cells without (FIG. 8C) and with serum (FIG. 8D).

FIG. 9. ADCP activity of EGFR×cMET×cMET trispecific antibodies on H1975 cells without (FIG. 9A) and with serum (FIG. 9B) and Hs746T without (FIG. 9C) and with serum (FIG. 9D).

FIG. 10. Anti-proliferation activity of EGFR×cMET×cMET trispecific antibodies on EGFR signaling-dependent cancer cell line H1975 (FIG. 10A) and H2073 (FIG. 10B) and cMET signaling-dependent cancer cell line Hs746T (FIG. 10C) and EBC-1 (FIG. 10D).

FIG. 11. Anti-proliferation activity of EGFR×cMET×cMET trispecific antibodies against HEKn cells.

FIG. 12. EGFR×cMET×cMET trispecific antibody TE-642 showed lower TGI and CR rate in HCC827 xenograft model (EGFR mutation driven model), compared with JNJ-372.

FIG. 13. EGFR×cMET×cMET trispecific antibody TE-647 showed comparable tumor growth inhibition to JNJ-372 in HCC827 xenograft model (EGFR mutation driven model).

FIG. 14. EGFR×cMET×cMET trispecific antibodies TE-642 and TE-646 showed tumor growth inhibition in H1975 xenograft model (EGFR mutation driven model).

FIG. 15. EGFR×cMET×cMET trispecific antibody TE-647 showed comparable tumor growth inhibition to JNJ-372 in H1975 xenograft model (EGFR mutation driven model).

FIG. 16. EGFR×cMET×cMET trispecific antibody TE-647 showed better tumor growth inhibition than JNJ-372 in Hs746T xenograft model (cMET amplification driven model), and the effect is dose dependent.

FIG. 17. EGFR×cMET×cMET antibody TE-647 showed better in vivo activity than JNJ-372 in EBC-1 (cMET amplification driven model) xenograft model, and the effect is dose dependent.

### DETAILED DESCRIPTION

The present disclosure provides for anti-human cMET antibodies or antigen-binding fragments thereof and multispecific antibody or antigen-binding fragment thereof which binds to two distinct epitopes of human cMET (e.g., non-overlapping), or wherein the first antigen binding domain thereof targeting human cMET does not compete with the second antigen binding domain thereof targeting human cMET. Also, the present disclosure provides antibodies that have desirable binding affinity, and desirable blocking activity on ligand independent signaling and/or ligand induced signaling. The antibodies can be used to construct multispecific antibodies with other modalities such as a second tumor associated antigens (TAAs), immune checkpoints or immune stimulators, or to construct antibody drug conjugates (ADC), or to fuse with other domains to form fusion proteins. Furthermore, the antibodies and the constructs thereof could be used for reducing the likelihood of or treating cancer and associated disorders.

The present disclosure also provides multispecific antibodies or antibody fragment thereof that specifically bind to human EGFR and two distinct epitopes of human cMET (e.g., non-overlapping) or multispecific antibodies or antigen-binding fragments thereof that specifically bind to human EGFR and human cMET, wherein the first antigen binding domain thereof targeting human cMET does not compete with the second antigen binding domain thereof targeting human cMET. Also, the present disclosure provides multispecific antibodies or antibody fragment thereof that have desirable binding affinity, desirable ADCC activity, desirable ADCP activity, desirable blocking activity on EGFR signaling and cMET signaling, desirable anti-proliferation activity and killing effect on EGFR signaling driven cancer cells and cMET signaling driven cancer cells. Furthermore, the multispecific antibodies and the constructs thereof could be used for reducing the likelihood of or treating cancer and associated disorders.

### I. Anti-cMET Antibodies

The present disclosure provides for antibodies or antigen-binding fragments thereof that specifically bind to human cMET. Antibodies or antigen-binding fragments of the present disclosure include, but are not limited to, the antibodies or antigen-binding fragments thereof, generated as described below.

### 1.1 First Group of Anti-cMET Antibodies

TABLE 1
Anti-cMET Monoclonal Antibody List 1

| | SEQ ID NO. | | | | | | | | | |
|---|---|---|---|---|---|---|---|---|---|---|
| | VH | VH | HCDR1 | HCDR2 | HCDR3 | VL | VL | LCDR1 | LCDR2 | LCDR3 |
| Antibody | (AA) | (DNA) | (Kabat) | (Kabat) | (Kabat) | (AA) | (DNA) | (Kabat) | (Kabat) | (Kabat) |
| 063Ab10910 | 4 | 5 | 6 | 7 | 8 | 64 | 65 | 66 | 67 | 68 |
| 063Ab10910-V1 | 72 | / | 6 | 7 | 8 | 64 | 65 | 66 | 67 | 68 |
| 063Ab10910 | / | / | 6 | 7 | 94 | 64 | 65 | 66 | 67 | 68 |
| variants | | | | | (formula) | | | | | |
| 063Ab10910-P28 | 85 | / | 6 | 7 | 84 | 64 | 65 | 66 | 67 | 68 |
| 063Ab10910-P27 | 87 | / | 6 | 7 | 86 | 64 | 65 | 66 | 67 | 68 |
| 063Ab10910-P37 | 89 | / | 6 | 7 | 88 | 64 | 65 | 66 | 67 | 68 |
| 063Ab10910-P26 | 91 | / | 6 | 7 | 90 | 64 | 65 | 66 | 67 | 68 |
| 063Ab10910-P19 | 93 | / | 6 | 7 | 92 | 64 | 65 | 66 | 67 | 68 |
| BGA-109 | 144 | 162 | 6 | 7 | 92 | 64 | 65 | 66 | 67 | 68 |
| 061Ab15310 | 9 | 10 | 11 | 12 | 13 | 64 | 65 | 66 | 67 | 68 |
| 061Ab15310-V1 | 73 | 1 | 11 | 12 | 13 | 64 | 65 | 66 | 67 | 68 |
| 063Ab16010 | 14 | 15 | 16 | 17 | 18 | 64 | 65 | 66 | 67 | 68 |
| 063Ab16010-V1 | 74 | / | 16 | 17 | 18 | 64 | 65 | 66 | 67 | 68 |
| 063Ab02110 | 19 | 20 | 21 | 22 | 23 | 64 | 65 | 66 | 67 | 68 |
| 063Ab02110-V1 | 75 | / | 21 | 22 | 23 | 64 | 65 | 66 | 67 | 68 |
| 063Ab15210 | 24 | 25 | 26 | 27 | 28 | 64 | 65 | 66 | 67 | 68 |
| 063Ab15210-V1 | 76 | / | 26 | 27 | 28 | 64 | 65 | 66 | 67 | 68 |
| 062Ab16310 | 29 | 30 | 31 | 32 | 33 | 64 | 65 | 66 | 67 | 68 |
| 062Ab16310-V1 | 77 | / | 31 | 32 | 33 | 64 | 65 | 66 | 67 | 68 |
| 063Ab05510 | 34 | 35 | 36 | 37 | 38 | 64 | 65 | 66 | 67 | 68 |
| 063Ab05510-V1 | 78 | / | 36 | 37 | 38 | 64 | 65 | 66 | 67 | 68 |
| 063Ab07710 | 39 | 40 | 41 | 42 | 43 | 64 | 65 | 66 | 67 | 68 |
| 063Ab07710-V1 | 79 | / | 41 | 42 | 43 | 64 | 65 | 66 | 67 | 68 |
| 063Ab14710 | 44 | 45 | 46 | 47 | 48 | 64 | 65 | 66 | 67 | 68 |
| 063Ab14710-V1 | 80 | / | 46 | 47 | 48 | 64 | 65 | 66 | 67 | 68 |
| 061Ab05110 | 49 | 50 | 51 | 52 | 53 | 64 | 65 | 66 | 67 | 68 |
| 061Ab05110-V1 | 81 | / | 51 | 52 | 53 | 64 | 65 | 66 | 67 | 68 |

The present disclosure provides for antibodies or antigen-binding fragments that specifically bind to human cMET, wherein said antibodies or antibody fragments (e.g., antigen-binding fragments) comprise a VH domain having an amino acid sequence of SEQ ID NO: 4, SEQ ID NO: 72, SEQ ID NO: 85, SEQ ID NO: 87, SEQ ID NO: 89, SEQ ID NO: 91, SEQ ID NO: 93, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81 or SEQ ID NO: 144 (Table 1). The present disclosure also provides antibodies or antigen-binding fragments that specifically bind human cMET, wherein said antibodies or antigen-binding fragments comprise a HCDR having an amino acid sequence of any one of the HCDRs listed in Table 1. In one aspect, the present disclosure provides antibodies or antigen-binding fragments that specifically bind to human cMET, wherein said antibodies comprise one, two, three, or more HCDRs having an amino acid sequence of any of the HCDRs listed in Table 1.

The present disclosure provides for antibodies or antigen-binding fragments that specifically bind to human cMET, wherein said antibodies or antibody fragments (e.g., antigen-binding fragments) comprise a VL domain having an amino acid sequence of SEQ ID NO: 64 (Table 1). The present disclosure also provides antibodies or antigen-binding fragments that specifically bind human cMET, wherein said antibodies or antigen-binding fragments comprise a LCDR having an amino acid sequence of any one of the LCDRs listed in Table 1. In one aspect, the present disclosure provides antibodies or antigen-binding fragments that specifically bind to human cMET, wherein said antibodies comprise one, two, three, or more LCDRs having an amino acid sequence of any of the LCDRs listed in Table 1.

In one embodiment, the antibody or an antigen-binding fragment thereof that specifically bind to human cMET comprises one or more complementarity determining regions (CDRs) comprising an amino acid sequence selected from a group consisting of SEQ ID NO: 6, SEQ ID NO: 7, SEQ ID NO: 8, SEQ ID NO: 94, SEQ ID NO: 84; SEQ ID NO: 86, SEQ ID NO: 88, SEQ ID NO: 90, SEQ ID NO: 92, SEQ ID NO: 11, SEQ ID NO: 12, SEQ ID NO: 13, SEQ ID NO: 16, SEQ ID NO: 17, SEQ ID NO: 18, SEQ ID NO: 21, SEQ ID NO: 22, SEQ ID NO: 23, SEQ ID NO: 26, SEQ ID NO: 27, SEQ ID NO: 28, SEQ ID NO: 31, SEQ ID NO: 32, SEQ ID NO: 33, SEQ ID NO: 36, SEQ ID NO: 37, SEQ ID NO: 38, SEQ ID NO: 41, SEQ ID NO: 42, SEQ ID NO: 43, SEQ ID NO: 46, SEQ ID NO: 47, SEQ ID NO: 48, SEQ ID NO: 51, SEQ ID NO: 52, SEQ ID NO: 53, SEQ ID NO: 66, SEQ ID NO: 67 and SEQ ID NO: 68.

In another embodiment, the antibody or an antigen-binding fragment thereof that specifically bind to human cMET comprises: (a) a heavy chain variable region comprising one or more complementarity determining regions (HCDRs) comprising an amino acid sequence selected from the group consisting of SEQ ID NO: 6, SEQ ID NO: 7, SEQ ID NO: 8, SEQ ID NO: 94, SEQ ID NO: 84; SEQ ID NO: 86, SEQ ID NO: 88, SEQ ID NO: 90, SEQ ID NO: 92, SEQ ID NO: 11, SEQ ID NO: 12, SEQ ID NO: 13, SEQ ID NO: 16, SEQ ID NO: 17, SEQ ID NO: 18, SEQ ID NO: 21, SEQ ID NO: 22, SEQ ID NO: 23, SEQ ID NO: 26, SEQ ID NO: 27, SEQ ID NO: 28, SEQ ID NO: 31, SEQ ID NO: 32, SEQ ID NO: 33, SEQ ID NO: 36, SEQ ID NO: 37, SEQ ID NO: 38, SEQ ID NO: 41, SEQ ID NO: 42, SEQ ID NO: 43, SEQ ID NO: 46, SEQ ID NO: 47, SEQ ID NO: 48, SEQ ID NO: 51, SEQ ID NO: 52, and SEQ ID NO: 53, and/or (b) a light chain variable region comprising one or more complementarity determining regions (LCDRs) comprising an amino acid sequence selected from the group consisting of SEQ ID NO: 66, SEQ ID NO: 67 and SEQ ID NO: 68.

In another embodiment, the antibody or an antigen-binding fragment thereof that specifically bind to human cMET comprises: (a) a heavy chain variable region comprising three complementarity determining regions (HCDRs) which are HCDR1 comprising an amino acid sequence of SEQ ID NO: 6, SEQ ID NO: 11, SEQ ID NO: 16, SEQ ID NO: 21, SEQ ID NO: 26, SEQ ID NO: 31, SEQ ID NO: 36, SEQ ID NO: 41, SEQ ID NO: 46, or SEQ ID NO: 51; HCDR2 comprising an amino acid sequence of SEQ ID NO: 7, SEQ ID NO: 12, SEQ ID NO: 17, SEQ ID NO: 22, SEQ ID NO: 27, SEQ ID NO: 32, SEQ ID NO: 37, SEQ ID NO: 42, SEQ ID NO: 47, or SEQ ID NO: 52; and HCDR3 comprising an amino acid sequence of SEQ ID NO: 8, SEQ ID NO: 94, SEQ ID NO: 84; SEQ ID NO: 86, SEQ ID NO: 88, SEQ ID NO: 90, SEQ ID NO: 92, SEQ ID NO: 13, SEQ ID NO: 18, SEQ ID NO: 23, SEQ ID NO: 28, SEQ ID NO: 33, SEQ ID NO: 38, SEQ ID NO: 43, SEQ ID NO: 48, or SEQ ID NO: 53; and/or (b) a light chain variable region comprising three complementarity determining regions (LCDRs) which are LCDR1 comprising an amino acid sequence of SEQ ID NO: 66; LCDR2 comprising an amino acid sequence of SEQ ID NO: 67; and LCDR3 comprising an amino acid sequence of SEQ ID NO: 68.

In another embodiment, the antibody or an antigen-binding fragment thereof that specifically bind to human cMET comprises: (a) a heavy chain variable region comprising three complementarity determining regions (HCDRs) which are: HCDR1 comprising an amino acid sequence of SEQ ID NO: 6, HCDR2 comprising an amino acid sequence of SEQ ID NO: 7, and HCDR3 comprising an amino acid sequence of SEQ ID NO: 8; HCDR1 comprising an amino acid sequence of SEQ ID NO: 6, HCDR2 comprising an amino acid sequence of SEQ ID NO: 7, and HCDR3 comprising an amino acid sequence of SEQ ID NO: 94; HCDR1 comprising an amino acid sequence of SEQ ID NO: SEQ ID NO: 6, HCDR2 comprising an amino acid sequence of SEQ ID NO: 7, and HCDR3 comprising an amino acid sequence of SEQ ID NO: 84; HCDR1 comprising an amino acid sequence of SEQ ID NO: SEQ ID NO: 6, HCDR2 comprising an amino acid sequence of SEQ ID NO: 7, and HCDR3 comprising an amino acid sequence of SEQ ID NO: 86; HCDR1 comprising an amino acid sequence of SEQ ID NO: 6, HCDR2 comprising an amino acid sequence of SEQ ID NO: 7, and HCDR3 comprising an amino acid sequence of SEQ ID NO: 88; HCDR1 comprising an amino acid sequence of SEQ ID NO: SEQ ID NO: 6, HCDR2 comprising an amino acid sequence of SEQ ID NO: 7, and HCDR3 comprising an amino acid sequence of SEQ ID NO: 90; HCDR1 comprising an amino acid sequence of SEQ ID NO: SEQ ID NO: 6, HCDR2 comprising an amino acid sequence of SEQ ID NO: 7, and HCDR3 comprising an amino acid sequence of SEQ ID NO: 92; HCDR1 comprising an amino acid sequence of SEQ ID NO: SEQ ID NO: 11, HCDR2 comprising an amino acid sequence of SEQ ID NO: 12, and HCDR3 comprising an amino acid sequence of SEQ ID NO: 13; HCDR1 comprising an amino acid sequence of SEQ ID NO: SEQ ID NO: 16, HCDR2 comprising an amino acid sequence of SEQ ID NO: 17, and HCDR3 comprising an amino acid sequence of SEQ ID NO: 18; HCDR1 comprising an amino acid sequence of SEQ ID NO: SEQ ID NO: 21, HCDR2 comprising an amino acid sequence of SEQ ID NO: 22, and HCDR3 comprising an amino acid sequence of SEQ ID NO: 23; HCDR1 comprising an amino acid sequence of SEQ ID NO: SEQ ID NO: 26, HCDR2 comprising an amino acid sequence of SEQ ID NO: 27, and HCDR3 comprising an amino acid sequence of SEQ ID NO: 28; HCDR1 comprising an amino acid sequence of SEQ ID NO: SEQ ID NO: 31, HCDR2 comprising an amino acid sequence of SEQ ID NO: 32, and HCDR3 comprising an amino acid sequence of SEQ ID NO: 33; HCDR1 comprising an amino acid sequence of SEQ ID NO: SEQ ID NO: 36, HCDR2 comprising an amino acid sequence of SEQ ID NO: 37, and HCDR3 comprising an amino acid sequence of SEQ ID NO: 38; HCDR1 comprising an amino acid sequence of SEQ ID NO: SEQ ID NO: 41, HCDR2 comprising an amino acid sequence of SEQ ID NO: 42, and HCDR3 comprising an amino acid sequence of SEQ ID NO: 43; HCDR1 comprising an amino acid sequence of SEQ ID NO: SEQ ID NO: 46, HCDR2 comprising an amino acid sequence of SEQ ID NO: 47, and HCDR3 comprising an amino acid sequence of SEQ ID NO: 48; or HCDR1 comprising an amino acid sequence of SEQ ID NO: SEQ ID NO: 51, HCDR2 comprising an amino acid sequence of SEQ ID NO: 52, and HCDR3 comprising an amino acid sequence of SEQ ID NO: 53, and/or (b) a light chain variable region comprising three complementarity determining regions (LCDRs) which are LCDR1 comprising an amino acid sequence of SEQ ID NO: 66, LCDR2 comprising an amino acid sequence of SEQ ID NO: 67, and LCDR3 comprising an amino acid sequence of SEQ ID NO: 68.

In one embodiment, the antibodies or antigen-binding fragments thereof that specifically bind to human cMET comprise: (1) a HCDR1 (Heavy Chain Complementarity Determining Region 1), a HCDR2 and a HCDR3 from the heavy chain variable region (VH) set forth in SEQ ID NO: 4 or SEQ ID NO: 72; (2) a HCDR1, a HCDR2 and a HCDR3 from the heavy chain variable region (VH) set forth in SEQ ID NO: 85; (3) a HCDR1, a HCDR2 and a HCDR3 from the heavy chain variable region (VH) set forth in SEQ ID NO: 87; (4) a HCDR1, a HCDR2 and a HCDR3 from the heavy chain variable region (VH) set forth in SEQ ID NO: 89; (5) a HCDR1, a HCDR2 and a HCDR3 from the heavy chain variable region (VH) set forth in SEQ ID NO: 91; (6) a HCDR1, a HCDR2 and a HCDR3 from the heavy chain variable region (VH) set forth in SEQ ID NO: 93; (7) a HCDR1, a HCDR2 and a HCDR3 from the heavy chain variable region (VH) set forth in SEQ ID NO: 9 or SEQ ID NO: 73; (8) a HCDR1, a HCDR2 and a HCDR3 from the heavy chain variable region (VH) set forth in SEQ ID NO: 14 or SEQ ID NO: 74; (9) a HCDR1, a HCDR2 and a HCDR3 from the heavy chain variable region (VH) set forth in SEQ ID NO: 19 or SEQ ID NO: 75; (10) a HCDR1, a HCDR2 and a HCDR3 from the heavy chain variable region (VH) set forth in SEQ ID NO: 24 or SEQ ID NO: 76; (11) a HCDR1, a HCDR2 and a HCDR3 from the heavy chain variable region (VH) set forth in SEQ ID NO: 29 or SEQ ID NO: 77; (12) a HCDR1, a HCDR2 and a HCDR3 from the heavy chain variable region (VH) set forth in SEQ ID NO: 34 or SEQ ID NO: 78; (13) a HCDR1, a HCDR2 and a HCDR3 from the heavy chain variable region (VH) set forth in SEQ ID NO: 39 or SEQ ID NO: 79; (14) a HCDR1, a HCDR2 and a HCDR3 from the heavy chain variable region (VH) set forth in SEQ ID NO: 44 or SEQ ID NO: 80; (15) a HCDR1, a HCDR2 and a HCDR3 from the heavy chain variable region (VH) set forth in SEQ ID NO: 49 or SEQ ID NO: 81; or (16) a HCDR1, a HCDR2 and a HCDR3 from the heavy chain variable region (VH) set forth in SEQ ID NO: 144; and/or (1) a LCDR1 (Light Chain Complementarity Determining Region 1), a LCDR2 and a LCDR3 from the light chain variable region (VL) set forth in SEQ ID NO: 64.

In one embodiment, the antibodies or antigen-binding fragments thereof that specifically bind to human cMET comprise:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(3) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(4) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(5) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(6) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68.
(7) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(13) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(14) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(15) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68; or
(16) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68, according to the Kabat definition.

In one embodiment, the antibody or an antigen-binding fragment thereof of the present disclosure comprises: (a) a heavy chain variable region comprising an amino acid sequence of SEQ ID NO: 4, SEQ ID NO: 72, SEQ ID NO: 85, SEQ ID NO: 87, SEQ ID NO: 89, SEQ ID NO: 91, SEQ ID NO: 93, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81 or SEQ ID NO: 144; or an amino acid sequence at least 90%, 91%, 92%, 93%, 94%, 95%, 96%, 97%, 98% or 99% identical to any one of SEQ ID NO: 4, SEQ ID NO: 72, SEQ ID NO: 85, SEQ ID NO: 87, SEQ ID NO: 89, SEQ ID NO: 91, SEQ ID NO: 93, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81 or SEQ ID NO: 144 and/or (b) a light chain variable region comprising an amino acid sequence of SEQ ID NO: 64, or an amino acid sequence at least 90%, 91%, 92%, 93%, 94%, 95%, 96%, 97%, 98% or 99% identical to SEQ ID NO: 64.

In one embodiment, the antibody or antigen-binding fragment thereof comprises:
(1) a heavy chain variable region (VH) comprising an amino acid sequence at least 90, 91, 92, 93, 94, 95, 96, 97, 98 or 99% identical to SEQ ID NO: 85, SEQ ID NO: 87, SEQ ID NO: 89, SEQ ID NO: 91, or SEQ ID NO: 93, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 4 or SEQ ID NO: 72, and a light chain variable region (VL) comprising an amino acid sequence at least 90, 91, 92, 93, 94, 95, 96, 97, 98 or 99% identical to SEQ ID NO: 64;
(3) a heavy chain variable region (VH) comprising an amino acid sequence at least 90, 91, 92, 93, 94, 95, 96, 97, 98 or 99% identical to SEQ ID NO: 9 or SEQ ID NO: 73, and a light chain variable region (VL) comprising an amino acid sequence at least 90, 91, 92, 93, 94, 95, 96, 97, 98 or 99% identical to SEQ ID NO: 64;
(4) a heavy chain variable region (VH) comprising an amino acid sequence at least 90, 91, 92, 93, 94, 95, 96, 97, 98 or 99% identical to SEQ ID NO: 14, or SEQ ID NO: 74, and a light chain variable region (VL) comprising an amino acid sequence at least 90, 91, 92, 93, 94, 95, 96, 97, 98 or 99% identical to SEQ ID NO: 64;
(5) a heavy chain variable region (VH) comprising an amino acid sequence at least 90, 91, 92, 93, 94, 95, 96, 97, 98 or 99% identical to SEQ ID NO: 19, or SEQ ID NO: 75, and a light chain variable region (VL) comprising an amino acid sequence at least 90, 91, 92, 93, 94, 95, 96, 97, 98 or 99% identical to SEQ ID NO: 64;
(6) a heavy chain variable region (VH) comprising an amino acid sequence at least 90, 91, 92, 93, 94, 95, 96, 97, 98 or 99% identical to SEQ ID NO: 24, or SEQ ID NO: 76, and a light chain variable region (VL) comprising an amino acid sequence at least 90, 91, 92, 93, 94, 95, 96, 97, 98 or 99% identical to SEQ ID NO: 64;
(7) a heavy chain variable region (VH) comprising an amino acid sequence at least 90, 91, 92, 93, 94, 95, 96, 97, 98 or 99% identical to SEQ ID NO: 29, or SEQ ID NO: 77, and a light chain variable region (VL) comprising an amino acid sequence at least 90, 91, 92, 93, 94, 95, 96, 97, 98 or 99% identical to SEQ ID NO: 64;
(8) a heavy chain variable region (VH) comprising an amino acid sequence at least 90, 91, 92, 93, 94, 95, 96, 97, 98 or 99% identical to SEQ ID NO: 34, or SEQ ID NO: 78, and a light chain variable region (VL) comprising an amino acid sequence at least 90, 91, 92, 93, 94, 95, 96, 97, 98 or 99% identical to SEQ ID NO: 64;
(9) a heavy chain variable region (VH) comprising an amino acid sequence at least 90, 91, 92, 93, 94, 95, 96, 97, 98 or 99% identical to SEQ ID NO: 39, or SEQ ID NO: 79, and a light chain variable region (VL) comprising an amino acid sequence at least 90, 91, 92, 93, 94, 95, 96, 97, 98 or 99% identical to SEQ ID NO: 64;
(10) a heavy chain variable region (VH) comprising an amino acid sequence at least 90, 91, 92, 93, 94, 95, 96, 97, 98 or 99% identical to SEQ ID NO: 44, or SEQ ID NO: 80, and a light chain variable region (VL) comprising an amino acid sequence at least 90, 91, 92, 93, 94, 95, 96, 97, 98 or 99% identical to SEQ ID NO: 64;
(11) a heavy chain variable region (VH) comprising an amino acid sequence at least 90, 91, 92, 93, 94, 95, 96, 97, 98 or 99% identical to SEQ ID NO: 49, or SEQ ID NO: 81, and a light chain variable region (VL) comprising an amino acid sequence at least 90, 91, 92, 93, 94, 95, 96, 97, 98 or 99% identical to SEQ ID NO: 64; or
(12) a heavy chain variable region (VH) comprising an amino acid sequence at least 90, 91, 92, 93, 94, 95, 96, 97, 98 or 99% identical to SEQ ID NO: 144, and a light chain variable region (VL) comprising an amino acid sequence at least 90, 91, 92, 93, 94, 95, 96, 97, 98 or 99% identical to SEQ ID NO: 64.

In one embodiment, the present disclosure provides the antibody or antigen-binding fragment thereof, wherein one, two, three, four, five, six, seven, eight, nine, or ten amino acids within SEQ ID NO: 85, SEQ ID NO: 87, SEQ ID NO: 89, SEQ ID NO: 91, SEQ ID NO: 93, SEQ ID NO: 4, SEQ ID NO: 72, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81, SEQ ID NO: 144, or SEQ ID NO: 64 have been inserted, deleted or substituted (optionally conservative amino acid substitutions), while retaining therapeutic activity/binding specificity/affinity, optionally the corresponding sequences of CDRs do not change.

In one embodiment, the present disclosure provides the antibody or antigen-binding fragment thereof, wherein one, two, three, four, five, six, seven, eight, nine, or ten amino acids within SEQ ID NO: 85 and SEQ ID NO: 64, SEQ ID NO: 87 and SEQ ID NO: 64, SEQ ID NO: 89 and SEQ ID NO: 64, SEQ ID NO: 91 and SEQ ID NO: 64, SEQ ID NO: 93 and SEQ ID NO: 64, SEQ ID NO: 4 and SEQ ID NO: 64, SEQ ID NO: 72 and SEQ ID NO: 64, SEQ ID NO: 9 and SEQ ID NO: 64, SEQ ID NO: 73 and SEQ ID NO: 64, SEQ ID NO: 14 and SEQ ID NO: 64, SEQ ID NO: 74 and SEQ ID NO: 64, SEQ ID NO: 19 and SEQ ID NO: 64, SEQ ID NO: 75 and SEQ ID NO: 64, SEQ ID NO: 24 and SEQ ID NO: 64, SEQ ID NO: 76 and SEQ ID NO: 64, SEQ ID NO: 29 and SEQ ID NO: 64, SEQ ID NO: 77 and SEQ ID NO: 64, SEQ ID NO: 34 and SEQ ID NO: 64, SEQ ID NO: 78 and SEQ ID NO: 64, SEQ ID NO: 39 and SEQ ID NO: 64, SEQ ID NO: 79 and SEQ ID NO: 64, SEQ ID NO: 44 and SEQ ID NO: 64, SEQ ID NO: 80 and SEQ ID NO: 64, SEQ ID NO: 49 and SEQ ID NO: 64, SEQ ID NO: 81 and SEQ ID NO: 64, or SEQ ID NO: 144 and SEQ ID NO: 64 have been inserted, deleted or substituted (optionally conservative amino acid substitutions), while retaining therapeutic activity/binding specificity/affinity, optionally the corresponding sequences of CDRs do not change.

In one embodiment, the antibody or antigen-binding fragment thereof, that comprises:
(1) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64;
(2) a heavy chain variable region (VH) comprising an amino acid sequence of SEQ ID NO: 4 or SEQ ID NO: 72, and a light chain variable region (VL) comprising an amino acid sequence of SEQ ID NO: 64;
(3) a heavy chain variable region (VH) comprising an amino acid sequence of SEQ ID NO: 9 or SEQ ID NO: 73, and a light chain variable region (VL) comprising an amino acid sequence of SEQ ID NO: 64;
(4) a heavy chain variable region (VH) comprising an amino acid sequence of SEQ ID NO: 14, or SEQ ID NO: 74, and a light chain variable region (VL) comprising an amino acid sequence of SEQ ID NO: 64;
(5) a heavy chain variable region (VH) comprising an amino acid sequence of SEQ ID NO: 19, or SEQ ID NO: 75, and a light chain variable region (VL) comprising an amino acid sequence of SEQ ID NO: 64;
(6) a heavy chain variable region (VH) comprising an amino acid sequence of SEQ ID NO: 24, or SEQ ID NO: 76, and a light chain variable region (VL) of SEQ ID NO: 64;
(7) a heavy chain variable region (VH) comprising an amino acid sequence of SEQ ID NO: 29, or SEQ ID NO: 77, and a light chain variable region (VL) comprising an amino acid sequence of SEQ ID NO: 64;
(8) a heavy chain variable region (VH) comprising an amino acid sequence of SEQ ID NO: 34, or SEQ ID NO: 78, and a light chain variable region (VL) comprising an amino acid sequence of SEQ ID NO: 64;
(9) a heavy chain variable region (VH) comprising an amino acid sequence of SEQ ID NO: 39, or SEQ ID NO: 79, and a light chain variable region (VL) comprising an amino acid sequence of SEQ ID NO: 64;
(10) a heavy chain variable region (VH) comprising an amino acid sequence of SEQ ID NO: 44, or SEQ ID NO: 80, and a light chain variable region (VL) comprising an amino acid sequence of SEQ ID NO: 64; or
(11) a heavy chain variable region (VH) comprising an amino acid sequence of SEQ ID NO: 49, or SEQ ID NO: 81, and a light chain variable region (VL) comprising an amino acid sequence of SEQ ID NO: 64; or
(12) a heavy chain variable region (VH) comprising an amino acid sequence of SEQ ID NO: 144, and a light chain variable region (VL) comprising an amino acid sequence of SEQ ID NO: 64.

Other antibodies or antigen-binding fragments thereof of the present disclosure include amino acids that have been changed, yet have at least 60%, 70%, 80%, 90%, 91%, 92%, 93%, 94%, 95% 96%, 97%, 98%, or 99% percent identity in the CDR regions compared with the CDR regions disclosed in Table 1. In some aspects, it includes amino acid changes (insertion, deletion or substitution, optionally conservative amino acid substitutions) wherein no more than 1, 2, 3, 4 or 5 amino acids have been changed in the CDR regions when compared with the CDR regions depicted in the sequence described in Table 1, while maintaining binding specificity and affinity.

Other antibodies of the present disclosure include those where the amino acids or nucleic acids encoding the amino acids have been changed in the variable regions (e.g., the frameworks regions of the variable regions); yet have at least 60, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% percent identity to the sequences of variable regions described in Table 1, while retaining binding specificity/affinity, optionally the corresponding sequences of CDRs do not change. In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions (e.g., the frameworks regions of the variable regions) when compared with the variable regions depicted in the sequence described in Table 1, while retaining binding specificity/affinity, optionally the corresponding sequences of CDRs do not change. The changes could be insertion, deletion or substitution, optionally conservative amino acid substitutions.

In another embodiment, the present disclosure provides for antibodies or antigen-binding fragments thereof that specifically bind to human cMET with a binding affinity (K_{D}) of from 1×10⁻⁶ M to 1×10⁻¹¹ M. In another embodiment, the anti-cMET antibodies or antigen-binding fragments thereof bind to human cMET with a binding affinity (K_{D}) of about 1×10⁻⁶ M, about 1×10⁻⁷ M, about 1×10⁻⁸ M, about 1×10⁻⁹ M, about 1×10⁻¹⁰ M or about 1×10⁻¹¹ M.

The present disclosure also provides nucleic acid sequences that encode VH or VL of the antibodies that specifically bind to human cMET. Such nucleic acid sequences can be optimized for expression in mammalian cells.

The present disclosure also provides for antibodies and antigen-binding fragments thereof that bind to the same epitope as do the anti-cMET antibodies described in Table 1. Additional antibodies and antigen-binding fragments thereof can therefore be identified based on their ability to cross-compete (e.g., to competitively inhibit the binding of, in a statistically significant manner) with the antibodies described in Table 1 in binding assays. The ability of a test antibody to inhibit the binding of antibodies and antigen-binding fragments thereof of the present disclosure to human cMET demonstrates that the test antibody can compete with that antibody or antigen-binding fragments thereof for binding to human cMET. Such an antibody can, without being bound to any one theory, bind to the same or a related (e.g., a structurally similar or spatially proximal) epitope on human cMET as the antibody or antigen-binding fragments thereof with which it competes. In a certain aspect, the antibody that binds to the same epitope on human cMET as the antibodies or antigen-binding fragments thereof of the present disclosure is a human or humanized monoclonal antibody. Such human or humanized monoclonal antibodies can be prepared and isolated as described herein.

In some embodiments, the antibody or antigen-binding fragment thereof is a monoclonal antibody, a human engineered antibody, a single chain antibody (scFv), a Fab fragment, a Fab′ fragment, or a F(ab′)2 fragment.

In one embodiment, the antibody or antigen-binding fragment thereof is in a scFv format comprising VH-VL in the direction of N-terminal to C-terminal, or VL-VH in the direction of N-terminal to C-terminal. In some embodiments, the VH and VL are connected via an amino acid linker described herein. In some embodiments, the VH comprises the amino acid sequence of any one of SEQ ID NO: 85, SEQ ID NO: 87, SEQ ID NO: 89, SEQ ID NO: 91, SEQ ID NO: 93, SEQ ID NO: 4, SEQ ID NO: 72, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81, or SEQ ID NO: 144. In some embodiments, the VL comprises the amino acid sequence of any one of SEQ ID NO: 64. In some embodiments, the VH is any one of VHs described in Table 1. In some embodiment, the VL is any one of VLs described in Table 1. In some embodiments, the amino acid linker has an amino acid sequence comprising any one of SEQ ID NO: 97-139. In some embodiments, the amino acid linker is any sequence of SEQ ID NO: 97-139.

### 1.2 Second Group of Anti-cMET Antibodies

TABLE 2
Anti-cMET Monoclonal Antibody List 2

| | SEQ ID NO. | | | | | | | | | |
|---|---|---|---|---|---|---|---|---|---|---|
| | VH | VH | HCDR1 | HCDR2 | HCDR3 | VL | VL | LCDR1 | LCDR2 | LCDR3 |
| Antibody | (AA) | (DNA) | (Kabat) | (Kabat) | (Kabat) | (AA) | (DNA) | (Kabat) | (Kabat) | (Kabat) |
| 063Ab03210 | 54 | 55 | 56 | 57 | 58 | 64 | 65 | 66 | 67 | 68 |
| 063Ab03210-V1 | 82 | / | 56 | 57 | 58 | 64 | 65 | 66 | 67 | 68 |
| BGA-032 | 145 | 163 | 56 | 57 | 58 | 64 | 65 | 66 | 67 | 68 |
| 063Ab16720 | 59 | 60 | 61 | 62 | 63 | 64 | 65 | 66 | 67 | 68 |
| 063Ab16720-V1 | 83 | / | 61 | 62 | 63 | 64 | 65 | 66 | 67 | 68 |

The present disclosure provides for antibodies or antigen-binding fragments that specifically bind to human cMET, wherein said antibodies or antibody fragments (e.g., antigen-binding fragments) comprise a VH domain having an amino acid sequence of SEQ ID NO: 54, SEQ ID NO: 82, SEQ ID NO: 145, SEQ ID NO: 59, or SEQ ID NO: 83 (Table 2). The present disclosure also provides antibodies or antigen-binding fragments that specifically bind human cMET, wherein said antibodies or antigen-binding fragments comprise a HCDR having an amino acid sequence of any one of the HCDRs listed in Table 2. In one aspect, the present disclosure provides antibodies or antigen-binding fragments that specifically bind to human cMET, wherein said antibodies comprise one, two, three, or more HCDRs having an amino acid sequence of any of the HCDRs listed in Table 2.

The present disclosure provides for antibodies or antigen-binding fragments that specifically bind to human cMET, wherein said antibodies or antibody fragments (e.g., antigen-binding fragments) comprise a VL domain having an amino acid sequence of SEQ ID NO: 64 (Table 2). The present disclosure also provides antibodies or antigen-binding fragments that specifically bind human cMET, wherein said antibodies or antigen-binding fragments comprise a LCDR having an amino acid sequence of any one of the LCDRs listed in Table 2. In one aspect, the present disclosure provides antibodies or antigen-binding fragments that specifically bind to human cMET, wherein said antibodies comprise one, two, three, or more LCDRs having an amino acid sequence of any of the LCDRs listed in Table 2.

In one embodiment, the antibody or an antigen-binding fragment thereof that specifically bind to human cMET comprises one or more complementarity determining regions (CDRs) comprising an amino acid sequence selected from a group consisting of SEQ ID NO: 56, SEQ ID NO: 57, SEQ ID NO: 58, SEQ ID NO: 61, SEQ ID NO: 62; SEQ ID NO: 63, SEQ ID NO: 66, SEQ ID NO: 67 and SEQ ID NO: 68.

In another embodiment, the antibody or an antigen-binding fragment thereof that specifically bind to human cMET comprises: (a) a heavy chain variable region comprising one or more complementarity determining regions (HCDRs) comprising an amino acid sequence selected from the group consisting of SEQ ID NO: 56, SEQ ID NO: 57, SEQ ID NO: 58, SEQ ID NO: 61, SEQ ID NO: 62; SEQ ID NO: 63, and/or (b) a light chain variable region comprising one or more complementarity determining regions (LCDRs) comprising an amino acid sequence selected from the group consisting of SEQ ID NO: 66, SEQ ID NO: 67 and SEQ ID NO: 68.

In another embodiment, the antibody or an antigen-binding fragment thereof that specifically bind to human cMET comprises: (a) a heavy chain variable region comprising three complementarity determining regions (HCDRs) which are HCDR1 comprising an amino acid sequence of SEQ ID NO: 56, or SEQ ID NO: 61; HCDR2 comprising an amino acid sequence of SEQ ID NO: 57, or SEQ ID NO: 62; and HCDR3 comprising an amino acid sequence of SEQ ID NO: 58, or SEQ ID NO: 63; and/or (b) a light chain variable region comprising three complementarity determining regions (LCDRs) which are LCDR1 comprising an amino acid sequence of SEQ ID NO: 66; LCDR2 comprising an amino acid sequence of SEQ ID NO: 67; and LCDR3 comprising an amino acid sequence of SEQ ID NO: 68.

In another embodiment, the antibody or an antigen-binding fragment thereof that specifically bind to human cMET comprises: (a) a heavy chain variable region comprising three complementarity determining regions (HCDRs) which are: HCDR1 comprising an amino acid sequence of SEQ ID NO: 56, HCDR2 comprising an amino acid sequence of SEQ ID NO: 57, and HCDR3 comprising an amino acid sequence of SEQ ID NO: 58; or HCDR1 comprising an amino acid sequence of SEQ ID NO: SEQ ID NO: 61, HCDR2 comprising an amino acid sequence of SEQ ID NO: 62, and HCDR3 comprising an amino acid sequence of SEQ ID NO: 63, and/or (b) a light chain variable region comprising three complementarity determining regions (LCDRs) which are LCDR1 comprising an amino acid sequence of SEQ ID NO: 66, LCDR2 comprising an amino acid sequence of SEQ ID NO: 67, and LCDR3 comprising an amino acid sequence of SEQ ID NO: 68.

In one embodiment, the antibodies or antigen-binding fragments thereof that specifically bind to human cMET comprise: (1) a HCDR1 (Heavy Chain Complementarity Determining Region 1), a HCDR2 and a HCDR3 from the heavy chain variable region (VH) set forth in SEQ ID NO: 54, SEQ ID NO: 82, or SEQ ID NO: 145; (2) a HCDR1, a HCDR2 and a HCDR3 from the heavy chain variable region (VH) set forth in SEQ ID NO: 59 or SEQ ID NO: 83; and/or (1) a LCDR1 (Light Chain Complementarity Determining Region 1), a LCDR2 and a LCDR3 from the light chain variable region (VL) set forth in SEQ ID NO: 64.

In one embodiment, the antibodies or antigen-binding fragments thereof that specifically bind to human cMET comprise:
(1) a heavy chain variable region (VH) that comprises (a) a HCDR1 (Heavy Chain Complementarity Determining Region 1) of SEQ ID NO: 56, (b) a HCDR2 of SEQ ID NO: 57, (c) a HCDR3 of SEQ ID NO: 58 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region (VH) that comprises (a) a HCDR1 (Heavy Chain Complementarity Determining Region 1) of SEQ ID NO: 61, (b) a HCDR2 of SEQ ID NO: 62, (c) a HCDR3 of SEQ ID NO: 63 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; according to the Kabat definition.

In one embodiment, the antibody or an antigen-binding fragment thereof of the present disclosure comprises: (a) a heavy chain variable region comprising an amino acid sequence of SEQ ID NO: 54, SEQ ID NO: 82, SEQ ID NO: 145, SEQ ID NO: 59, or SEQ ID NO: 83; or an amino acid sequence at least 90%, 91%, 92%, 93%, 94%, 95%, 96%, 97%, 98% or 99% identical to any one of SEQ ID NO: 54, SEQ ID NO: 82, SEQ ID NO: 145, SEQ ID NO: 59, or SEQ ID NO: 83 and/or (b) a light chain variable region comprising an amino acid sequence of SEQ ID NO: 64, or an amino acid sequence at least 90%, 91%, 92%, 93%, 94%, 95%, 96%, 97%, 98% or 99% identical to SEQ ID NO: 64.

In one embodiment, the antibody or antigen-binding fragment thereof comprises:
(1) a heavy chain variable region (VH) comprising an amino acid sequence at least 90, 91, 92, 93, 94, 95, 96, 97, 98 or 99% identical to SEQ ID NO: 54, or SEQ ID NO: 82, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 59 or SEQ ID NO: 83, and a light chain variable region (VL) comprising an amino acid sequence at least 90, 91, 92, 93, 94, 95, 96, 97, 98 or 99% identical to SEQ ID NO: 64; or
(3) a heavy chain variable region (VH) comprising an amino acid sequence at least 90, 91, 92, 93, 94, 95, 96, 97, 98 or 99% identical to SEQ ID NO: 145, and a light chain variable region (VL) comprising an amino acid sequence at least 90, 91, 92, 93, 94, 95, 96, 97, 98 or 99% identical to SEQ ID NO: 64.

In one embodiment, the present disclosure provides the antibody or antigen-binding fragment thereof, wherein one, two, three, four, five, six, seven, eight, nine, or ten amino acids within SEQ ID NO: 54, SEQ ID NO: 82, SEQ ID NO: 145, SEQ ID NO: 59, or SEQ ID NO: 83, or SEQ ID NO: 64 have been inserted, deleted or substituted (optionally conservative amino acid substitutions), while retaining therapeutic activity/binding specificity/affinity, optionally the corresponding sequences of CDRs do not change.

In one embodiment, the present disclosure provides the antibody or antigen-binding fragment thereof, wherein one, two, three, four, five, six, seven, eight, nine, or ten amino acids within SEQ ID NO: 54 and SEQ ID NO: 64, SEQ ID NO: 82 and SEQ ID NO: 64, SEQ ID NO: 145 and SEQ ID NO: 64, SEQ ID NO: 59 and SEQ ID NO: 64, or SEQ ID NO: 83 and SEQ ID NO: 64 have been inserted, deleted or substituted (optionally conservative amino acid substitutions), while retaining therapeutic activity/binding specificity/affinity, optionally the corresponding sequences of CDRs do not change.

In one embodiment, the antibody or antigen-binding fragment thereof, that comprises:
(1) a heavy chain variable region (VH) comprising an amino acid sequence of SEQ ID NO: 54, or SEQ ID NO: 82, and a light chain variable region (VL) comprising an amino acid sequence of SEQ ID NO: 64;
(2) a heavy chain variable region (VH) comprising an amino acid sequence of SEQ ID NO: 59 or SEQ ID NO: 83, and a light chain variable region (VL) comprising an amino acid sequence of SEQ ID NO: 64; or
(3) a heavy chain variable region (VH) comprising an amino acid sequence of SEQ ID NO: 145, and a light chain variable region (VL) comprising an amino acid sequence of SEQ ID NO: 64.

Other antibodies or antigen-binding fragments thereof of the present disclosure include amino acids that have been changed, yet have at least 60%, 70%, 80%, 90%, 91%, 92%, 93%, 94%, 95% 96%, 97%, 98%, or 99% percent identity in the CDR regions compared with the CDR regions disclosed in Table 2. In some aspects, it includes amino acid changes (insertion, deletion or substitution, optionally conservative amino acid substitutions) wherein no more than 1, 2, 3, 4 or 5 amino acids have been changed in the CDR regions when compared with the CDR regions depicted in the sequence described in Table 2, while maintaining binding specificity and affinity.

Other antibodies of the present disclosure include those where the amino acids or nucleic acids encoding the amino acids have been changed in the variable regions (e.g., the frameworks regions of the variable regions); yet have at least 60, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% percent identity to the sequences of variable regions described in Table 2, while retaining binding specificity/affinity, optionally the corresponding sequences of CDRs do not change. In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions (e.g., the frameworks regions of the variable regions) when compared with the variable regions depicted in the sequence described in Table 2, while retaining binding specificity/affinity, optionally the corresponding sequences of CDRs do not change. The changes could be insertion, deletion or substitution, optionally conservative amino acid substitutions.

In another embodiment, the present disclosure provides for antibodies or antigen-binding fragments thereof that specifically bind to human cMET with a binding affinity (K_{D}) of from 1×10⁻⁶ M to 1×10⁻¹¹ M. In another embodiment, the anti-cMET antibodies or antigen-binding fragments thereof bind to human cMET with a binding affinity (K_{D}) of about 1×10⁻⁶ M, about 1×10⁻⁷ M, about 1×10⁻⁸ M, about 1×10⁻⁹ M, about 1×10⁻¹⁰ M or about 1×10⁻¹¹ M.

The present disclosure also provides nucleic acid sequences that encode VH or VL of the antibodies that specifically bind to human cMET. Such nucleic acid sequences can be optimized for expression in mammalian cells.

The present disclosure also provides for antibodies and antigen-binding fragments thereof that bind to the same epitope as do the anti-cMET antibodies described in Table 2. Additional antibodies and antigen-binding fragments thereof can therefore be identified based on their ability to cross-compete (e.g., to competitively inhibit the binding of, in a statistically significant manner) with the antibodies described in Table 2 in binding assays. The ability of a test antibody to inhibit the binding of antibodies and antigen-binding fragments thereof of the present disclosure to human cMET demonstrates that the test antibody can compete with that antibody or antigen-binding fragments thereof for binding to human cMET. Such an antibody can, without being bound to any one theory, bind to the same or a related (e.g., a structurally similar or spatially proximal) epitope on human cMET as the antibody or antigen-binding fragments thereof with which it competes. In a certain aspect, the antibody that binds to the same epitope on human cMET as the antibodies or antigen-binding fragments thereof of the present disclosure is a human or humanized monoclonal antibody. Such human or humanized monoclonal antibodies can be prepared and isolated as described herein.

In some embodiments, the antibody or antigen-binding fragment thereof is a monoclonal antibody, a human engineered antibody, a single chain antibody (scFv), a Fab fragment, a Fab′ fragment, or a F(ab′)2 fragment.

In one embodiment, the antibody or antigen-binding fragment thereof is in a scFv format comprising VH-VL in the direction of N-terminal to C-terminal, or VL-VH in the direction of N-terminal to C-terminal. In some embodiments, the VH and VL are connected via an amino acid linker described herein. In some embodiments, the VH comprises the amino acid sequence of any one of SEQ ID NO: 54, SEQ ID NO: 82, SEQ ID NO: 145, SEQ ID NO: 59, or SEQ ID NO: 83. In some embodiments, the VL comprises the amino acid sequence of any one of SEQ ID NO: 64. In some embodiments, the VH is any one of VHs described in Table 2. In some embodiment, the VL is any one of VLs described in Table 2. In some embodiments, the amino acid linker has an amino acid sequence comprising any one of SEQ ID NO: 97-139. In some embodiments, the amino acid linker is any sequence of SEQ ID NO: 97-139.

### II. Anti-EGFR Antibodies

The present disclosure provides for antibodies or antigen-binding fragments thereof that specifically bind to human EGFR. In one embodiment, the anti-EGFR antibodies or antigen-binding fragments thereof specifically bind to human EGFR with a binding affinity (K_{D}) of from 1×10⁻⁶ M to 1×10⁻¹⁰ M. In another embodiment, the anti-EGFR antibodies or antigen-binding fragments thereof bind to human EGFR with a binding affinity (K_{D}) of about 1×10⁻⁶ M, about 1×10⁻⁷ M, about 1×10⁻⁸ M, about 1×10⁻⁹ M or about 1×10⁻¹⁰ M.

In one embodiment, the anti-EGFR antibodies or antigen-binding fragments thereof comprise: a heavy chain variable region (VH) that comprises (a) a HCDR1 of SEQ ID NO: 155, (b) a HCDR2 of SEQ ID NO: 156, (c) a HCDR3 of SEQ ID NO: 157; and a light chain variable region (VL) that comprises (d) a LCDR1 of SEQ ID NO: 158, (e) a LCDR2 of SEQ ID NO: 159, (f) a LCDR3 of SEQ ID NO: 160, according to the Kabat numbering.

In another embodiment, the anti-EGFR antibodies or antigen-binding fragments thereof comprise: a HCDR1, a HCDR2 and a HCDR3 from the heavy chain variable region (VH) set forth in SEQ ID NO: 142; and a LCDR1, a LCDR2 and a LCDR3 from the light chain variable region (VL) set forth in SEQ ID NO: 143.

In another embodiment, the anti-EGFR antibodies or antigen-binding fragments thereof further comprise no more than one, two, three, four or five amino acid deletions, insertions or substitutions in the CDR, preferably the amino acid substitutions are conservative amino acid substitutions, while maintaining binding specificity and affinity.

In another embodiment, the anti-EGFR antibodies or antigen-binding fragments thereof comprise a heavy chain variable region (VH) comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% identical to SEQ ID NO: 142, and a light chain variable region (VL) comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% identical to SEQ ID NO: 143. In another embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids within SEQ ID NO: 142 or SEQ ID NO: 143 have been inserted, deleted or substituted (optionally conservative amino acid substitutions). In another embodiment, such variations are in the framework region of the variable region. In another embodiment, anti-EGFR antibodies or antigen-binding fragments thereof having such variations maintains binding specificity and affinity.

In another embodiment, the anti-EGFR antibodies or antigen-binding fragments thereof comprise a heavy chain variable region (VH) that comprises SEQ ID NO: 142, and a light chain variable region (VL) that comprises SEQ ID NO: 143.

In one embodiment, the anti-EGFR antibody or antigen-binding fragment thereof is in a scFv format comprising VH-VL in the direction of N-terminal to C-terminal, or VL-VH in the direction of N-terminal to C-terminal. In some embodiments, the VH and VL are connected via an amino acid linker described herein. In some embodiments, the VH comprises the amino acid sequence of SEQ ID NO: 142. In some embodiments, the VL comprises the amino acid sequence of SEQ ID NO: 143. In some embodiments, the amino acid linker has an amino acid sequence comprising any one of SEQ ID NO: 97-139. In some embodiments, the amino acid linker is any sequence of SEQ ID NO: 97-139. In one embodiment, the amino acid linker is SEQ ID NO: 139.

In one embodiment, the antibody or antigen-binding fragment thereof comprises a scFv having the amino acid sequence of SEQ ID NO: 161. In another embodiment, the antibody or antigen-binding fragment thereof comprises a scFv of SEQ ID NO: 161.

### III. Multispecific Antibodies

### 3.1 Anti-cMET Bispecific Antibodies

The present disclosure provides for bispecific antibodies or antigen-binding fragments that specifically bind to human cMET, comprising a first antigen binding domain that specifically binds a first epitope of human cMET, and a second antigen binding domain that specifically binds to a second epitope of human cMET, wherein the first antigen binding domain is distinct from the second antigen binding domain.

In one embodiment, the first epitope is distinct from the second epitope (e.g., non-overlapping), or the first antigen binding domain does not compete with the second antigen binding domain.

In one aspect, the first antigen binding domain that specifically binds the first epitope of human cMET and the second antigen binding domain that specifically binds the second epitope of human cMET are selected from any anti-cMET antibody described in Section I and are distinct from each other.

In one aspect, the first antigen binding domain that specifically binds the first epitope of human cMET and the second antigen binding domain that specifically binds the second epitope of human cMET are selected from any anti-cMET antibody described in Section I and do not compete with each other.

In another aspect, the first antigen binding domain that specifically binds the first epitope of human cMET could be any antibody or antigen-binding fragment selected from those described in Section 1.1 “first group of anti-cMET antibodies” including Table 1. The second antigen binding domain that specifically binds the second epitope of human cMET could be any antibody or antigen-binding fragment selected from those described in Section 1.2 “second group of anti-cMET antibodies” including Table 2.

In some embodiments, the bispecific antibody comprises antigen binding fragments, wherein the antigen-binding fragment can be a Fab, F(ab′)₂, Fv, a single chain Fv (scFv), or a single domain antibody.

In one embodiment, the bispecific antibody of the present disclosure binds to human cMET with a binding affinity (K_{D}) of from 1×10⁻⁶ M to 1×10⁻¹⁰ M, or even 1×10⁻¹¹ M. In another embodiment, the bispecific antibody of the present disclosure binds to human cMET with a binding affinity (K_{D}) of about 1×10⁻⁶ M, about 1×10⁻⁷ M, about 1×10⁻⁸ M, about 1×10⁻⁹ M, about 1×10⁻¹⁰ M, or about 1×10⁻¹¹ M.

In some embodiments, the bispecific antibodies of the present disclosure specifically binding to two distinct epitopes (e.g., non-overlapping or non-competing) of human cMET exhibit better blocking activity on ligand independent signaling and/or ligand induced signaling, compared with those of the antibodies comprising only one antigen binding domain that specifically binds to human cMET.

In one embodiment, the present disclosure provides for a bispecific antibody or antigen-binding fragment thereof, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68; or
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68; and
wherein the second antigen binding domain that specifically binds to the second epitope of human cMET comprises:
a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68.

In one embodiment, the present disclosure provides for a bispecific antibody or antigen-binding fragment thereof, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(3) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(4) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(5) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; and
wherein the second antigen binding domain that specifically binds to the second epitope of human cMET comprises:
a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68.

In one embodiment, the present disclosure provides for a bispecific antibody or antigen-binding fragment thereof, wherein the first antigen binding domain that specifically binds to the first epitope of human cMET comprises:
(1) a heavy chain variable region (VH) comprising an amino acid sequence of SEQ ID NO: 4 or SEQ ID NO: 72, and a light chain variable region (VL) comprising an amino acid sequence of SEQ ID NO: 64;
(2) a heavy chain variable region (VH) comprising an amino acid sequence of SEQ ID NO: 9 or SEQ ID NO: 73, and a light chain variable region (VL) comprising an amino acid sequence of SEQ ID NO: 64;
(3) a heavy chain variable region (VH) comprising an amino acid sequence of SEQ ID NO: 14, or SEQ ID NO: 74, and a light chain variable region (VL) comprising an amino acid sequence of SEQ ID NO: 64;
(4) a heavy chain variable region (VH) comprising an amino acid sequence of SEQ ID NO: 19, or SEQ ID NO: 75, and a light chain variable region (VL) comprising an amino acid sequence of SEQ ID NO: 64;
(5) a heavy chain variable region (VH) comprising an amino acid sequence of SEQ ID NO: 24, or SEQ ID NO: 76, and a light chain variable region (VL) of SEQ ID NO: 64;
(6) a heavy chain variable region (VH) comprising an amino acid sequence of SEQ ID NO: 29, or SEQ ID NO: 77, and a light chain variable region (VL) comprising an amino acid sequence of SEQ ID NO: 64;
(7) a heavy chain variable region (VH) comprising an amino acid sequence of SEQ ID NO: 34, or SEQ ID NO: 78, and a light chain variable region (VL) comprising an amino acid sequence of SEQ ID NO: 64;
(8) a heavy chain variable region (VH) comprising an amino acid sequence of SEQ ID NO: 39, or SEQ ID NO: 79, and a light chain variable region (VL) comprising an amino acid sequence of SEQ ID NO: 64;
(9) a heavy chain variable region (VH) comprising an amino acid sequence of SEQ ID NO: 44, or SEQ ID NO: 80, and a light chain variable region (VL) comprising an amino acid sequence of SEQ ID NO: 64; or
(10) a heavy chain variable region (VH) comprising an amino acid sequence of SEQ ID NO: 49 or SEQ ID NO: 81, and a light chain variable region (VL) comprising an amino acid sequence of SEQ ID NO: 64; and
wherein the second antigen binding domain that specifically binds to the second epitope of human cMET comprises:
a heavy chain variable region (VH) comprising an amino acid sequence of SEQ ID NO: 54, SEQ ID NO: 82 or SEQ ID NO: 145, and a light chain variable region (VL) comprising an amino acid sequence of SEQ ID NO: 64.

In one embodiment, the present disclosure provides for a bispecific antibody or antigen-binding fragment thereof, wherein the first antigen binding domain that specifically binds to the first epitope of human cMET comprises
a heavy chain variable region (VH) comprising an amino acid sequence of SEQ ID NO: 85, SEQ ID NO: 87, SEQ ID NO: 89, SEQ ID NO: 91, SEQ ID NO: 93, or SEQ ID NO: 144, and a light chain variable region (VL) comprising an amino acid sequence of SEQ ID NO: 64; and
wherein the second antigen binding domain that specifically binds to the second epitope of human cMET comprises:
a heavy chain variable region (VH) comprising an amino acid sequence of SEQ ID NO: 54, SEQ ID NO: 82 or SEQ ID NO: 145, and a light chain variable region (VL) comprising an amino acid sequence of SEQ ID NO: 64.

In some embodiments, in the above-mentioned bispecific antibodies or the antigen-binding fragments thereof, the second antigen binding domain that specifically binds to the second epitope of human cMET could be changed.

In one embodiment, the second antigen binding domain that specifically binds to the second epitope of human cMET comprises:
a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68.

In one embodiment, the second antigen binding domain that specifically binds to the second epitope of human cMET comprises a heavy chain variable region (VH) comprising an amino acid sequence of SEQ ID NO: 59, or SEQ ID NO: 83, and a light chain variable region (VL) comprising an amino acid sequence of SEQ ID NO: 64.

In another embodiment, the bispecific antibody further comprises an amino acid linker described herein. In some embodiments, the amino acid linker has an amino acid sequence comprising any one of SEQ ID NO: 97-139. In some embodiments, the amino acid linker is any sequence of SEQ ID NO: 97-139.

### 3.2 Anti-cMET×Antigen Multispecific Antibodies

In one embodiment, the anti-cMET antibodies disclosed herein (including Section I “anti-cMET antibodies” and Section 3.1 “anti-cMET bispecific antibodies) can be used to construct multispecific antibodies with other modalities such as a human tumor associated antigen (TAA), immune checkpoints or immune stimulators.

In one embodiment, the anti-cMET antibodies as disclosed herein can be incorporated into an anti-cMET×TAA multispecific antibody, wherein anti-TAA is an antibody or fragment thereof directed to any human tumor associated antigen (TAA) other than cMET. An antibody molecule is a multispecific antibody molecule, for example, it comprises at least two antigen binding domains, wherein at least one antigen binding domain sequence specifically binds cMET as a first epitope and a second antigen binding domain sequence specifically binds a TAA as a second epitope.

In another embodiment, two antigen binding domain sequences specifically binds two distinct epitopes (e.g., non-overlapping or non-competing) of human cMET as a first epitope and a second epitope, respectively, and the third antigen binding domain sequence specifically binds a TAA as a third epitope. In one embodiment, the multispecific antibody comprises a third, fourth or fifth antigen binding domain. In one embodiment, the multispecific antibody is a bispecific antibody, a trispecific antibody, or tetraspecific antibody.

In one embodiment, the multispecific antibody is a bispecific antibody. As used herein, a bispecific antibody specifically binds only two antigens. The bispecific antibody comprises a first antigen binding domain which specifically binds human cMET and a second antigen binding domain that specifically binds a TAA. This includes a bispecific antibody comprising a second heavy chain variable domain and a second light chain variable domain which specifically binds a TAA and a first heavy chain variable domain and a first light chain variable domain which specifically binds human cMET. In some embodiments, the bispecific antibody comprises antigen binding fragments, wherein the antigen-binding fragment can be a Fab, F(ab′)₂, Fv, a single chain Fv (scFv), or a single domain antibody.

In one embodiment, the multispecific antibody is a trispecific antibody. As used herein, a trispecific antibody specifically binds at least three antigens or epitopes. The trispecific antibody comprises a first antigen binding domain which specifically binds a first epitope of human cMET, a second antigen binding domain which specifically binds a second epitope of human cMET, and a third antigen binding domain that specifically binds a TAA. This includes a trispecific antibody comprising a third heavy chain variable domain and a third light chain variable domain which specifically binds a TAA, a first heavy chain variable domain and a first light chain variable domain which specifically binds the first epitope of human cMET, and a second heavy chain variable domain and a second light chain variable domain which specifically binds the second epitope of human cMET. In some embodiments, the trispecific antibody comprises antigen binding fragments, wherein the antigen-binding fragment can be a Fab, F(ab′)₂, Fv, a single chain Fv (scFv), or a single domain antibody.

In one embodiment, the multispecific antibody of the present disclosure binds to a human TAA and/or human cMET with a binding affinity (K_{D}) of from 1×10⁻⁶ M to 1×10⁻¹⁰ M, or even 1×10⁻¹¹ M. In another embodiment, the multispecific antibody of the present disclosure binds to a human TAA and at least one epitope on human cMET with a binding affinity (K_{D}) of about 1×10⁻⁶ M, about 1×10⁻⁷ M, about 1×10⁻⁸ M, about 1×10⁻⁹ M, about 1×10⁻¹⁰ M, or about 1×10⁻¹¹ M.

Previous experimentation (Coloma and Morrison Nature Biotech. 15: 159-163 (1997), incorporated by reference in its entirety) described a tetravalent bispecific antibody which was engineered by fusing DNA encoding a single chain anti-dansyl antibody Fv (scFv) after the C terminus (CH3-scFv) or after the hinge (hinge-scFv) of an lgG3 anti-dansyl antibody. The present disclosure provides multivalent antibodies (e.g., tetravalent antibodies) with at least two antigen binding domains, which can be readily produced by recombinant expression of nucleic acid encoding the polypeptide chains of the antibody. The multivalent antibody herein comprises three to eight, but preferably three or four antigen binding domains, which specifically bind at least two antigens.

In one embodiment, the multispecific antibody is a bispecific antibody, trispecific antibody or tetraspecific antibody. In another embodiment, the multispecific antibody further comprises an amino acid linker described herein. In some embodiments, the amino acid linker has an amino acid sequence comprising any one of SEQ ID NO: 97-139. In some embodiments, the amino acid linker is any sequence of SEQ ID NO: 97-139.

### 3.3 EGFR×cMET Multispecific Antibodies

In one aspect, the present disclosure provides a multispecific antibody or antigen-binding fragment thereof, comprising a first antigen binding domain that specifically binds a first epitope of human cMET; a second antigen binding domain that specifically binds to a second epitope of human cMET; and a third antigen binding domain that specifically binds to human EGFR.

In one embodiment, the first epitope is distinct from the second epitope, or wherein the first antigen binding domain does not compete with the second antigen binding domain.

In one aspect, EGFR×cMET multispecific antibodies further comprises a third antigen binding domain that specifically binds to human EGFR on the top of anti-cMET bispecific antibodies described in Section 3.1

In one aspect, in the anti-cMET×Antigen multispecific antibodies described in Section 3.2, the further antigen (TAA) is human EGFR.

In one embodiment, the first antigen binding domain that specifically binds a first epitope of human cMET is selected from any anti-cMET antibody described in Section 1.1.

In one embodiment, the second antigen binding domain that specifically binds a second epitope of human cMET is selected from any anti-cMET antibody described in Section 1.2.

In one embodiment, the third antigen binding domain that specifically binds to human EGFR is selected from any anti-EGFR antibody described in Section II.

In one embodiment, in the EGFR×cMET multispecific antibody or antigen-binding fragment thereof, the first antigen binding domain that specifically binds to the first epitope of human cMET comprises: a heavy chain variable region that comprises (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68; the second antigen binding domain that specifically binds to the second epitope of human cMET comprises: a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; and the third antigen binding domain that specifically binds to human EGFR comprises: a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

In one embodiment, in the EGFR×cMET multispecific antibody or antigen-binding fragment thereof, the first antigen binding domain that specifically binds to the first epitope of human cMET comprises: a heavy chain variable region (VH) comprising an amino acid sequence of SEQ ID NO: 144, and a light chain variable region (VL) comprising an amino acid sequence of SEQ ID NO: 64; the second antigen binding domain that specifically binds to the second epitope of human cMET comprises: a heavy chain variable region (VH) comprising an amino acid sequence of SEQ ID NO: 145, and a light chain variable region (VL) comprising an amino acid sequence of SEQ ID NO: 64; and the third antigen binding domain that specifically binds to human EGFR comprises: a heavy chain variable region (VH) comprising an amino acid sequence of SEQ ID NO: 142, and a light chain variable region (VL) comprising an amino acid sequence of SEQ ID NO: 143.

In one embodiment, the first antigen binding domain that specifically binds to the first epitope of human cMET is a monoclonal antibody, a human engineered antibody, a single chain antibody (scFv), a Fab fragment, a Fab′ fragment, or a F(ab′)2 fragment; the second antigen binding domain that specifically binds to the second epitope of human cMET is a monoclonal antibody, a human engineered antibody, a single chain antibody (scFv), a Fab fragment, a Fab′ fragment, or a F(ab′)2 fragment; and the third antigen binding domain that specifically binds to human EGFR is a monoclonal antibody, a human engineered antibody, a single chain antibody (scFv), a Fab fragment, a Fab′ fragment, or a F(ab′)2 fragment.

In one embodiment, the EGFR×cMET multispecific antibody or antigen-binding fragment thereof comprises a heavy chain constant region of the subclass of IgG1, IgG2, IgG3, or IgG4, and/or a light chain constant region of the type of kappa or lambda.

In one embodiment, the EGFR×cMET multispecific antibody or antigen-binding fragment thereof comprises a heavy chain constant region of the subclass of IgG1, and a light chain constant region of the type of kappa.

In one embodiment, the EGFR×cMET multispecific antibody or antigen-binding fragment thereof has antibody dependent cellular cytotoxicity (ADCC), antibody-dependent cellular phagocytosis (ADCP) or complement dependent cytotoxicity (CDC).

In one embodiment, the EGFR×cMET multispecific antibody or antigen-binding fragment thereof has reduced glycosylation or no glycosylation or is hypofucosylated. In another embodiment, the multispecific antibody or antigen-binding fragment thereof is afucosylated.

In one embodiment, the EGFR×cMET multispecific antibody or antigen-binding fragment thereof comprises increased bisecting GlcNac structures.

In one embodiment, the EGFR×cMET multispecific antibody or antigen-binding fragment thereof comprises a Fc domain, and wherein the Fc domain is an IgG1 Fc with extended half-life.

In one embodiment, the EGFR×cMET multispecific antibody or antigen-binding fragment thereof adopts knob into hole (KIH) to form heterodimer.

In one embodiment, the first antigen binding domain that specifically binds to the first epitope of human cMET comprises a first heavy chain constant region comprising SEQ ID NO: 95, and the second antigen binding domain that specifically binds to the second epitope of human cMET comprises a second heavy chain constant region comprising SEQ ID NO: 96.

In another embodiment, the first antigen binding domain that specifically binds to the first epitope of human cMET comprises a first heavy chain constant region comprising SEQ ID NO: 96, and the second antigen binding domain that specifically binds to the second epitope of human cMET comprises a second heavy chain constant region comprising SEQ ID NO: 95.

In another embodiment, the first antigen binding domain that specifically binds to the first epitope of human cMET and the second antigen binding domain that specifically binds to the second epitope of human cMET share common light chain.

In one embodiment, the first light chain constant region and the second light chain constant region are different. In another embodiment, the first light chain constant region and the second light chain constant region are the same.

In one embodiment, the light chain constant region has an amino acid sequence of SEQ ID NO: 71. In one embodiment, the light chain constant region is SEQ ID NO: 71.

In one embodiment, the first light chain variable region and the second light chain variable region are different. In another embodiment, the first light chain variable region and the second light chain variable region are the same.

In one embodiment, the EGFR×cMET multispecific antibody or antigen-binding fragment thereof further comprises amino acid linker herein.

In one embodiment, the EGFR×cMET multispecific antibody or antigen-binding fragment thereof could be in different format with different EGFR scFv valency and orientation to cMET arms. The format could be any format shown in FIG. 7, represented by TE-644, TE-645, TE-646, TE-647, TE-648 or TE-642.

In one embodiment, the EGFR×cMET multispecific antibody or antigen-binding fragment thereof comprises a first polypeptide, a second polypeptide, a third polypeptide, and a fourth polypeptide, wherein (1) a VL of the third antigen binding domain that specifically binds to human EGFR, optionally the first amino acid linker, a VH of the third antigen binding domain that specifically binds to human EGFR, optionally the second amino acid linker, a VH of the second antigen binding domain that specifically binds to the second epitope of human cMET, and the second heavy chain constant region are arranged in the first polypeptide in the direction of N terminal to C terminal; (2) a VH of the first antigen binding domain that specifically binds to the first epitope of human cMET, and the first heavy chain constant region are arranged in the second polypeptide in the direction of N terminal to C terminal; or a VL of the third antigen binding domain that specifically binds to human EGFR, optionally the first amino acid linker, a VH of the third antigen binding domain that specifically binds to human EGFR, optionally the second amino acid linker, a VH of the first antigen binding domain that specifically binds to the first epitope of human cMET, the first heavy chain constant region, are arranged in the second polypeptide in the direction of N terminal to C terminal; (3) a VL of the second antigen binding domain that specifically binds to the second epitope of human cMET, a second light chain constant region, are arranged in the third polypeptide in the direction of N terminal to C terminal; and (4) a VL of the first antigen binding domain that specifically binds to the first epitope of human cMET, a first light chain constant region, are arranged in the fourth polypeptide in the direction of N terminal to C terminal.

In one embodiment, the EGFR×cMET multispecific antibody or antigen-binding fragment thereof comprises a first polypeptide, a second polypeptide, a third polypeptide, and a fourth polypeptide, wherein (1) a VL of the third antigen binding domain that specifically binds to human EGFR, optionally the first amino acid linker, a VH of the third antigen binding domain that specifically binds to human EGFR, optionally the second amino acid linker, a VH of the second antigen binding domain that specifically binds to the second epitope of human cMET, and the second heavy chain constant region are arranged in the first polypeptide in the direction of N terminal to C terminal; (2) a VH of the first antigen binding domain that specifically binds to the first epitope of human cMET, and the first heavy chain constant region are arranged in the second polypeptide in the direction of N terminal to C terminal; (3) a VL of the second antigen binding domain that specifically binds to the second epitope of human cMET, a second light chain constant region, are arranged in the third polypeptide in the direction of N terminal to C terminal; and (4) a VL of the first antigen binding domain that specifically binds to the first epitope of human cMET, a first light chain constant region, are arranged in the fourth polypeptide in the direction of N terminal to C terminal.

In one embodiment, the second heavy chain constant region is SEQ ID NO: 95, and the first heavy chain constant region is SEQ ID NO: 96; or the second heavy chain constant region is SEQ ID NO: 96, and the first heavy chain constant region is SEQ ID NO: 95.

In one embodiment, the first amino acid linker is SEQ ID NO: 139. In another embodiment, the second amino acid linker is SEQ ID NO: 139.

In one embodiment, a VL of the first antigen binding domain and a VL of the second antigen binding domain are the same.

In another embodiment, the first light chain constant region and the second light chain constant region are the same. In another embodiment, the first light chain constant region and the second light chain constant region are SEQ ID NO: 71.

In one embodiment, the third polypeptide and the fourth polypeptide are the same.

In another embodiment, the position of the first antigen binding domain that specifically binds to the first epitope of human cMET (such as BGA-109) and the second antigen binding domain that specifically binds to the second epitope of human cMET (such as BGA-032) could be exchanged.

In one embodiment, the multispecific antibody or antigen-binding fragment thereof comprises a first polypeptide, a second polypeptide, and a third polypeptide, wherein
(1) the first polypeptide has an amino acid sequence of SEQ ID NO: 153, the second polypeptide has an amino acid sequence of SEQ ID NO: 150, and the third polypeptide has an amino acid sequence of SEQ ID NO: 148;
(2) the first polypeptide has an amino acid sequence of SEQ ID NO: 146, the second polypeptide has an amino acid sequence of SEQ ID NO: 147, and the third polypeptide has an amino acid sequence of SEQ ID NO: 148;
(3) the first polypeptide has an amino acid sequence of SEQ ID NO: 149, the second polypeptide has an amino acid sequence of SEQ ID NO: 150, and the third polypeptide has an amino acid sequence of SEQ ID NO: 151;
(4) the first polypeptide has an amino acid sequence of SEQ ID NO: 149, the second polypeptide has an amino acid sequence of SEQ ID NO: 150, and the third polypeptide has an amino acid sequence of SEQ ID NO: 152;
(5) the first polypeptide has an amino acid sequence of SEQ ID NO: 153, the second polypeptide has an amino acid sequence of SEQ ID NO: 154, and the third polypeptide has an amino acid sequence of SEQ ID NO: 148; or
(6) the first polypeptide has an amino acid sequence of SEQ ID NO: 146, the second polypeptide has an amino acid sequence of SEQ ID NO: 150, and the third polypeptide has an amino acid sequence of SEQ ID NO: 148.

In another embodiment, the multispecific antibody or antigen-binding fragment thereof of comprises
(1) a first polypeptide of SEQ ID NO: 153, the second polypeptide of SEQ ID NO: 150, and the third polypeptide of SEQ ID NO: 148;
(2) the first polypeptide of SEQ ID NO: 146, the second polypeptide of SEQ ID NO: 147, and the third polypeptide of SEQ ID NO: 148;
(3) the first polypeptide of SEQ ID NO: 149, the second polypeptide of SEQ ID NO: 150, and the third polypeptide of SEQ ID NO: 151;
(4) the first polypeptide of SEQ ID NO: 149, the second polypeptide of SEQ ID NO: 150, and the third polypeptide of SEQ ID NO: 152;
(5) the first polypeptide of SEQ ID NO: 153, the second polypeptide of SEQ ID NO: 154, and the third polypeptide of SEQ ID NO: 148; or
(6) the first polypeptide of SEQ ID NO: 146, the second polypeptide of SEQ ID NO: 150, and the third polypeptide of SEQ ID NO: 148.
   IV. Anti-Human cMET Antibodies Conjugated with a Cytotoxin

The anti-human cMET antibodies disclosed herein (including Section I “anti-cMET antibodies” and Section III “multispecific antibodies, e.g., Section 3.1 anti-cMET bispecific antibodies, 3.2 Anti-cMET×Antigen multispecific antibodies, or 3.3 EGFR×cMET multispecific antibodies) can be used to construct antibody drug conjugate (ADC). In one embodiment, the antibody or antigen-binding fragment thereof is conjugated to a cytotoxin. In another embodiment, the antibody or antigen-binding fragment thereof is conjugated to a cytotoxin via a cytotoxin linker.

### Cytotoxin

Cytotoxin or cytotoxic agents include any agent that is detrimental to the growth, viability or propagation of cells, including, but not limited to, tubulin-interacting agents and DNA-damaging agents. Examples of suitable cytotoxic agents and chemotherapeutic agents that can be conjugated to the antibodies of the present disclosure include, e.g., 1-(2chloroethyl)-1,2-dimethanesulfonylhydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins (e.g., calicheamicin gammal), camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing.

### Cytotoxin Linker

Cytotoxin linkers or linkers for ADC are any group or moiety that links, connects, or bonds the antibody or antigen-binding proteins described herein with a therapeutic moiety, e.g., cytotoxic agent. Suitable linkers may be found, for example, in Antibody-Drug Conjugates and Immunotoxins; Phillips, G. L, Ed.; Springer Verlag: New York, 2013; Antibody-Drug Conjugates; Ducry, L, Ed.; Humana Press, 2013; Antibody-Drug Conjugates; Wang, J., Shen, W.-C, and Zaro, J. L, Eds.; Springer International Publishing, 2015, the contents of each incorporated herein in their entirety by reference.

Generally, suitable binding agent or cytotoxin linkers for the antibody conjugates described herein are those that are sufficiently stable to exploit the circulating half-life of the antibody and, at the same time, capable of releasing its payload after antigen-mediated internalization of the conjugate. Linkers can be cleavable or non-cleavable. Cleavable linkers include linkers that are cleaved by intracellular metabolism following internalization, e.g., cleavage via hydrolysis, reduction, or enzymatic reaction. Non-cleavable linkers include linkers that release an attached payload via lysosomal degradation of the antibody following internalization. Suitable linkers include, but are not limited to, acid-labile linkers, hydrolysis-labile linkers, enzymatically cleavable linkers, reduction labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citruline units, and para-aminobenzyl (PAB) units.

Any cytotoxin linker molecule or linker technology known in the art can be used to create or construct an ADC of the present disclosure. In certain embodiments, the cytotoxin linker is a cleavable linker. According to other embodiments, the linker is a non-cleavable linker. Exemplary linkers that can be used in the context of the present disclosure include, linkers that comprise or consist of e.g., GGFG, MC (6-maleimidocaproyl), MP (maleimidopropanoyl), val-cit (valine-citrulline), val-ala (valine-alanine), dipeptide site in protease-cleavable linker, ala-phe (alanine-phenylalanine), dipeptide site in protease-cleavable linker, PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate), and variants and combinations thereof. Additional examples of linkers that can be used in the context of the present disclosure are provided, e.g., in U.S. Pat. No. 7,754,681 and in Ducry, Bioconjugate Chem., 2010, 27:5-13, and the references cited therein, the contents of which are incorporated by reference herein in their entireties.

In certain embodiments, the cytotoxin linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker.

In some embodiments, the cytotoxin linker comprises a non-cleavable moiety.

Suitable cytotoxin linkers also include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the cytotoxin linker comprises one or more amino acids. Suitable amino acids include natural, non-natural, standard, non-standard, proteinogenic, non-proteinogenic, and L- or D- -amino acids. In some embodiments, the cytotoxin linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In certain embodiments, one or more side chains of the amino acids is linked to a side chain group, described below. In some embodiments, the linker comprises valine and citrulline. In some embodiments, the cytotoxin linker comprises lysine, valine, and citrulline. In some embodiments, the linker comprises lysine, valine, and alanine. In some embodiments, the linker comprises valine and alanine.

V. Fusion Protein Targeting Human cMET

The anti-human cMET antibodies can be used to fuse with other proteins or other functional domains to form fusion proteins or chimeric proteins.

### VI. Other Modifications

### Constant Region and Fe Region

The heavy chain constant region could be the wild type sequences of heavy chain constant region from the subclass of IgG1, IgG2, IgG3, or IgG4. The light chain constant region could be the wild type sequences of light chain from kappa or lambda type. In one embodiment, the heavy chain constant region is the wild type sequence of constant region from IgG1. The light chain constant region is the wild type sequence of light chain from kappa chain. In one embodiment, the heavy chain constant region has the amino acid sequences of SEQ ID NO: 70 and the light chain constant region has the amino acid sequences of SEQ ID NO: 71. In one embodiment, the Fc region could be wild type Fc region of the subclass of IgG1, IgG2, IgG3, or IgG4.

In one embodiment, the antibody or antigen-binding fragment thereof comprises a Fc domain of IgG1 or IgG4 with reduced effector function. In another embodiment, the heavy chain constant region comprises mutations E233P, L234A, L235A, G236del, and P329A.

In one embodiment, the antibody or antigen-binding fragment thereof comprises a Fc domain with extended half-life. In another embodiment, the antibody or antigen-binding fragment thereof comprises a Fc domain of IgG1, wherein YTE mutation (M252Y/S254T/T256E, EU numbering, as described in U.S. Pat. No. 7,658,921, incorporated by reference in its entirety) located at CH2 of IgG Fc region were introduced.

In another embodiment, antibodies of the present disclosure have strong Fc-mediated effector functions, and the antibodies mediate antibody-dependent cellular cytotoxicity (ADCC) against target cells.

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al, each incorporated by reference in their entirety.

In another aspect, one or more amino acid residues can be replaced with one or more different amino acid residues such that the antibody has altered Clq binding and/or reduced or abolished complement dependent cytotoxicity (CDC). This approach is described in, e.g., U.S. Pat. No. 6,194,551 by Idusogie et al, incorporated by reference in its entirety.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009), incorporated by reference in its entirety.

In another aspect, the Fc region is modified to increase the ability of the antibody to mediate antibody dependent cellular cytotoxicity (ADCC) and/or to increase the affinity of the antibody for an Fc7 receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001), incorporated by reference in its entirety.

In still another aspect, the glycosylation of the antibody is modified. For example, an aglycosylated antibody can be made (i.e., the antibody lacks or has reduced glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al, incorporated by reference in its entirety.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., incorporated by reference in its entirety, describes a cell line with a functionally disrupted FUT8 gene, which encodes a fucosyl transferase, such that antibodies expressed in such a cell line exhibit hypofucosylation. Publication WO 03/035835, incorporated by reference in its entirety, by Presta describes a variant CHO cell line, LecI3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740), incorporated by reference in its entirety. WO99/54342 by Umana et al., incorporated by reference in its entirety, describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999, incorporated by reference in its entirety).

WO2003085107A1 by Naoko Ohnuki et al., describes an engineered CHO cell line in which the activity of α1, 6-fucosyltransferase is lower or disappeared, so that the produced antibodies or antigen-binding fragment thereof is afucosylated.

In another aspect, if a reduction of ADCC is desired, human antibody subclass IgG4 was shown in many previous reports to have only modest ADCC and almost no CDC effector function (Moore G L, et al., 2010 MAbs, 2:181-189, incorporated by reference in its entirety). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19, each incorporated by reference in their entirety). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157, incorporated by reference in its entirety). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19, each incorporated by reference in their entirety). Some of the amino acid residues in the hinge and 7Fc region were reported to have impact on antibody interaction with Fc7 receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev immunol, 25:21-50, each incorporated by reference in their entirety). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19, each incorporated by reference in their entirety). To generate multispecific antibodies with low ADCC and CDC but with good stability, it is possible to modify the hinge and Fc region of human IgG4 and introduce a number of alterations. These modified IgG4 Fc molecules can be found in SEQ ID NOs: 83-88 of U.S. Pat. No. 8,735,553 to Li et al, each incorporated by reference in their entirety.

In another embodiment, the antibody of the present disclosure comprises Fc domain of human IgG4 with S228P and/or R409K substitutions (according to EU numbering system).

“Knob-into-hole” mutations could be incorporated into the Fc: Fc binding interfaces. In some embodiments, knob-into-holes insure the correct pairing of two different heavy chains together during the manufacture of multispecific antibodies. In one embodiment, the first heavy chain constant region or the first Fc of the multispecific antibodies herein comprises SEQ ID NO: 95, and the second heavy chain constant region or the second Fc of the multispecific antibodies herein comprises SEQ ID NO: 96. In another embodiment, the first heavy chain constant region or the first Fc of the multispecific antibodies herein comprises SEQ ID NO: 96, and the second heavy chain constant region or the second Fc of the multispecific antibodies herein comprises SEQ ID NO: 95.

In another embodiment, the first heavy chain constant region or the first Fc of the multispecific antibodies herein comprises variant of human IgG1 constant region comprising T366W, and second heavy chain constant region or the second Fc of the multispecific antibodies herein comprises variant of human IgG1 constant region comprising T366S, L368A, and Y407V (EU Numbering).

In another embodiment, the first heavy chain constant region or the first Fc of the multispecific antibodies herein comprises variant of human IgG1 constant region comprising T366S, L368A, and Y407V, and second heavy chain constant region or the second Fc of the multispecific antibodies herein comprises variant of human IgG1 constant region comprising T366W (EU Numbering).

### Amino Acid Linkers

It is also understood that the domains and/or regions of the polypeptide chains of the antibody or protein can be separated by linker regions of various lengths. In some embodiments, the antigen binding domains are separated from each other, a CL, CH1, hinge, CH2, CH3, or the entire Fc region by a linker region. For example, VL1-CL-(linker) VH2-CH1. Such linker region may comprise a random assortment of amino acids, or a restricted set of amino acids. Such linker regions can be flexible or rigid (see e.g., US2009/0155275, incorporated by reference in its entirety).

Multispecific antibodies have been constructed by genetically fusing two single chain Fv (scFv) or Fab fragments with or without the use of flexible linkers (Mallender et al., J. Biol. Chem. 1994 269:199-206; Mack et. al.., Proc. Natl. Acad. Sci. USA. 1995 92:7021-5; Zapata et al., Protein Eng. 1995 8.1057-62), via a dimerization device such as leucine Zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Pack et. al.., Biochemistry 1992.31:1579-84; Pack et. al.., Bio/Technology 1993 11:1271-7). Each reference mentioned in this paragraph incorporated by reference in their entirety.

The antibodies or proteins as disclosed herein comprise an amino acid linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its antigen binding domains, CL domains, CH1 domains, Hinge region, CH2 domains, CH3 domains, or Fc regions. In some embodiments, the amino acids glycine and serine are comprised within the linker region. In another embodiment, the linker can be GS (SEQ ID NO: 97), GGS (SEQ ID NO: 98), GSG (SEQ ID NO: 99), SGG (SEQ ID NO: 100), GGG (SEQ ID NO: 101), GGGS (SEQ ID NO: 102), SGGG (SEQ ID NO: 103), GGGGS (SEQ ID NO: 104), GGGGSGS (SEQ ID NO: 105), GGGGSGS (SEQ ID NO: 106), GGGGSGGS (SEQ ID NO: 107), GGGGSGGGGS (SEQ ID NO: 108), GGGGSGGGGSGGGGS (SEQ ID NO: 109), AKTTPKLEEGEFSEAR (SEQ ID NO: 110), AKTTPKLEEGEFSEARV (SEQ ID NO: 111), AKTTPKLGG (SEQ ID NO: 112), SAKTTPKLGG (SEQ ID NO: 113), AKTTPKLEEGEFSEARV (SEQ ID NO: 114), SAKTTP (SEQ ID NO: 115), SAKTTPKLGG (SEQ ID NO: 116), RADAAP (SEQ ID NO: 117), RADAAPTVS (SEQ ID NO: 118), RADAAAAGGPGS (SEQ ID NO: 119), RADAAAA(G₄S)₄ (SEQ ID NO: 120), SAKTTP (SEQ ID NO: 121), SAKTTPKLGG (SEQ ID NO: 122), SAKTTPKLEEGEFSEARV (SEQ ID NO: 123), ADAAP (SEQ ID NO: 124), ADAAPTVSIFPP (SEQ ID NO: 125), TVAAP (SEQ ID NO: 126), TVAAPSVFIFPP (SEQ ID NO: 127), QPKAAP (SEQ ID NO: 128), QPKAAPSVTLFPP (SEQ ID NO: 129), AKTTPP (SEQ ID NO: 130), AKTTPPSVTPLAP (SEQ ID NO: 131), AKTTAP (SEQ ID NO: 132), AKTTAPSVYPLAP (SEQ ID NO: 133), ASTKGP (SEQ ID NO: 134), ASTKGPSVFPLAP (SEQ ID NO: 135), GENKVEYAPALMALS (SEQ ID NO: 136), GPAKELTPLKEAKVS (SEQ ID NO: 137), and GHEAAAVMQVQYPAS (SEQ ID NO: 138), GGGGSGGGGSGGGGSGGGGS (SEQ ID NO: 139) or any combination thereof (see WO2007/024715).

### Dimerization Specific Amino Acids

In one embodiment, the multivalent antibody comprises at least one dimerization specific amino acid change. The dimerization specific amino acid changes result in “knobs into holes” interactions, and increases the assembly of correct multivalent antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 family and WO2017059551, each incorporated by reference in their entirety. The dimerization specific amino acids can also be within the Fc domain. Also, the dimerization specific amino acids within the Fc domain can be in combination with dimerization specific amino acids within the CH1 or CL domains. In one embodiment, the disclosure provides a multispecific antibody comprising at least one dimerization specific amino acid pair.

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies or proteins described herein, e.g., polynucleotides encoding heavy chain variable regions or light chain variable regions comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from SEQ ID NO: 5, SEQ ID NO: 162, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, SEQ ID NO: 55, SEQ ID NO: 163, or SEQ ID NO: 60. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from SEQ ID NO: 65.

The present disclosure also provides polynucleotides encoding the scFv described herein.

The polynucleotides of the present disclosure can encode the variable region sequence of the multispecific antibody described herein. They can also encode both a variable region and a constant region of the multispecific antibody. In another embodiment, the polynucleotides of the present disclosure can encode the amino acid sequences of fusion proteins described herein.

In some embodiments, the polynucleotides described herein could be codon-optimized for expression in host cells, e.g., eukaryotic cells, more specifically mammalian cells (e.g., CHO cells).

In some embodiment, the present disclosure provides the polynucleotides encoding polypeptides (e.g., all polypeptides recited in Table 21 and Table 22) of the multispecific antibodies herein, e.g., EGFR×cMET multispecific antibodies described herein.

In one embodiment, the polynucleotide encoding the first polypeptide of EGFR×cMET multispecific antibody has at least 85%, 89%, 90%, 91%, 92%, 93%, 94%, 95%, 96%, 97%, 98%, 99%, or 100% nucleic acid sequence identity with a polynucleotide having SEQ ID NO: 164, the polynucleotide encoding the second polypeptide of EGFR×cMET multispecific antibody has at least 85%, 89%, 90%, 91%, 92%, 93%, 94%, 95%, 96%, 97%, 98%, 99%, or 100% nucleic acid sequence identity with a polynucleotide having SEQ ID NO: 165, and the polynucleotide encoding the third polypeptide of EGFR×cMET multispecific antibody has at least 85%, 89%, 90%, 91%, 92%, 93%, 94%, 95%, 96%, 97%, 98%, 99%, or 100% nucleic acid sequence identity with a polynucleotide having SEQ ID NO: 166.

In one embodiment, the polynucleotide encoding the first polypeptide of EGFR×cMET multispecific antibody has at least 85%, 89%, 90%, 91%, 92%, 93%, 94%, 95%, 96%, 97%, 98%, 99%, or 100% nucleic acid sequence identity with a polynucleotide of SEQ ID NO: 164, the polynucleotide encoding the second polypeptide of EGFR×cMET multispecific antibody has at least 85%, 89%, 90%, 91%, 92%, 93%, 94%, 95%, 96%, 97%, 98%, 99%, or 100% nucleic acid sequence identity with a polynucleotide of SEQ ID NO: 165, and the polynucleotide encoding the third polypeptide of EGFR×cMET multispecific antibody has at least 85%, 89%, 90%, 91%, 92%, 93%, 94%, 95%, 96%, 97%, 98%, 99%, or 100% nucleic acid sequence identity with a polynucleotide of SEQ ID NO: 166.

In one embodiment, the first polynucleotide encoding the first polypeptide of EGFR×cMET multispecific antibody comprises a DNA sequence having SEQ ID NO: 164, the second polynucleotide encoding the second polypeptide of EGFR×cMET multispecific antibody comprises a DNA sequence having SEQ ID NO: 165, and the third polynucleotide encoding the third polypeptide of EGFR×cMET multispecific antibody comprises a DNA sequence having SEQ ID NO: 166.

In one embodiment, the first polynucleotide encoding the first polypeptide of EGFR×cMET multispecific antibody comprises a DNA sequence of SEQ ID NO: 164, the second polynucleotide encoding the second polypeptide of EGFR×cMET multispecific antibody comprises a DNA sequence of SEQ ID NO: 165, and the third polynucleotide encoding the third polypeptide of EGFR×cMET multispecific antibody comprises a DNA sequence of SEQ ID NO: 166.

In some embodiments, the polynucleotides described herein could be codon-optimized for expression in host cells, e.g., eukaryotic cells, more specifically mammalian cells (e.g., CHO cells).

In one embodiment, the fucosyltransferase inhibitor 2F-Peracetyl-Fucose (Sigma-Aldrich) for inhibition of fucosylation is added in the production system for effector function enhancement.

In one embodiment, an engineered CHO cell line is used, in which the activity of α1, 6-fucosyltransferase is lower or disappeared, so that the produced antibodies or antigen-binding fragment thereof is afucosylated. Refer to WO2003085107A1 for more details.

Also provided in the present disclosure are expression vectors and host cells for producing the antibodies herein. The choice of expression vector depends on the intended host cells in which the vector is to be expressed. Typically, the expression vectors contain a promoter and other regulatory sequences (e.g., enhancers) that are operably linked to the polynucleotides encoding antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987), each incorporated by reference in their entirety. For example, the SV40 enhancer or CMV enhancer can be used to increase expression in mammalian host cells.

The host cells for harboring and expressing the antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express antibodies. Insect cells in combination with baculovirus vectors can also be used. In other aspects, mammalian host cells are used to express and produce the antibodies of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987, each incorporated by reference in their entirety. Expression vectors for mammalian host cells can include expression control sequences, such as an origin of replication, a promoter, and an enhancer (see, e.g., Queen et al., Immunol. Rev. 89:49-68, 1986, incorporated by reference in its entirety), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Methods of Detection and Diagnosis

The antibodies or antigen-binding fragments of the present disclosure are useful in a variety of applications including, but not limited to, methods for the detection of cMET/EGFR (i.e., cMET and/or EGFR). In one aspect, the antibodies or antigen-binding fragments are useful for detecting the presence of cMET/EGFR in a biological sample. The term “detecting” as used herein includes quantitative or qualitative detection. In certain aspects, a biological sample comprises a cell or tissue. In other aspects, such tissues include normal and/or cancerous tissues that express cMET/EGFR at higher levels relative to other tissues.

In one aspect, the present disclosure provides a method of detecting the presence of cMET in a biological sample. In certain aspects, the method comprises contacting the biological sample with an anti-cMET/EGFR antibody under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of cMET/EGFR. In certain aspects, the method comprises contacting a test cell with an anti-cMET/EGFR antibody; determining the level of expression (either quantitatively or qualitatively) of cMET/EGFR expressed by the test cell by detecting binding of the anti-cMET/EGFR antibody to the cMET polypeptide/EGFR polypeptide; and comparing the level of expression by the test cell with the level of cMET/EGFR expression in a control cell (e.g., a normal cell of the same tissue origin as the test cell or a non-cMET/EGFR expressing cell), wherein a higher level of cMET/EGFR expression in the test cell as compared to the control cell indicates the presence of a disorder associated with expression of cMET/EGFR.

### Methods of Treatment

In some embodiments, the antibodies or antigen-binding fragments of the present disclosure are useful in a variety of applications including, but not limited to, methods for the treatment of a cMET-associated disorder or disease. In one aspect, the cMET-associated disorder or disease is a cancer. In some embodiments, the cancer is cMET positive.

The antibodies or antigen-binding fragments of the present disclosure are useful in a variety of applications including, but not limited to, methods for the treatment of a EGFR/cMET-associated disorder or disease. In one aspect, the EGFR/cMET-associated disorder or disease is a cancer. In some embodiments, the cancer is EGFR/cMET positive.

In one aspect, the present disclosure provides a method of treating cancer. In certain aspects, the method comprises administering to a patient in need an effective amount of an anti-cMET antibody or antigen-binding fragment or cMET antibody containing multispecific antibody.

In another aspect, the present disclosure provides anti-cMET antibody or antigen-binding fragment or the multispecific antibody, or the pharmaceutical composition for use in the treatment of cancer. In another aspect, the present disclosure provides the use of the anti-cMET antibody or antigen-binding fragment, multispecific antibody or antigen-binding fragment thereof, or the pharmaceutical composition in the manufacture of a medicament for the treatment of cancer.

In one aspect, the present disclosure provides a method of treating cancer. In certain aspects, the method comprises administering to a patient in need an effective amount of multispecific antibody herein (e.g., multispecific antibodies described in Section 3). In another aspect, the present disclosure provides multispecific antibody herein, or the pharmaceutical composition for use in the treatment of cancer. In another aspect, the present disclosure provides the use of the multispecific antibody or antigen-binding fragment thereof, or the pharmaceutical composition in the manufacture of a medicament for the treatment of cancer.

In one embodiment, the cancer harbors a cMET genetic alteration, and/or the cMET genetic alteration results in constitutively active cMET signaling. In one embodiment, the growth of the cancer is driven by cMET signaling. In one embodiment, the cancer harbors a cMET genetic alteration and the growth of the cancer is driven by cMET signaling.

In one embodiment, the cMET signaling is ligand dependent. In another embodiment, the cMET signaling is ligand independent.

In another embodiment, the cMET genetic alteration includes cMET overexpression, genomic amplification, mutation, and/or alternative splicing, which results in constitutively active cMET signaling.

In another embodiment, the cancer harbors an EGFR activating mutation and/or the growth of the cancer cell is driven by EGFR signaling. In another embodiment, the EGFR activating mutation is deletion or point mutation, including EGFR exon 19 deletion (E19del) and/or EGFR L858R/T790M.

In another embodiment, the EGFR signaling is ligand independent. In another embodiment, the EGFR signaling is ligand dependent.

In another embodiment, the EGFR×cMET multispecific antibodies herein induce the internalization of EGFR and/or cMET receptor, thereby reducing the receptors on the cell surface. Thus, the treatment of EGFR×cMET multispecific antibodies are not limited to a specific EGFR mutation or cMET amplification. The cell lines used in the Examples section are only used as an example to illustrate, and should not limit the scope of the treatment herein.

The cancer can include, without limitation, gastric cancer, colorectal cancer, lung cancer, liver cancer, head and neck cancer, kidney cancer, breast cancer, or brain cancer.

In one embodiment, the lung cancer is non-small cell lung cancer (NSCLC) or small cell lung cancer (SCLC). In another embodiment, the non-small cell lung cancer is squamous non-small cell lung cancer. In another embodiment, the liver cancer is hepatocellular carcinoma. In another embodiment, the head and neck cancer is head and neck squamous cell carcinoma. In another embodiment, the gastric cancer is alpha-fetoprotein positive (AFP+) gastric cancer.

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules include but are not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician.

### Combination Therapy

In one aspect, the antibodies of the present disclosure (including the anti-cMET bispecific antibodies and EGFRxcMET multispecific antibodies) can be used in combination with other therapeutic agents.

The antibodies of the present disclosure can be used in combination with other therapeutics, for example, other immune checkpoint antibodies. Such immune checkpoint antibodies can include anti-PD1 antibodies. Anti-PD1 antibodies can include, without limitation, Tislelizumab, Pembrolizumab or Nivolumab. Tislelizumab (SEQ ID Nos: 140 and 141 in Table 3) is disclosed in U.S. Pat. No. 8,735,553. Pembrolizumab (formerly MK-3475), is disclosed in U.S. Pat. Nos. 8,354,509 and 8,900,587 and is a humanized lgG4-K immunoglobulin which targets the PD1 receptor and inhibits binding of the PD1 receptor ligands PD-L1 and PD-L2. Nivolumab (as disclosed by Bristol-Meyers Squibb) is a fully human lgG4-K monoclonal antibody. Nivolumab (clone 5C4) is disclosed in U.S. Pat. No. 8,008,449 and WO 2006/121168.

TABLE 3
Tislelizumab sequences

| VH of | SEQ | AA | QVQLQESGPGLVKPSETLSLTCTVSGFS |
|---|---|---|---|
| Tislelizumab | ID | | LTSYGVHWIRQPPGKGLEWIGVIYADGS |
| | NO: | | TNYNPSLKSRVTISKDTSKNQVSLKLSS |
| | 140 | | VTAADTAVYYCARAYGNYWYIDVWGQGT |
| | | | TVTVSS |
| VL of | SEQ | AA | DIVMTQSPDSLAVSLGERATINCKSSES |
| Tislelizumab | ID | | VSNDVAWYQQKPGQPPKLLINYAFHRFT |
| | NO: | | GVPDRESGSGYGTDFTLTISSLQAEDVA |
| | 141 | | VYYCHQAYSSPYTFGQGTKLEIK |

In one embodiment, the present disclosure provides a use of the combination of the antibody herein and anti-PD-1 antibody (such as Tislelizumab or other anti-PD-1 antibody mentioned above) in the manufacture of a medicament for the treatment of cancer, such as the cancers mentioned above. In another embodiment, the present disclosure provides the combination of the antibody of the present disclosure (including the anti-cMET bispecific antibodies and EGFRxcMET multispecific antibodies) and anti-PD-1 antibody (such as Tislelizumab or other anti-PD-1 antibody mentioned above) for use in the treatment of cancer, such as the cancers mentioned above.

The combination therapy is intended to mean, and does refer to and include any one of the following:
simultaneous administration of such combination therapy to a patient in need of treatment, when such components are formulated together into a single dosage form which releases said components at substantially the same time to said patient,
substantially simultaneous administration of such combination to a patient in need of treatment, when such components are formulated apart from each other into separate dosage forms which are taken at substantially the same time by said patient, whereupon said components are released at substantially the same time to said patient,
sequential administration of such combination therapy to a patient in need of treatment, when such components are formulated apart from each other into separate dosage forms which are taken at consecutive times by said patient with a significant time interval between each administration, whereupon said components are released at substantially different times to said patient; and
sequential administration of such combination to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

### Pharmaceutical Compositions

Also provided are compositions, including pharmaceutical formulations, comprising antibodies or multispecific antibodies herein, or polynucleotides comprising sequences encoding the antibody or antigen-binding fragment herein. In certain embodiments, compositions comprise one or more antibodies or multispecific antibodies or antigen-binding fragments, or one or more polynucleotides comprising sequences encoding one or more antibodies or antigen-binding fragments. These compositions can further comprise suitable carriers, such as pharmaceutically acceptable excipients including buffers, which are well known in the art.

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “human cMET” refers to the receptor tyrosine kinase mesenchymal-epithelial transition factor in human. The amino acid sequence of human cMET (SEQ ID NO: 69) could also be found in GenBank: AAI30421.1.

The term “human EGFR” refers to the epidermal growth factor receptor in human. The amino acid sequence of human EGFR (SEQ ID NO: 167) could be found in https://www.uniprot.org/uniprotkb/P00533/entry #sequences.

EGFR amino acid sequence
SEQ ID NO: 167
MRPSGTAGAALLALLAALCPASRALEEKKVCQGTSNKLTQLGTFEDHFLSLQRMENNCE
VVLGNLEITYVQRNYDLSFLKTIQEVAGYVLIALNTVERIPLENLQIIRGNMYYENSYAL
AVLSNYDANKTGLKELPMRNLQEILHGAVRFSNNPALCNVESIQWRDIVSSDFLSNMSM
DFQNHLGSCQKCDPSCPNGSCWGAGEENCQKLTKIICAQQCSGRCRGKSPSDCCHNQC
AAGCTGPRESDCLVCRKFRDEATCKDTCPPLMLYNPTTYQMDVNPEGKYSFGATCVKK
CPRNYVVTDHGSCVRACGADSYEMEEDGVRKCKKCEGPCRKVCNGIGIGEFKDSLSIN
ATNIKHFKNCTSISGDLHILPVAFRGDSFTHTPPLDPQELDILKTVKEITGFLLIQAWPENR
TDLHAFENLEIIRGRTKQHGQFSLAVVSLNITSLGLRSLKEISDGDVIISGNKNLCYANTIN
WKKLFGTSGQKTKIISNRGENSCKATGQVCHALCSPEGCWGPEPRDCVSCRNVSRGREC
VDKCNLLEGEPREFVENSECIQCHPECLPQAMNITCTGRGPDNCIQCAHYIDGPHCVKTC
PAGVMGENNTLVWKYADAGHVCHLCHPNCTYGCTGPGLEGCPTNGPKIPSIATGMVGA
LLLLLVVALGIGLFMRRRHIVRKRTLRRLLQERELVEPLTPSGEAPNQALLRILKETEFKKI
KVLGSGAFGTVYKGLWIPEGEKVKIPVAIKELREATSPKANKEILDEAYVMASVDNPHV
CRLLGICLTSTVQLITQLMPFGCLLDYVREHKDNIGSQYLLNWCVQIAKGMNYLEDRRL
VHRDLAARNVLVKTPQHVKITDFGLAKLLGAEEKEYHAEGGKVPIKWMALESILHRIYT
HQSDVWSYGVTVWELMTFGSKPYDGIPASEISSILEKGERLPQPPICTIDVYMIMVKCW
MIDADSRPKFRELIIEFSKMARDPQRYLVIQGDERMHLPSPTDSNFYRALMDEEDMDDV
VDADEYLIPQQGFFSSPSTSRTPLLSSLSATSNNSTVACIDRNGLQSCPIKEDSFLQRYSSD
PTGALTEDSIDDTFLPVPEYINQSVPKRPAGSVONPVYHNQPLNPAPSRDPHYQDPHSTAV
GNPEYLNTVQPTCVNSTFDSPAHWAQKGSHQISLDNPDYQQDFFPKEAKPNGIFKGSTA
ENAEYLRVAPQSSEFIGA

The term “cMET biparatopic antibodies or antigen-binding fragments thereof” means in the multispecific antibodies or antigen-binding fragments thereof, the first antigen binding domain thereof targeting human cMET and the second antigen binding domain thereof targeting human cMET recognize non-overlapping epitopes on human cMET, or the first antigen binding domain thereof targeting human cMET does not compete with the second antigen binding domain thereof targeting human cMET.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient comprising, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, chimeric antibodies, and anti-idiotypic (anti-Id) antibodies, a human engineered antibody, a single chain antibody (scFv), a single domain antibody, a Fab fragment, a Fab′ fragment, or a F(ab′)₂ fragment. The antibodies can be of any isotype/class (e.g., IgG, IgE, IgM, IgD, IgA and IgY), or subclass (e.g., IgG1, IgG2, IgG3, IgG4, IgA1 and IgA2). In addition, the antibody includes the derivative agents thereof, such as fusion protein, multispecific antibody, or antibody-drug conjugate (ADC). In addition, the antibody includes the derivative agents thereof by linking to another agent (such as other drug or antibody) directly or indirectly or forming a complex with another agent.

The term “chimeric antibody” means molecules made up of domains from different species, i.e., fusing the variable domain of an antibody from one host species (e.g. mouse, rabbit, llama, etc.) with the constant domain of an antibody from a different species (e.g. human).

In some embodiments, the anti-cMET antibodies comprise at least one antigen-binding site, at least a variable region. In some embodiments, the anti-cMET antibodies comprise an antigen-binding fragment from an cMET antibody described herein. In some embodiments, the anti-cMET antibody is isolated or recombinant. In some embodiments, the anti-cMET antibodies also encompass multispecific antibodies targeting a first epitope of cMET as a first arm and targeting a second epitope of cMET as a second arm. In some embodiments, the anti-cMET antibodies also encompass multispecific antibodies targeting cMET as at least one arm (e.g., two arms targeting distinct epitopes respectively) and targeting other antigen(s) as another arm(s).

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as a, 6, F, 7, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

“Antigen-binding domain” as used herein, means the portion on an antibody that specifically binds to an antigen. In some embodiments, it comprise at least six CDRs and specifically bind to an epitope (or three CDRs in terms of single domain antibody). An “antigen-binding domain” of a multispecific antibody (e.g., a bispecific antibody) comprises a first antigen binding domain that specifically binds to a first epitope and a second antigen binding domain specifically binds to a second epitope. Multispecific antibodies can be bispecific, trispecific, tetraspecific etc., with antigen binding domains directed to each specific epitope. Multispecific antibodies can be multivalent (e.g., a bispecific tetravalent antibody) that comprises multiple antigen binding domains, for example, 2, 3, 4 or more antigen binding domains that specifically bind to a first epitope and 2, 3, 4 or more antigen binding domains that specifically bind a second epitope.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine, rabbit, llama, etc.) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent/camelid antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to cMET. Specifically, common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H} interfaces or V_{H}/V_{L} interfaces of antibodies (see, e.g., US 2011/0287009, US2007/0178552, WO 96/027011, WO 98/050431, and Zhu et al., 1997, Protein Science 6:781-788). In some embodiments, knob-into-holes insure the correct pairing of two different heavy chains together during the manufacture of multispecific antibodies. For example, multispecific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also insure correct pairing.

The term “knob” as used herein in the context of “knob-into-hole” technology refers to an amino acid change that introduces a protuberance (knob) into a polypeptide at an interface in which the polypeptide interacts with another polypeptide. In some embodiments, the other polypeptide has a hole mutation.

The term “hole” as used herein in the context of “knob-into-hole” refers to an amino acid change that introduces a socket or cavity (hole) into a polypeptide at an interface in which the polypeptide interacts with another polypeptide. In some embodiments, the other polypeptide has a knob mutation.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4: 11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-cMET multispecific antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-cMET antibody is administered to the subject at the same time as, just before, or just after administration of an additional therapeutic agent. In certain embodiments, an anti-cMET antibody is administered as a co-formulation with an additional therapeutic agent.

### Equivalent

It is to be understood that while the present invention has been described in conjunction with the detailed description thereof, the foregoing description is intended to illustrate and not limit the scope of the invention, which is defined by the scope of the appended claims. Other aspects, advantages, and modifications are within the scope of the following claims.

It is to be understood that one, some, any or all of the features of the various embodiments described herein may be combined to form further embodiments of the present disclosure. These and other aspects of the present disclosure will become apparent to those skilled in the art.

### EXAMPLES

### Example 1. Generation of Anti-cMET Antibodies

### Mouse Immunization

To generate anti-cMET antibodies, genetically engineered mice (RenLite®, Biocytogen) were immunized by human cMET extracellular domain (amino acids 1-932 of SEQ ID NO: 69 which is full length human cMET) fused to his tag or mouse Fc (internal generated, SEQ ID NOs: 1 and 2) mixed with adjuvants (Complete Freund's Adjuvant (F5881, Sigma) for prime immune and Freund's Adjuvant, Incomplete (F5506, Sigma) for the following immune). Animals were injected bi- or tni-weekly intraperitoneally and subcutaneously. The mice used comprise DNA encoding different human immunoglobulin heavy chain and one shared human light chain variable region.

Serum titers against cMET protein were determined by ELISA to monitor the humoral immune response. Animals with sufficient cMET specific antibody titers were administered a final boost of cMET protein for the antibody screen.

TABLE 4
Antigen Sequence List

| Antigen | SEQ ID NO | | SEQUENCE |
|---|---|---|---|
| Human | SEQ ID NO: 1 | AA | ECKEALAKSEMNVNMKYQLPNFTAETPIQNVILHEH |
| cMET-ECD- | | sequence | HIFLGATNYIYVLNEEDLQKVAEYKTGPVLEHPDCFP |
| his | | | CQDCSSKANLSGGVWKDNINMALVVDTYYDDQLIS |
| | | | CGSVNRGTCQRHVFPHNHTADIQSEVHCIFSPQIEEPS |
| | | | QCPDCVVSALGAKVLSSVKDRFINFFVGNTINSSYFP |
| | | | DHPLHSISVRRLKETKDGFMFLTDQSYIDVLPEFRDS |
| | | | YPIKYVHAFESNNFIYFLTVQRETLDAQTFHTRIIRFC |
| | | | SINSGLHSYMEMPLECILTEKRKKRSTKKEVFNILQA |
| | | | AYVSKPGAQLARQIGASLNDDILFGVFAQSKPDSAEP |
| | | | MDRSAMCAFPIKYVNDFFNKIVNKNNVRCLQHFYG |
| | | | PNHEHCFNRTLLRNSSGCEARRDEYRTEFTTALQRVD |
| | | | LFMGQFSEVLLTSISTFIKGDLTIANLGTSEGRFMQVV |
| | | | VSRSGPSTPHVNFLLDSHPVSPEVIVEHTLNQNGYTL |
| | | | VITGKKITKIPLNGLGCRHFQSCSQCLSAPPFVQCGW |
| | | | CHDKCVRSEECLSGTWTQQICLPAIYKVFPNSAPLEG |
| | | | GTRLTICGWDFGFRRNNKFDLKKTRVLLGNESCTLTL |
| | | | SESTMNTLKCTVGPAMNKHFNMSIIISNGHGTTQYST |
| | | | FSYVDPVITSISPKYGPMAGGTLLTLTGNYLNSGNSR |
| | | | HISIGGKTCTLKSVSNSILECYTPAQTISTEFAVKLKID |
| | | | LANRETSIFSYREDPIVYEIHPTKSFISGGSTITGVGKN |
| | | | LNSVSVPRMVINVHEAGRNFTVACQHRSNSEIICCTT |
| | | | PSLQQLNLQLPLKTKAFFMLDGILSKYFDLIYVHNPV |
| | | | FKPFEKPVMISMGNENVLEIKGNDIDPEAVKGEVLKV |
| | | | GNKSCENIHLHSEAVLCTVPNDLLKLNSELNIEWKQ |
| | | | AISSTVLGKVIVQPDQNFTHHHHHH |
| Human | SEQ ID NO: 2 | AA | ECKEALAKSEMNVNMKYQLPNFTAETPIQNVILHEH |
| cMET-ECD- | | sequence | HIFLGATNYIYVLNEEDLQKVAEYKTGPVLEHPDCFP |
| mFc | | | CQDCSSKANLSGGVWKDNINMALVVDTYYDDQLIS |
| | | | CGSVNRGTCQRHVFPHNHTADIQSEVHCIFSPQIEEPS |
| | | | QCPDCVVSALGAKVLSSVKDRFINFFVGNTINSSYFP |
| | | | DHPLHSISVRRLKETKDGFMFLTDQSYIDVLPEFRDS |
| | | | YPIKYVHAFESNNFIYFLTVQRETLDAQTFHTRIIRFC |
| | | | SINSGLHSYMEMPLECILTEKRKKRSTKKEVFNILQA |
| | | | AYVSKPGAQLARQIGASLNDDILFGVFAQSKPDSAEP |
| | | | MDRSAMCAFPIKYVNDFFNKIVNKNNVRCLQHFYG |
| | | | PNHEHCFNRTLLRNSSGCEARRDEYRTEFTTALQRVD |
| | | | LFMGQFSEVLLTSISTFIKGDLTIANLGTSEGRFMQVV |
| | | | VSRSGPSTPHVNFLLDSHPVSPEVIVEHTLNQNGYTL |
| | | | VITGKKITKIPLNGLGCRHFQSCSQCLSAPPFVQCGW |
| | | | CHDKCVRSEECLSGTWTQQICLPAIYKVFPNSAPLEG |
| | | | GTRLTICGWDFGFRRNNKFDLKKTRVLLGNESCTLTL |
| | | | SESTMNTLKCTVGPAMNKHFNMSIIISNGHGTTQYST |
| | | | FSYVDPVITSISPKYGPMAGGTLLTLTGNYLNSGNSR |
| | | | HISIGGKTCTLKSVSNSILECYTPAQTISTEFAVKLKID |
| | | | LANRETSIFSYREDPIVYEIHPTKSFISGGSTITGVGKN |
| | | | LNSVSVPRMVINVHEAGRNFTVACQHRSNSEIICCTT |
| | | | PSLQQLNLQLPLKTKAFFMLDGILSKYFDLIYVHNPV |
| | | | FKPFEKPVMISMGNENVLEIKGNDIDPEAVKGEVLKV |
| | | | GNKSCENIHLHSEAVLCTVPNDLLKLNSELNIEWKQ |
| | | | AISSTVLGKVIVQPDQNFTGGGGSCPPCKCPAPNLLG |
| | | | GPSVFIFPPKIKDVLMISLSPIVTCVVVDVSEDDPDVQ |
| | | | ISWFVNNVEVHTAQTQTHREDYNSTLRVVSALPIQH |
| | | | QDWMSGKEFKCKVNNKDLPAPIERTISKPKGSVRAP |
| | | | QVYVLPPPEEEMTKKQVTLTCMVTDFMPEDIYVEW |
| | | | TNNGKTELNYKNTEPVLDSDGSYFMYSKLRVEKKN |
| | | | WVERNSYSCSVVHEGLHNHHTTKSFSRTPGK |
| cyno cMET- | SEQ ID NO: 3 | AA | ECKEALAKSEMNVNMKYQLPNFTAETAIQNVILHEH |
| ECD-his | | sequence | HIFLGATNYIYVLNEEDLQKVAEYKTGPVLEHPDCFP |
| | | | CQDCSSKANLSGGVWKDNINMALVVDTYYDDQLIS |
| | | | CGSVNRGTCQRHVFPHNHTADIQSEVHCIFSPQIEEP |
| | | | NQCPDCVVSALGAKVLSSVKDRFINFFVGNTINSSYF |
| | | | PHHPLHSISVRRLKETKDGFMFLTDQSYIDVLPEFRD |
| | | | SYPIKYIHAFESNNFIYFLTVQRETLNAQTFHTRIIRFC |
| | | | SLNSGLHSYMEMPLECILTEKRKKRSTKKEVFNILQA |
| | | | AYVSKPGAQLARQIGASLNDDILFGVFAQSKPDSAEP |
| | | | MDRSAMCAFPIKYVNDFFNKIVNKNNVRCLQHFYG |
| | | | PNHEHCFNRTLLRNSSGCEARRDEYRAEFTTALQRV |
| | | | DLFMGQFSEVLLTSISTFVKGDLTIANLGTSEGRFMQ |
| | | | VVVSRSGPSTPHVNFLLDSHPVSPEVIVEHPLNQNGY |
| | | | TLVVTGKKITKIPLNGLGCRHFQSCSQCLSAPPFVQC |
| | | | GWCHDKCVRSEECPSGTWTQQICLPAIYKVFPTSAPL |
| | | | EGGTRLTICGWDFGFRRNNKFDLKKTRVLLGNESCT |
| | | | LTLSESTMNTLKCTVGPAMNKHFNMSIIISNGHGTTQ |
| | | | YSTFSYVDPIITSISPKYGPMAGGTLLTLTGNYLNSGN |
| | | | SRHISIGGKTCTLKSVSNSILECYTPAQTISTEFAVKLK |
| | | | IDLANRETSIFSYREDPIVYEIHPTKSFISGGSTITGVG |
| | | | KNLHSVSVPRMVINVHEAGRNFTVACQHRSNSEIICC |
| | | | TTPSLQQLNLQLPLKTKAFFMLDGILSKYFDLIYVHN |
| | | | PVFKPFEKPVMISMGNENVLEIKGNDIDPEAVKGEVL |
| | | | KVGNKSCENIHLHSEAVLCTVPNDLLKLNSELNIEW |
| | | | KQAISSTVLGKVIVQPDQNFTHHHHHH |
| Human | SEQ ID NO: 69 | AA | MKAPAVLAPGILVLLFTLVQRSNGECKEALAKSEMN |
| cMET full | | sequence | VNMKYQLPNFTAETPIQNVILHEHHIFLGATNYIYVL |
| length | | | NEEDLQKVAEYKTGPVLEHPDCFPCQDCSSKANLSG |
| | | | GVWKDNINMALVVDTYYDDQLISCGSVNRGTCQRH |
| | | | VFPHNHTADIQSEVHCIFSPQIEEPSQCPDCVVSALGA |
| | | | KVLSSVKDRFINFFVGNTINSSYFPDHPLHSISVRRLK |
| | | | ETKDGFMFLTDQSYIDVLPEFRDSYPIKYVHAFESNN |
| | | | FIYFLTVQRETLDAQTFHTRIIRFCSINSGLHSYMEMP |
| | | | LECILTEKRKKRSTKKEVFNILQAAYVSKPGAQLARQ |
| | | | IGASLNDDILFGVFAQSKPDSAEPMDRSAMCAFPIKY |
| | | | VNDFFNKIVNKNNVRCLQHFYGPNHEHCFNRTLLRN |
| | | | SSGCEARRDEYRTEFTTALQRVDLFMGQFSEVLLTSIS |
| | | | TFIKGDLTIANLGTSEGRFMQVVVSRSGPSTPHVNFL |
| | | | LDSHPVSPEVIVEHTLNQNGYTLVITGKKITKIPLNGL |
| | | | GCRHFQSCSQCLSAPPFVQCGWCHDKCVRSEECLSG |
| | | | TWTQQICLPAIYKVFPNSAPLEGGTRLTICGWDFGFR |
| | | | RNNKFDLKKTRVLLGNESCTLTLSESTMNTLKCTVG |
| | | | PAMNKHFNMSIIISNGHGTTQYSTFSYVDPVITSISPK |
| | | | YGPMAGGTLLTLTGNYLNSGNSRHISIGGKTCTLKSV |
| | | | SNSILECYTPAQTISTEFAVKLKIDLANRETSIFSYRED |
| | | | PIVYEIHPTKSFISGGSTITGVGKNLNSVSVPRMVINV |
| | | | HEAGRNFTVACQHRSNSEIICCTTPSLQQLNLQLPLK |
| | | | TKAFFMLDGILSKYFDLIYVHNPVFKPFEKPVMISMG |
| | | | NENVLEIKGNDIDPEAVKGEVLKVGNKSCENIHLHSE |
| | | | AVLCTVPNDLLKLNSELNIEWKQAISSTVLGKVIVQP |
| | | | DQNFTGLIAGVVSISTALLLLLGFFLWLKKRKQIKDL |
| | | | GSELVRYDARVHTPHLDRLVSARSVSPTTEMVSNES |
| | | | VDYRATFPEDQFPNSSQNGSCRQVQYPLTDMSPILTS |
| | | | GDSDISSPLLQNTVHIDLSALNPELVQAVQHVVIGPSS |
| | | | LIVHFNEVIGRGHFGCVYHGTLLDNDGKKIHCAVKS |
| | | | LNRITDIGEVSQFLTEGIIMKDFSHPNVLSLLGICLRSE |
| | | | GSPLVVLPYMKHGDLRNFIRNETHNPTVKDLIGFGL |
| | | | QVAKGMKYLASKKFVHRDLAARNCMLDEKFTVKV |
| | | | ADFGLARDMYDKEYYSVHNKTGAKLPVKWMALES |
| | | | LQTQKFTTKSDVWSFGVLLWELMTRGAPPYPDVNT |
| | | | FDITVYLLQGRRLLQPEYCPDPLYEVMLKCWHPKAE |
| | | | MRPSFSELVSRISAIFSTFIGEHYVHVNATYVNVKCVA |
| | | | PYPSLLSSEDNADDEVDTRPASFWETS |

### Plasma Cell Screening by the Beacon Optofluidic System

At 3-5 days post final boost, spleens were collected and mashed into single cell suspensions. Plasma cells were isolated with a mouse CD138 positive selection kit (STEMCELL™) according to the manufacturer's instructions. Enriched plasma cells with a density of 6.25×10⁶/ml were imported into the channel and penned into the NanoPen chambers of OptoSelect 14K Chip™ (Berkeley Lights) according to the manufacturer's instructions. To screen human cMET specific plasma cells, human cMET protein (MET-H82E1, Acrobiosystems) conjugated beads (520-00053, Berkeley Lights) and Alexa Fluor 488 goat anti-mouse IgG secondary antibody (Jackson ImmunoResearch) with a concentration of 5 μg/ml were imported into the channel. Following import, the freeze valve was turned on and the exposure time of FITC channel for Alexa Fluor 488 fluorophore was set to 1000 ins. Bloom-like positive signals were captured by time lapse imaging with settings of 3-minute period and 10 cycles. After completion of human cMET specific plasma cell screening, cyno cMET specific plasma cells were screened with cyno cMET-ECD-his (in house generated, SEQ ID NO: 3) conjugated beads. An assay to remove non-specific binding signals using irrelevant His-tagged protein-conjugated beads was performed as a counter screen. Plasma cells showing both human and cyno cMET specific positive signals were individually exported into 96-well plates filled with lysis buffer.

### Example 2. Antibody VH and VL Gene Cloning, Sequencing, and Expression

First-strand cDNA was synthesized, and total cDNA was amplified with Opto Plasma B Discovery cDNA Synthesis Kit™ (Berkeley Lights) according to the manufacturer's instructions. Antibody VH and VL genes were amplified with Opto Plasma B Discovery Sanger Prep Kit™ (Berkeley Lights) according to the manufacturer's instructions. The amplified VH and VL genes were cloned into mammalian expression vector containing human IgG1 constant region (SEQ ID NO: 70) and human kappa chain constant region (SEQ ID NO: 71) genes and sequenced, respectively. The amino acid sequences of the 3 HCDRs, 3 LCDRs, VH and VL and the DNA sequences of the VH and VL for representative antibodies are listed in Table 5 and Table 6 as SEQ ID Nos: 4 to 68. Antibodies were expressed by Expi293™ cells and purified by affinity chromatography.

TABLE 5
Anti-cMET Monoclonal Antibody List

| | SEQ ID NO. | | | | | | | | | |
|---|---|---|---|---|---|---|---|---|---|---|
| | VH | VH | HCDR1 | HCDR2 | HCDR3 | VL | VL | LCDR1 | LCDR2 | LCDR3 |
| Antibody | (AA) | (DNA) | (Kabat) | (Kabat) | (Kabat) | (AA) | (DNA) | (Kabat) | (Kabat) | (Kabat) |
| 063Ab10910 | 4 | 5 | 6 | 7 | 8 | 64 | 65 | 66 | 67 | 68 |
| 061Ab15310 | 9 | 10 | 11 | 12 | 13 | 64 | 65 | 66 | 67 | 68 |
| 063Ab16010 | 14 | 15 | 16 | 17 | 18 | 64 | 65 | 66 | 67 | 68 |
| 063Ab02110 | 19 | 20 | 21 | 22 | 23 | 64 | 65 | 66 | 67 | 68 |
| 063Ab15210 | 24 | 25 | 26 | 27 | 28 | 64 | 65 | 66 | 67 | 68 |
| 062Ab16310 | 29 | 30 | 31 | 32 | 33 | 64 | 65 | 66 | 67 | 68 |
| 063Ab05510 | 34 | 35 | 36 | 37 | 38 | 64 | 65 | 66 | 67 | 68 |
| 063Ab07710 | 39 | 40 | 41 | 42 | 43 | 64 | 65 | 66 | 67 | 68 |
| 063Ab14710 | 44 | 45 | 46 | 47 | 48 | 64 | 65 | 66 | 67 | 68 |
| 061Ab05110 | 49 | 50 | 51 | 52 | 53 | 64 | 65 | 66 | 67 | 68 |
| 063Ab03210 | 54 | 55 | 56 | 57 | 58 | 64 | 65 | 66 | 67 | 68 |
| 063Ab16720 | 59 | 60 | 61 | 62 | 63 | 64 | 65 | 66 | 67 | 68 |

TABLE 6
Anti-cMET Monoclonal Antibody Sequence List

| | SEQ ID | | |
|---|---|---|---|
| Antibody | NO | Description | SEQUENCE |
| 063Ab10910 | SEQ ID | VH (AA) | QVQLVESGGGVVQPGRSLRLSCAASGFTFSSHGMHWVRQAP |
| | NO: 4 | | GKGLEWVAVISYDESDKYYADSVKGRFTISRDHSKNTLYLQM |
| | | | NSLRAEDTAVYYCVKDRNMGYSGWLDYWGQGTLVTVSS |
| | SEQ ID | VH (DNA) | CAGGTGCAGCTGGTGGAGTCTGGGGGAGGCGTGGTCCAGC |
| | NO: 5 | | CTGGGAGGTCCCTGAGACTCTCCTGTGCAGCCTCTGGATTC |
| | | | ACCTTCAGTAGCCATGGCATGCACTGGGTCCGCCAGGCTCC |
| | | | AGGCAAGGGGCTGGAGTGGGTGGCAGTTATATCATATGAT |
| | | | GAAAGTGATAAATATTATGCAGACTCCGTGAAGGGCCGAT |
| | | | TCACCATCTCCAGAGACCATTCCAAGAACACGCTGTATCTG |
| | | | CAAATGAACAGCCTGAGAGCTGAGGACACGGCTGTATATT |
| | | | ACTGTGTGAAAGATCGGAATATGGGATATAGTGGCTGGCTT |
| | | | GACTACTGGGGCCAGGGAACCCTGGTCACCGTCTCCTCA |
| | SEQ ID | HCDR1 | SHGMH |
| | NO: 6 | (Kabat) | |
| | SEQ ID | HCDR2 | VISYDESDKYYADSVKG |
| | NO: 7 | (Kabat) | |
| | SEQ ID | HCDR3 | DRNMGYSGWLDY |
| | NO: 8 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 061Ab15310 | SEQ ID | VH (AA) | QVQLVESGGGVVQPGKSLRLSCAASGFTFSSSGLHWVRQAPG |
| | NO: 9 | | KGLEWVAVISYDTSNKYYADSVKGRFTISRDNSKNTLYLQMN |
| | | | SLRAEDTAVYYCAKDLNRGYDWGFDYWGQGTLVTVSS |
| | SEQ ID | VH (DNA) | CAGGTGCAGCTGGTGGAGTCTGGGGGAGGCGTGGTCCAGC |
| | NO: 10 | | CTGGGAAGTCCCTGAGACTCTCCTGTGCAGCCTCTGGATTC |
| | | | ACCTTCAGTAGTTCTGGCTTGCACTGGGTCCGCCAGGCTCC |
| | | | AGGCAAGGGACTGGAGTGGGTGGCAGTTATATCATATGAT |
| | | | ACAAGTAATAAATACTATGCAGACTCCGTGAAGGGCCGAT |
| | | | TCACCATCTCCAGAGACAATTCCAAGAACACGCTGTATCTG |
| | | | CAAATGAACAGCCTGAGAGCTGAGGACACGGCTGTATATT |
| | | | ACTGTGCGAAAGATCTAAATCGTGGCTATGATTGGGGGTTT |
| | | | GACTACTGGGGCCAGGGAACCCTGGTCACCGTCTCCTCA |
| | SEQ ID | HCDR1 | SSGLH |
| | NO: 11 | (Kabat) | |
| | SEQ ID | HCDR2 | VISYDTSNKYYADSVKG |
| | NO: 12 | (Kabat) | |
| | SEQ ID | HCDR3 | DLNRGYDWGFDY |
| | NO: 13 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab16010 | SEQ ID | VH (AA) | QVQLVESGGGVVQPGRSLRLSCAASGFTFSNYGMHWVRQAP |
| | NO: 14 | | GKGLEWVAVISYDGVDKYYADSVKGRFTISRDNSKNTLYLQ |
| | | | MNSLRAEDTAMYYCAKDRISYNWNLDYWGQGTLVTVSS |
| | SEQ ID | VH (DNA) | CAGGTGCAGCTGGTGGAGTCTGGGGGAGGCGTGGTCCAGC |
| | NO: 15 | | CTGGGAGGTCCCTGAGACTCTCCTGTGCAGCCTCTGGATTC |
| | | | ACCTTCAGTAACTATGGCATGCACTGGGTCCGCCAGGCTCC |
| | | | AGGCAAGGGGCTGGAGTGGGTGGCAGTTATATCATATGAT |
| | | | GGCGTTGATAAGTACTATGCAGACTCCGTGAAGGGCCGATT |
| | | | CACCATCTCCAGAGACAATTCCAAGAACACGCTGTATCTGC |
| | | | AAATGAACAGCCTGAGAGCTGAGGACACGGCTATGTATTA |
| | | | CTGTGCGAAAGATCGGATTTCCTATAACTGGAACCTTGACT |
| | | | ACTGGGGCCAGGGAACCCTGGTCACCGTCTCCTCA |
| | SEQ ID | HCDR1 | NYGMH |
| | NO: 16 | (Kabat) | |
| | SEQ ID | HCDR2 | VISYDGVDKYYADSVKG |
| | NO: 17 | (Kabat) | |
| | SEQ ID | HCDR3 | DRISYNWNLDY |
| | NO: 18 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab02110 | SEQ ID | VH (AA) | QVQLVESGGGVVQPGRSLRLSCVASGFTFSNYGLHWVRQAP |
| | NO: 19 | | GKGLEWVAAISYDGSDKYYADPVKGRFTISRDTTKNTLSLQM |
| | | | NSLRAEDTAVYYCAKERYRGYDWDFDYWGQGTLVTVSS |
| | SEQ ID | VH (DNA) | CAGGTGCAGTTGGTGGAGTCTGGGGGAGGCGTGGTCCAGC |
| | NO: 20 | | CTGGGAGGTCCCTGAGACTCTCCTGTGTAGCCTCTGGATTC |
| | | | ACCTTCAGTAACTATGGCCTACACTGGGTCCGCCAGGCTCC |
| | | | AGGCAAGGGGCTGGAGTGGGTGGCAGCTATATCATATGAT |
| | | | GGAAGTGATAAATACTATGCAGACCCCGTGAAGGGCCGAT |
| | | | TCACCATCTCCAGAGACACGACCAAGAACACGCTGTCTCTG |
| | | | CAAATGAACAGTCTGAGAGCTGAGGACACGGCTGTGTATT |
| | | | ACTGTGCGAAAGAAAGATATCGTGGCTACGATTGGGACTTT |
| | | | GACTACTGGGGCCAGGGAACCCTGGTCACCGTCTCCTCA |
| | SEQ ID | HCDR1 | NYGLH |
| | NO: 21 | (Kabat) | |
| | SEQ ID | HCDR2 | AISYDGSDKYYADPVKG |
| | NO: 22 | (Kabat) | |
| | SEQ ID | HCDR3 | ERYRGYDWDFDY |
| | NO: 23 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab15210 | SEQ ID | VH (AA) | QVQLVESGGGVVQPGRSLRLSCAASGFTFSSYGLHWVRQAPG |
| | NO: 24 | | KGLEWVAVISFDGSNKYYADSVKGRFTISRDTSKNTLSLQMN |
| | | | SLRAEDTAVYYCAKERSRGYDWDFDYWGQGILVTVSS |
| | SEQ ID | VH (DNA) | CAGGTGCAGCTGGTGGAGTCTGGGGGAGGCGTGGTCCAGC |
| | NO: 25 | | CTGGGAGGTCCCTGAGACTCTCCTGTGCAGCCTCTGGATTC |
| | | | ACCTTCAGTAGCTATGGCCTACACTGGGTCCGCCAGGCTCC |
| | | | AGGCAAGGGGCTGGAGTGGGTGGCAGTTATATCATTTGAT |
| | | | GGAAGTAATAAATACTATGCAGACTCCGTGAAGGGCCGAT |
| | | | TCACCATCTCCAGAGACACTTCCAAGAACACGCTGTCTCTG |
| | | | CAAATGAACAGCCTGAGAGCTGAGGACACGGCTGTGTATT |
| | | | ACTGTGCGAAAGAAAGATCTCGTGGCTACGATTGGGACTTT |
| | | | GACTACTGGGGCCAGGGAATCCTGGTCACCGTCTCCTCA |
| | SEQ ID | HCDR1 | SYGLH |
| | NO: 26 | (Kabat) | |
| | SEQ ID | HCDR2 | VISFDGSNKYYADSVKG |
| | NO: 27 | (Kabat) | |
| | SEQ ID | HCDR3 | ERSRGYDWDFDY |
| | NO: 28 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 062Ab16310 | SEQ ID | VH (AA) | QVRLVESGGGVVQPGRSLRLSCAASGFTFSNYGMHWVRQAP |
| | NO: 29 | | GKGLEWVAVISYDGSDKYYPDSVKGRFTISRDNSRNTLYLQM |
| | | | NSLSTEDTAVYYCAKDQYRGYDGTFDYWGQGTLVTVSS |
| | SEQ ID | VH (DNA) | CAGGTGCGGCTGGTGGAGTCGGGGGGAGGCGTGGTCCAGC |
| | NO: 30 | | CTGGGAGGTCCCTGAGACTCTCCTGTGCAGCCTCTGGATTC |
| | | | ACCTTCAGTAACTATGGCATGCACTGGGTCCGCCAGGCTCC |
| | | | AGGCAAGGGGCTGGAGTGGGTGGCAGTTATATCATATGAT |
| | | | GGAAGTGATAAGTACTATCCAGACTCCGTGAAGGGCCGCTT |
| | | | CACCATCTCCAGAGACAATTCCAGAAACACGCTGTATCTGC |
| | | | AAATGAACAGCCTGAGTACTGAGGACACGGCTGTTTATTAC |
| | | | TGTGCGAAAGATCAATATCGTGGCTACGATGGGACTTTTGA |
| | | | CTACTGGGGCCAGGGAACCCTGGTCACCGTCTCCTCA |
| | SEQ ID | HCDR1 | NYGMH |
| | NO: 31 | (Kabat) | |
| | SEQ ID | HCDR2 | VISYDGSDKYYPDSVKG |
| | NO: 32 | (Kabat) | |
| | SEQ ID | HCDR3 | DQYRGYDGTFDY |
| | NO: 33 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab05510 | SEQ ID | VH (AA) | QVQLVESGGGVVQPGRSLRLSCAASGFIFSSYNMHWVRQAPG |
| | NO: 34 | | KGLEWVAVISYDRVNKYYADSVKGRFTISRDNSKNTLYLQM |
| | | | NSLRVEDTAVYYCAKERERYFDWLDYWGQGTLVTVSS |
| | SEQ ID | VH (DNA) | CAGGTGCAGCTGGTGGAGTCTGGGGGAGGCGTGGTCCAGC |
| | NO: 35 | | CTGGGAGGTCCCTGAGACTCTCCTGTGCAGCCTCTGGATTC |
| | | | ATCTTCAGTAGCTATAACATGCACTGGGTCCGCCAGGCTCC |
| | | | AGGCAAGGGGCTGGAGTGGGTGGCAGTTATATCATATGAT |
| | | | AGAGTTAATAAATACTATGCAGACTCCGTGAAGGGCCGATT |
| | | | CACCATCTCCAGAGACAATTCCAAGAACACGCTGTATCTGC |
| | | | AAATGAACAGCCTGAGAGTTGAGGACACGGCTGTGTATTA |
| | | | CTGTGCGAAAGAAAGGGAACGATATTTTGACTGGTTGGACT |
| | | | ACTGGGGCCAGGGAACCCTGGTCACCGTCTCCTCA |
| | SEQ ID | HCDR1 | SYNMH |
| | NO: 36 | (Kabat) | |
| | SEQ ID | HCDR2 | VISYDRVNKYYADSVKG |
| | NO: 37 | (Kabat) | |
| | SEQ ID | HCDR3 | ERERYFDWLDY |
| | NO: 38 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab07710 | SEQ ID | VH (AA) | QVQLVESGGGVVQPGRSLRLSCAASGFTFSSYGMHWVRQAP |
| | NO: 39 | | GKGLEWVAVISNDGSYKYYVDSVTGRFTISRDNSKNTLYLQM |
| | | | NSLRAEDTAVYYCAKDRNMGYSGWFDYWGQGSLVTVSS |
| | SEQ ID | VH (DNA) | CAGGTGCAGCTGGTGGAGTCTGGGGGAGGCGTGGTCCAGC |
| | NO: 40 | | CTGGGAGGTCCCTGAGACTCTCCTGTGCAGCCTCTGGATTC |
| | | | ACCTTCAGTAGCTATGGCATGCACTGGGTCCGCCAGGCTCC |
| | | | AGGCAAGGGGCTGGAGTGGGTGGCAGTTATATCAAATGAT |
| | | | GGAAGTTATAAATACTATGTAGACTCCGTGACGGGCCGATT |
| | | | CACCATCTCCAGAGACAATTCCAAGAACACGCTGTATCTGC |
| | | | AAATGAACAGCCTGAGAGCTGAGGACACGGCTGTGTATTA |
| | | | CTGTGCGAAAGATCGGAATATGGGATATAGTGGCTGGTTTG |
| | | | ACTACTGGGGCCAGGGATCCCTGGTCACCGTCTCCTCA |
| | SEQ ID | HCDR1 | SYGMH |
| | NO: 41 | (Kabat) | |
| | SEQ ID | HCDR2 | VISNDGSYKYYVDSVTG |
| | NO: 42 | (Kabat) | |
| | SEQ ID | HCDR3 | DRNMGYSGWFDY |
| | NO: 43 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab14710 | SEQ ID | VH (AA) | QVQLVESGGGVVQPGRSLRLSCAASGFTFSSHGMHWVRQAP |
| | NO: 44 | | GKGLEWVAVISNDGSYKYYADSVTGRFTISRDNSKNTLYLQM |
| | | | NSLRAEDTAVYYCAKDRNMGYSGWFDYWGQGSLVTVSS |
| | SEQ ID | VH (DNA) | CAGGTGCAGCTGGTGGAGTCTGGGGGAGGCGTGGTCCAGC |
| | NO: 45 | | CTGGGAGGTCCCTGAGACTCTCCTGTGCAGCCTCTGGATTC |
| | | | ACCTTCAGTAGCCATGGCATGCACTGGGTCCGCCAGGCTCC |
| | | | AGGCAAGGGGCTGGAGTGGGTGGCAGTTATATCAAATGAT |
| | | | GGAAGTTATAAATACTATGCAGACTCCGTGACGGGCCGATT |
| | | | CACCATCTCCAGAGACAATTCCAAGAACACGCTGTATCTGC |
| | | | AAATGAACAGCCTGAGAGCTGAGGACACGGCTGTGTATTA |
| | | | CTGTGCGAAAGATCGGAATATGGGATACAGTGGCTGGTTTG |
| | | | ACTACTGGGGCCAGGGATCCCTGGTCACCGTCTCCTCA |
| | SEQ ID | HCDR1 | SHGMH |
| | NO: 46 | (Kabat) | |
| | SEQ ID | HCDR2 | VISNDGSYKYYADSVTG |
| | NO: 47 | (Kabat) | |
| | SEQ ID | HCDR3 | DRNMGYSGWFDY |
| | NO: 48 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 061Ab05110 | SEQ ID | VH (AA) | QVQLVESGGGVVQPGRSLRLSCAASGFTFSNYGMHWVRQAP |
| | NO: 49 | | GKGLEWVAVISNDGSEKYYADSVKGRFTISRDSSKNTLYLQM |
| | | | NSLRAEDTAVYYCAKAPWGSGTTGRLDYWGQGTLVTVSS |
| | SEQ ID | VH (DNA) | CAGGTGCAGCTGGTGGAGTCTGGGGGAGGCGTGGTCCAGC |
| | NO: 50 | | CTGGGAGGTCCCTGAGACTCTCCTGTGCAGCCTCTGGATTC |
| | | | ACCTTCAGTAACTATGGCATGCACTGGGTCCGCCAGGCTCC |
| | | | AGGCAAGGGGCTGGAGTGGGTGGCTGTTATATCAAATGAT |
| | | | GGAAGTGAAAAATACTATGCAGACTCCGTGAAGGGCCGAT |
| | | | TCACCATCTCCAGAGACAGTTCCAAGAACACGCTCTATCTA |
| | | | CAGATGAACAGCCTGAGAGCTGAGGACACGGCTGTGTATT |
| | | | ACTGTGCGAAAGCCCCTTGGGGAAGTGGAACGACGGGGAG |
| | | | GCTTGACTACTGGGGCCAGGGAACCCTGGTCACCGTCTCCT |
| | | | CA |
| | SEQ ID | HCDR1 | NYGMH |
| | NO: 51 | (Kabat) | |
| | SEQ ID | HCDR2 | VISNDGSEKYYADSVKG |
| | NO: 52 | (Kabat) | |
| | SEQ ID | HCDR3 | APWGSGTTGRLDY |
| | NO: 53 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab03210 | SEQ ID | VH (AA) | QFQLQESGPGLVKPSETLSLTCTVSGGSISTSSYYWGWIRQPPG |
| | NO: 54 | | KGLEWIGTIYYSGTTYYNPSLKSRVTISVDTSKNQFSLKLKSVT |
| | | | AADTSIYYCARHWPKYNSWFDPWGQGILVTVSS |
| | SEQ ID | VH (DNA) | CAATTTCAGCTACAGGAGTCGGGCCCAGGACTGGTGAAGC |
| | NO: 55 | | CTTCGGAGACCCTGTCCCTCACCTGCACTGTCTCTGGTGGCT |
| | | | CCATCAGCACTAGTAGTTACTACTGGGGCTGGATCCGCCAG |
| | | | CCCCCAGGGAAGGGGCTGGAGTGGATTGGGACTATCTATT |
| | | | ATAGTGGGACCACCTACTACAACCCGTCCCTCAAGAGTCGA |
| | | | GTCACCATATCCGTAGACACGTCCAAGAACCAGTTCTCCCT |
| | | | GAAGCTGAAGTCTGTGACCGCCGCAGACACGTCTATCTATT |
| | | | ACTGTGCGAGACATTGGCCCAAGTACAACTCCTGGTTCGAC |
| | | | CCCTGGGGCCAGGGAATTCTGGTCACCGTCTCCTCA |
| | SEQ ID | HCDR1 | TSSYYWG |
| | NO: 56 | (Kabat) | |
| | SEQ ID | HCDR2 | TIYYSGTTYYNPSLKS |
| | NO: 57 | (Kabat) | |
| | SEQ ID | HCDR3 | HWPKYNSWFDP |
| | NO: 58 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab16720 | SEQ ID | VH (AA) | QFQLQESGPGLVKPSETLSLTCTVSGGSISTSSYYWGWIRQPPG |
| | NO: 59 | | KGLEWIGTIYFSGTTYYNPSLTSRVSMSVDTSKNQFSLKLNSV |
| | | | TAADTSIYYCARHWPKYNSWFDPWGQGFLVTVSS |
| | SEQ ID | VH (DNA) | CAATTTCAACTACAGGAGTCGGGCCCAGGACTGGTGAAGC |
| | NO: 60 | | CTTCGGAGACCCTGTCCCTCACCTGCACTGTCTCTGGTGGCT |
| | | | CCATCAGCACTAGTAGTTACTACTGGGGCTGGATCCGCCAG |
| | | | CCCCCAGGGAAGGGGCTGGAGTGGATTGGGACTATCTATTT |
| | | | TAGTGGGACCACCTACTACAACCCGTCCCTCACGAGTCGAG |
| | | | TCAGCATGTCCGTAGACACGTCCAAGAACCAGTTCTCCCTG |
| | | | AAGCTGAACTCTGTGACCGCCGCAGACACGTCTATCTATTA |
| | | | CTGTGCGAGACATTGGCCCAAGTACAACTCCTGGTTCGACC |
| | | | CCTGGGGCCAGGGATTTCTGGTCACCGTCTCCTCA |
| | SEQ ID | HCDR1 | TSSYYWG |
| | NO: 61 | (Kabat) | |
| | SEQ ID | HCDR2 | TIYFSGTTYYNPSLTS |
| | NO: 62 | (Kabat) | |
| | SEQ ID | HCDR3 | HWPKYNSWFDP |
| | NO: 63 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| human | SEQ ID | AA | ASTKGPSVFPLAPSSKSTSGGTAALGCLVKDYFPEPVTVSWNS |
| IgG1 | NO: 70 | | GALTSGVHTFPAVLQSSGLYSLSSVVTVPSSSLGTQTYICNVNH |
| constant | | | KPSNTKVDKKVEPKSCDKTHTCPPCPAPELLGGPSVFLFPPKP |
| region | | | KDTLMISRTPEVTCVVVDVSHEDPEVKFNWYVDGVEVHNAK |
| | | | TKPREEQYNSTYRVVSVLTVLHQDWLNGKEYKCKVSNKALP |
| | | | APIEKTISKAKGQPREPQVYTLPPSREEMTKNQVSLTCLVKGFY |
| | | | PSDIAVEWESNGQPENNYKTTPPVLDSDGSFFLYSKLTVDKSR |
| | | | WQQGNVFSCSVMHEALHNHYTQKSLSLSPGK |
| human | SEQ ID | AA | RTVAAPSVFIFPPSDEQLKSGTASVVCLLNNFYPREAKVQWKV |
| kappa | NO: 71 | | DNALQSGNSQESVTEQDSKDSTYSLSSTLTLSKADYEKHKVY |
| chain | | | ACEVTHQGLSSPVTKSFNRGEC |
| constant | | | |
| region | | | |
| Variant of | SEQ ID | AA | ASTKGPSVFPLAPSSKSTSGGTAALGCLVKDYFPEPVTVSWNS |
| human | NO: 95 | | GALTSGVHTFPAVLQSSGLYSLSSVVTVPSSSLGTQTYICNVNH |
| IgG1 | | | KPSNTKVDKKVEPKSCDKTHTCPPCPAPELLGGPSVFLFPPKP |
| constant | | | KDTLMISRTPEVTCVVVDVSHEDPEVKFNWYVDGVEVHNAK |
| region | | | TKPREEQYNSTYRVVSVLTVLHQDWLNGKEYKCKVSNKALP |
| with Knob | | | APIEKTISKAKGQPREPQVYTLPPSREEMTKNQVSLWCLVKGF |
| mutation | | | YPSDIAVEWESNGQPENNYKTTPPVLDSDGSFFLYSKLTVDKS |
| T366W | | | RWQQGNVFSCSVMHEALHNHYTQKSLSLSPGK |
| Variant of | SEQ ID | AA | ASTKGPSVFPLAPSSKSTSGGTAALGCLVKDYFPEPVTVSWNS |
| human | NO: 96 | | GALTSGVHTFPAVLQSSGLYSLSSVVTVPSSSLGTQTYICNVNH |
| IgG1 | | | KPSNTKVDKKVEPKSCDKTHTCPPCPAPELLGGPSVFLFPPKP |
| constant | | | KDTLMISRTPEVTCVVVDVSHEDPEVKFNWYVDGVEVHNAK |
| region | | | TKPREEQYNSTYRVVSVLTVLHQDWLNGKEYKCKVSNKALP |
| with hole | | | APIEKTISKAKGQPREPQVYTLPPSREEMTKNQVSLSCAVKGF |
| mutations | | | YPSDIAVEWESNGQPENNYKTTPPVLDSDGSFFLVSKLTVDKS |
| T366S, | | | RWQQGNVFSCSVMHEALHNHYTQKSLSLSPGK |
| L368A, | | | |
| and | | | |
| Y407V | | | |

### Example 3. Determination of the Binding Affinity and Specificity of Anti-cMET Antibodies

The binding affinity and specificity of purified anti-cMET antibodies were determined by ELISA and FACS. Briefly, 2 μg/ml of human or cyno monomer cMET fusion protein human cMET-ECD-his or cyno cMET-ECD-his (in house generated, SEQ ID Nos: 1 and 3) or human dimer cMET protein formed by human cMET-ECD-mFc (in house generated, SEQ ID NO: 2) was coated in the 96 well ELISA plates, and 50 μL of serially diluted antibodies were co-incubated for 30-60 min, washed, and incubated with goat anti-human IgG secondary antibody conjugated to HRP (Abcam, ab98624). After incubation and washing, the plates were developed with HRP substrate and absorbance was measured. Candidates positive for ELISA binding were serially diluted and incubated with Hs746T cells, a human gastric carcinoma line which has a putative oncogenic mutation with amplification of cMET resulting in high cMET expression, for 30 minutes at 4° C. After washing twice with FACS buffer, diluted Alexa Fluor 647 goat anti-human IgG secondary antibody was added and incubated with Hs746T cells for 30 minutes at 4° C. in the dark. After washing twice with FACS buffer, cells were resuspended with FACS buffer and read using a Becton Dickinson LSR Fortessa™ cell analyzer. Non-specific bindings of these antibodies were evaluated by FACS binding against Jurkat cells (cMET negative cells). Titration curves were generated using a sigmoidal dose-response of nonlinear fit from GraphPad™ Software by Dotmatics.

As shown in Table 7 and Table 8, all selected candidates specifically bind to cMET monomer and dimer protein in high affinity, and they all specifically bind to human cMET expressing cancer cells and cross bind to cyno cMET.

TABLE 7
Binding affinity of anti-cMET antibodies
against human and cyno cMET protein

| | EC50 | EC50 | EC50 against |
|---|---|---|---|
| | against | against | human |
| | human cMET | cyno cMET | cMET |
| Antibodies | monomer (nM) | momoner (nM) | dimer (nM) |
| 063Ab10910 | 0.01741 | 0.009868 | 0.0543 |
| 061Ab15310 | 0.07772 | 0.1846 | 0.3797 |
| 063Ab16010 | 0.1113 | 0.04456 | 0.6866 |
| 063Ab02110 | 0.02074 | 0.009973 | 0.0705 |
| 063Ab15210 | 0.06627 | 0.05644 | 0.8757 |
| 062Ab16310 | 0.1269 | 0.09184 | 0.04956 |
| 063Ab05510 | 0.5898 | 0.5662 | 2.364 |
| 063Ab07710 | 0.2169 | 0.2971 | 1.346 |
| 063Ab14710 | 0.1453 | 0.1078 | 0.5235 |
| 061Ab05110 | 0.573 | 4.264 | 0.2883 |
| 063Ab03210 | 0.03022 | 0.01385 | 0.05504 |
| 063Ab16720 | 0.02154 | 0.01365 | 0.0362 |

TABLE 8
Binding affinity of anti-cMET antibodies
against Hs746T cells and Jurkat cells

| | EC50 | Emax | EC50 | Emax |
|---|---|---|---|---|
| | against | against | against | against |
| | Hs746T | Hs746T | Jurkat | Jurkat |
| | cell line | cell line | cell line | cell line |
| Antibodies | (nM) | (MFI) | (EC50) | (MFI) |
| 063Ab10910 | 0.5171 | 6.30E+06 | n.d. | 1.06E+04 |
| 061Ab15310 | 0.6303 | 5.91E+06 | n.d. | 3.48E+03 |
| 063Ab16010 | 0.638 | 6.31E+06 | n.d. | 1.02E+04 |
| 063Ab02110 | 0.3061 | 5.83E+06 | n.d. | 1.25E+04 |
| 063Ab15210 | 0.5425 | 5.77E+06 | n.d. | 8.51E+03 |
| 062Ab16310 | 0.2851 | 4.39E+06 | n.d. | 3.60E+03 |
| 063Ab05510 | 0.2497 | 5.07E+06 | n.d. | 4.78E+03 |
| 063Ab07710 | 1.215 | 4.89E+06 | n.d. | 8.36E+03 |
| 063Ab14710 | 1.252 | 5.93E+06 | n.d. | 1.24E+04 |
| 061Ab05110 | 0.9413 | 5.39E+06 | n.d. | 3.57E+03 |
| 063Ab03210 | 0.4364 | 3.22E+06 | n.d. | 6.20E+03 |
| 063Ab16720 | 0.4065 | 4.16E+06 | n.d. | 5.90E+03 |
| hIgG | n.d. | 2.75E+03 | n.d. | 3.78E+03 |

n.d.: non detected

### Example 4. Binding Kinetics of Anti-cMET Antibodies

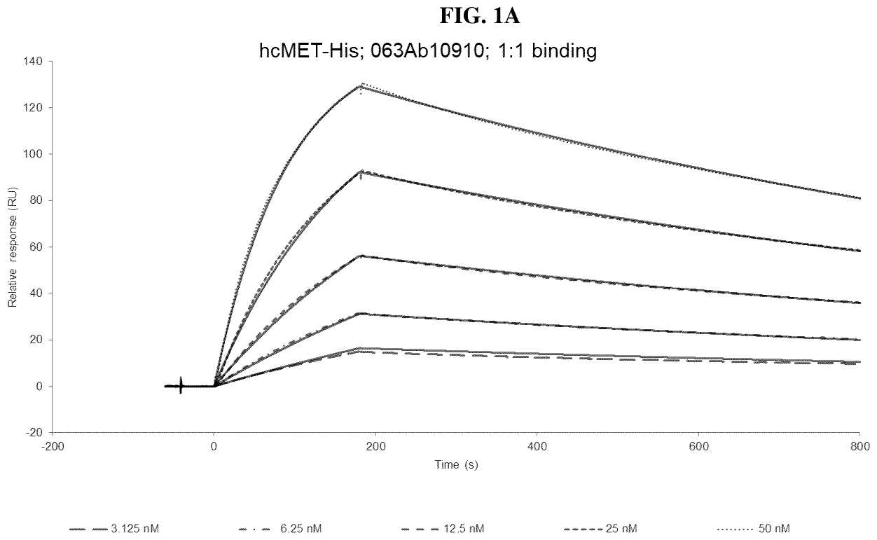

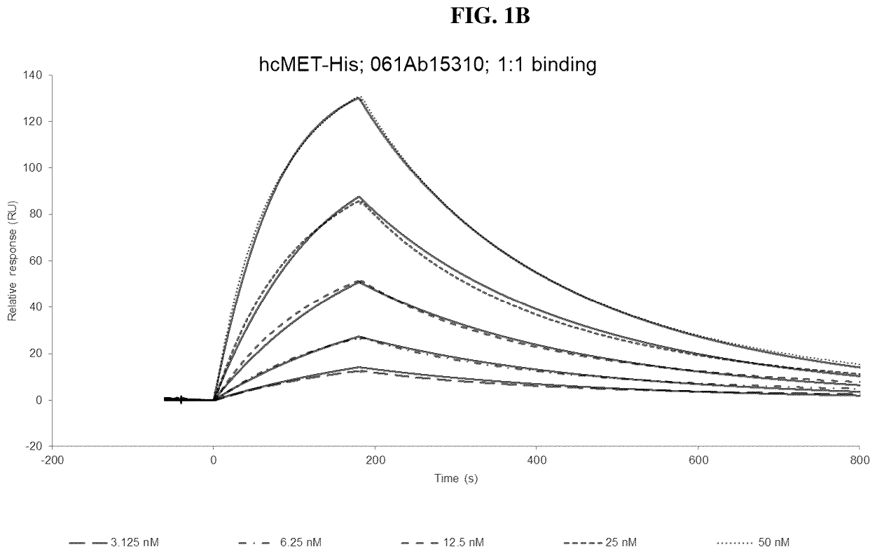

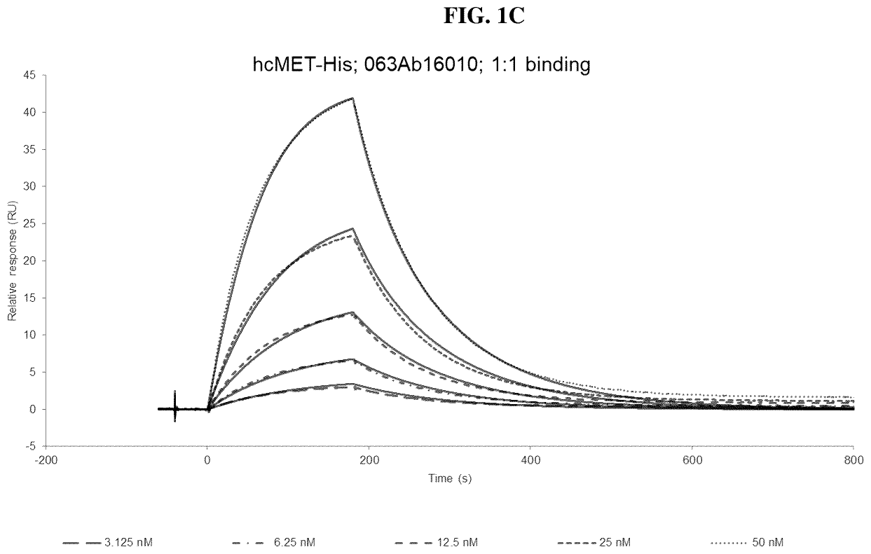

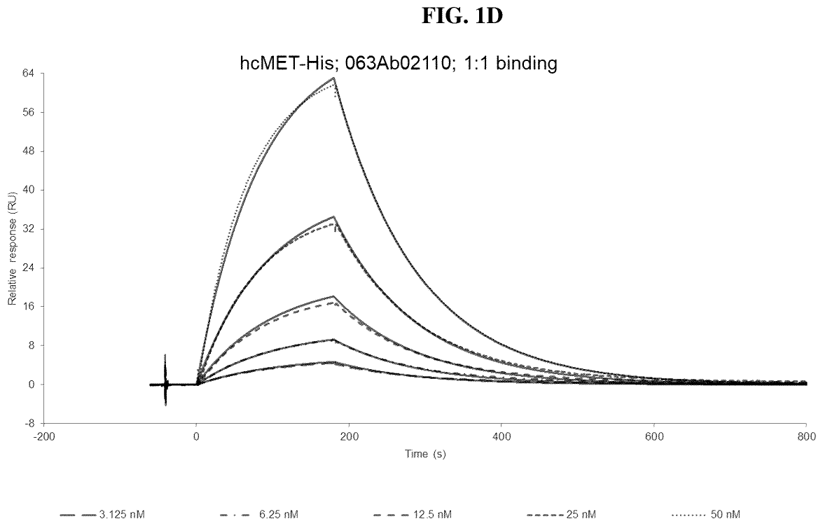

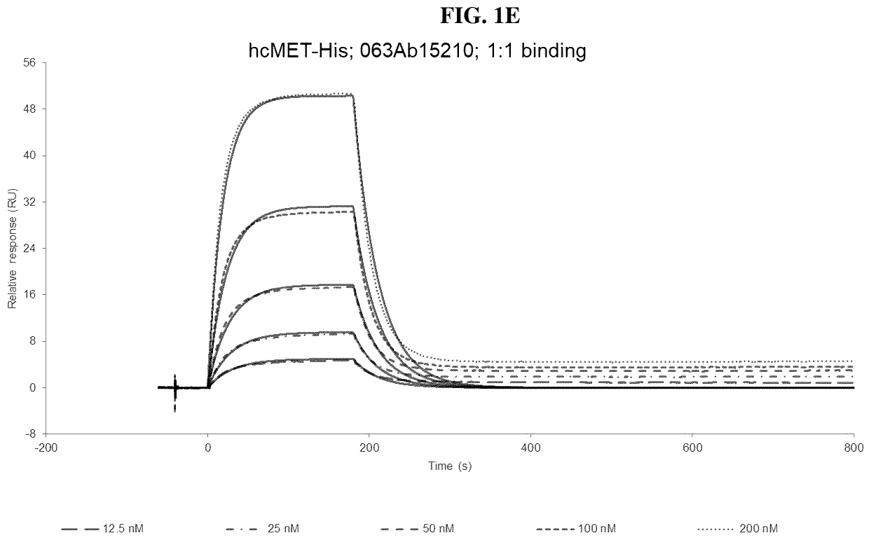

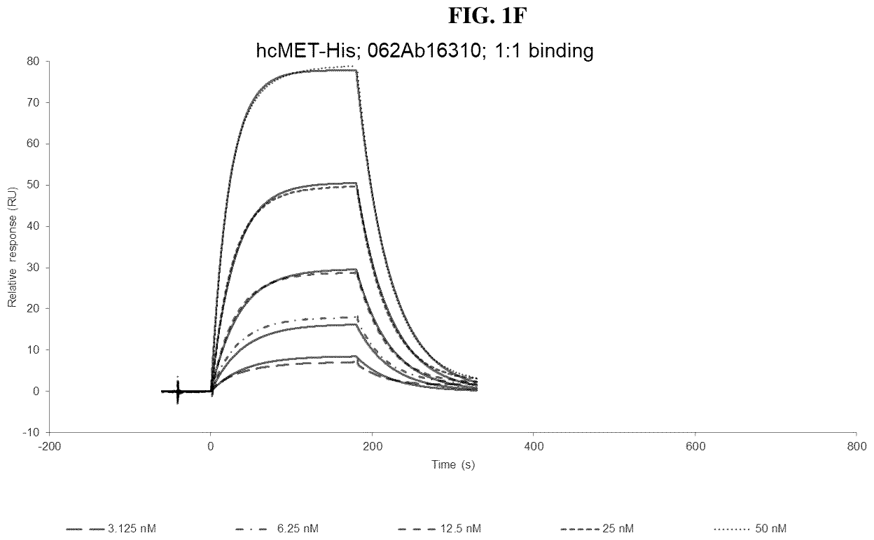

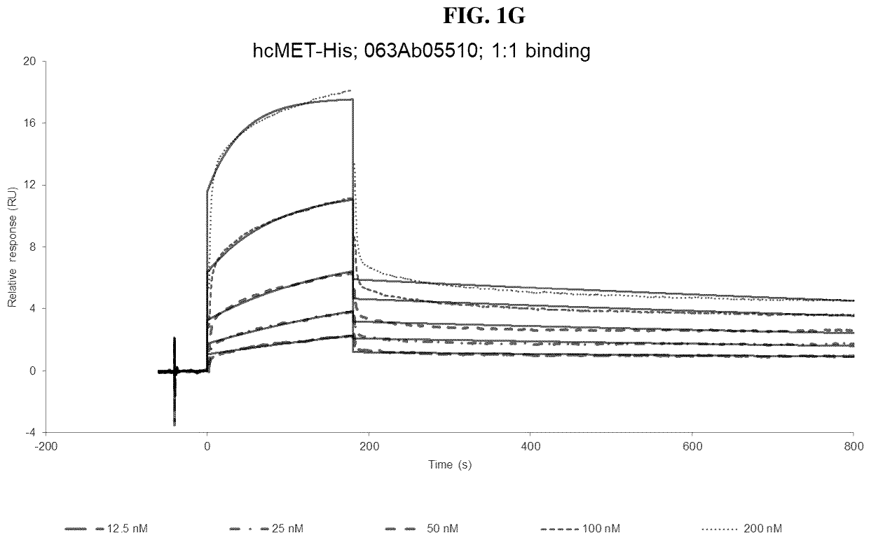

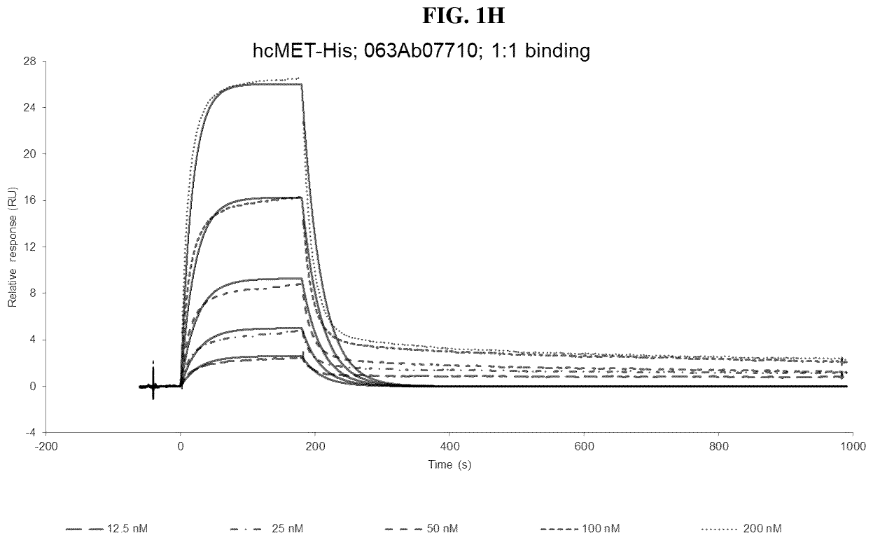

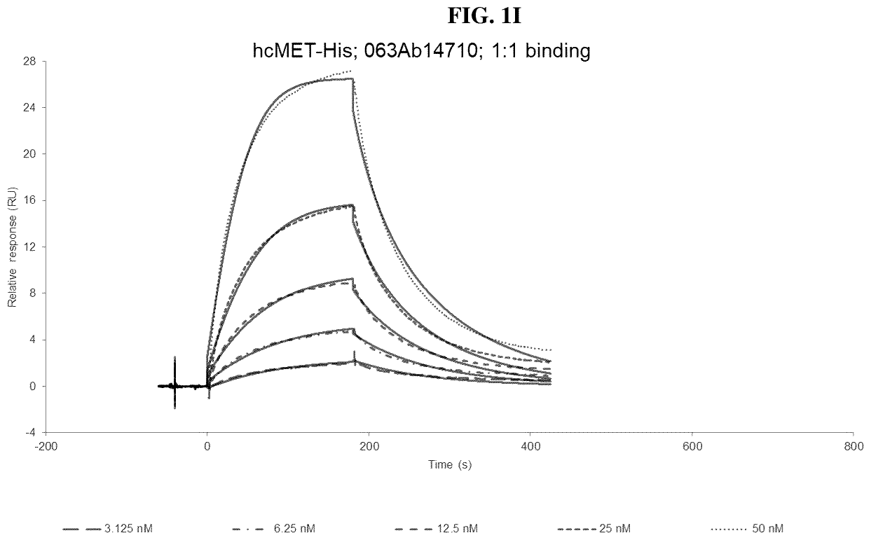

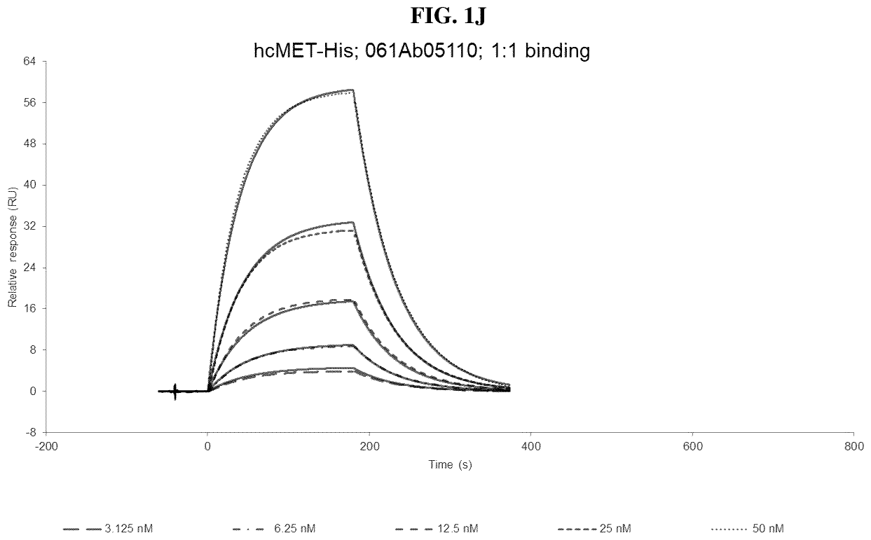

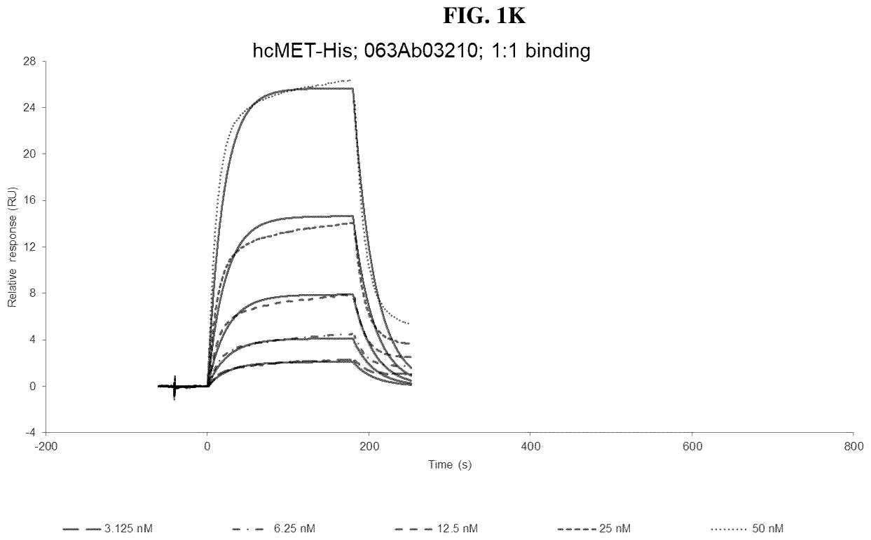

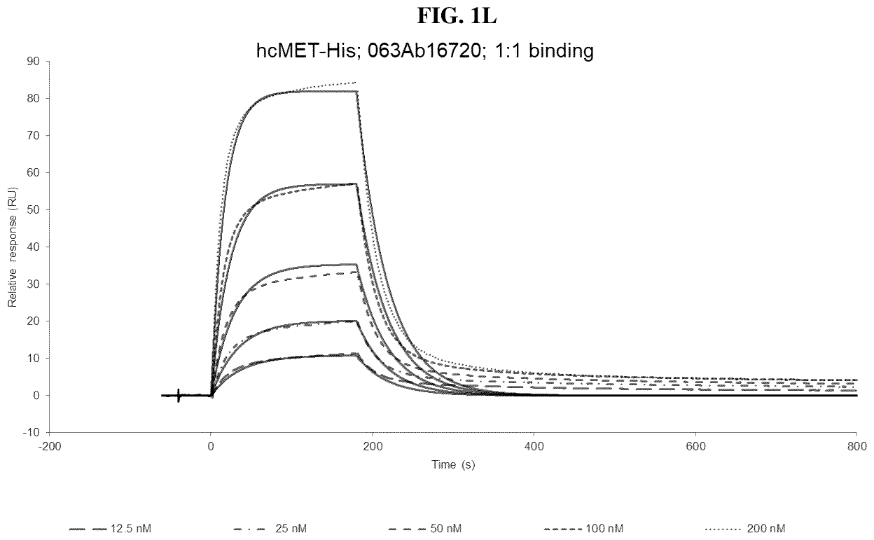

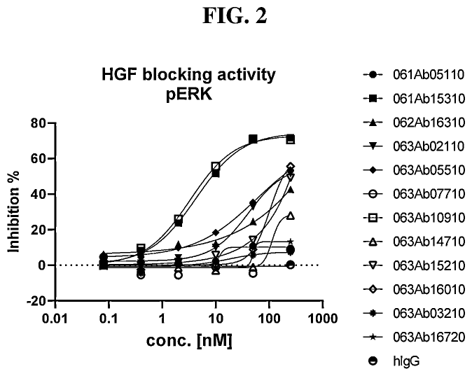

The binding affinity and kinetics of purified anti-cMET antibodies were determined by Surface plasmon resonance (Biacore 8K, GE Life Sciences) at room temperature. Briefly, mouse anti-human IgG Fc antibody was immobilized via amine coupling onto a activated CM5 biosensor chip (Cat. No. BR100530, GE Life Sciences). Purified monoclonal antibody candidates were flowed over the chip surface and captured by anti-human IgG antibody. Then a serial dilution of soluble monomeric human cMET fusion proteins with his-tag (in house generated, SEQ ID NO: 1) were injected over the antibodies captured surface and changes in surface plasmon resonance signals were analyzed to calculate the association rates (kₒₙ) and dissociation rates (k_{off}) by using the one-to-one Langmuir binding model (BIA Evaluation Software™, GE Life Sciences). The equilibrium dissociation constant (K_{D}) was calculated as the ratio k_{off}/kₒₙ. The binding affinity and kinetics profiles of selected monoclonal anti-cMET antibodies are listed below in Table 9, and the detail binding curves of selected candidates are presented in FIGS. 1A-1L.

TABLE 9
Binding kinetics of anti-cMET antibodies
against monomeric cMET protein

| | 1:1 binding ka | | |
|---|---|---|---|
| Antibodies | (1/Ms) | kd (1/s) | KD (M) |
| 063Ab10910 | 2.28E+05 | 7.78E−04 | 3.40E−09 |
| 061Ab15310 | 3.06E+05 | 5.35E−03 | 1.75E−08 |
| 063Ab16010 | 6.30E+05 | 5.06E−02 | 8.04E−08 |
| 063Ab02110 | 6.14E+04 | 9.50E−03 | 1.55E−07 |
| 063Ab15210 | 1.03E+05 | 3.22E−02 | 3.11E−07 |
| 062Ab16310 | 4.55E+05 | 2.66E−02 | 5.85E−08 |
| 063Ab05510 | 1.38E+05 | 2.35E−02 | 1.70E−07 |
| 063Ab07710 | 1.00E+05 | 3.63E−02 | 3.62E−07 |
| 063Ab14710 | 1.56E+06 | 5.02E−02 | 3.23E−08 |
| 061Ab05110 | 1.17E+05 | 1.96E−02 | 1.68E−07 |
| 063Ab03210 | 2.03E+05 | 3.40E−02 | 1.68E−07 |
| 063Ab16720 | 1.65E+05 | 2.59E−02 | 1.56E−07 |

### Example 5. Blocking Activity on Ligand-Induced Signaling of Monoclonal Anti-cMET Antibodies

To assess the activity of anti-cMET antibodies to interfere with the HGF-cMET interaction and inhibit the downstream signaling, the inhibition of HGF-induced ERK phosphorylation at T202/Y204 upon antibody treatment was used as a cellular readout. Briefly, H596 cells (epithelial-like cell line isolated from human lung cancer) were seeded to one 96-well assay plate and incubated at 37° C., 5% CO2. After 24 hours, cells were starved with medium without FBS for 4 hours. Then, a series of dilutions of anti-cMET antibodies were added to each well and incubated at 37° C. for 1 hour. 100 pM human HGF was added to each well and incubated at 37° C. for 15 minutes. Then, the medium was discarded and 1× lysis buffer was added to the assay plate and incubated at room temperature for 30 minutes. Cellular phosphor-ERK was measured using ERK phospho-T202/Y204 Kit (PerkinElmer). IC50 values were determined by fitting the dose-response data to the four-parameter logistic model with GraphPad Prism. Imax was calculated using the equation: % inhibition=100*[1−(X−MIN)/(MAX−MIN)]. X is the FRET signal at a given compound concentration. MAX is the signal in the presence of H596 cells and 100 pM hHGF. MIN is the signal in the presence of starvation medium as the background signal. IC50 and Imax of monoclonal anti-cMET antibodies are shown in Table 10 and FIG. 2.

TABLE 10
Comparison of blocking activity on ligand-induced
signaling of monoclonal anti-cMET antibodies

| Antibody | IC50 (nM) | Imax (%) |
|---|---|---|
| 061Ab05110 | 11.75 | 10.84 |
| 061Ab15310 | 4.26 | 71.60 |
| 062Ab16310 | 65.62 | 42.74 |
| 063Ab02110 | 40.75 | 51.40 |
| 063Ab05510 | 58.02 | 53.66 |
| 063Ab07710 | >250 | 8.71 |
| 063Ab10910 | 3.30 | 70.95 |
| 063Ab14710 | 113.00 | 28.06 |
| 063Ab15210 | 65.73 | 49.19 |
| 063Ab16010 | 102.90 | 55.64 |
| 063Ab03210 | >250 | 7.41 |
| 063Ab16720 | 9.57 | 13.20 |
| hIgG | N/A | 0.33 |

### Example 6. Agonist Activity of Monoclonal Anti-cMET Antibodies

To assess the transient agonistic activity of anti-cMET antibodies, the activation of cMET downstream signaling: upregulation of ERK phospho-T202/Y204 was measured upon antibody treatment. Briefly, H596 cells were seeded to the 96-well assay plate and incubated at 37° C., 5% CO2. After 24 hours, cells were starved with medium without FBS for 4 hours. Then, 50 nM of anti-cMET antibodies were added to each well and incubated at 37° C. for 2 hours. Then, the medium was discarded and 1× lysis buffer was added to the assay plate and incubated at room temperature for 30 minutes. Cellular phosphor-ERK was measured using ERK phospho-T202/Y204 Kit (PerkinElmer). Stimulation fold was determined by comparing the p-ERK signal in the presence of 50 nM antibody with that in the presence of the vehicle (buffer PBS). The agonist activity of monoclonal anti-cMET antibodies are shown in Table 11.

TABLE 11
Agonist activity of monoclonal anti-cMET antibodies

| Antibody | Stimulation fold at 50 nM |
|---|---|
| 061Ab05110 | 3.43 |
| 061Ab15310 | 4.06 |
| 062Ab16310 | 5.47 |
| 063Ab02110 | 2.88 |
| 063Ab05510 | 3.50 |
| 063Ab07710 | 4.57 |
| 063Ab10910 | 1.21 |
| 063Ab14710 | 3.62 |
| 063Ab15210 | 3.66 |
| 063Ab16010 | 4.09 |
| 063Ab03210 | 5.72 |
| 063Ab16720 | 5.18 |
| hIgG | 0.96 |

### Example 7. Anti-cMET Antibodies Bind to Different Epitopes

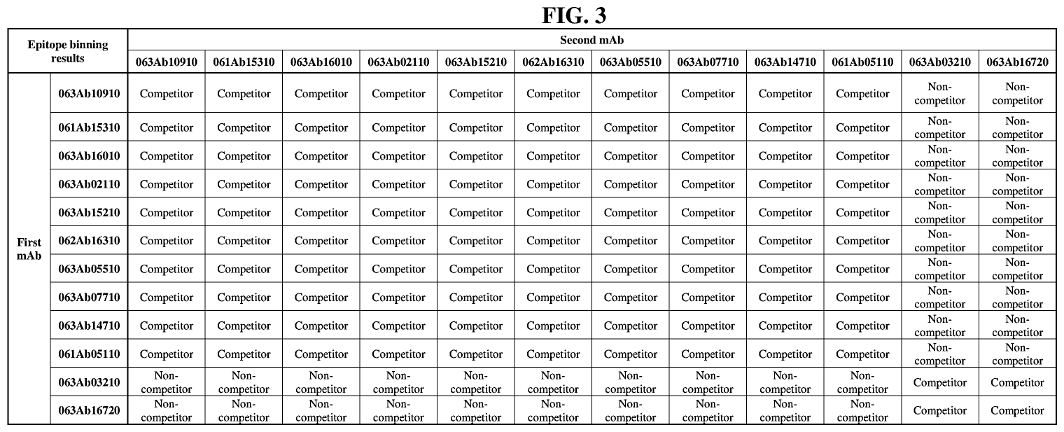

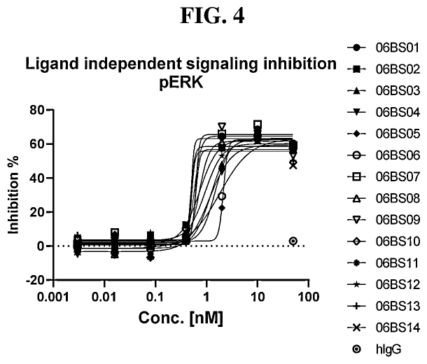

To assess whether anti-cMET antibodies compete with each other, an epitope binning assay was performed using Surface plasmon resonance (Biacore 8K, GE Life Sciences) at room temperature. Briefly, human cMET-his (in house generated, SEQ ID NO: 1) is captured to the sensor surface of anti-his antibody immobilized CM5 chip (Cat. No. 29234602, GE Life Sciences). The first antibody was injected at 300 nM to saturate antigen binding, followed by the injection of the second antibodies. Data was analyzed utilizing Biacore Insight Epitope Binning Extension. Epitope binning results of representative monoclonal antibodies are listed in FIG. 3. Based on FIG. 3, the Anti-cMET antibodies are divided into two non-competitive groups: (1) 063Ab10910, 061Ab15310, 063Ab16010, 063Ab02110, 063Ab15210, 062Ab16310, 063Ab05510, 063Ab07710, 063Ab14710 or 061Ab05110; and (2) 063Ab03210, or 063Ab16720.

### Example 8. Construction of Bispecific Antibodies have 2 Different Antigen Binding Domains Against Distinct Epitopes of cMET

In order to generate bi-paratopic antibodies against cMET, we constructed bispecific antibodies comprising two different arms (Arm1 and Arm2), wherein Arm1 and Arm2 are derived from different anti-cMET monospecific antibodies which bind to separate epitopes and are non-competitive.

The individual anti-cMET monospecific antibodies were derived from candidates described in Examples 1 to 7 herein. All anti-cMET antibodies described here share the common light chain (SEQ ID NO: 64). Bispecific antibodies were expressed by Expi293™ cells and purified by MabSelect™ SuRe affinity chromatography. Selected representatives are listed in Table 12 to illustrate the pairing algorithm and bispecific antibody sequence ID.

TABLE 12
Anti-cMET bispecific antibodies
pairing algorithm and sequence ID

| BsAb_seq ID and | | Arm2 | |
|---|---|---|---|
| pairing | | 063Ab03210 | 063Ab16720 |
| Arm1 | 063Ab10910 | 06BS01 | 06BS02 |
| | 061Ab15310 | 06BS03 | 06BS04 |
| | 063Ab16010 | 06BS05 | 06BS06 |
| | 063Ab02110 | 06BS07 | 06BS08 |
| | 063Ab15210 | 06BS09 | 06BS10 |
| | 062Ab16310 | 06BS11 | 06BS12 |
| | 063Ab05510 | 06BS13 | 06BS14 |
| | 063Ab07710 | 06BS15 | 06BS16 |
| | 063Ab14710 | 06BS17 | 06BS18 |
| | 061Ab05110 | 06BS19 | 06BS20 |

### Example 9. Inhibition of Ligand-Independent Signaling by Bispecific Anti-cMET Antibodies

For cancer cell lines with cMET amplification, like Hs746T (human gastric cancer cell), the cMET signaling is constitutively active to support cell proliferation without the need for ligand engagement. To assess the activity of anti-cMET bispecific antibodies to inhibit the ligand-independent cMET signaling, the inhibition of ERK phospho-T202/Y204 was measured in Hs746T cells upon anti-cMET bispecific antibodies treatment. Briefly, cell Hs746T was seeded in a 96-well assay plate and incubated at 37° C., 5% CO2. After 24 hours, cells were treated with 10 nM of anti-cMET bispecific antibody and incubated at 37° C. for 18 hours. Then, the medium was discarded and 1× lysis buffer was added to the assay plate and incubated at room temperature for 30 minutes. Cellular phosphor-ERK was measured using ERK phospho-T202/Y204 Kit (PerkinElmer). The percentage of inhibition was calculated using the equation: % inhibition=100*[1−(X−MIN)/(MAX−MIN)]. X is the FRET signal at a given compound concentration. MAX is the signal in the presence of Hs746T and PBS only. MIN is the signal in the presence of medium only as the background signal. As shown in Table 13, all paired bispecific antibodies exhibited inhibitory activity on ligand-independent cMET signaling at 10 nM.

TABLE 13
Inhibition of ligand-independent signaling
by bispecific anti-cMET antibodies

| Antibodies | | | |
|---|---|---|---|
| | | Bispecific | PERK inhibition |
| Arm 1 | Arm2 | antibody | % (at 10 nM) |
| 063Ab10910 | 063Ab03210 | 06BS01 | 75.54 |
| 063Ab10910 | 063Ab16720 | 06BS02 | 67.97 |
| 063Ab14710 | 063Ab03210 | 06BS17 | 68.94 |
| 063Ab14710 | 063Ab16720 | 06BS18 | 56.1 |
| 063Ab07710 | 063Ab03210 | 06BS15 | 73.88 |
| 063Ab07710 | 063Ab16720 | 06BS16 | 60.14 |
| 063Ab02110 | 063Ab03210 | 06BS07 | 75.01 |
| 063Ab02110 | 063Ab16720 | 06BS08 | 69.03 |
| 063Ab15210 | 063Ab03210 | 06BS09 | 73.86 |
| 063Ab15210 | 063Ab16720 | 06BS10 | 71.17 |
| 062Ab16310 | 063Ab03210 | 06BS11 | 74.51 |
| 062Ab16310 | 063Ab16720 | 06BS12 | 69.13 |
| 061Ab15310 | 063Ab03210 | 06BS03 | 72.8 |
| 061Ab15310 | 063Ab16720 | 06BS04 | 67.78 |
| 063Ab16010 | 063Ab03210 | 06BS05 | 72.36 |
| 063Ab16010 | 063Ab16720 | 06BS06 | 57.59 |
| 063Ab05510 | 063Ab03210 | 06BS13 | 70.16 |
| 063Ab05510 | 063Ab16720 | 06BS14 | 73.29 |
| 061Ab05110 | 063Ab03210 | 06BS19 | 67.18 |
| 061Ab05110 | 063Ab16720 | 06BS20 | 66.85 |

| | |
|---|---|
| hIgG | 2.35 |

### Example 10. Inhibitory Activity of Ligand-Independent Signaling and Anti-Proliferation Activity of Bispecific Anti-cMET Antibodies

The ligand-independent signaling inhibitory activity of anti-cMET bispecific antibodies was measured by the method described in Example 9. Briefly, Hs746T cells were seeded to 96-well assay plate and treated with a series of dilutions of anti-cMET antibodies and incubated at 37° C. for 18 hours. Then, cellular phosphor-ERK was measured using ERK phospho-T202/Y204 Kit (PerkinElmer). IC50 values were determined by fitting the dose-response data to the four-parameter logistic model with GraphPad Prism. As shown in Table 14 and FIG. 4, all bispecific antibodies tested below 06BS01-06BS14 exhibited potent ligand-independent signaling inhibitory activity.

TABLE 14
Inhibition of ligand-independent signaling
by bispecific anti-cMET antibodies

| Antibodies | IC50 (nM) | Imax % |
|---|---|---|
| 06BS01 | 0.58 | 68.85 |
| 06BS02 | 0.73 | 67.57 |
| 06BS03 | 0.5 | 66.19 |
| 06BS04 | 0.49 | 63.94 |
| 06BS05 | 1.49 | 67.22 |
| 06BS06 | 0.94 | 63.53 |
| 06BS07 | 1.24 | 66.16 |
| 06BS08 | 1.15 | 63.07 |
| 06BS09 | 2.15 | 68.98 |
| 06BS10 | 1.82 | 64.89 |
| 06BS11 | 0.62 | 71.43 |
| 06BS12 | 0.72 | 66.7 |
| 06BS13 | 0.62 | 63.46 |
| 06BS14 | 0.51 | 61.93 |
| hIgG | N/A | 2.93 |

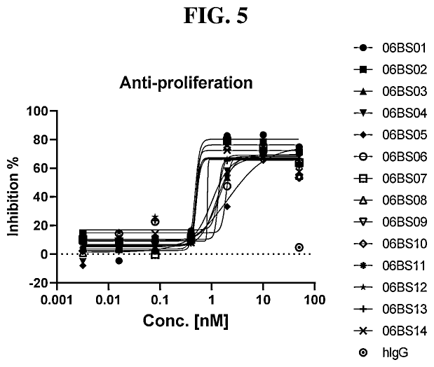

The anti-proliferation activity of anti-cMET bispecific antibodies was measured on cMET signaling dependent cell Hs746T. Cell-Titer-Glo assay (Promega) was used as a cell-based assay to evaluate the cell viability after anti-cMET antibodies treatment. Briefly, Hs746T cells were seeded to 96-well assay plate, cells were treated with a series of dilutions of anti-cMET bispecific antibodies and incubated at 37° C., 5% CO₂ for 6 days. After the antibodies treatment, 50 μl of Cell-Titer-Glo reagent was added in each well. Mixture was mixed on an orbital shaker for 10 min to allow cell lysis. Luminescent signal was measured using PheraStar (BMG Labtech) with luminescent protocol. IC50 values were determined by fitting the dose-response data to the four-parameter logistic model with GraphPad Prism. As shown in Table 15 and FIG. 5, all bispecific antibodies tested below 06BS01-06BS014 exhibited potent anti-proliferation activity on Hs746T, a cell line relies on constitutively activated cMET signaling.

TABLE 15
Anti-proliferation activity of bispecific
anti-cMET antibodies on Hs746T

| Antibodies | IC50(nM) | Imax % |
|---|---|---|
| 06BS01 | 0.48 | 83.24 |
| 06BS02 | 0.51 | 79.09 |
| 06BS03 | 0.51 | 77.17 |
| 06BS04 | 0.51 | 80.78 |
| 06BS05 | 1.2 | 70.8 |
| 06BS06 | 1.37 | 73.5 |
| 06BS07 | 1.39 | 75.39 |
| 06BS08 | 1.02 | 74.93 |
| 06BS09 | 2.08 | 71.29 |
| 06BS10 | 1.91 | 71.62 |
| 06BS11 | 0.49 | 79.26 |
| 06BS12 | 0.48 | 74.91 |
| 06BS13 | 1.34 | 70.28 |
| 06BS14 | 0.89 | 68.31 |
| hIgG | N/A | 4.75 |

### Example 11. Blocking Activity on Ligand-Induced Signaling by Bispecific Anti-cMET Antibodies

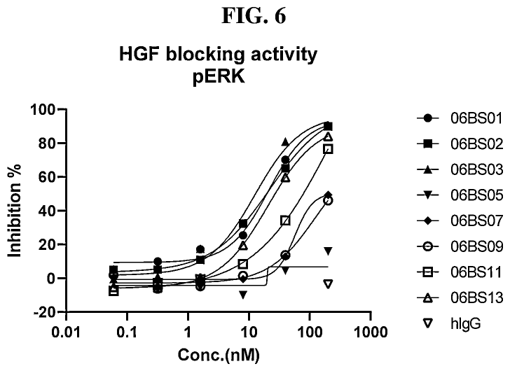

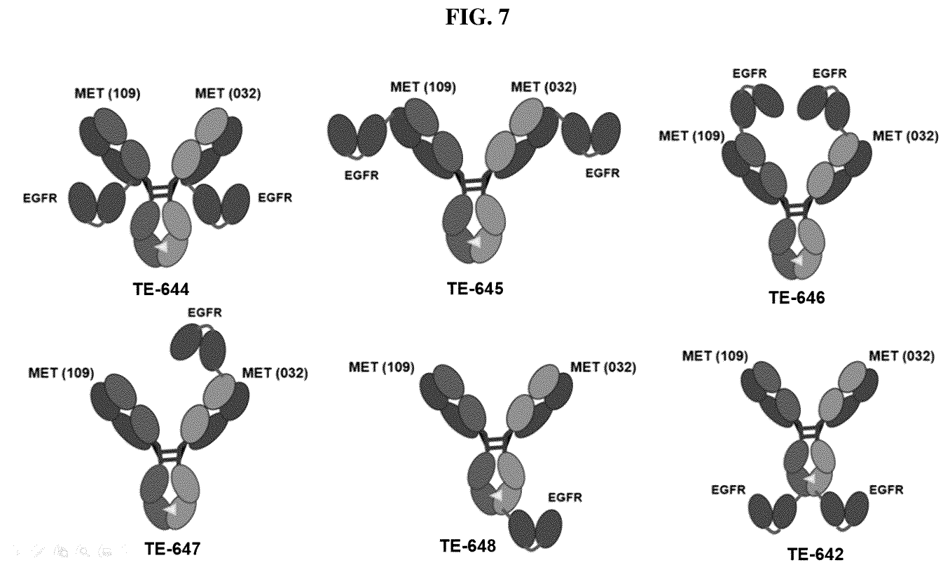

To assess the activity of anti-cMET bispecific antibodies to interfere with the HGF-cMET interaction and inhibit downstream signaling, the inhibition of HGF-induced ERK phospho-T202/Y204 upon antibody treatment was used as a readout. Briefly, H596 cells (epithelial-like cell line isolated from the lung cancer) were seeded to the 96-well assay plate and incubated at 37° C., 5% CO₂. After 24 hours, cells were starved with medium without FBS for 4 hours. Then, a series of dilutions of anti-cMET antibodies were added to each well and incubated at 37° C. for 1 hour. 100 pM human HGF was added to each well and incubated at 37° C. for 15 minutes. Then, the medium was discarded and 1× lysis buffer was added to the assay plate and incubated at room temperature for 30 minutes. Cellular phosphor-ERK was measured using ERK phospho-T202/Y204 Kit (PerkinElmer). IC50 values were determined by fitting the dose-response data to the four-parameter logistic model with GraphPad Prism. As shown in Table 16 and FIG. 6, bispecific anti-cMET antibodies 06BS01, 06BS02. 06BS03, 06BS11, 06BS13 exhibited strong activities of blocking HGF-induced cMET signaling.

TABLE 16
Blocking activity on ligand-induced signaling
of bispecific anti-cMET antibodies

| Antibodies | IC50 (nM) | Imax % |
|---|---|---|
| 06BS01 | 21.08 | 90.25 |
| 06BS02 | 21.7 | 89.99 |
| 06BS03 | 12.49 | 90.72 |
| 06BS05 | 40.23 | 16.04 |
| 06BS07 | 56.75 | 49.32 |
| 06BS09 | 50.75 | 50.75 |
| 06BS11 | 39.9 | 76.62 |
| 06BS13 | 20.67 | 84.18 |
| hIgG | N/A | −3.55 |

### Example 12. Removal of Post-Translational Modification Sites of Anti-cMET Antibodies

Point mutation was performed to remove some potential post-translational modification (PTM) sites of cMET antibodies.

For cMET antibodies 063Ab03210, 061Ab15310, and 063Ab10910, N terminal Glutamine (Q) of VH was mutated to Glutamic acid (E) to avoid the heterogeneity caused by cyclization of N terminal Q, resulting cMET antibodies 063Ab03210-V1 (see SEQ ID NOs: 82 and 64), 061Ab15310-V1 (SEQ ID NOs: 73 and 64), and 063Ab10910-V1 (see SEQ ID Nos: 72 and 64). As shown in Table 17, cMET antibodies 063Ab03210-V1 showed similar KD as parental antibody 063Ab03210, cMET antibodies 061Ab15310-V1 showed similar KD as parental antibody 061Ab15310, and cMET antibodies 063Ab10910-V1 showed similar KD as parental antibody 063Ab10910. The engineered VH sequences of cMET antibodies are shown in Table 18.

TABLE 17
SPR binding affinity of cMET antibodies 063Ab03210,
061Ab15310, 063Ab10910 and the variants thereof.

| Antibody variants | ka (1/Ms) | kd (1/s) | KD (M) |
|---|---|---|---|
| 063Ab03210 | 2.72E+5 | 2.01E−2 | 7.37E−8 |
| 063Ab03210-V1 | 2.69E+5 | 1.90E−2 | 7.05E−8 |
| 061Ab15310 | 9.73E+4 | 3.81E−3 | 3.92E−8 |
| 061Ab15310-V1 | 9.98E+4 | 3.61E−3 | 3.61E−8 |
| 063Ab10910 | 1.27E+5 | 7.14E−4 | 5.61E−9 |
| 063Ab10910-V1 | 1.26E+5 | 6.94E−4 | 5.49E−9 |

TABLE 18
Engineered VH sequences of cMET antibodies

| | SEQ ID | | |
|---|---|---|---|
| Antibody | NO | Description | SEQUENCE |
| 063Ab1091-0 | SEQ ID | VH (AA) | EVQLVESGGGVVQPGRSLRLSCAASGFTFSSHGMHWVRQA |
| V1 | NO: 72 | | PGKGLEWVAVISYDESDKYYADSVKGRFTISRDHSKNTLYL |
| | | | QMNSLRAEDTAVYYCVKDRNMGYSGWLDYWGQGTLVTV |
| | | | SS |
| 061Ab15310- | SEQ ID | VH (AA) | EVQLVESGGGVVQPGKSLRLSCAASGFTFSSSGLHWVRQA |
| V1 | NO: 73 | | PGKGLEWVAVISYDTSNKYYADSVKGRFTISRDNSKNTLYL |
| | | | QMNSLRAEDTAVYYCAKDLNRGYDWGFDYWGQGTLVTV |
| | | | SS |
| 063Ab16010- | SEQ ID | VH (AA) | EVQLVESGGGVVQPGRSLRLSCAASGFTFSNYGMHWVRQ |
| V1 | NO: 74 | | APGKGLEWVAVISYDGVDKYYADSVKGRFTISRDNSKNTL |
| | | | YLQMNSLRAEDTAMYYCAKDRISYNWNLDYWGQGTLVT |
| | | | VSS |
| 063Ab02110- | SEQ ID | VH (AA) | EVQLVESGGGVVQPGRSLRLSCVASGFTFSNYGLHWVRQA |
| V1 | NO: 75 | | PGKGLEWVAAISYDGSDKYYADPVKGRFTISRDTTKNTLSL |
| | | | QMNSLRAEDTAVYYCAKERYRGYDWDFDYWGQGTLVTV |
| | | | SS |
| 063Ab15210- | SEQ ID | VH (AA) | EVQLVESGGGVVQPGRSLRLSCAASGFTFSSYGLHWVRQA |
| V1 | NO: 76 | | PGKGLEWVAVISFDGSNKYYADSVKGRFTISRDTSKNTLSL |
| | | | QMNSLRAEDTAVYYCAKERSRGYDWDFDYWGQGILVTVS |
| | | | S |
| 062Ab16310- | SEQ ID | VH (AA) | EVRLVESGGGVVQPGRSLRLSCAASGFTFSNYGMHWVRQA |
| V1 | NO: 77 | | PGKGLEWVAVISYDGSDKYYPDSVKGRFTISRDNSRNTLYL |
| | | | QMNSLSTEDTAVYYCAKDQYRGYDGTFDYWGQGTLVTVS |
| | | | S |
| 063Ab05510- | SEQ ID | VH (AA) | EVQLVESGGGVVQPGRSLRLSCAASGFIFSSYNMHWVRQA |
| V1 | NO: 78 | | PGKGLEWVAVISYDRVNKYYADSVKGRFTISRDNSKNTLY |
| | | | LQMNSLRVEDTAVYYCAKERERYFDWLDYWGQGTLVTVS |
| | | | S |
| 063Ab07710- | SEQ ID | VH (AA) | EVQLVESGGGVVQPGRSLRLSCAASGFTFSSYGMHWVRQA |
| V1 | NO: 79 | | PGKGLEWVAVISNDGSYKYYVDSVTGRFTISRDNSKNTLYL |
| | | | QMNSLRAEDTAVYYCAKDRNMGYSGWFDYWGQGSLVTV |
| | | | SS |
| 063Ab14710- | SEQ ID | VH (AA) | EVQLVESGGGVVQPGRSLRLSCAASGFTFSSHGMHWVRQA |
| V1 | NO: 80 | | PGKGLEWVAVISNDGSYKYYADSVTGRFTISRDNSKNTLYL |
| | | | QMNSLRAEDTAVYYCAKDRNMGYSGWFDYWGQGSLVTV |
| | | | SS |
| 061Ab05110- | SEQ ID | VH (AA) | EVQLVESGGGVVQPGRSLRLSCAASGFTFSNYGMHWVRQ |
| V1 | NO: 81 | | APGKGLEWVAVISNDGSEKYYADSVKGRFTISRDSSKNTLY |
| | | | LQMNSLRAEDTAVYYCAKAPWGSGTTGRLDYWGQGTLVT |
| | | | VSS |
| 063Ab03210- | SEQ ID | VH (AA) | EFQLQESGPGLVKPSETLSLTCTVSGGSISTSSYYWGWIRQP |
| V1 | NO: 82 | | PGKGLEWIGTIYYSGTTYYNPSLKSRVTISVDTSKNQFSLKL |
| | | | KSVTAADTSIYYCARHWPKYNSWFDPWGQGILVTVSS |
| 063Ab16720- | SEQ ID | VH (AA) | EFQLQESGPGLVKPSETLSLTCTVSGGSISTSSYYWGWIRQP |
| V1 | NO: 83 | | PGKGLEWIGTIYFSGTTYYNPSLTSRVSMSVDTSKNQFSLKL |
| | | | NSVTAADTSIYYCARHWPKYNSWFDPWGQGFLVTVSS |

CDR Engineering of cMET Antibody 063Ab10910

For 063Ab10910, the potential deamidation and oxidation sites of NM motif in HCDR3 were mutated to various amino acid residues. Surface plasmon resonance (Biacore 8K, GE Life Sciences) was used to characterize the relative binding affinity to cMET LCD protein (Cat #MET-H5227, ACRO Biosystems) after PTM sites removal. The HCDR and VH sequences for each variant are summarized in Table 19. The LCDR and light chain sequences (SEQ ID NO: 64) remain the same common light chain as that of parental antibody. The variants 063Ab10910-P28, 063Ab10910-P27, 063Ab10910-P37, 063Ab10910-P26, and 063Ab10910-P19 showed comparable binding affinity as that of parental cMET antibody 063Ab10910, as shown in Table 20.

TABLE 19
HCDR and VH sequences of 063Ab10910 and the variants thereof.

| Antibody | SEQ ID NO | Description | SEQUENCE |
|---|---|---|---|
| 063Ab10910 | SEQ ID NO: 6 | HCDR1 (Kabat) | SHGMH |
| | SEQ ID NO: 7 | HCDR2 (Kabat) | VISYDESDKYYADSVKG |
| | SEQ ID NO: 8 | HCDR3 (Kabat) | DRNMGYSGWLDY |
| | SEQ ID NO: 4 | VH (AA) | QVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRN |
| | | | MGYSGWLDYWGQGTLVTVSS |
| 063Ab10910- | SEQ ID NO: 6 | HCDR1 (Kabat) | SHGMH |
| P28 | SEQ ID NO: 7 | HCDR2 (Kabat) | VISYDESDKYYADSVKG |
| | SEQ ID NO: 84 | HCDR3 (Kabat) | DRWEGYSGWLDY |
| | SEQ ID NO: 85 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDR |
| | | | WEGYSGWLDYWGQGTLVTVSS |
| 063Ab10910- | SEQ ID NO: 6 | HCDR1 (Kabat) | SHGMH |
| P27 | SEQ ID NO: 7 | HCDR2 (Kabat) | VISYDESDKYYADSVKG |
| | SEQ ID NO: 86 | HCDR3 (Kabat) | DRYEGYSGWLDY |
| | SEQ ID NO: 87 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRY |
| | | | EGYSGWLDYWGQGTLVTVSS |
| 063Ab10910- | SEQ ID NO: 6 | HCDR1 (Kabat) | SHGMH |
| P37 | SEQ ID NO: 7 | HCDR2 (Kabat) | VISYDESDKYYADSVKG |
| | SEQ ID NO: 88 | HCDR3 (Kabat) | DRHEGYSGWLDY |
| | SEQ ID NO: 89 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRH |
| | | | EGYSGWLDYWGQGTLVTVSS |
| 063Ab10910- | SEQ ID NO: 6 | HCDR1 (Kabat) | SHGMH |
| P26 | SEQ ID NO: 7 | HCDR2 (Kabat) | VISYDESDKYYADSVKG |
| | SEQ ID NO: 90 | HCDR3 (Kabat) | DRFEGYSGWLDY |
| | SEQ ID NO: 91 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRF |
| | | | EGYSGWLDYWGQGTLVTVSS |
| 063Ab109 | SEQ ID NO: 6 | HCDR1 (Kabat) | SHGMH |
| 10-P19 | SEQ ID NO: 7 | HCDR2 (Kabat) | VISYDESDKYYADSVKG |
| | SEQ ID NO: 92 | HCDR3 (Kabat) | DRNEGYSGWLDY |
| | SEQ ID NO: 93 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRN |
| | | | EGYSGWLDYWGQGTLVTVSS |
| 063Ab10910 | SEQ ID NO: 6 | HCDR1 (Kabat) | SHGMH |
| variants | SEQ ID NO: 7 | HCDR2 (Kabat) | VISYDESDKYYADSVKG |
| | SEQ ID NO: 94 | HCDR3 (Kabat) | DRX1X2GYSGWLDY, wherein X1 = W, Y, H, |
| | | | F or N; and X2 = E. |

TABLE 20
SPR binding affinity of cMET antibodies
063Ab10910 and the variants thereof.

| Antibody variants | ka (1/Ms) | kd (1/s) | KD (M) |
|---|---|---|---|
| 063Ab10910 | 7.10E+04 | 6.07E−04 | 8.55E−09 |
| 063Ab10910-P28 | 7.00E+04 | 3.96E−04 | 5.66E−09 |
| 063Ab10910-P27 | 7.41E+04 | 4.96E−04 | 6.69E−09 |
| 063Ab10910-P37 | 7.05E+04 | 5.52E−04 | 7.83E−09 |
| 063Ab10910-P26 | 6.90E+04 | 5.48E−04 | 7.94E−09 |
| 063Ab10910-P19 | 5.14E+04 | 1.06E−03 | 2.06E−08 |

### Example 13. Construction of Anti-EGFR×cMET×cMET Trispecific Antibody

Anti-EGFR×cMET×cMET trispecific antibodies, wherein two cMET aims binds to different anti-EMET epitopes, were constructed with VH and VL sequences from anti-cMET antibodies 063Ab10910-P19 and 063Aba32t)-V1, and anti-EGFR antibody (SEQ ID No: 142 for VH and SEQ ID No: 143 for VL). The Fc region employed “knob into hole” technology to facilitate Fc heterodimerization to form bispecific cMET antibodies comprised of 063Ab10910-P19 and 063Ab03210-V1 arms. Anti-EGFR antibody was re-formatted to scFv and then fused to Fc C terminal of biparatopic anti-cMET antibody with GS linker sequence (GGGGS)₄ (SEQ ID NO: 139). The framework of two anti-cMET arms (063Ab10910-P19 and 063Ab3210-V1) were further engineered with multiple positive charge or negative charge amino acid to bring in pI difference between Fc heterodimer and homodimer thus to facilitate purification of target trispecific molecule in ion exchange chromatography, resulting in BGA-109 (VH: SEQ ID NO: 144) and BGA-032 (VH: SEQ ID NO: 145). The sequences of constructed trispecific antibody

TABLE 21
Sequences for construction of anti-EGFR x cMET x cMET trispecific antibody
TE-642

| | SEQ ID NO | Description | SEQUENCE |
|---|---|---|---|
| EGFR arm | SEQ ID NO: 142 | VH sequence | QVQLVESGGGVVQPGRSLRLSCAASGFTFSTYG |
| | | | MHWVRQAPGKGLEWVAVIWDDGSYKYYGDS |
| | | | VKGRFTISRDNSKNTLYLQMNSLRAEDTAVYY |
| | | | CARDGITMVRGVMKDYFDYWGQGTLVTVSS |
| | SEQ ID NO: 155 | HCDR1 (Kabat) | TYGMH |
| | SEQ ID NO: 156 | HCDR2 (Kabat) | VIWDDGSYKYYGDSVKG |
| | SEQ ID NO: 157 | HCDR3 (Kabat) | DGITMVRGVMKDYFDY |
| | SEQ ID NO: 143 | VL sequence | AIQLTQSPSSLSASVGDRVTITCRASQDISSALV |
| | | | WYQQKPGKAPKLLIYDASSLESGVPSRFSGSES |
| | | | GTDFTLTISSLQPEDFATYYCQQFNSYPLTFGGG |
| | | | TKVEIK |
| | SEQ ID NO: 158 | LCDR1 (Kabat) | RASQDISSALV |
| | SEQ ID NO: 159 | LCDR2 (Kabat) | DASSLES |
| | SEQ ID NO: 160 | LCDR3 (Kabat) | QQFNSYPLT |
| EGFR arm | SEQ ID NO: 161 | ScFv (VL-VH) | AIQLTQSPSSLSASVGDRVTITCRASQDISSALV |
| | | | WYQQKPGKAPKLLIYDASSLESGVPSRFSGSES |
| | | | GTDFTLTISSLQPEDFATYYCQQFNSYPLTFGGG |
| | | | TKVEIKGGGGSGGGGSGGGGSGGGGSQVQLVE |
| | | | SGGGVVQPGRSLRLSCAASGFTFSTYGMHWVR |
| | | | QAPGKGLEWVAVIWDDGSYKYYGDSVKGRFTI |
| | | | SRDNSKNTLYLQMNSLRAEDTAVYYCARDGIT |
| | | | MVRGVMKDYFDYWGQGTLVTVSS |
| cMET arm 1 | SEQ ID NO: 144 | VH (AA) | EVQLVESGGEVVQPGESLRLSCAASGFTFSSHG |
| BGA-109 | | (063Ab10910-P19 + | MHWVRQAPGKGLEWVAVISYDESDKYYADSV |
| | | charge | KGRFTISRDHSKNTLYLEMNSLRAEDTAVYYCV |
| | | engineering in FR) | KDRNEGYSGWLDYWGQGTLVTVSS |
| | SEQ ID NO: 162 | VH (DNA) | GAAGTGCAGCTGGTGGAGTCCGGGGGCGAGG |
| | | | TGGTCCAACCCGGGGAGAGCCTGAGACTGAG |
| | | | CTGCGCCGCTAGCGGCTTCACCTTCAGCAGCC |
| | | | ACGGCATGCACTGGGTGAGACAAGCCCCCGG |
| | | | CAAGGGCCTGGAGTGGGTGGCCGTGATCAGC |
| | | | TACGACGAGAGCGACAAGTACTACGCCGACA |
| | | | GCGTGAAGGGCAGATTCACCATCAGCAGAGA |
| | | | CCACAGCAAGAACACCCTGTACCTGGAGATG |
| | | | AACAGCCTGAGAGCCGAGGACACCGCCGTGT |
| | | | ACTACTGCGTGAAGGACAGAAACGAGGGCTA |
| | | | CAGCGGCTGGCTGGACTACTGGGGCCAAGGC |
| | | | ACCCTGGTGACCGTGAGCAGC |
| | SEQ ID NO: 6 | HCDR1 (Kabat) | SHGMH |
| | SEQ ID NO: 7 | HCDR2 (Kabat) | VISYDESDKYYADSVKG |
| | SEQ ID NO: 92 | HCDR3 (Kabat) | DRNEGYSGWLDY |
| | SEQ ID NO: 64 | VL | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQRSNWPPTFGQ |
| | | | GTKVEIK |
| | SEQ ID NO: 66 | LCDR1 (Kabat) | RASQSVSSYLA |
| | SEQ ID NO: 67 | LCDR2 (Kabat) | DASNRAT |
| | SEQ ID NO: 68 | LCDR3 (Kabat) | QQRSNWPPT |
| cMET arm 2 | SEQ ID NO: 145 | VH (AA) | EFQLQESGPGLVKPSETLRLTCTVSGGSISTSSY |
| BGA-032 | | (063Ab03210-V1 + | YWGWIRQPPGKGLEWIGTIYYSGTTYYNPSLKS |
| | | charge | RVTISVDTSKNQFSLKLKSVRAADTSIYYCARH |
| | | engineering) | WPKYNSWFDPWGQGILVTVSS |
| | SEQ ID NO: 163 | VH (DNA) | GAGTTTCAGCTGCAAGAGAGCGGCCCCGGCC |
| | | | TGGTGAAGCCTAGCGAGACCCTGAGACTGAC |
| | | | CTGCACCGTGAGCGGCGGCAGCATCAGCACA |
| | | | AGCAGCTACTACTGGGGCTGGATCAGACAGC |
| | | | CCCCCGGCAAGGGCCTGGAGTGGATCGGCAC |
| | | | CATCTACTACAGCGGCACCACCTACTACAACC |
| | | | CTAGCCTGAAGAGCAGAGTGACCATCAGCGT |
| | | | GGACACAAGCAAGAATCAGTTCAGCCTGAAG |
| | | | CTGAAGAGCGTGAGAGCCGCCGACACAAGCA |
| | | | TCTACTACTGCGCTAGACACTGGCCCAAGTAC |
| | | | AACAGCTGGTTCGACCCCTGGGGCCAAGGCA |
| | | | TCCTGGTGACCGTGAGCAGC |
| | SEQ ID NO: 56 | HCDR1 (Kabat) | TSSYYWG |
| | SEQ ID NO: 57 | HCDR2 (Kabat) | TIYYSGTTYYNPSLKS |
| | SEQ ID NO: 58 | HCDR3 (Kabat) | HWPKYNSWFDP |
| | SEQ ID NO: 64 | VL | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQRSNWPPTFGQ |
| | | | GTKVEIK |
| | SEQ ID NO: 66 | LCDR1 (Kabat) | RASQSVSSYLA |
| | SEQ ID NO: 67 | LCDR2 (Kabat) | DASNRAT |
| | SEQ ID NO: 68 | LCDR3 (Kabat) | QQRSNWPPT |
| TE-642 | SEQ ID NO: 146 | Polypeptide 1 | EFQLQESGPGLVKPSETLRLTCTVSGGSISTSSY |
| | | (Knob chain) with | YWGWIRQPPGKGLEWIGTIYYSGTTYYNPSLKS |
| | | BGA-032 and | RVTISVDTSKNQFSLKLKSVRAADTSIYYCARH |
| | | EGFR arm | WPKYNSWFDPWGQGILVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGTQ |
| | | | TYICNVNHKPSNTKVDKKVEPKSCDKTHTCPPC |
| | | | PAPELLGGPSVFLFPPKPKDTLMISRTPEVTCVV |
| | | | VDVSHEDPEVKFNWYVDGVEVHNAKTKPREE |
| | | | QYNSTYRVVSVLTVLHQDWLNGKEYKCKVSN |
| | | | KALPAPIEKTISKAKGQPREPQVYTLP |
| | | | PSREEMTKNQVSLWCLVKGFYPSDIAVEWESN |
| | | | GQPENNYKTTPPVLDSDGSFFLYSKLTVDKSRW |
| | | | QQGNVFSCSVMHEALHNHYTQKSLSLSPGKGG |
| | | | GGSGGGGSGGGGSGGGGSAIQLTQSPSSLSASV |
| | | | GDRVTITCRASQDISSALVWYQQKPGKAPKLLI |
| | | | YDASSLESGVPSRFSGSESGTDFTLTISSLQPEDF |
| | | | ATYYCQQFNSYPLTFGGGTKVEIKGGGGSGGG |
| | | | GSGGGGSGGGGSQVQLVESGGGVVQPGRSLRL |
| | | | SCAASGFTFSTYGMHWVRQAPGKGLEWVAVI |
| | | | WDDGSYKYYGDSVKGRFTISRDNSKNTLYLQM |
| | | | NSLRAEDTAVYYCARDGITMVRGVMKDYFDY |
| | | | WGQGTLVTVSS |
| | SEQ ID NO: 147 | Polypeptide 2 | EVQLVESGGEVVQPGESLRLSCAASGFTFSSHG |
| | | (Hole chain) with | MHWVRQAPGKGLEWVAVISYDESDKYYADSV |
| | | BGA-109 and | KGRFTISRDHSKNTLYLEMNSLRAEDTAVYYCV |
| | | EGFR arm | KDRNEGYSGWLDYWGQGTLVTVSSASTKGPSV |
| | | | FPLAPSSKSTSGGTAALGCLVKDYFPEPVTVSW |
| | | | NSGALTSGVHTFPAVLQSSGLYSLSSVVTVPSSS |
| | | | LGTQTYICNVNHKPSNTKVDKKVEPKSCDKTH |
| | | | TCPPCPAPELLGGPSVFLFPPKPKDTLMISRTPEV |
| | | | TCVVVDVSHEDPEVKFNWYVDGVEVHNAKTK |
| | | | PREEQYNSTYRVVSVLTVLHQDWLNGKEYKCK |
| | | | VSNKALPAPIEKTISKAKGQPREPQVYTLPPSRE |
| | | | EMTKNQVSLSCAVKGFYPSDIAVEWESNGQPE |
| | | | NNYKTTPPVLDSDGSFFLVSKLTVDKSRWQQG |
| | | | NVFSCSVMHEALHNHYTQKSLSLSPGKGGGGS |
| | | | GGGGSGGGGSGGGGSAIQLTQSPSSLSASVGDR |
| | | | VTITCRASQDISSALVWYQQKPGKAPKLLIYDA |
| | | | SSLESGVPSRFSGSESGTDFTLTISSLQPEDFATY |
| | | | YCQQFNSYPLTFGGGTKVEIKGGGGSGGGGSG |
| | | | GGGSGGGGSQVQLVESGGGVVQPGRSLRLSCA |
| | | | ASGFTFSTYGMHWVRQAPGKGLEWVAVIWDD |
| | | | GSYKYYGDSVKGRFTISRDNSKNTLYLQMNSL |
| | | | RAEDTAVYYCARDGITMVRGVMKDYFDYWGQ |
| | | | GTLVTVSS |
| | SEQ ID NO: 148 | Polypeptide 3 | EIVLTQSPATLSLSPGERATLSCRASQSVSSYLA |
| | | (Common LC) | WYQQKPGQAPRLLIYDASNRATGIPARFSGSGS |
| | | | GTDFTLTISSLEPEDFAVYYCQQRSNWPPTFGQ |
| | | | GTKVEIKRTVAAPSVFIFPPSDEQLKSGTASVVC |
| | | | LLNNFYPREAKVQWKVDNALQSGNSQESVTEQ |
| | | | DSKDSTYSLSSTLTLSKADYEKHKVYACEVTHQ |
| | | | GLSSPVTKSFNRGEC |

The antibody was constructed with in-house IgG1/Cx eukaryotic expression vector, produced with ExpiCHO expression system (Thermofisher Scientific) with 30 mg/L (0.1 mM) fucosyltransferase inhibitor 2F-Peracetyl-Fucose (Sigma-Aldrich) for inhibition of fucosylation for effector function enhancement, and purified with MabSelect SuRe protein A chromatography resin (Cytiva) followed by HP-SP cation exchange chromatography (Cytiva). Obtained antibody with desired purity was then used for further characterization.

### Example 14. Construction of Anti-EGFR×cMET×cMET Trispecific Antibodies with Multiple Formats

Besides TE-642 with two anti-EGFR scFv fused to Fc C terminal, multiple trispecific antibody formats were constructed with different EGFR arm valency and different orientations of EGFR arm for further optimization of functionality of these trispecific antibodies. Specifically, single valent or bivalent EGFR scFv was fused to Fc C terminal, light chain C terminal, light chain N terminal, and heavy chain N terminal. These different combination of valency and orientations gave rise to trispecific antibodies TE-644, TE-645, TE-646, TE-647, and TE-648. The format of these five antibodies, as well as TE-642, were illustrated as FIG. 7. The sequences of these antibodies were summarized in Table 22.

TABLE 22
Sequences for construction of anti-EGFR x cMET x cMET trispecific antibodies
with different formats

| | SEQ ID | | |
|---|---|---|---|
| Antibody | NO | Description | SEQUENCE |
| TE-644 | SEQ ID | Polypeptide 1 | EFQLQESGPGLVKPSETLRLTCTVSGGSISTSSYYWGWIRQP |
| | NO: 149 | (Knob chain) | PGKGLEWIGTIYYSGTTYYNPSLKSRVTISVDTSKNQFSLKL |
| | | with BGA-032 | KSVRAADTSIYYCARHWPKYNSWFDPWGQGILVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKVEPKSCDKTHTCPPCPAPELLGGPSVFLFP |
| | | | PKPKDTLMISRTPEVTCVVVDVSHEDPEVKFNWYVDGVEV |
| | | | HNAKTKPREEQYNSTYRVVSVLTVLHQDWLNGKEYKCKV |
| | | | SNKALPAPIEKTISKAKGQPREPQVYTLPPSREEMTKNQVS |
| | | | LWCLVKGFYPSDIAVEWESNGQPENNYKTTPPVLDSDGSF |
| | | | FLYSKLTVDKSRWQQGNVFSCSVMHEALHNHYTQKSLSLS |
| | | | PGK |
| | SEQ ID | Polypeptide 2 | EVQLVESGGEVVQPGESLRLSCAASGFTFSSHGMHWVRQA |
| | NO: 150 | (Hole chain) | PGKGLEWVAVISYDESDKYYADSVKGRFTISRDHSKNTLY |
| | | with BGA-109 | LEMNSLRAEDTAVYYCVKDRNEGYSGWLDYWGQGTLVT |
| | | | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKVEPKSCDKTHTCPPCPAPELLGG |
| | | | PSVFLFPPKPKDTLMISRTPEVTCVVVDVSHEDPEVKFNWY |
| | | | VDGVEVHNAKTKPREEQYNSTYRVVSVLTVLHQDWLNGK |
| | | | EYKCKVSNKALPAPIEKTISKAKGQPREPQVYTLPPSREEM |
| | | | TKNQVSLSCAVKGFYPSDIAVEWESNGQPENNYKTTPPVL |
| | | | DSDGSFFLVSKLTVDKSRWQQGNVFSCSVMHEALHNHYT |
| | | | QKSLSLSPGK |
| | SEQ ID | Polypeptide 3 | EIVLTQSPATLSLSPGERATLSCRASQSVSSYLAWYQQKPG |
| | NO: 151 | with common | QAPRLLIYDASNRATGIPARFSGSGSGTDFTLTISSLEPEDFA |
| | | LC and EGFR | VYYCQQRSNWPPTFGQGTKVEIKRTVAAPSVFIFPPSDEQL |
| | | arm | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGECGGGGSGGGGSGGGGSGGGGSAIQLTQSPSS |
| | | | LSASVGDRVTITCRASQDISSALVWYQQKPGKAPKLLIYDA |
| | | | SSLESGVPSRFSGSESGTDFTLTISSLQPEDFATYYCQQFNSY |
| | | | PLTFGGGTKVEIKGGGGSGGGGSGGGGSGGGGSQVQLVES |
| | | | GGGVVQPGRSLRLSCAASGFTFSTYGMHWVRQAPGKGLE |
| | | | WVAVIWDDGSYKYYGDSVKGRFTISRDNSKNTLYLQMNS |
| | | | LRAEDTAVYYCARDGITMVRGVMKDYFDYWGQGTLVTV |
| | | | SS |
| TE-645 | SEQ ID | Polypeptide 1 | EFQLQESGPGLVKPSETLRLTCTVSGGSISTSSYYWGWIRQP |
| | NO: 149 | (Knob chain) | PGKGLEWIGTIYYSGTTYYNPSLKSRVTISVDTSKNQFSLKL |
| | | with BGA-032 | KSVRAADTSIYYCARHWPKYNSWFDPWGQGILVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKVEPKSCDKTHTCPPCPAPELLGGPSVFLFP |
| | | | PKPKDTLMISRTPEVTCVVVDVSHEDPEVKFNWYVDGVEV |
| | | | HNAKTKPREEQYNSTYRVVSVLTVLHQDWLNGKEYKCKV |
| | | | SNKALPAPIEKTISKAKGQPREPQVYTLPPSREEMTKNQVS |
| | | | LWCLVKGFYPSDIAVEWESNGQPENNYKTTPPVLDSDGSF |
| | | | FLYSKLTVDKSRWQQGNVFSCSVMHEALHNHYTQKSLSLS |
| | | | PGK |
| | SEQ ID | Polypeptide 2 | EVQLVESGGEVVQPGESLRLSCAASGFTFSSHGMHWVRQA |
| | NO: 150 | (Hole chain) | PGKGLEWVAVISYDESDKYYADSVKGRFTISRDHSKNTLY |
| | | with BGA-109 | LEMNSLRAEDTAVYYCVKDRNEGYSGWLDYWGQGTLVT |
| | | | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKVEPKSCDKTHTCPPCPAPELLGG |
| | | | PSVFLFPPKPKDTLMISRTPEVTCVVVDVSHEDPEVKENWY |
| | | | VDGVEVHNAKTKPREEQYNSTYRVVSVLTVLHQDWLNGK |
| | | | EYKCKVSNKALPAPIEKTISKAKGQPREPQVYTLPPSREEM |
| | | | TKNQVSLSCAVKGFYPSDIAVEWESNGQPENNYKTTPPVL |
| | | | DSDGSFFLVSKLTVDKSRWQQGNVFSCSVMHEALHNHYT |
| | | | QKSLSLSPGK |
| | SEQ ID | Polypeptide 3 | AIQLTQSPSSLSASVGDRVTITCRASQDISSALVWYQQKPG |
| | NO: 152 | EGFR arm and | KAPKLLIYDASSLESGVPSRFSGSESGTDFTLTISSLQPEDFA |
| | | common LC | TYYCQQFNSYPLTFGGGTKVEIKGGGGSGGGGSGGGGSGG |
| | | | GGSQVQLVESGGGVVQPGRSLRLSCAASGFTFSTYGMHW |
| | | | VRQAPGKGLEWVAVIWDDGSYKYYGDSVKGRFTISRDNS |
| | | | KNTLYLQMNSLRAEDTAVYYCARDGITMVRGVMKDYFD |
| | | | YWGQGTLVTVSSGGGGSGGGGSGGGGSGGGGSEIVLTQSP |
| | | | ATLSLSPGERATLSCRASQSVSSYLAWYQQKPGQAPRLLIY |
| | | | DASNRATGIPARFSGSGSGTDFTLTISSLEPEDFAVYYCQQR |
| | | | SNWPPTFGQGTKVEIKRTVAAPSVFIFPPSDEQLKSGTASVV |
| | | | CLLNNFYPREAKVQWKVDNALQSGNSQESVTEQDSKDST |
| | | | YSLSSTLTLSKADYEKHKVYACEVTHQGLSSPVTKSFNRGE |
| | | | C |
| TE-646 | SEQ ID | Polypeptide 1 | AIQLTQSPSSLSASVGDRVTITCRASQDISSALVWYQQKPG |
| | NO: 153 | (Knob chain) | KAPKLLIYDASSLESGVPSRFSGSESGTDFTLTISSLQPEDFA |
| | | with EGFR | TYYCQQFNSYPLTFGGGTKVEIKGGGGSGGGGSGGGGSGG |
| | | arm and BGA- | GGSQVQLVESGGGVVQPGRSLRLSCAASGFTFSTYGMHW |
| | | 032 | VRQAPGKGLEWVAVIWDDGSYKYYGDSVKGRFTISRDNS |
| | | | KNTLYLQMNSLRAEDTAVYYCARDGITMVRGVMKDYFD |
| | | | YWGQGTLVTVSSGGGGSGGGGSGGGGSGGGGSEFQLQES |
| | | | GPGLVKPSETLRLTCTVSGGSISTSSYYWGWIRQPPGKGLE |
| | | | WIGTIYYSGTTYYNPSLKSRVTISVDTSKNQFSLKLKSVRA |
| | | | ADTSIYYCARHWPKYNSWFDPWGQGILVTVSSASTKGPSV |
| | | | FPLAPSSKSTSGGTAALGCLVKDYFPEPVTVSWNSGALTSG |
| | | | VHTFPAVLQSSGLYSLSSVVTVPSSSLGTQTYICNVNHKPS |
| | | | NTKVDKKVEPKSCDKTHTCPPCPAPELLGGPSVFLFPPKPK |
| | | | DTLMISRTPEVTCVVVDVSHEDPEVKFNWYVDGVEVHNA |
| | | | KTKPREEQYNSTYRVVSVLTVLHQDWLNGKEYKCKVSNK |
| | | | ALPAPIEKTISKAKGQPREPQVYTLPPSREEMTKNQVSLWC |
| | | | LVKGFYPSDIAVEWESNGQPENNYKTTPPVLDSDGSFFLYS |
| | | | KLTVDKSRWQQGNVFSCSVMHEALHNHYTQKSLSLSPGK |
| | SEQ ID | Polypeptide 2 | AIQLTQSPSSLSASVGDRVTITCRASQDISSALVWYQQKPG |
| | NO: 154 | (Hole chain) | KAPKLLIYDASSLESGVPSRFSGSESGTDFTLTISSLQPEDFA |
| | | with EGFR | TYYCQQFNSYPLTFGGGTKVEIKGGGGSGGGGSGGGGSGG |
| | | arm and BGA- | GGSQVQLVESGGGVVQPGRSLRLSCAASGFTFSTYGMHW |
| | | 109 | VRQAPGKGLEWVAVIWDDGSYKYYGDSVKGRFTISRDNS |
| | | | KNTLYLQMNSLRAEDTAVYYCARDGITMVRGVMKDYFD |
| | | | YWGQGTLVTVSSGGGGSGGGGSGGGGSGGGGSEVQLVES |
| | | | GGEVVQPGESLRLSCAASGFTFSSHGMHWVRQAPGKGLE |
| | | | WVAVISYDESDKYYADSVKGRFTISRDHSKNTLYLEMNSL |
| | | | RAEDTAVYYCVKDRNEGYSGWLDYWGQGTLVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKVEPKSCDKTHTCPPCPAPELLGGPSVFLFP |
| | | | PKPKDTLMISRTPEVTCVVVDVSHEDPEVKFNWYVDGVEV |
| | | | HNAKTKPREEQYNSTYRVVSVLTVLHQDWLNGKEYKCKV |
| | | | SNKALPAPIEKTISKAKGQPREPQVYTLPPSREEMTKNQVS |
| | | | LSCAVKGFYPSDIAVEWESNGQPENNYKTTPPVLDSDGSFF |
| | | | LVSKLTVDKSRWQQGNVFSCSVMHEALHNHYTQKSLSLSP |
| | | | GK |
| | SEQ ID | Polypeptide 3 | EIVLTQSPATLSLSPGERATLSCRASQSVSSYLAWYQQKPG |
| | NO: 148 | (Common LC) | QAPRLLIYDASNRATGIPARFSGSGSGTDFTLTISSLEPEDFA |
| | | | VYYCQQRSNWPPTFGQGTKVEIKRTVAAPSVFIFPPSDEQL |
| | | | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGEC |
| TE-647 | SEQ ID | Polypeptide 1 | AIQLTQSPSSLSASVGDRVTITCRASQDISSALVWYQQKPG |
| | NO: 153 | (Knob chain) | KAPKLLIYDASSLESGVPSRFSGSESGTDFTLTISSLQPEDFA |
| | | with EGFR | TYYCQQFNSYPLTFGGGTKVEIKGGGGSGGGGSGGGGSGG |
| | | arm and BGA- | GGSQVQLVESGGGVVQPGRSLRLSCAASGFTFSTYGMHW |
| | | 032 | VRQAPGKGLEWVAVIWDDGSYKYYGDSVKGRFTISRDNS |
| | | | KNTLYLQMNSLRAEDTAVYYCARDGITMVRGVMKDYFD |
| | | AA | YWGQGTLVTVSSGGGGSGGGGSGGGGSGGGGSEFQLQES |
| | | | GPGLVKPSETLRLTCTVSGGSISTSSYYWGWIRQPPGKGLE |
| | | | WIGTIYYSGTTYYNPSLKSRVTISVDTSKNQFSLKLKSVRA |
| | | | ADTSIYYCARHWPKYNSWFDPWGQGILVTVSSASTKGPSV |
| | | | FPLAPSSKSTSGGTAALGCLVKDYFPEPVTVSWNSGALTSG |
| | | | VHTFPAVLQSSGLYSLSSVVTVPSSSLGTQTYICNVNHKPS |
| | | | NTKVDKKVEPKSCDKTHTCPPCPAPELLGGPSVFLFPPKPK |
| | | | DTLMISRTPEVTCVVVDVSHEDPEVKFNWYVDGVEVHNA |
| | | | KTKPREEQYNSTYRVVSVLTVLHQDWLNGKEYKCKVSNK |
| | | | ALPAPIEKTISKAKGQPREPQVYTLPPSREEMTKNQVSLWC |
| | | | LVKGFYPSDIAVEWESNGQPENNYKTTPPVLDSDGSFFLYS |
| | | | KLTVDKSRWQQGNVFSCSVMHEALHNHYTQKSLSLSPGK |
| | SEQ ID | Polypeptide 1 | GCCATTCAGCTGACACAGAGCCCTAGCAGCCTGAGCGCT |
| | NO: 164 | (Knob chain) | AGCGTGGGCGACAGAGTGACAATCACCTGCAGAGCTAG |
| | | with EGFR | CCAAGACATCAGCAGCGCCCTGGTGTGGTATCAGCAGA |
| | | arm and BGA- | AGCCCGGCAAGGCCCCCAAGCTGCTGATCTACGACGCTA |
| | | 032 | GCAGCCTCGAAAGCGGCGTGCCTAGCAGATTTAGCGGC |
| | | DNA | AGCGAGAGCGGCACCGACTTCACCCTGACCATCAGCAG |
| | | | CCTGCAGCCCGAGGACTTCGCCACCTACTACTGTCAGCA |
| | | | GTTCAACAGCTACCCCCTGACCTTCGGCGGGGGCACAAA |
| | | | AGTGGAAATTAAGGGTGGAGGCGGTTCAGGCGGAGGTG |
| | | | GCTCTGGCGGTGGCGGATCGGGTGGCGGAGGCTCACAA |
| | | | GTGCAACTGGTCGAAAGCGGGGGGGGGTGGTGCAGCC |
| | | | TGGGAGAAGCCTGAGACTGAGCTGCGCCGCTAGCGGCT |
| | | | TCACCTTCAGCACCTACGGCATGCACTGGGTGAGACAAG |
| | | | CCCCCGGCAAAGGCCTGGAATGGGTGGCCGTGATCTGG |
| | | | GACGACGGCAGCTACAAGTACTACGGCGACAGCGTGAA |
| | | | GGGCAGATTCACCATCAGCAGAGACAACAGCAAGAACA |
| | | | CCCTGTACCTGCAGATGAACAGCCTGAGAGCCGAGGAC |
| | | | ACCGCCGTGTACTACTGCGCTAGAGACGGCATCACCATG |
| | | | GTGAGAGGCGTGATGAAGGACTACTTTGACTATTGGGG |
| | | | GCAAGGCACCCTGGTGACCGTGAGCAGCGGTGGAGGCG |
| | | | GTTCAGGCGGAGGTGGCTCTGGCGGTGGCGGATCGGGT |
| | | | GGCGGAGGCTCAGAGTTTCAGCTGCAAGAGTCCGGCCC |
| | | | CGGCCTGGTGAAGCCTAGCGAGACCCTGAGACTGACCT |
| | | | GCACCGTGAGCGGCGGCAGCATCAGCACAAGCAGCTAC |
| | | | TACTGGGGCTGGATCAGACAGCCTCCCGGCAAGGGCCT |
| | | | GGAGTGGATCGGCACCATCTACTACAGCGGCACCACCTA |
| | | | CTACAACCCTAGCCTGAAGAGCAGAGTGACCATCAGCG |
| | | | TGGACACAAGCAAGAATCAGTTCAGCCTGAAGCTGAAG |
| | | | AGCGTGAGAGCCGCCGACACAAGCATCTATTACTGCGCC |
| | | | CGGCACTGGCCCAAGTACAACAGCTGGTTCGACCCCTGG |
| | | | GGCCAAGGCATCCTGGTGACCGTCAGCAGCGCCTCCACA |
| | | | AAGGGGCCTAGCGTGTTCCCTCTGGCCCCTAGCAGCAAA |
| | | | AGCACAAGCGGCGGCACCGCTGCCCTGGGCTGCCTGGTC |
| | | | AAGGATTACTTCCCCGAGCCCGTGACCGTGAGCTGGAAT |
| | | | AGCGGCGCTCTCACAAGCGGCGTGCACACCTTCCCCGCC |
| | | | GTGCTGCAGAGCAGCGGCCTGTACAGCCTGAGCAGCGT |
| | | | GGTGACCGTGCCTAGCAGCAGCCTGGGCACACAGACCT |
| | | | ACATCTGCAACGTGAACCACAAGCCTAGCAACACCAAG |
| | | | GTGGACAAAAAGGTGGAGCCCAAGTCCTGTGACAAGAC |
| | | | CCACACCTGCCCCCCCTGTCCCGCCCCCGAACTCCTGGG |
| | | | CGGCCCTAGCGTGTTCCTGTTCCCCCCCAAGCCCAAGGA |
| | | | CACCCTGATGATCAGCAGAACCCCCGAGGTGACCTGCGT |
| | | | GGTCGTGGACGTGAGCCACGAGGACCCCGAGGTGAAGT |
| | | | TCAACTGGTACGTGGACGGCGTGGAGGTGCACAACGCC |
| | | | AAGACCAAGCCTAGAGAGGAGCAGTACAACAGCACCTA |
| | | | CAGAGTGGTGAGCGTGCTGACCGTGCTGCACCAAGACT |
| | | | GGCTGAACGGCAAGGAGTACAAGTGCAAGGTGAGCAAC |
| | | | AAGGCCCTGCCCGCCCCCATCGAGAAGACCATCAGCAA |
| | | | GGCCAAGGGGCAGCCTAGAGAGCCCCAAGTGTACACCC |
| | | | TGCCCCCTAGCAGAGAGGAGATGACCAAGAACCAAGTG |
| | | | AGCCTGTGGTGCCTGGTGAAGGGGTTTTACCCTAGCGAC |
| | | | ATCGCCGTGGAGTGGGAGAGCAACGGGCAGCCCGAGAA |
| | | | CAACTACAAGACCACCCCCCCCGTGCTGGACAGCGACG |
| | | | GCAGCTTCTTCCTGTACAGCAAGCTGACCGTGGACAAGA |
| | | | GCAGATGGCAGCAAGGCAACGTGTTCAGCTGCAGCGTG |
| | | | ATGCACGAGGCCCTGCACAACCACTACACACAGAAGAG |
| | | | CCTGAGCCTGAGCCCCGGCAAG |
| | SEQ ID | Polypeptide 2 | EVQLVESGGEVVQPGESLRLSCAASGFTFSSHGMHWVRQA |
| | NO: 150 | (Hole chain) | PGKGLEWVAVISYDESDKYYADSVKGRFTISRDHSKNTLY |
| | | with BGA-109 | LEMNSLRAEDTAVYYCVKDRNEGYSGWLDYWGQGTLVT |
| | | AA | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKVEPKSCDKTHTCPPCPAPELLGG |
| | | | PSVFLFPPKPKDTLMISRTPEVTCVVVDVSHEDPEVKFNWY |
| | | | VDGVEVHNAKTKPREEQYNSTYRVVSVLTVLHQDWLNGK |
| | | | EYKCKVSNKALPAPIEKTISKAKGQPREPQVYTLPPSREEM |
| | | | TKNQVSLSCAVKGFYPSDIAVEWESNGQPENNYKTTPPVL |
| | | | DSDGSFFLVSKLTVDKSRWQQGNVFSCSVMHEALHNHYT |
| | | | QKSLSLSPGK |
| | SEQ ID | Polypeptide 2 | GAGGTGCAGCTGGTCGAGAGCGGGGGCGAAGTGGTCCA |
| | NO: 165 | (Hole chain) | ACCCGGCGAGAGCCTGAGACTGTCCTGCGCCGCTAGCG |
| | | with BGA-109 | GCTTCACCTTCAGCAGCCACGGCATGCACTGGGTGAGAC |
| | | DNA | AAGCCCCCGGCAAGGGCCTGGAGTGGGTGGCCGTGATC |
| | | | AGCTACGACGAGAGCGACAAGTACTACGCCGACAGCGT |
| | | | GAAGGGCAGATTCACCATCAGCAGAGACCACAGCAAGA |
| | | | ACACCCTGTACCTGGAGATGAACAGCCTGAGAGCCGAG |
| | | | GACACCGCCGTGTACTACTGCGTGAAGGACAGAAACGA |
| | | | GGGCTACAGCGGCTGGCTGGACTACTGGGGCCAAGGCA |
| | | | CCCTGGTGACCGTGTCCTCCGCTAGCACCAAGGGCCCTA |
| | | | GCGTGTTTCCCCTGGCCCCTAGCAGCAAGTCCACCTCCG |
| | | | GCGGGACAGCCGCTCTGGGCTGCCTGGTGAAGGACTACT |
| | | | TCCCCGAGCCCGTGACAGTGAGCTGGAACTCCGGCGCTC |
| | | | TGACAAGCGGCGTGCATACCTTCCCCGCCGTGCTGCAGA |
| | | | GCAGCGGCCTGTACAGCCTGAGCAGCGTGGTGACCGTG |
| | | | CCTAGCAGCAGCCTGGGCACACAGACCTACATCTGCAAC |
| | | | GTGAACCACAAGCCTAGCAACACCAAGGTGGACAAGAA |
| | | | GGTGGAGCCCAAATCCTGCGATAAAACCCACACATGCC |
| | | | CCCCCTGCCCCGCCCCCGAGCTGCTGGGCGGCCCCTCCG |
| | | | TCTTCCTGTTCCCCCCCAAGCCCAAGGACACCCTGATGA |
| | | | TCAGCAGAACCCCCGAGGTGACCTGCGTGGTCGTGGAC |
| | | | GTGAGCCACGAGGACCCCGAGGTGAAGTTCAACTGGTA |
| | | | CGTGGACGGCGTGGAGGTGCACAACGCCAAGACCAAGC |
| | | | CTAGAGAGGAGCAGTACAACAGCACCTACAGAGTGGTG |
| | | | AGCGTGCTGACCGTGCTGCACCAAGACTGGCTGAACGG |
| | | | CAAGGAGTACAAGTGCAAGGTGAGCAACAAGGCCCTGC |
| | | | CCGCCCCCATCGAGAAGACCATCAGCAAGGCCAAGGGG |
| | | | CAGCCTAGAGAGCCCCAAGTGTACACCCTGCCCCCTAGC |
| | | | AGAGAGGAGATGACCAAGAACCAAGTGAGCCTGAGCTG |
| | | | CGCCGTGAAGGGCTTCTACCCTAGCGACATCGCCGTGGA |
| | | | GTGGGAGAGCAACGGGCAGCCCGAGAACAACTACAAGA |
| | | | CCACCCCCCCCGTGCTGGACAGCGACGGCAGCTTCTTCC |
| | | | TGGTGAGCAAGCTGACCGTGGACAAGAGCAGATGGCAG |
| | | | CAAGGCAACGTGTTCAGCTGCAGCGTGATGCACGAGGC |
| | | | CCTGCACAACCACTACACACAGAAGAGCCTGAGCCTGA |
| | | | GCCCCGGCAAG |
| | SEQ ID | Polypeptide 3 | EIVLTQSPATLSLSPGERATLSCRASQSVSSYLAWYQQKPG |
| | NO: 148 | (Common LC) | QAPRLLIYDASNRATGIPARFSGSGSGTDFTLTISSLEPEDFA |
| | | AA | VYYCQQRSNWPPTFGQGTKVEIKRTVAAPSVFIFPPSDEQL |
| | | | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGEC |
| | SEQ ID | Polypeptide 3 | GAGATTGTGCTGACACAGAGCCCCGCCACCCTGTCCCTG |
| | NO: 166 | (Common LC) | TCCCCCGGGGAGAGAGCCACCCTGAGCTGCAGAGCTAG |
| | | DNA | CCAAAGCGTGAGCAGCTACCTGGCCTGGTATCAGCAGA |
| | | | AGCCCGGCCAAGCCCCTAGACTGCTGATCTACGACGCTA |
| | | | GCAACAGAGCCACCGGCATCCCCGCTAGATTCAGCGGC |
| | | | AGCGGCAGCGGCACCGACTTCACCCTGACCATCAGCAG |
| | | | CCTGGAGCCCGAGGACTTCGCCGTGTACTACTGTCAGCA |
| | | | GAGAAGCAACTGGCCCCCCACCTTCGGCCAAGGCACCA |
| | | | AGGTGGAGATCAAGCGTACGGTGGCTGCACCATCTGTCT |
| | | | TCATCTTCCCGCCATCTGATGAGCAGTTGAAATCTGGAA |
| | | | CTGCCTCTGTTGTGTGCCTGCTGAATAACTTCTATCCCAG |
| | | | AGAGGCCAAAGTACAGTGGAAGGTGGATAACGCCCTCC |
| | | | AATCGGGTAACTCCCAGGAGAGTGTCACAGAGCAGGAC |
| | | | AGCAAGGACAGCACCTACAGCCTCAGCAGCACCCTGAC |
| | | | GCTGAGCAAAGCAGACTACGAGAAACACAAAGTCTACG |
| | | | CCTGCGAAGTCACCCATCAGGGCCTGAGCTCGCCCGTCA |
| | | | CAAAGAGCTTCAACAGGGGAGAGTGT |
| TE-648 | SEQ ID | Polypeptide 1 | EFQLQESGPGLVKPSETLRLTCTVSGGSISTSSYYWGWIRQP |
| | NO: 146 | (Knob chain) | PGKGLEWIGTIYYSGTTYYNPSLKSRVTISVDTSKNQFSLKL |
| | | | KSVRAADTSIYYCARHWPKYNSWFDPWGQGILVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKVEPKSCDKTHTCPPCPAPELLGGPSVFLFP |
| | | | PKPKDTLMISRTPEVTCVVVDVSHEDPEVKFNWYVDGVEV |
| | | | HNAKTKPREEQYNSTYRVVSVLTVLHQDWLNGKEYKCKV |
| | | | SNKALPAPIEKTISKAKGQPREPQVYTLPPSREEMTKNQVS |
| | | | LWCLVKGFYPSDIAVEWESNGQPENNYKTTPPVLDSDGSF |
| | | | FLYSKLTVDKSRWQQGNVFSCSVMHEALHNHYTQKSLSLS |
| | | | PGKGGGGSGGGGSGGGGSGGGGSAIQLTQSPSSLSASVGD |
| | | | RVTITCRASQDISSALVWYQQKPGKAPKLLIYDASSLESGV |
| | | | PSRFSGSESGTDFTLTISSLQPEDFATYYCQQFNSYPLTFGG |
| | | | GTKVEIKGGGGSGGGGSGGGGSGGGGSQVQLVESGGGVV |
| | | | QPGRSLRLSCAASGFTFSTYGMHWVRQAPGKGLEWVAVI |
| | | | WDDGSYKYYGDSVKGRFTISRDNSKNTLYLQMNSLRAED |
| | | | TAVYYCARDGITMVRGVMKDYFDYWGQGTLVTVSS |
| | SEQ ID | Polypeptide 2 | EVQLVESGGEVVQPGESLRLSCAASGFTFSSHGMHWVRQA |
| | NO: 150 | (Hole chain) | PGKGLEWVAVISYDESDKYYADSVKGRFTISRDHSKNTLY |
| | | | LEMNSLRAEDTAVYYCVKDRNEGYSGWLDYWGQGTLVT |
| | | | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKVEPKSCDKTHTCPPCPAPELLGG |
| | | | PSVFLFPPKPKDTLMISRTPEVTCVVVDVSHEDPEVKFNWY |
| | | | VDGVEVHNAKTKPREEQYNSTYRVVSVLTVLHQDWLNGK |
| | | | EYKCKVSNKALPAPIEKTISKAKGQPREPQVYTLPPSREEM |
| | | | TKNQVSLSCAVKGFYPSDIAVEWESNGQPENNYKTTPPVL |
| | | | DSDGSFFLVSKLTVDKSRWQQGNVFSCSVMHEALHNHYT |
| | | | QKSLSLSPGK |
| | SEQ ID | Polypeptide 3 | EIVLTQSPATLSLSPGERATLSCRASQSVSSYLAWYQQKPG |
| | NO: 148 | (Common LC) | QAPRLLIYDASNRATGIPARFSGSGSGTDFTLTISSLEPEDFA |
| | | | VYYCQQRSNWPPTFGQGTKVEIKRTVAAPSVFIFPPSDEQL |
| | | | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGEC |

These five antibodies were constructed with in house IgG1/Cx eukaryotic expression vector, produced with ExpiCHO expression system (Thermofisher Scientific) with 30 mg/i (0.1 mM) fucosyltransferase inhibitor 2F-Peracetyl-Fucose (Sigma-Aldrich) for inhibition of fucosylation for effector function enhancement, and purified with MabSelect SuRe protein A chromatography resin (Cytiva) followed by HP-SP cation exchange chromatography (Cytiva). Obtained antibodies with desired purity was then used for further characterization.

### Example 15. Binding Affinity of TE-647 to Human EGFR and cMET Proteins

Surface plasmon resonance (Biacore 8K, GE Life Sciences) was used to characterize the binding affinity of trispecific antibody TE-647, as well as control antibodies JNJ-372 (Amivantamab, sequences downloaded from https://www.imgt.org/mAb-DB/, internally generated with “controlled Fab-arm exchange” procedure¹⁰ and with 0.1 mM fucosyltransferase inhibitor 2F-Peracetyl-Fucose to inhibit fucosylation for effector function enhancement) and LY3164530 (Sequences extracted from patent WO2015100104A1), to human cMET ECD protein (Cat #10692-H08H, Sino Biological) and human EGFR protein (Cat #10001-H08H, Sino Biological). Briefly, anti-human IgG Fc antibody (Cat #29234600, GE Life Sciences) was immobilized via amine coupling onto an activated CM5 biosensor chip (Cat #29149603, GE Life Sciences). Trispecific antibody TE-647, or control antibodies JNJ-372 and LY3164530 were flowed over the chip surface and captured by anti-human IgG antibody. Then a serial dilution of human cMET and human EGFR proteins were injected over the antibodies captured surface and changes in surface plasmon resonance signals were analyzed to calculate the association rates (kₐ) and dissociation rates (kd) by using the one-to-one Langmuir binding model (Biacore™ Insight Evaluation Software, GE Life Sciences). The equilibrium dissociation constant (KD) was calculated as the ratio ka/k_{d}. The binding affinity is summarized as in Table 23.

TABLE 23
SPR binding affinity of trispecific antibody TE-647 and
control antibodies to human cMET and human EGFR proteins.

| Test Ab | Test Antigen | ka (1/Ms) | kd (1/s) | KD (M) |
|---|---|---|---|---|
| TE-647 | Human cMet | 1.67E+05 | 2.49E−04 | 1.49E−09 |
| JNJ-372 | Human cMet | 1.85E+05 | 1.56E−04 | 8.45E−10 |
| LY3164530 | Human cMet | 7.84E+04 | 2.32E−04 | 2.96E−09 |
| TE-647 | Human EGFR | 1.50E+05 | 1.06E−03 | 7.08E−09 |
| JNJ-372 | Human EGFR | 9.49E+04 | 8.72E−04 | 9.19E−09 |
| LY3164530 | Human EGFR | 7.97E+05 | 8.65E−04 | 1.09E−09 |

### Example 16. ADCC Activity of EGFR×cMET×cMET Trispecific Antibodies

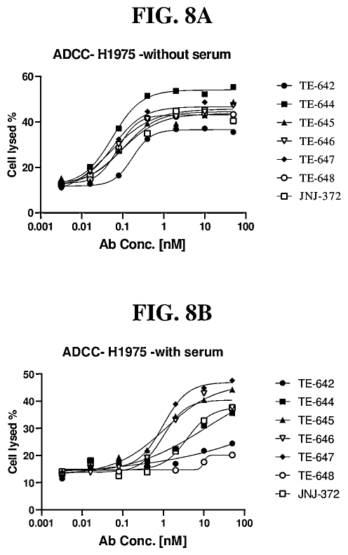

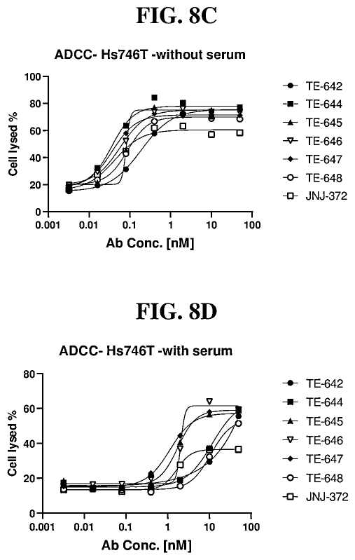

ADCC is one of the mechanisms contributing the anti-tumor activity of the constructed trispecific antibodies. The ADCC activity was evaluated against EGFR signaling-dependent cell line H1975 (cell line from non-small cell lung cancer, which is EGFR signaling dependent and ligand independent) and c-MET signaling-dependent cell line Hs746T (human gastric cancer cell, which is cMET signaling dependent and ligand independent) by co-culturing with human primary PBMC. H1975 and Hs746T cells were engineered to express nano-luciferase which was trapped in cytoplasm and will be released when cells are lysed. Briefly, human PBMC (1×10⁵) were co-cultured with H1975/Nluc (5×10³ per well) or Hs746T/Nluc (5×10³ per well) in the presence of test antibodies with series dilution (0.0032˜50 nM) for 16 hours in 96-well V-bottom plates. Then, cell supernatants were collected and the luciferase signals were measured by Nano-Glo Luciferase Assay System (Promega, #N1120). The activities were measured in the presence or absence of whole human serum in cell culture medium. IC₅₀ values were determined by fitting the dose-response data to the four-parameter logistic model with GraphPad Prism. As shown in Table 24 and FIG. 8, the TE-644, TE-645, TE-646 and TE-647 exhibited better ADCC activity than JNJ-372 on H1975 (FIGS. 8A and 8B); and all below trispecific antibodies showed better activity than JNJ-372 on Hs746T (FIGS. 8C and 8D), either with serum or without serum.

TABLE 24
ADCC activity of trispecific antibodies

| | H1975 | | | | Hs746T | | | |
|---|---|---|---|---|---|---|---|---|
| | without | | with | | without | | with | |
| | serum | | serum | | serum | | serum | |
| | IC₅₀ | Iₘₐₓ | IC₅₀ | Iₘₐₓ | IC₅₀ | Iₘₐₓ | IC₅₀ | Iₘₐₓ |
| Antibody | (nM) | (%) | (nM) | (%) | (nM) | (%) | (nM) | (%) |
| TE-642 | 0.17 | 37.14 | 6.09 | 24.43 | 0.19 | 76.04 | 12.54 | 55.49 |
| TE-644 | 0.06 | 55.31 | 2.84 | 35.62 | 0.04 | 77.23 | 11.62 | 58.69 |
| TE-645 | 0.1 | 48.79 | 1.1 | 44.23 | 0.08 | 75.55 | 1.74 | 59.95 |
| TE-646 | 0.05 | 47.31 | 1 | 42.91 | 0.06 | 73.7 | 2.05 | 63.98 |
| TE-647 | 0.06 | 48.69 | 0.97 | 47.59 | 0.04 | 71.21 | 1.13 | 57.99 |
| TE-648 | 0.07 | 43.57 | 9.88 | 20.14 | 0.09 | 68.45 | 10.78 | 51.4 |
| JNJ-372 | 0.09 | 45.12 | 3.84 | 37.51 | 0.04 | 63.41 | 1.65 | 36.49 |

### Example 17. ADCP Activity of EGFR×cMET×cMET Trispecific Antibodies

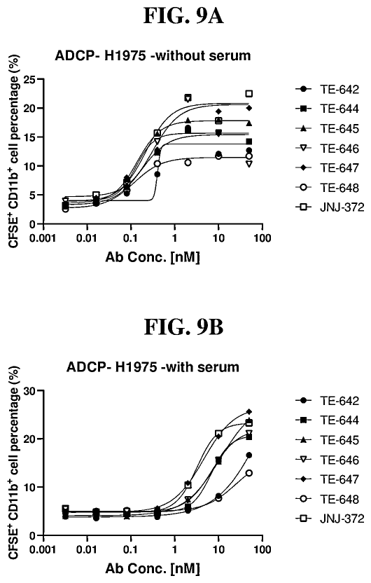

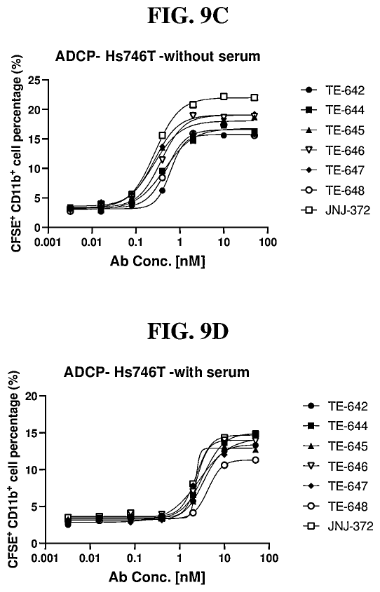

ADCP is another Fc-mediated function contributing to the anti-tumor activity of the constructed trispecific antibodies. The ADCP activity on H1975 and Hs756T was measured by using human macrophages. Briefly, human macrophage was derived and differentiated from human PBMC. The human macrophages (1×10⁵ per well) were co-cultured with CFSE pre-labeled H1975 (5×10⁴) or Hs746T (5×10⁴) in the presence of antibodies (0.0032˜50 nM) for 2 hours in 96-well U-bottom plates, followed by flow cytometry to measure the percentage of CFSE-labeled and CD11b positive macrophages, which reflect the ADCP activity. The activities were measured in the presence or absence of whole human serum in cell culture medium. EC₅₀ values were determined by fitting the dose-response data to the four-parameter logistic model with GraphPad Prism. As shown in Table 25 and FIG. 9, all trispecific antibodies exhibited ADCP activity against tumor cells. TE-646 and TE-647 exhibited comparable ADCP activity as JNJ-372 on both H1975 (FIGS. 9A and 9B) and Hs746T (FIGS. 9C and 9D).

TABLE 25
ADCP activity of trispecific antibodies

| | H1975 | | | | Hs746t | | | |
|---|---|---|---|---|---|---|---|---|
| | without | | with | | without | | with | |
| | serum | | serum | | serum | | serum | |
| | EC₅₀ | Eₘₐₓ | EC₅₀ | Eₘₐₓ | EC₅₀ | Eₘₐₓ | EC₅₀ | Eₘₐₓ |
| Antibody | (nM) | (%) | (nM) | (%) | (nM) | (%) | (nM) | (%) |
| TE-642 | 0.4 | 16.6 | 12.81 | 16.6 | 0.6 | 15.8 | 3.35 | 13.3 |
| TE-644 | 0.17 | 16.1 | 6.78 | 20.4 | 0.45 | 17.3 | 3.35 | 14.9 |
| TE-645 | 0.14 | 18 | 7.7 | 23.9 | 0.25 | 18.7 | 2.26 | 13.1 |
| TE-646 | 0.13 | 21.5 | 7.1 | 21.1 | 0.37 | 18.8 | 2.44 | 14 |
| TE-647 | 0.32 | 21.9 | 4.55 | 25.6 | 0.26 | 19.2 | 2.75 | 14.3 |
| TE-648 | 0.12 | 11.7 | 12.47 | 12.9 | 0.5 | 17.4 | 4.32 | 11.3 |
| JNJ-372 | 0.21 | 22.5 | 3.21 | 23.3 | 0.27 | 22 | 2.34 | 14.8 |

### Example 18. Anti-Proliferation Activity of EGFR×cMET×cMET Trispecific Antibodies on EGFR or cMET Signaling-Dependent Cancer Cell Lines

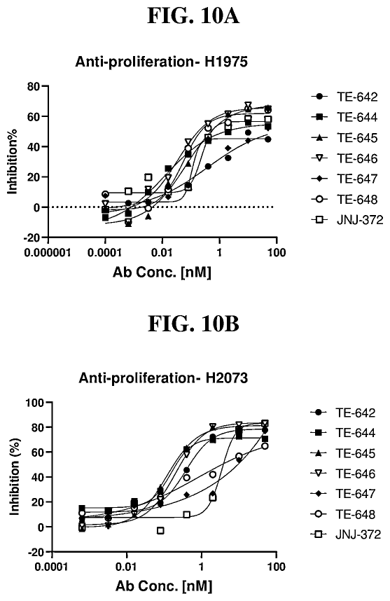

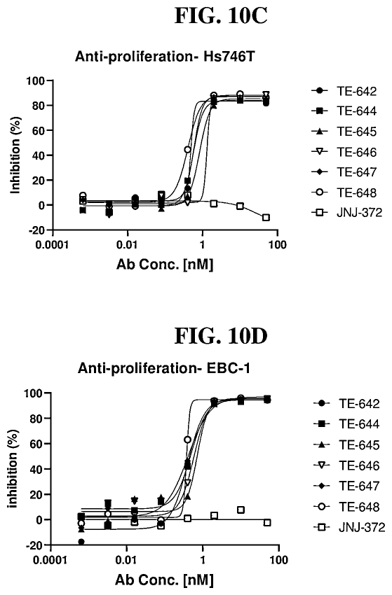

Mechanistically, EGFR×cMET×cMET trispecific antibodies inhibit either EGFR or c-MET signaling by down-regulating these kinase receptors on the cell surface, by which they inhibit proliferation on both c-MET and EGFR signaling-dependent cell lines. Cell-Titer-Glo assay (Promega) was used to evaluate the proliferation inhibition activities of these trispecific antibodies on both types of cell lines. Non-small cell lung cancer cell lines that depend on EGFR signaling (ligand independent): H1975 and H2073, and cell lines that depend on c-MET signaling (ligand independent): Hs746t and EBC-1 (human squamous lung cancer cell line) were selected as models for the evaluation. Briefly, cells were seeded to 96-well assay plates, treated with a series of dilutions of antibodies, and incubated at 37° C., 5% CO₂ for 6 days. After the treatment, 50 μl of Cell-Titer-Glo reagent was added in each well. The plates were shaken on an orbital shaker for 10 min to allow cell lysis. Luminescent signals were measured using BMG PheraStar with the luminescent protocol. IC50 values were determined by fitting the dose-response data to the four-parameter logistic model with GraphPad Prism. As shown in Table 26 and FIG. 10A-10B, all trispecific antibodies exhibited comparable or better anti-proliferation activity on EGFR signaling-dependent cell lines (H1975 and H2073) compared to JNJ-372. Strikingly, as shown in Table 26 and FIG. 10C-10D, all trispecific antibodies exhibited strong anti-proliferation activity on c-MET signaling-driven cell lines (Hs746t and EBC-1), while JNJ-372 did not showed such activity.

TABLE 26
Anti-proliferation activity of trispecific antibodies
on EGFR or c-MET signaling-dependent cancer cell lines

| | EGFR signaling- | | | | c-MET signaling- | | | |
|---|---|---|---|---|---|---|---|---|
| | dependent cells | | | | dependent cell | | | |
| | H1975 | | H2073 | | Hs746t | | EBC-1 | |
| | IC₅₀ | Iₘₐₓ | IC₅₀ | Iₘₐₓ | IC₅₀ | Iₘₐₓ | IC₅₀ | Iₘₐₓ |
| Antibody | (nM) | (%) | (nM) | (%) | (nM) | (%) | (nM) | (%) |
| TE-642 | 0.18 | 49.26 | 0.38 | 77.76 | 0.47 | 84.86 | 0.4 | 95.24 |
| TE-644 | 0.02 | 55.93 | 0.16 | 72.47 | 0.57 | 83.93 | 0.49 | 94.47 |
| TE-645 | 0.07 | 65.84 | 0.12 | 80.36 | 0.83 | 85.56 | 0.72 | 94.62 |
| TE-646 | 0.03 | 67.33 | 0.2 | 82.81 | 1.31 | 88.6 | 0.58 | 95.39 |
| TE-647 | 0.11 | 52.24 | 4.89 | 76.73 | 0.59 | 87.98 | 0.41 | 95.28 |
| TE-648 | 0.05 | 65.12 | 0.42 | 64.89 | 0.4 | 89.17 | 0.38 | 95.95 |
| JNJ-372 | 0.23 | 58.44 | 3.46 | 82.99 | N/A | 0 | N/A | 0 |

### Example 19. Anti-Proliferation Activity of EGFR×cMET×cMET Trispecific Antibodies on Human Epidermal Keratinocytes, Neonatal (HEKn)

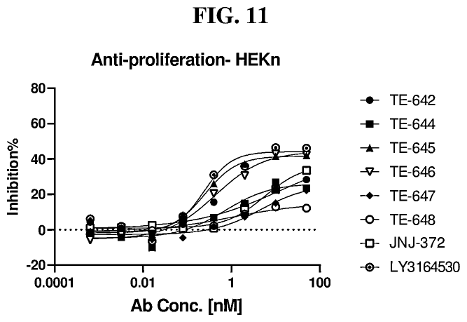

The skin-related adverse event is a typical on-target effect caused by hitting WT EGFR in skin cells, specifically, epidermal keratinocytes. To assess the skin toxicity risk of EGFR×cMET×cMET trispecific antibodies on normal keratinocytes, an anti-proliferation assay was performed on HEKn cells. The clinically approved drug JNJ-372 didn't exhibit serious skin-related risks in patients. The clinic stage LY3164530 led to a substantial percentage of skin-related adverse events in the phase I clinical trial, including maculopapular rash/dermatitis acneiform (83%, Grade 3/4 17%)¹¹. Both antibodies were internally generated and used as controls in the assay. Experimentally, HEKn cells were seeded to a 96-well assay plate, cells were treated with a series of dilutions of antibodies and incubated at 37° C., 5% CO₂ for 6 days. After treatment, 50 μl of Cell-Titer-Glo reagent (Promega) was added to each well. The plate was shaken on an orbital shaker for 10 min to allow cell lysis. The luminescent signal was measured using BMG PheraStar with the luminescent protocol. IC₅₀ values were determined by fitting the dose-response data to the four-parameter logistic model with GraphPad Prism. As shown in Table 27 and FIG. 11, TE-642, TE-644, TE-647 and TE-648 exhibited weaker anti-proliferation activity on HEKn cells than LY3164530. TE-647 exhibited a comparable potency (IC₅₀) and lower efficacy (Iₘₐₓ) than JNJ-372, which infers less toxicity on normal human keratinocytes than JNJ-372.

TABLE 27
Anti-proliferation activity of trispecific antibodies on HEKn

| Antibody | IC₅₀ (nM) | Iₘₐₓ(%) |
|---|---|---|
| TE-642 | 1.84 | 28.36 |
| TE-644 | 0.71 | 26.74 |
| TE-645 | 0.25 | 41.94 |
| TE-646 | 0.43 | 42.26 |
| TE-647 | 4.58 | 22.22 |
| TE-648 | 1 | 12.84 |
| JNJ-372 | 5.7 | 33.53 |
| LY3164530 | 0.24 | 46.16 |

### Example 20. EGFR×cMET×cMET Trispecific Antibodies TE-642, TE-646 and TE-647 are Active In Vivo Against Human Lung Tumor Xenografts

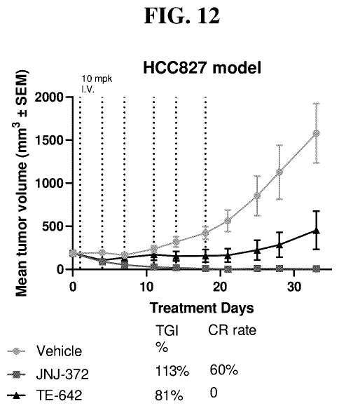

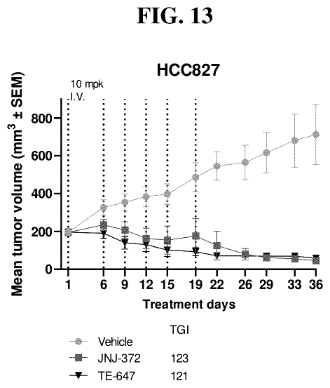

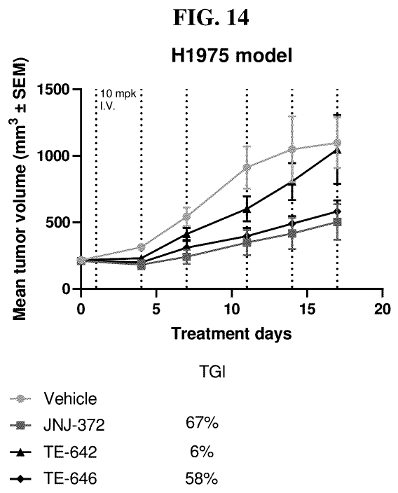

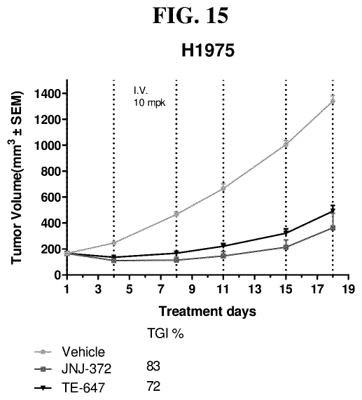

The in vivo efficacy of TE-642, TE-646, TE-647 and JNJ-372 were evaluated in mice transplanted with HCC827 (lung adenocarcinoma cell line with EGFR exon 19 deletion (E19del)) (FIG. 12 and FIG. 13) and H1975 (with EGFR L858R/T790M and without cMET amplification) (FIG. 14 and FIG. 15) human lung cancer cells.

Female BALB/c nude mice were subcutaneously implanted with 5×10⁶ HCC827 or H1975 cells (EGFR mutation driven model with EGFR L858R/T790M and without cMET amplification) per 200 μL PBS with 50% Matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 200-250 mm³ in size, mice with HCC827 or H1975 tumors were randomly allocated into 3 groups with 6 or 8 animals in each group, and were intravenously treated with vehicle, JNJ-372 or trispecific antibody (TE-647, TE-646, or TE-642) at 10 mg/kg biweekly (BIW) for 3 weeks. Partial regression (PR) was defined as tumor volume smaller than 50% of the starting tumor volume measured on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

$$treated t = treated tumor volume at time t$$

$$treated {t}_{0} = treated tumor volume at time 0$$

$$placebo t = placebo tumor volume at time t$$

$$placebo {t}_{0} = placebo tumor volume at time 0$$

In xenografts without c-MET amplification (EGFR mutation driven model, HCC827 model and H1975 model), TE-642 showed lower TGI (81% vs 113%) and lower CR rate (0 vs 60%) in HCC827 model (as shown in FIG. 12) and lower TGI (6% vs 67%) in H1975 model (as shown in FIG. 14), compared with JNJ-372.

TE-646 is comparable to JNJ-372 in tumor growth inhibition in xenografts without c-MET amplification (EGFR mutation driven model, H1975 model), as shown in FIG. 14.

TE-647 is comparable to JNJ-372 in tumor growth inhibition in xenografts without c-MET amplification (EGFR mutation driven model), i.e., in HCC827 model (as shown in FIG. 13) and in H1975 model (as shown in FIG. 15).

### Example 21. Trispecific Antibody TE-647 Inhibits Tumor Growth in Human Lung and Gastric Tumors in Animal Models

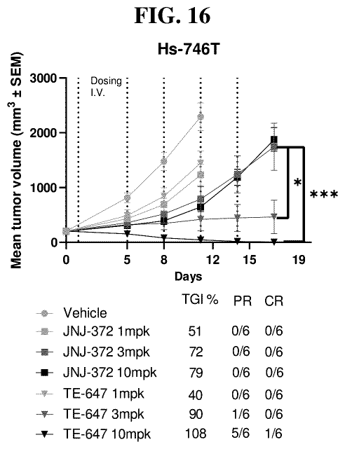

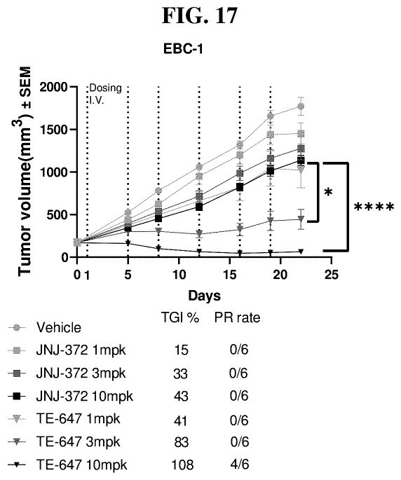

The in vivo efficacy of TE-647 and JNJ-372 were evaluated in mice transplanted with Hs746T (human gastric cancer cell, FIG. 16) and EBC-1 (human squamous lung cancer cell, FIG. 17).

Female BALB/c nude mice were subcutaneously implanted with 5×10⁶ Hs756T or EBC-1 (cMET amplification driven model without EGFR mutation) per 200 L PBS with 50% Matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 200-250 mm³ in size, mice with Hs746T or EBC-1 tumors were randomly allocated into 7 groups with 6 animals in each group, and were intravenously treated with vehicle, JNJ-372 or TE-647 at 1, 3, 10 mg/kg BIW for 3 weeks. Partial regression (PR) was defined as tumor volume smaller than 50% of the starting tumor volume measured on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

$$treated t = treated tumor volume at time t$$

$$treated {t}_{0} = treated tumor volume at time 0$$

$$placebo t = placebo tumor volume at time t$$

$$placebo {t}_{0} = placebo tumor volume at time 0$$

Trispecific antibody TE-647 has shown robust and reproducible antitumor effects in human gastric and lung tumor xenografts (cMET amplification driven model). In xenografts with c-MET amplification (Hs746T and EBC-1), TE-647 showed better tumor growth inhibition than JNJ-372 and the effect is dose-dependent (FIG. 16 and FIG. 17), indicating the superior anti-tumor activity of TE-647 in models with c-MET amplification.

### Example 22. Physiochemical Property of TE-647

### Hydrophobicity Assessment

To determine the hydrophobicity of TE-647 using HIC, 50 g of sample at 1 mg/ml was diluted with a mobile phase A solution (1.5 M ammonium sulfate, 50 mM sodium phosphate, pH 7.0) to achieve a final ammonium sulfate concentration of about 1M before analysis. A MABPac HIC-10 column was used with a liner gradient of mobile phase A and mobile phase B solution (50 mM sodium phosphate, pH 7.0) over 29 min at a flow rate of 0.5 mg/min. Peak retention times were monitored at A280 absorbance. The results were summarized in Table 28. TE-647 showed no hydrophobicity risk.

### Thermostability Assessment (Tm and Tagg)

Fluorescence coupled with static light scattering was used to evaluate the thermal unfolding transition midpoint Tm (° C.) and the onset temperature of aggregation (Tagg). A UNit® (Unchained labs, Pleasanton, CA) system was used to measure the fluorescence and static light scattering simultaneously. During the measurement, 9 L protein sample at 1 mg/mL was loaded to the cuvette; the samples were held at 20° C. for 120 s and then ramped to 95° C. at the rate of 0.3° C./min. Both fluorescence and static light scattering (at 266 nm) were collected after excitation at 266 nm. After measurement, the data were loaded onto Unit analysis software. Tm was gotten from the fluorescence and the Tagg was gotten from the static light scattering. The results were summarized in Table 28. Tm and Tagg of TE-647 were 67.3° C. and 60.0° C. respectively.

### Self-Association Assessment

The AC-SINS assay measures protein self-interaction by capturing the mAb on the surface of a gold colloid that displays surface resonance oscillations in frequency with visible light. As the immobilized antibodies self-interact, the colloids aggregate, changing the oscillation frequency to absorb at a longer wavelength. Gold nanoparticles were incubated with an 80/20 (v/v) capture antibody/non-capture antibody mixture. The coated gold nanoparticles were then spun down and resuspended into PBS. The samples were diluted to 0.05 mg/ml into the conjugation buffers and 45 μl of each dilution was loaded onto a 384-well plate. Five μl of previously prepared gold nanoparticles were then added to each well of the plate including mAbs and buffer controls. The plate was then covered with an aluminum lid, incubated at room temperature for 2 hours, and quickly spun down at 3000 rpm prior to reading the absorbance spectra of each well from 450 to 650 nm using a plate reader. Each sample spectra were recorded and analyzed for red-shifting of the maximum of absorption peak compared to buffers and mAb controls, the red-shifting and its strength is indicative of the self-interaction propensity of the tested mAb sample. The results were summarized in Table 28. The wavelength shift was 4.8 nm, which showed no self-association risk.

TABLE 28
Biophysical properties of TE-647

| Sample | Retention Time | Tm | Tagg | AC-SINS |
|---|---|---|---|---|
| Name | (min) | (° C.) | (° C.) | (Δλ) |
| TE-647 | 13.7 | 67.3 | 60.0 | 4.8 |

### REFERENCES

1. Structural basis of the activation of c-MET receptor. Nature communications (2021), 12: 4074
2. c-MET as a potential therapeutic target and biomarker in cancer. Therapeutic advances in medical oncology (2011), 3(1 Suppl): S21-S35
3. HGF/c-MET pathway in cancer: from molecular characterization to clinical evidence. Oncogene (2021), 40: 4625-4651
4. Screening for Epidermal Growth Factor Receptor Mutations in Lung Cancer. N Engl J Med (2009), 361:958-67
5. A Prospective, Molecular Epidemiology Study of EGFR Mutations in Asian Patients with Advanced Non-Small-Cell Lung Cancer of Adenocarcinoma Histology (PIONEER). J Thorac Oncol (2014), 9: 154-162
6. Acquired resistance mechanisms to osimertinib: The constant battle. Cancer Treatment Reviews (2023), 116: 102557
7. Mechanisms of acquired resistance to first- and second-generation EGFR tyrosine kinase inhibitors. Annals of Oncology (2018), 29 (Supplement 1): il0-il9
8. Resistance mechanisms to osimertinib in EGFR-mutated non-small cell lung cancer. British Journal of Cancer (2019), 121:725-737
9. Amivantamab and lazertinib in patients with EGFR-mutant non-small cell lung (NSCLC) after progression on osimertinib and platinum-based chemotherapy: Updated results from CHRYSALIS-2. Journal of Clinical Oncology 40, no. 16_suppl (Jun. 1, 2022) 9006-9006.
10. Controlled Fab-arm exchange for the generation of stable bispecific IgG1. Nature Protocol (2014), 9: 2450-2463
11. A phase I study of LY3164530, a bispecific antibody targeting MET and EGFR, in patients with advanced or metastatic cancer. Cancer Chemotherapy and Pharmacology (2018) 82:407-418

1. An antibody or antigen-binding fragment thereof which specifically binds human cMET, comprising:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(13) a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68;
(14) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(15) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(16) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(17) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(18) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68.

2.-6. (canceled)

7. A multispecific antibody or antigen-binding fragment thereof, comprising a first antigen binding domain that specifically binds a first epitope of human cMET, and a second antigen binding domain that specifically binds to a second epitope of human cMET, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(13) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(14) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(15) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(16) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68.

8.-64. (canceled)

65. A multispecific antibody or antigen-binding fragment thereof, comprising a first antigen binding domain that specifically binds to a first epitope of human cMET: a second antigen binding domain that specifically binds to a second epitope of human cMET; and a third antigen binding domain that specifically binds to human EGFR, wherein
the first antigen binding domain that specifically binds to the first epitope of human cMET comprises a heavy chain variable region (VH) that comprises (a) a HCDR1 of SEQ ID NO: 6, (b) a HCDR2 of SEQ ID NO: 7, (c) a HCDR3 of SEQ ID NO: 92 and a light chain variable region (VL) that comprises: (d) a LCDR1 of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises a heavy chain variable region (VH) that comprises (a) a HCDR1 of SEQ ID NO: 56, (b) a HCDR2 of SEQ ID NO: 57, (c) a HCDR3 of SEQ ID NO: 58 and a light chain variable region (VL) that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises a heavy chain variable region (VH) that comprises (a) a HCDR1 of SEQ ID NO: 155, (b) a HCDR2 of SEQ ID NO: 156, (c) a HCDR3 of SEQ ID NO: 157 and a light chain variable region (VL) that comprises: (d) a LCDR1 of SEQ ID NO: 158, (e) a LCDR2 of SEQ ID NO: 159, and (f) a LCDR3 of SEQ ID NO: 160.

66. The multispecific antibody or antigen-binding fragment thereof of claim 65, wherein
the first antigen binding domain that specifically binds to the first epitope of human cMET comprises a heavy chain variable region (VH) comprising the amino acid sequence at least 95% identical to SEQ ID NO: 144, and a light chain variable region (VL) comprising the amino acid sequence at least 95% identical to SEQ ID NO: 64;
the second antigen binding domain that specifically binds to the second epitope of human cMET comprises: a heavy chain variable region (VH) comprising the amino acid sequence at least 95% identical to SEQ ID NO: 145, and a light chain variable region (VL) comprising the amino acid sequence at least 95% identical to SEQ ID NO: 64; and
the third antigen binding domain that specifically binds to human EGFR comprises: a heavy chain variable region (VH) comprising the amino acid sequence at least 95% identical to of SEQ ID NO: 142, and a light chain variable region (VL) comprising the amino acid sequence at least 95% identical to SEQ ID NO: 143.

67. The multispecific antibody or antigen-binding fragment thereof of claim 65, wherein the first antigen binding domain that specifically binds to the first epitope of human cMET comprises a heavy chain variable region (VH) comprising the amino acid sequence of SEQ ID NO: 144, and a light chain variable region (VL) comprising the amino acid sequence of SEQ ID NO: 64:
the second antigen binding domain that specifically binds to the second epitope of human cMET comprises: a heavy chain variable region (VH) comprising the amino acid sequence of SEQ ID NO: 145, and a light chain variable region (VL) comprising the amino acid sequence of SEQ ID NO: 64; and
the third antigen binding domain that specifically binds to human EGFR comprises: a heavy chain variable region (VH) comprising the amino acid sequence of SEQ ID NO: 142, and a light chain variable region (VL) comprising the amino acid sequence of SEQ ID NO: 143.

68-69. (canceled)

70. The multispecific antibody or antigen-binding fragment thereof of claim 65, wherein the third antigen binding domain that specifically binds to human EGFR is a scFv having the amino acid sequence of SEQ ID NO: 161.

71. The multispecific antibody or antigen-binding fragment thereof of claim 65, wherein the multispecific antibody is a trispecific antibody.

72. (canceled)

73. The multispecific antibody or antigen-binding fragment thereof of claim 65, wherein the multispecific antibody or antigen-binding fragment thereof comprises a heavy chain constant region of the subclass of IgG1, and a light chain constant region of the type of kappa.

74-75. (canceled)

76. The multispecific antibody or antigen-binding fragment thereof of claim 73, wherein the multispecific antibody or antigen-binding fragment thereof is afucosylated.

77-78. (canceled)

79. The multispecific antibody or antigen-binding fragment thereof of claim 65,
wherein the first antigen binding domain that specifically binds to the first epitope of human cMET comprises a first heavy chain constant region comprising SEQ ID NO: 96, and the second antigen binding domain that specifically binds to the second epitope of human cMET comprises a second heavy chain constant region comprising SEQ ID NO: 95.

80-84. (canceled)

85. The multispecific antibody or antigen-binding fragment thereof of claim 65, comprising a first polypeptide, a second polypeptide, and a third polypeptide, wherein
the first polypeptide comprises the amino acid sequence of SEQ ID NO: 153, the second polypeptide comprises the amino acid sequence of SEQ ID NO: 150, and the third polypeptide comprises the amino acid sequence of SEQ ID NO: 148.

86. The multispecific antibody or antigen-binding fragment thereof of claim 65, wherein the multispecific antibody or antigen-binding fragment thereof comprises
a first polypeptide consisting of SEQ ID NO: 153, a second polypeptide consisting of SEQ ID NO: 150, and a third polypeptide consisting of SEQ ID NO: 148.

87-108. (canceled)

109. A multispecific antibody or antigen-binding fragment thereof, comprising a first polypeptide, a second polypeptide, and a third polypeptide, wherein
the first polypeptide has the amino acid sequence of SEQ ID NO: 153, the second polypeptide has the amino acid sequence of SEQ ID NO: 150, and the third polypeptide has the amino acid sequence of SEQ ID NO: 148.

110. A pharmaceutical composition comprising the multispecific antibody or antigen-binding fragment thereof of claim 109 and a pharmaceutically acceptable carrier.

111. A method of treating a cancer comprising administering to a patient in need a therapeutically effective amount of the antibody, antibody or antigen-binding fragment thereof of claim 109.

112. An isolated nucleic acid that encodes the multispecific antibody or antigen-binding fragment thereof of claim 109.

113. A vector comprising the isolated nucleic acid of claim 112.

114. A host cell comprising the nucleic acid of claim 112.

115. A process for producing a multispecific antibody or antigen-binding fragment thereof comprising cultivating the host cell of claim 114 and recovering the multispecific antibody or antibody fragment from the culture.

116. An isolated nucleic acid that encodes the antibody or antigen-binding fragment thereof of claim 1.
